(19) United States
(12) Patent Application Publication
(10) Pub. No.: US 2024/0156446 A1
(43) Pub. Date: May 16, 2024

(54) TRANSCATHETER DEVICES AND METHODS FOR REPAIRING A LEAFLET OF A HEART VALVE OF A SUBJECT

(71) Applicant: EDWARDS LIFESCIENCES CORPORATION, Irvine, CA (US)

(72) Inventors: Mauricio Guerrero, Hawthorne, CA (US); Brian Patrick Murphy, Irvine, CA (US); Travis Zenyo Oba, Yorba Linda, CA (US); Sam Shafigh, Irvine, CA (US); Mark Chau, Laguna Hills, CA (US); Kevin Gantz, Laguna Beach, CA (US); Meena Francis, Trabuco Canyon, CA (US); Murrad Mirza Kazalbash, Tustin, CA (US)

(21) Appl. No.: 18/512,009

(22) Filed: Nov. 16, 2023

Related U.S. Application Data
(63) Continuation of application No. PCT/US2022/029219, filed on May 13, 2022.
(60) Provisional application No. 63/311,919, filed on Feb. 18, 2022.
(60) Provisional application No. 63/285,948, filed on Dec. 3, 2021.
(60) Provisional application No. 63/192,829, filed on May 25, 2021.

(51) Int. Cl. A61B 17/00; A61B 17/04; A61B 17/06; A61B 17/32

(52) U.S. Cl. CPC A61B 17/00234; A61B 17/0469; A61B 17/06066; A61B 17/32; A61B 2017/00243; A61B 2017/003; A61B 2017/00353; A61B 2017/0038; A61B 2017/06052; A61B 2017/06076; A61B 2017/061; A61B 2017/32006

(57) ABSTRACT

A system, for use with a leaflet of a valve of a subject's heart, comprises a catheter, a cutting device, a helical needle, and a suture. The catheter is transluminally advanceable toward the heart. The cutting device is advanceable distally out of the catheter, and is adapted to cut through tissue of the leaflet to form cut tissue and cut edges at the cardiac valve. The helical needle defines a helical lumen, the helical needle configured to be advanced distally out of the catheter and to rotationally extend through, and secure together, two tissue segments of the leaflet. The suture extends through the helical lumen of the helical needle. The helical needle and the suture are configured to extend along the two tissue segments of the leaflet to suture the two tissue segments to each other. Other embodiments are also described.

### CROSS-REFERENCES TO RELATED APPLICATIONS

The present application is a Continuation of International Patent Application PCT/US2022/029219 to Guerrero et al., filed May 13, 2022, and titled “Transcatheter devices for repairing a leaflet of a heart valve of a subject;” which claims priority to U.S. Provisional Patent Application 63/192,829 to Guerrero et al., filed May 25, 2021, and titled “Transcatheter device and methods for repairing a leaflet of a heart valve of a subject;” U.S. Provisional Patent Application 63/285,948 to Guerrero et al., filed Dec. 3, 2021, and titled “Transcatheter device and methods for repairing a leaflet of a heart valve of a subject;” and U.S. Provisional Patent Application 63/311,919 to Guerrero et al., filed Feb. 18, 2022, and titled “Transcatheter devices and methods for repairing a leaflet of a heart valve of a subject.”

Each of the above references is incorporated herein by reference in its entirety.

### BACKGROUND

The function of mammalian hearts is based on heart muscles contracting and creating pressure, causing the blood to flow from the heart ventricles to the desired arteries. The blood from the body returns to the heart atria via veins. Valves, between each heart atrium and the corresponding heart ventricle, as well as between the heart ventricles and the corresponding arteries, ensure that no backflow occurs when the heart muscles contract and create pressure in the ventricles. The valves between the atria and the ventricles of the heart are known as the mitral valve and the tricuspid valve, and are each formed of a plurality of leaflets which coapt with each other when the valve is closed, and have a space formed therebetween when the valve is open.

Proper closing of the mitral and tricuspid valves is critical for proper function of the heart, and many medical conditions, some life threatening, result from improper closing of the valves which creates backflow (regurgitation) from the ventricles into the corresponding atria. One cause of such improper closing of the valves is excess tissue in one or more of the leaflets of the valve(s), which may result in leaflet prolapse (e.g., Carpentier type II regurgitation). Excess tissue in the leaflet, and improper closing thereof, are schematically shown in FIG. 1. Specifically, FIG. 1 shows a mitral valve 10 having a first leaflet 12 and a second leaflet 14, surrounded by an annulus 15. Second leaflet 14 has excess tissue 16, such that leaflets 12 and 14 coapt at point (or edge) 17 with gaps 18 along the coaptation line.

Existing techniques for treating such a condition include surgical removal of excess tissue of the leaflet, in an invasive open-heart procedure. Such a procedure requires a surgeon to cut, for example using scissors or a suitable scalpel, the excess portion of the tissue 16, and then to hold together the cut edges of the tissue using a suture. In addition to being invasive, this process requires a high degree of skill of the surgeon.

There is thus a need for improved techniques and devices for removal of excess tissue from a heart leaflet, in order to repair functionality of the leaflet.

### SUMMARY

In accordance with some applications herein, flail or prolapse of a valve leaflet can be treated by constraining, moving, or removing excess tissue from the valve leaflet.

In some applications, excess tissue of the valve leaflet is removed in a transluminal or transcatheter procedure. The excess tissue is cut away using a cutting device, such as a suitably configured cutting wire (e.g., a wire loop, etc.), thereby forming cut edges.

In some applications, as the cut edges are formed, they are secured together by an attaching device (e.g., an edge-attaching device). The attaching device can be, for example, a helical needle. In some applications, the helical needle follows the cutting device. As such, the cut edges are caught before they become distant from one another. The cut edges can then be more permanently secured together, for example by suturing which allows the cut edges to heal. In some applications, a suture may be present in a helical lumen of the helical needle and may be left in place during and after retraction of the helical needle.

In some applications, during cutting of the tissue, the tissue can be held by another transluminally introduced tool, such as a clamp, snare, hook, etc. The excess tissue and the clamp can be removed, for example through a catheter, following detachment of the excess tissue from the leaflet.

In some applications, the prolapse or flail caused by excess tissue of the valve leaflet is nullified by an implantable element, in a transluminal or transcatheter procedure.

In some applications, the excess tissue can be nullified by constraining the excess tissue, or a portion of the leaflet, to another portion of the leaflet or another cardiac tissue, using an implantable element. In some applications, such constraining results in presentation of a portion of the leaflet, different from the lip of the leaflet, as a replacement coaptation surface.

In some applications, the excess tissue can be nullified by causing the leaflet, including the excess tissue, to follow a tortuous path about an implant, thereby to “use up” the excess tissue.

In accordance with some applications, there is provided a system for use with a subject, the system including a longitudinal catheter configured to be transluminally advanced toward an anatomical site of the subject. The longitudinal catheter has a proximal part and a steerable distal part, and a longitudinal axis therebetween.

The system can include a wire, advanceable distally out of the catheter, the wire forming a wire loop having a closed distal end.

A tightening element can be slidably coupled to the wire. Distal sliding of the tightening element over the wire can cause contraction of the wire loop and cutting of tissue disposed within the wire loop, thereby forming cut tissue and cut edges at the anatomical site.

The system can include a helical needle defining a helical lumen. The helical needle can be configured to be advanced distally out of the longitudinal catheter and to rotationally extend through, and secure together, the cut edges at the anatomical site. A suture can extend through the helical lumen of the helical needle.

The helical needle and the suture can be configured to extend along the cut edges of the anatomical site to suture the cut edges during distal sliding of the tightening element.

In some applications, the system can include a tissue anchor, configured to be anchored to tissue at the anatomical site. At least one of the wire loop and the helical needle can be attachable to the tissue anchor.

In some applications, the helical needle can include a needle body terminating at a detachable distal tip. The detachable distal tip can be configured to attach to the tissue anchor and to detach from the needle body.

In some applications, a distal end of the suture is attached to the detachable distal tip of the helical needle.

In some applications, following detachment of the detachable distal tip of the helical needle from the needle body, the needle body is retractable helically away from the detachable distal tip while sliding over and along the suture.

In some applications, the system can include a pushing-wire extending through the helical needle and configured to push the detachable distal tip of the helical needle distally relative to the needle body, thereby to detach the detachable distal tip from the needle body.

In some applications, the wire and the helical needle are adapted to be removed from the anatomical site following cutting of the tissue disposed within the wire loop, while leaving the suture in the anatomical site for suturing of the cut edges.

In some applications, the system includes a clamp having a distal clamping end. The clamp can be advanceable distally out of the longitudinal catheter to engage the cut tissue, prior to complete detachment of the cut tissue from the anatomical site.

In some applications, the clamp is adapted to be removed from the anatomical site, with the cut tissue clamped thereto following removal of the wire and the helical needle from the anatomical site.

In some applications, the clamp is configured to be advanced through the longitudinal catheter alongside the helical needle.

In some applications, the helical needle follows the tightening element at a fixed distance therefrom, at least during distal sliding of the tightening element.

In some applications, the anatomical site includes a cardiac valve, and the cut tissue includes a portion of a leaflet of the cardiac valve.

In some applications, the helical needle and the suture can be configured to rotationally extend through the cut edges at the anatomical site as the cut edges are formed.

In some applications, in at least a first state of the system, the tightening element can be operably coupled to the helical needle. In some applications, the tightening element can be advanced distally over the wire only when followed by advancement of the helical needle.

In some applications, the system includes a user interface enabling a user to control the system. The user interface can include a unified engagement element that, when actuated, controls motion of the tightening element and of the helical needle in unison.

In some applications, the unified engagement element, when actuated, controls motion of the tightening element and of the helical needle at a fixed distance from one another.

In some applications, the user interface includes a first engagement element that, when actuated, control motion of the tightening element, and can include a second engagement element that, when actuated, controls motion of the helical needle.

In some applications, the unified engagement element can include a third engagement element, distinct from the first and second engagement elements.

In some applications, the user interface includes a coupling element, functionally associated with the first and second engagement elements.

In some applications, in a first operative state of the coupling element, the first and second engagement elements can be coupled to form the unified engagement element. In some applications, actuation of one of the first and second engagement elements controls motion of the tightening element and of the helical needle in unison or at the fixed distance from one another.

In some applications, in a second operative state of the coupling element, the first and second engagement elements can be decoupled. In some applications, actuation of the first engagement element controls motion only of the tightening element, and actuation of the second engagement element controls motion only of the helical needle.

In some applications, at least one of the wire and the helical needle is adapted to be advanced through the longitudinal catheter to the anatomical site, or removed from the anatomical site via the longitudinal catheter.

In some applications, the system includes a longitudinal mount, adapted to be advanced distally out of the longitudinal catheter, such that the helical needle is adapted to rotate about, and be stabilized or guided by the mount during rotation of the helical needle.

In some applications, the system can include a tissue-engaging tool including first, second, and third beams, advanceable distally out of the longitudinal catheter, the tissue-engaging tool adapted to position the first and third beams on a first side of the tissue and the second beam on an opposing side of the tissue. In some applications, the tissue-engaging tool is adapted to form a bulge in the tissue by movement of the second beam with respect to the first beam and the third beam, and the wire loop is adapted to circumscribe the bulge on the first side of the tissue.

In accordance with some applications, there is provided a system for use with a subject, the system including a cutting device. The cutting device can be advanceable and steerable toward an anatomical site of the subject. Motion of the cutting device at the anatomical site causes cutting of tissue engaged by the cutting device, thereby forming cut edges at the anatomical site.

The system can include an edge-attaching device configured to at least temporarily secure the cut edges at the anatomical site.

In some applications, in at least a first state of the system, the edge-attaching device can be operably coupled to the cutting device. In some applications, the cutting device can be advanced distally at the anatomical site only when followed by advancement of the edge-attaching device, to secure the cut edges to one another as the cut edges are formed.

In some applications, in the first state of the system, the edge-attaching device can be operably coupled to the cutting device such that the cutting device can be advanced distally only when followed by advancement of the edge-attaching device at a fixed distance from the cutting device.

In some applications, the system can include a user interface enabling a user to control the system. The user interface can include a unified engagement element that, when actuated, controls motion of the cutting device and of the edge-attaching device in unison.

In some applications, the unified engagement element, when actuated, controls motion of the cutting device and of the edge-attaching device at a fixed distance from one another.

In some applications, the user interface includes a first engagement element that, when actuated, controls motion of the cutting device. In some applications, the user interface can include a second engagement element that, when actuated, controls motion of the edge-attaching device.

In some applications, the unified engagement element includes a third engagement element, distinct from the first and second engagement elements.

In some applications, the user interface can include a coupling element, functionally associated with the first and second engagement elements.

In some applications, in a first operative state of the coupling element, the first and second engagement elements can be coupled to form the unified engagement element. In some applications, actuation of one of the first and second engagement elements can control motion of the cutting device and of the edge-attaching device in unison.

In some applications, in a second operative state of the coupling element, the first and second engagement elements can be decoupled. In some applications, actuation of the first engagement element controls motion only of the cutting device, and actuation of the second engagement element controls motion only of the edge-attaching device.

In accordance with some applications, there is provided a system for use with a subject, the system including a cutting device, advanceable and steerable toward an anatomical site of the subject. Motion of the cutting device at the anatomical site can cause cutting of tissue engaged by the cutting device, thereby forming cut edges at the anatomical site.

The system can include an edge-attaching device configured to at least temporarily secure the cut edges at the anatomical site as the cut edges are formed.

The system can include a user interface enabling a user to control the system. The user interface can include a unified engagement element that, when actuated, controls motion of the cutting device and of the edge-attaching device in unison.

In some applications, the unified engagement element, when actuated, controls motion of the cutting device and of the edge-attaching device at a fixed distance from one another.

In some applications, the user interface includes a first engagement element that, when actuated, controls motion of the cutting device. In some applications, the user interface can include a second engagement element that, when actuated, controls motion of the edge-attaching device.

In some applications, the unified engagement element includes a third engagement element, distinct from the first and second engagement elements.

In some applications, the user interface includes a coupling element, functionally associated with the first and second engagement elements.

In some applications, in a first operative state of the coupling element, the first and second engagement elements can be coupled to form the unified engagement element. In some applications, actuation of one of the first and second engagement elements controls motion of the cutting device and of the edge-attaching device in unison.

In some applications, in a second operative state of the coupling element, the first and second engagement elements can be decoupled. In some applications, actuation of the first engagement element controls motion only of the cutting device, and actuation of the second engagement element controls motion only of the edge-attaching device.

In some applications, the system can include a longitudinal catheter configured to be transluminally advanced toward the anatomical site of the subject. The longitudinal catheter can have a proximal part and a steerable distal part. The cutting device and the edge-attaching device can be adapted to be advanceable and steerable to the anatomical site distally out of the longitudinal catheter.

In some applications, the cutting device and/or the edge-attaching device are adapted to be advanced to the anatomical site through the longitudinal catheter.

In some applications, the cutting device and/or the edge-attaching device are adapted to be removed from the anatomical site following cutting of tissue and securing together of the cut edges.

In some applications, the system can include a permanent edge-attaching device adapted to permanently attach the cut edges at the anatomical site.

In some applications, the edge-attaching device is adapted to temporarily secure the cut edges at the anatomical site, and the permanent edge-attaching device is adapted to permanently attach the cut edges.

In some applications, the edge-attaching device includes a needle, and the permanent edge-attaching device includes a suture, extending through a lumen of the needle.

In some applications, the needle is a helical needle and the lumen is a helical lumen.

In some applications, the cutting device includes a wire forming a wire loop having a closed distal end. A tightening element can be slidably coupled to the wire, such that distal sliding of the tightening element over the wire causes contraction of the wire loop and cutting of tissue disposed within the wire loop.

In some applications, the system can include a clamp having a distal clamping end. The clamp can be advanceable to the anatomical site to engage cut tissue cut by the cutting device, prior to complete detachment of the cut tissue from the anatomical site.

In some applications, the clamp is adapted to be removed from the anatomical site, with the cut tissue clamped thereto, following removal of at least one of the cutting device and the edge-attaching device from the anatomical site.

In some applications, the system can include a tissue anchor, configured to be anchored to tissue at the anatomical site for anchoring of at least one of the cutting device and the edge-attaching device.

In some applications, the anatomical site includes a cardiac valve, and the tissue engaged by the cutting device and cut thereby includes a portion of a leaflet of the cardiac valve.

In accordance with some applications, there is provided a method of removing tissue from an anatomical site of a subject. The method includes transluminally advancing a longitudinal catheter toward the anatomical site, the longitudinal catheter having a proximal part and a steerable distal part.

The method can include advancing a wire loop distally out of the longitudinal catheter. The wire loop can have a closed distal end, a proximal end. A tightening element can be slidably coupled to the wire loop.

The method can include advancing a helical needle distally out of the longitudinal catheter. The helical needle can include a helical lumen. A suture can extend through the helical lumen.

The method can include placing the wire loop about tissue at the anatomical site.

The method can include distally sliding the tightening element relative to the wire loop, thereby cutting the tissue disposed within the wire loop and forming cut tissue and cut edges at the anatomical site.

The method can include, during the distally sliding of the tightening element, advancing the helical needle and the suture along the cut edges to secure the cut edges to one another.

In some applications, the method can include advancing a tissue anchor through the longitudinal catheter to the anatomical site. The tissue anchor can be anchored to tissue at the anatomical site.

In some applications, the method includes, following the advancing of the helical needle anchoring a detachable distal tip of the helical needle to the tissue anchor. The detachable distal tip can have a distal end of the suture attached thereto.

The method can include detaching the detachable distal tip of the helical needle from a main body of the helical needle.

The method can include helically retracting the main body of the helical needle away from the detachable distal tip while sliding over the suture, thereby removing the helical needle from the anatomical site while leaving the suture in the anatomical site securing the cut edges.

In some applications, the detaching includes using a pushing-wire extending through the helical needle, pushing the detachable distal tip of the helical needle distally relative to the needle body, thereby to detach the detachable distal tip from the needle body.

In some applications, the method can include, following the cutting of the tissue, removing the wire loop from the anatomical site.

In some applications, the method can include, prior to completion of the cutting of the tissue disposed within the wire loop, advancing a clamp having a distal clamping end to the anatomical site and clamping the cut tissue in the clamping end of the clamp. The clamp and the cut clamped tissue can be removed from the anatomical site.

In some applications, the advancing of the clamp is through the longitudinal catheter. In some applications, during the advancing of the clamp, the clamp is alongside the helical needle.

In some applications, during the sliding of the tightening element and the advancing of the helical needle, the helical needle remains at a fixed distance from the tightening element.

In some applications, anatomical site includes a cardiac valve, and the tissue disposed within the wire loop includes a portion of a leaflet of the cardiac valve.

In some applications, the advancing of the helical needle and the suture is carried out as the cut edges are formed.

In some applications, the distally sliding of the tightening element relative to the wire loop can be operably coupled to the advancing of the helical needle. In some applications, the tightening element can be advanced distally relative to the wire loop only when followed by advancement of the helical needle.

In some applications, the method can include controlling of the sliding of the tightening element relative to the wire loop and of the advancing of the helical needle by a user actuating a unified engagement element of a user interface, to control motion of the tightening element and of the helical needle in unison.

In some applications, the controlling of the sliding of the tightening element relative to the wire loop and of the advancing of the helical needle by the user actuating the unified engagement element includes controlling motion of the tightening element and of the helical needle at a fixed distance from one another.

In some applications, the user interface includes a first engagement element that, when actuated, controls motion of the tightening element. In some applications, the user interface can include a second engagement element that, when actuated, controls motion of the helical needle.

In some applications, the user interface can include a coupling element, functionally associated with the first and second engagement elements.

In some applications, in a first operative state of the coupling element, the first and second engagement elements can be coupled to form the unified engagement element. In some applications, actuation of one of the first and second engagement elements can control motion of the tightening element and of the helical needle in unison.

In some applications, in a second operative state of the coupling element, the first and second engagement elements can be decoupled. In some applications, actuation of the first engagement element controls motion only of the tightening element, and actuation of the second engagement element controls motion only of the helical needle.

In some applications, the method includes, prior to the distally sliding of the tightening element relative to the wire loop and prior to the advancing of the helical needle, ensuring that the coupling element of the user interface is in the first operative state.

In some applications, at least one of the advancing of the wire loop and the advancing of the helical needle is through the longitudinal catheter.

The above method(s) can be performed on a living animal or on a simulation, such as on a cadaver, cadaver heart, simulator (e.g., with the body parts, heart, tissue, etc. being simulated), etc.

In accordance with some applications, there is provided a method of removing tissue from a valve leaflet of a heart valve of a subject. The method includes excising tissue from the valve leaflet, with a wire loop, to form cut edges. The method can include securing together the cut edges as the cut edges are formed, and prior to complete detachment of the tissue from the valve leaflet.

In some applications, the securing together of the cut edges includes advancing a helical needle through the cut edges.

In some applications, the method can include, prior to the excising, advancing the wire loop to the heart valve via a transluminal longitudinal catheter.

In some applications, the advancing of the helical needle includes advancing the helical needle at a fixed distance with relationship to a tightening element of the wire loop.

In some applications, the helical needle includes a helical lumen and has a suture extending through the helical lumen. In some applications, the method can include removing the helical needle from the heart valve while leaving the suture attaching the cut edges.

The above method(s) can be performed on a living animal or on a simulation, such as on a cadaver, cadaver heart, simulator (e.g., with the body parts, heart, tissue, etc. being simulated), etc.

In accordance with some applications, there is provided a method of removing tissue from an anatomical site of a subject. The method includes using a cutting device disposed at the anatomical site, cutting tissue engaged by the cutting device at the anatomical site, thereby forming cut tissue and cut edges.

The method can include, at least temporarily, securing together the cut edges with an edge-attaching device as the cut edges are formed and prior to complete detachment of the cut tissue from the anatomical site.

The cutting can be operably coupled to the securing together of the cut edges. In some applications, the cutting device can cut tissue at the anatomical site only when followed by advancement of the edge-attaching device, to secure the cut edges to one another during forming of the cut edges by the cutting device.

In some applications, the cutting can be operably coupled to the securing together of the cut edges. In some applications, the cutting device can cut tissue at the anatomical site only when followed by advancement of the edge-attaching device at a fixed distance from the cutting device.

In some applications, the method can include controlling of the cutting and of the securing together, by a user actuating a unified engagement element of a user interface, to control motion of the cutting device and of the edge-attaching device, in unison.

In some applications, the controlling of the cutting and of the securing together by the user actuating the unified engagement element includes controlling motion of the cutting device and of the edge-attaching device at a fixed distance from one another.

In some applications, the user interface includes a first engagement element that, when actuated, controls motion of the cutting device. The user interface can include a second engagement element that, when actuated, controls motion of the edge-attaching device.

In some applications, the user interface can include a coupling element, functionally associated with the first and second engagement elements.

In some applications, in a first operative state of the coupling element, the first and second engagement elements can be coupled to form the unified engagement element. In some applications, actuation of one of the first and second engagement elements controls motion of the cutting device and of the edge-attaching device in unison.

In some applications, in a second operative state of the coupling element, the first and second engagement elements can be decoupled. In some applications, actuation of the first engagement element controls motion only of the cutting device, and actuation of the second engagement element controls motion only of the edge-attaching device.

In some applications, the method can include, prior to the cutting and prior to the securing together, ensuring that the coupling element of the user interface is in the first operative state.

The above method(s) can be performed on a living animal or on a simulation, such as on a cadaver, cadaver heart, simulator (e.g., with the body parts, heart, tissue, etc. being simulated), etc.

In accordance with some applications, there is provided a method of removing tissue from an anatomical site of a subject. The method includes using a cutting device disposed at the anatomical site, cutting tissue engaged by the cutting device at the anatomical site, thereby forming cut tissue and cut edges.

The method can include, at least temporarily, securing together the cut edges with an edge-attaching device as the cut edges are formed and prior to complete detachment of the cut tissue from the anatomical site.

The method can include controlling the cutting and the securing together, by a user actuating a unified engagement element of a user interface, to control motion of the cutting device and of the edge-attaching device, in unison.

In some applications, the controlling of the cutting and of the securing together includes actuating the unified engagement element to control motion of the cutting device and of the edge-attaching device at a fixed distance from one another.

In some applications, the user interface includes a first engagement element that, when actuated, controls motion of the cutting device. The user interface can include a second engagement element that, when actuated, controls motion of the edge-attaching device.

In some applications, the user interface can include a coupling element, functionally associated with the first and second engagement elements.

In some applications, a first operative state of the coupling element, the first and second engagement elements can be coupled to form the unified engagement element. In some applications, actuation of one of the first and second engagement elements controls motion of the cutting device and of the edge-attaching device in unison.

In some applications, in a second operative state of the coupling element, the first and second engagement elements can be decoupled. In some applications, actuation of the first engagement element controls motion only of the cutting device, and actuation of the second engagement element controls motion only of the edge-attaching device.

In some applications, the method can include, prior to the cutting and prior to the securing together, ensuring that the coupling element of the user interface is in the first operative state.

In some applications, the method can include transluminally advancing a longitudinal catheter toward the anatomical site. The longitudinal catheter can have a proximal part and a steerable distal part.

In some applications, the method can include advancing and steering the cutting device and the edge-attaching device to the anatomical site distally out of the longitudinal catheter.

In some applications, the advancing and steering of the cutting device and of the edge-attaching device are through the longitudinal catheter.

In some applications, the method can include removing the cutting device and/or the edge-attaching device from the anatomical site following the cutting and the securing together.

In some applications, the securing together includes temporarily securing together of the cut edges at the anatomical site by the edge-attaching device. In some applications, the method can include permanently attaching the cut edges at the anatomical site.

In some applications, the temporarily securing together includes advancing a needle through the cut edges to hold them together. In some applications, the permanently attaching includes extending a suture through the needle.

In some applications, the needle is a helical needle.

In some applications, the cutting device includes a wire loop having a closed distal end. In some applications, a tightening element can be slidably coupled to the wire loop. The cutting of the tissue can include sliding of the tightening element distally relative to the wire loop contracting the wire loop and cutting tissue disposed within the wire loop.

In some applications, the method can include using a clamp having a distal clamping end advanced to the anatomical site, engaging the cut tissue, prior to complete detachment of the cut tissue from the anatomical site.

In some applications, the method can include removing the clamp from the anatomical site, with the cut tissue clamped thereto, following removal of at least one of the cutting device and the edge-attaching device from the anatomical site.

In some applications, the method can include anchoring a tissue anchor to tissue at the anatomical site, and anchoring at least one of the cutting device and the edge-attaching device thereto.

In some applications, the anatomical site includes a cardiac valve, and the cut tissue includes a portion of a leaflet of the cardiac valve.

The above method(s) can be performed on a living animal or on a simulation, such as on a cadaver, cadaver heart, simulator (e.g., with the body parts, heart, tissue, etc. being simulated), etc.

In accordance with some applications, there is provided a system for use with a leaflet of a cardiac valve of a subject, the leaflet having a root attached to an annulus of the cardiac valve and a lip adapted to coapt with at least one other leaflet of the valve. The system includes at least one longitudinal catheter configured to be transluminally advanced toward a heart chamber of the subject adjacent the cardiac valve. The longitudinal catheter can have a proximal part and a steerable distal part, and a longitudinal axis therebetween.

In some applications, the system includes a longitudinal mount, advanceable distally out of the longitudinal catheter. The mount can have a first curved surface and a second surface.

In some applications, the system can include a helical needle defining a helical lumen. The helical needle can be configured to be advanced distally out of the longitudinal catheter and to axially and rotationally extend about the first curved surface of the longitudinal mount and through tissue of the leaflet, drawing the tissue against the second surface of the longitudinal mount.

In some applications, the system can include a suture extending through the helical lumen of the helical needle. In some applications, the system can include a suture that does not extend through a helical lumen, but is otherwise attached or coupled to the helical needle (the needle need not have a helical lumen in this case).

In some applications, the at least one longitudinal catheter is adapted to transluminally advance the mount adjacent tissue of the leaflet in an advancement direction. In some applications, the at least one longitudinal catheter is further adapted to transluminally advance the helical needle and the suture about the mount along and through the leaflet, thereby drawing tissue of the leaflet against the second surface of the mount. In some applications, the at least one longitudinal catheter is further adapted to secure a distal end of the suture. In some applications, the at least one longitudinal catheter is also adapted to remove the mount and the helical needle from the cardiac valve, while maintaining the suture threaded helically through the tissue of the leaflet. In some applications, the at least one longitudinal catheter is additionally adapted to tension the suture, thereby deforming the tissue of the leaflet disposed within the suture.

In some applications, the system includes at least one locking element, configured to be applied to the suture by the at least one catheter, following tensioning of the suture, to secure the suture in its tensioned state.

In some applications, the helical needle includes a needle body terminating at a detachable distal tip, and wherein a distal end of the suture is attached to the detachable distal tip of the helical needle.

In some applications, the system includes a pushing-wire extending through the helical needle and configured to push the detachable distal tip of the helical needle distally relative to the needle body, thereby to detach the detachable distal tip from the needle body.

In some applications, the catheter is adapted to remove the helical needle, following detachment of the detachable distal tip of the helical needle from the needle body, by retracting the needle body helically away from the detachable distal tip while sliding over and along the suture.

In some applications, the system includes a user interface enabling a user to control motion of the catheter and operation thereof.

In some applications, at least one of the mount and the helical needle is adapted to be advanced through the longitudinal catheter to the heart chamber, or removed from the heart chamber via the longitudinal catheter. In some applications, mount and the helical needle are adapted to be advanced through a single lumen of the longitudinal catheter. In some applications, the longitudinal catheter includes at least two lumens, and each of the mount and the helical needle is adapted to be advanced through a different one of the at least two lumens of the longitudinal catheter.

In some applications, the mount has a crescent-shaped cross section in a direction perpendicular to a longitudinal axis of the mount, the crescent-shape defining an exterior curved surface as the first curved surface, and an interior curved surface defining a cavity as the second curved surface. In some applications, the helical needle is adapted, during rotation thereof, to draw tissue into the cavity.

In some applications, the first curved surface is an arc of a first circle having a first radius, and the second curved surface is an arc of a second circle having a second radius. In some applications, the second radius is not smaller than the first radius. In some applications, when the mount is circumscribed by the helical needle, there is a gap between the mount and the helical needle along at least one portion of the mount.

In some applications, the helical needle includes shape memory materials.

In accordance with some applications, there is provided a method of deforming a leaflet of a cardiac valve of a subject, the leaflet extending between a root attached to a cardiac wall and a lip adapted to coapt with at least one other leaflet. The method includes transluminally advancing a longitudinal catheter toward the cardiac valve. The longitudinal catheter can have a proximal part and a steerable distal part.

In some applications, the method can include advancing a longitudinal mount distally out of the longitudinal catheter. The longitudinal mount can have a first curved surface and a second curved surface.

In some applications, the method can include advancing a helical needle distally out of the longitudinal catheter about the first curved surface of the mount, along and through the leaflet, thereby drawing tissue of the leaflet against the second surface of the mount. In some applications, the helical needle includes a helical lumen and a suture extending through the helical lumen.

In some applications, the method can include, following the advancing of the helical needle, securing a distal end of the suture adjacent the leaflet.

In some applications, the method can include removing the mount and the helical needle from the cardiac valve, while maintaining the suture threaded helically through the tissue of the leaflet.

In some applications, the method can include tensioning the suture, thereby to deform the tissue disposed within the suture.

In some applications, the securing of the distal end of the suture includes sliding a locking element onto the distal end of the suture to secure the distal end of the suture.

In some applications, the method includes, following the tensioning of the suture, sliding another locking element onto the suture to secure the suture in its tensioned state.

In some applications, the method includes, following the advancing of the helical needle, detaching a detachable distal tip of the helical needle from a main body of the helical needle. In some applications, the removing of the helical needle includes helically retracting the main body of the helical needle away from the detachable distal tip while sliding over the suture.

In some applications, at least one of the advancing of the mount, the advancing of the helical needle, and the removing of the mount and the helical needle is carried out via the longitudinal catheter. In some applications, the advancing of the mount and the advancing of the helical needle are carried out via a single lumen of the longitudinal catheter. In some applications, the advancing of the mount is carried out via a first lumen of the longitudinal catheter, and the advancing of the helical needle is carried out via a second lumen of the longitudinal catheter, the first lumen being different from the second lumen.

In some applications, the mount has a crescent-shaped cross section in a direction perpendicular to a longitudinal axis of the mount, the crescent-shape defining an exterior curved surface as the first curved surface, and an interior curved surface defining a cavity as the second curved surface. In some applications, the advancing of the helical needle includes drawing tissue of the leaflet into the cavity, during rotation of the helical needle.

In some applications, the first curved surface is an arc of a first circle having a first radius, and the second curved surface is an arc of a second circle having a second radius, the second radius being not smaller than the first radius. In some applications, the advancing of the helical needle includes drawing tissue of the leaflet into a gap between the mount and the helical needle, during rotation of the helical needle.

In some applications, the advancing of the mount includes advancing the mount along the leaflet in a direction substantially perpendicular to the lip of the leaflet, thereby deforming the tissue of the leaflet disposed within the suture to shorten a width of the leaflet.

In some applications, the advancing of the mount includes advancing the mount along the leaflet in a direction substantially parallel to the lip of the leaflet or to the root of the leaflet, thereby deforming the tissue of the leaflet disposed within the suture to shorten a length of the leaflet between the root and the lip.

In some applications, the advancing of the mount includes advancing the mount along the root of the leaflet. In some applications, the advancing of the helical needle about the mount draws tissue of the root of the leaflet, adjoining a cardiac wall surrounding the cardiac valve, such that deforming the tissue disposed within the suture to shorten a length of the leaflet along the root of the leaflet.

In some applications, the advancing of the longitudinal catheter is via a coronary artery surrounding the leaflet. In some applications, the advancing of the helical needle about the mount includes advancing the helical needle through the wall of the coronary artery and through the leaflet adjacent the root of the leaflet, such that deforming the tissue disposed within the suture to shorten a length of the leaflet along the root of the leaflet.

The above method(s) can be performed on a living animal or on a simulation, such as on a cadaver, cadaver heart, simulator (e.g., with the body parts, heart, tissue, etc. being simulated), etc.

In accordance with some applications, there is provided a system for repairing billowing, prolapse, or flailing of a leaflet of a cardiac valve of a subject, the leaflet having a root attached to an annulus of the cardiac valve and a lip adapted to coapt with at least one other leaflet of the valve. The system includes at least one longitudinal catheter configured to be transluminally advanced toward a heart chamber of the subject adjacent the cardiac valve. The longitudinal catheter can have a proximal part and a steerable distal part, and a longitudinal axis therebetween.

In some applications, the system includes an implant including first and second leaflet engagement portions adapted to engage the lip of the leaflet. In some applications, the implant includes at least one tether extending between the first and second leaflet engagement portions.

In some applications, the at least one catheter is adapted to transluminally deliver the implant to the heart chamber adjacent the cardiac valve. In some applications, the at least one catheter is adapted to engage the first leaflet engagement portion to a first location on the lip of the leaflet. In some applications, the at least one catheter is adapted to engage the second leaflet engagement portion to a second location on the lip of the leaflet. In some applications, the at least one catheter is adapted to draw the first and second leaflet engagement portions toward each other by tensioning the at least one tether between the first and second leaflet engagement portions.

In some applications, the system includes at least one locking element, wherein the catheter is further adapted to slide the at least one locking element onto at least one end of the at least one tether, following tensioning thereof, to maintain the tension in the at least one tether.

In some applications, the implant includes a unitary frame, having the first and second leaflet engagement portions as ends thereof. In some applications, the unitary frame includes a central portion, and first and second arm portions connecting the central portion to each of the first and second leaflet engagement portions, respectively. In some applications, the at least one tether includes a single tether. In some applications, a hollow lumen extends through the unitary frame, and the tether extends through the lumen, and out of the first and second leaflet engaging portions.

In some applications, the unitary frame has a rest state, in which a first distance between ends of the first and second arm portions which connect to the first and second leaflet engaging portions, respectively, is greater than a second distance between ends of the first and second arm portions which engage the central portion. In some applications, a third distance between edges of the first and second leaflet engaging portions is smaller than the second distance.

In some applications, when a force is applied to the leaflet engaging portions pushing them toward each other while the unitary frame is in the rest state, the first and second arm portions are adapted to pivot relative to the central portion, to decrease the second and third distances.

In some applications, the unitary frame is substantially planar. In some applications, the unitary frame is at least one of flexible and resilient. In some applications, the unitary frame is formed of a shape memory material.

In some applications, the catheter is adapted to engage the first and second leaflet engagement portions to the lip of the leaflet when the unitary frame is in a rest state of the unitary frame. In some applications, tensioning of the tether causes the unitary frame to transition from the rest state to a second operative state, in which edges of the first and second leaflet engagement portions are drawn toward each other relative to their positions in the rest state.

In some applications, the catheter is adapted to engage the first and second leaflet engagement portions to the leaflet by placing the first and second arm portions, and the central portion, against a downstream surface of the leaflet, such that the first and second leaflet engagement portions extend over the lip of the leaflet to the upstream surface thereof.

In some applications, the catheter is adapted, by tensioning of the tether, to cause a portion of the lip of the leaflet, between the first and second leaflet engagement portions, to deform and create a bulge in the downstream surface of the leaflet. In some applications, following creation of the bulge, the first and second arm portions are adapted to be substantially perpendicular to the lip of the leaflet.

In some applications, the first and second leaflet engagement portions include first and second clips, and the catheter is adapted to engage the first and second leaflet engagement portions to the lip of the leaflet by clipping the first and second clips to the lip of the leaflet, at the first and second positions along the lip, respectively.

In some applications, the catheter is adapted to engagement the first and second leaflet engagement portions to the lip in a direction substantially perpendicular to lip of the leaflet.

In some applications, the at least one tether includes multiple tethers, each connecting the first and second leaflet engagement portions to each other, and wherein the catheter is adapted to tension each of the multiple tethers.

In accordance with some applications, there is provided a method for repairing billowing, prolapse, or flailing of a leaflet of a cardiac valve of a subject, the leaflet having a root attached to an annulus of the cardiac valve and a lip adapted to coapt with at least one other leaflet of the valve. The method includes transluminally delivering to a heart chamber, adjacent the cardiac valve, an implant including a first leaflet engagement portion, a second leaflet engagement portion, and at least one tether extending between the first and second leaflet engagement portions.

In some applications, the method includes engaging the first leaflet engagement portion with a first location on the lip of the leaflet.

In some applications, the method includes engaging the second leaflet engagement portion with a second location on the lip of the leaflet.

In some applications, the method includes drawing the first and second leaflet engagement portions toward each other by tensioning the at least one tether between the first and second leaflet engagement portions.

In some applications, the method includes securing the at least one tether following tensioning thereof, to maintain the first and second leaflet engagement portions drawn toward each other.

In some applications, the implant includes a unitary frame, having the first and second leaflet engagement portions as ends thereof. In some applications, the unitary frame includes a central portion, and first and second arm portions connecting the central portion to each of the first and second leaflet engagement portions, respectively. In some applications, the at least one tether includes a single tether. In some applications, a hollow lumen extends through the unitary frame, and the tether extends through the lumen, and out of the first and second leaflet engaging portions. In some applications, tensioning of the tether reduces a distance between ends of the first and second leaflet engagement portions, thereby deforming the leaflet and create a bulge in the downstream surface of the leaflet.

In some applications, the engaging of the first and second leaflet engagement portion includes placing the first and second arm portions, and the central portion, against a downstream surface of the leaflet, such that the first and second leaflet engagement portions extend over the lip of the leaflet to the upstream surface thereof.

In some applications, the first and second leaflet engagement portions include first and second clips, the engaging of the first leaflet engagement portion includes clipping the first clip onto the lip of the leaflet at a first position, and clipping the second clip onto the lip at a second position.

In some applications, the clipping of the first and second clips is in a direction substantially perpendicular to lip of the leaflet.

In some applications, the at least one tether includes multiple tethers, each connecting the first and second leaflet engagement portions to each other, and wherein the tensioning includes tensioning each of the multiple tethers.

The above method(s) can be performed on a living animal or on a simulation, such as on a cadaver, cadaver heart, simulator (e.g., with the body parts, heart, tissue, etc. being simulated), etc.

In accordance with some applications, there is provided a system for repairing billowing, prolapse, or flailing of a leaflet of a cardiac valve of a subject, the leaflet having a root attached to an annulus of the cardiac valve and a lip adapted to coapt with at least one other leaflet of the valve. The system includes at least one longitudinal catheter configured to be transluminally advanced toward a heart chamber of the subject adjacent the cardiac valve. In some applications, the longitudinal catheter can have a proximal part and a steerable distal part, and a longitudinal axis therebetween.

In some applications, the system includes a piercing implant including a piercing element including a base from which extends a piercing tip and a securing element, adapted to secure the piercing tip of the piercing element.

In some applications, the at least one catheter is adapted to transluminally deliver the piercing implant to the heart chamber. In some applications, the at least one implant can be adapted to create a folded region of the leaflet by folding the leaflet. In some applications, the at least one implant can be adapted to insert the piercing tip of the piercing element through at least two layers of the leaflet at the folded region. In some applications, the at least one implant can be adapted to secure the at least one fold at the folded region by connecting the securing element to the piercing tip of the piercing element, such that the base of the piercing element and the securing element are on the same side of the leaflet, with the at least two layers disposed therebetween.

In some applications, the securing element includes a cylindrical housing defining a hollow, the hollow being adapted to receive the piercing tip. In some applications, the securing element includes an elastomer.

In some applications, the at least one catheter is adapted to transluminally delivery the piercing implant to the heart chamber while the piercing tip is secured by the securing element. In some applications, the at least one catheter is further adapted, prior to inserting the piercing tip of the piercing element, to separate the piercing element or the piercing tip from the securing element.

In some applications, the at least one catheter is adapted to transluminally delivery the piercing implant to the heart chamber while the piercing element is separated from the securing element.

In accordance with some applications, there is provided a method for repairing billowing, prolapse, or flailing of a leaflet of a cardiac valve of a subject, the leaflet having a root attached to an annulus of the cardiac valve and a lip adapted to coapt with at least one other leaflet of the valve. The method includes transluminally delivering to a heart chamber, adjacent the cardiac valve, a piercing implant including a piercing element having a base from which extends a piercing tip, and a securing element, the securing element adapted to secure the piercing tip of the piercing element.

In some applications, the method includes creating a folded region of the leaflet by folding the leaflet into at least one fold.

In some applications, the method includes inserting the piercing tip of the piercing element through at least two layers of the leaflet at the folded region.

In some applications, the method includes securing the at least one fold at the folded region by connecting the securing element to the piercing tip of the piercing element.

In some applications, following the securing, the base of the piercing element and the securing element are on the same side of the leaflet, with the at least two layers disposed therebetween.

In some applications, the securing element includes a cylindrical housing defining a hollow. In some applications, wherein the securing includes inserting the piercing tip into the hollow.

In some applications, the securing element includes an elastomer. In some applications, the securing includes piercing the piercing tip into the elastomer.

In some applications, the transluminally delivering includes transluminally delivering the piercing implant to the heart chamber while the piercing tip is secured by the securing element. In some applications, the method includes, following the transluminally delivering and prior to the inserting, separating the piercing element or the piercing tip from the securing element.

In some applications, the transluminally delivering includes transluminally delivery the piercing implant to the heart chamber while the piercing element is separated from the securing element.

In some applications, the creating a folded region includes creating a folded region disposed at an upstream side of the cardiac valve. In some applications, following the securing, the base of the piercing element and the securing element are on the upstream side of the leaflet.

In some applications, the creating a folded region includes creating a folded region disposed at a downstream side of the cardiac valve. In some applications, following the securing, the base of the piercing element and the securing element are on the downstream side of the leaflet.

The above method(s) can be performed on a living animal or on a simulation, such as on a cadaver, cadaver heart, simulator (e.g., with the body parts, heart, tissue, etc. being simulated), etc.

In accordance with some applications, there is provided a system for repairing billowing, prolapse, or flailing of a leaflet of a cardiac valve of a subject, the leaflet having a root attached to an annulus of the cardiac valve and a lip adapted to coapt with at least one other leaflet of the valve, the leaflet including a first indentation and a second indentation. The system includes at least one longitudinal catheter configured to be transluminally advanced toward a heart chamber of the subject adjacent the cardiac valve. The longitudinal catheter can have a proximal part and a steerable distal part, and a longitudinal axis therebetween.

In some applications, the system includes a tether implant including a tether having a proximal end and a distal end and at least one locking element, adapted to secure at least one end of the tether.

In some applications, the at least one catheter adapted to transluminally deliver the tether to the heart chamber. In some applications, the at least one catheter can be adapted to thread the distal end of the tether through the first indentation in the leaflet, from a first side of the leaflet to a second side of the leaflet. In some applications, the at least one catheter can be adapted to additionally thread the distal end of the tether through the second indentation in the leaflet, from the second side of the leaflet to the first side of the leaflet. In some applications, the at least one catheter can be adapted to tension the tether threaded through the first and second indentations, to draw the first and second indentations toward each other. In some applications, the at least one catheter can be adapted to secure the proximal and distal ends of the tether, using the at least one locking element, to maintain the tension in the tether.

In some applications, the system includes a gripping tool extending distally out of the at least one longitudinal catheter. In some applications, the gripping tool is adapted to grip the distal end of the tether during threading of the distal end by the catheter.

In some applications, the tether implant includes multiple tethers, each having a corresponding proximal end and a corresponding distal end. In some applications, the tether implant includes multiple locking elements adapted to secure at least one end of each of the multiple tethers. In some applications, the catheter is adapted to deliver, thread, tension, and secure, each of the multiple tethers.

In accordance with some applications, there is provided a method for repairing billowing, prolapse, or flailing of a leaflet of a cardiac valve of a subject, the leaflet having a root attached to an annulus of the cardiac valve and a lip adapted to coapt with at least one other leaflet of the valve, the leaflet including a first indentation and a second indentation. The method includes transluminally delivering to a heart chamber, adjacent the cardiac valve, a tether having a proximal end and a distal end.

In some applications, the method includes threading the distal end of the tether through the first indentation in the leaflet, from a first side of the leaflet to a second side of the leaflet.

In some applications, the method includes additionally threading the distal end of the tether through the second indentation in the leaflet, from the second side of the leaflet to the first side of the leaflet.

In some applications, the method includes tensioning the tether threaded through the first and second indentations, to draw the first and second indentations toward each other.

In some applications, the method includes, following the tensioning, securing the proximal and distal ends of the tether, using at least one locking element, to maintain the tension in the tether.

In some applications, at least one of the threading and the additionally threading includes gripping the distal end of the tether using a gripping tool extending distally out of the at least one longitudinal catheter.

In some applications, the steps of transluminally delivering, threading, additionally threading, tensioning, and securing, are carried out for each of multiple tethers.

The above method(s) can be performed on a living animal or on a simulation, such as on a cadaver, cadaver heart, simulator (e.g., with the body parts, heart, tissue, etc. being simulated), etc.

In accordance with some applications, there is provided a system for repairing billowing, prolapse, or flailing of a leaflet of a cardiac valve of a subject, the leaflet having a root attached to an annulus of the cardiac valve and a lip adapted to coapt with at least one other leaflet of the valve. The system includes at least one longitudinal catheter configured to be transluminally advanced toward a heart chamber of the subject adjacent the cardiac valve. In some applications, the longitudinal catheter has a proximal part and a steerable distal part, and a longitudinal axis therebetween.

In some applications, the system includes an implant including first, second, and third beams and having a first operative state and a second operative state.

In some applications, the at least one catheter is adapted to transluminally deliver the implant to the heart chamber. In some applications, the at least one catheter is adapted to place the implant onto the leaflet in the first operative state. In some applications, the at least one catheter is adapted to transform the implant from the first operative state toward the second operative state, thereby causing the leaflet to follow a tortuous path between the first and third beams.

In some applications, at least two of the first, second, and third beams are substantially parallel to each other, in at least one of the first operative state and the second operative state.

In some applications, the implant is at least one of flexible and resilient. In some applications, the implant includes a resilient metal. In some applications, the implant includes a shape memory material. In some applications, the implant includes an elastic component.

In some applications, the catheter is adapted to place the implant onto the leaflet in the first operative state by placing the implant such that the lip of the leaflet engages a portion of the implant, and the first, second, and third beams are substantially perpendicular to the lip of the implant.

In some applications, the catheter is adapted to place the implant onto the leaflet, in the first operative state, such two of the first, second, and third beams are disposed on or adjacent one side of the leaflet, and a remaining one of the first second and third beams is disposed on or adjacent the opposing side of the leaflet.

In some applications, the implant includes a base, from which extend a plurality of beams including the first, second, and third beams. In some applications, each of the plurality of beams has a terminus distal to the base. In some applications, the first, second, and third beams are substantially parallel to one another. In some applications, in the first operative state, the terminus of one of the first, second, and third beams is disposed outside of a plane defined by the base and termini of the other two of the first, second, and third beams, the one of the first, second, and third beams being disposed between the other two of the first, second, and third beams. In some applications, in the second operative state, the base, as well as the termini of the first, second, and third beams, are in a single plane.

In some applications, the plurality of beams includes at least five beams. A first subset of the plurality of beams includes the one of the first, second, and third beams, and a second subset of the plurality of beams includes the other two of the first, second, and third beams. In some applications, the first and second subsets of the plurality of beams being mutually exclusive. In some applications, in the first operative state, the plane is defined by the termini all beams in the second subset and by the base, and the termini of all beams in the first subset are disposed outside of the plane. In some applications, in the second operative state, the base, as well as the termini of the beams in the first and second subsets, are in the single plane.

In some applications, in the first operative state, the termini of all beams in the first subset are disposed in a second plane, the second plane being angled relative to the plane.

In some applications, the first, second, and third beams are substantially perpendicular to the base, in at least one of the first and second operative states.

In some applications, the catheter is adapted to slide the implant onto the lip of the leaflet, when the implant is in the first operative state, such that the base extends along the lip of the leaflet, thereby to place the implant onto the leaflet.

In some applications, the implant includes first and second U-shaped portions, each including a base and first and second beams extending substantially perpendicularly from the base, and substantially parallel to each other. In some applications, the implant includes an engaging element holding together the second beam of each of the first and second U-shaped portions to form a unified beam.

In some applications, the unified beam is a central beam, disposed between the first beam of the first U-shaped portion and the first beam of the second U-shaped portion. In some applications, the second beams of the first and second U-shaped portion are pivotable relative to the engaging element, about respective longitudinal axes of the second beams, such that the first and second U-shaped portions are pivotable relative to each other.

In some applications, the first and second U-shaped portions are identical to each other.

In some applications, the engaging element includes a longitudinally extending cylinder.

In some applications, in the first operative state, the first and second U-shaped portions are substantially in a single plane, and in the second operative state, the unified beam is disposed outside of a plane formed by the first beams of the first and second U-shaped portions.

In some applications, in the first operative state, a first distance exists between the first beams of the first and second U-shaped portions, and in the second operative state a second distance exists between the first beams of the first and second U-shaped portions, the second distance being smaller than the first distance.

In some applications, the catheter is adapted to rotate the first and second U-shaped portions relative to each other, thereby transforming the implant from the first operative state to the second operative state.

In some applications, the catheter is adapted to slide the implant onto the lip of the leaflet, when the implant is in the first operative state, such that the bases of the first and second U-shaped portions extend along the lip of the leaflet, the unified beam is disposed on or adjacent one side of the leaflet, and the first beams of the first and second U-shaped portions are disposed on or adjacent the opposing side of the leaflet, thereby to place the implant onto the leaflet.

In some applications, the implant includes a central U-shaped portion, including a pair of central beams and a connecting segment. In some applications, the implant includes first and second side portions, each including at least one beam and being connected to one of the central beams by a corresponding one of first and second bends. In some applications, the at least one beam of each of the first and second side portions is substantially parallel to at least one of the central beams. In some applications, the first and second bends facilitate motion of corresponding first and second side portions, relative to the pair of central beams, in at least two directions.

In some applications, in the first operative state, the central U-shaped portion is in a first plane, and each of the first and second side portions is in plane different from the first plane, and is disposed exteriorly to the central U-shaped portion. In some applications, in the second operative state, the central U-shaped portion and the first and second side portions are in the first plane, with the first and second side portions being disposed within the central U-shaped portion.

In some applications, the implant is formed of a single length of a flexible or resilient material.

In some applications, each of the first and second side portions includes a pair of side beams, connected to each other by an additional bend, the pair of side beams being substantially parallel to each other.

In some applications, the implant is side-to-side symmetrical about a central longitudinal axis of the central U-shaped portion.

In some applications, in the first operative state, a first distance exists between the first and second side portions, and in the second operative state a second distance exists between the first and second side portions, the second distance being smaller than the first distance.

In some applications, the implant has an intermediate operative state, in which the first and second side portions are in the first plane, and are disposed exteriorly to the central U-shaped portion. In some applications, for transforming the implant from the first operative state to the second operative state, the catheter is adapted to transform the implant from the first operative state to the intermediate operative state, by pivoting the first and second side portions in a first direction relative to the central U-shaped portion, to place the first and second side portions in the first plane, and to further transform the implant from the intermediate operative state to the second operative state by revolving the first and second side portions in a second direction relative to the central U-shaped portion, to move the first and second side portions to the interior of the central U-shaped portion.

In some applications, the first direction is about an axis perpendicular to longitudinal axes of the central beams, and the second direction is about axes parallel to the longitudinal axes of the central beams.

In some applications, the catheter is adapted to slide the implant onto the lip of the leaflet, when the implant is in the first operative state, such that the first and second bends engage the lip of the leaflet and the connecting segment of the U-shaped portion is distal to the lip of the leaflet, with the U-shaped portion being disposed on or adjacent one side of the leaflet, and the first and second side portions being disposed on or adjacent the opposing side of the leaflet, thereby to place the implant onto the leaflet.

In some applications, the catheter is adapted to transluminally deliver the implant to the heart chamber when the implant is in the first operative state.

In some applications, the catheter is adapted to transluminally deliver the implant to the heart chamber when the implant is in a preparatory operative state, in which the first and second side portions are in the first plane and are exterior to the central U-shaped portion. In some applications, the catheter is further adapted, prior to placing the implant onto the leaflet, to transition the implant from the preparatory operative state to the first operative state, by pivoting the first and second side portions relative to the U-shaped portion about an axis perpendicular to longitudinal axes of the central beams.

In some applications, the implant includes a tubular body arranged along a central longitudinal axis. In some applications, the implant includes first and second substantially U-shaped clips, each having a first elongate portion disposed on, or near, an exterior surface of the tubular body, and a second elongate portion disposed on, or near, an interior surface of the tubular body, the first and second elongate portions connected by an end portion. In some applications, the implant includes an elastic element holding together the second elongate portions of the first and second clips.

In some applications, in the first operative state, the clips are disposed on a first side of the tubular body, and in the second operative state the clips are disposed on a second side of the tubular body, the second side being opposite the first side.

In some applications, the catheter is adapted to slide the implant onto the lip of the leaflet, when the implant is in the first operative state, such that the end portions engage the lip of the leaflet, with the tubular body and the second elongate portions of the first and second clips being disposed on or adjacent one side of the leaflet, and the first elongate portions of the first and second clips being disposed on or adjacent the opposing side of the leaflet, thereby to place the implant onto the leaflet.

In some applications, the catheter is adapted to transluminally deliver the implant to the heart chamber when the implant is in the first operative state.

In some applications, the catheter is adapted to transluminally deliver the implant to the heart chamber when the U-shaped clips are separate from the tubular body. In some applications, the catheter is further adapted, prior to or during placing the implant onto the leaflet, to place the first and second clips onto the tubular body.

In some applications, the catheter is adapted to transform the implant from the first operative state to the second operative state, by drawing the first and second clips away from each other, about the perimeter of the tubular body and against the force of the elastic element.

In accordance with some applications, there is provided a method for repairing billowing, prolapse, or flailing of a leaflet of a cardiac valve of a subject, the leaflet having a root attached to an annulus of the cardiac valve and a lip adapted to coapt with at least one other leaflet of the valve. The method includes transluminally delivering to a heart chamber, adjacent the cardiac valve, an implant having a first operative state and a second operative state, the implant including first, second, and third beams.

In some applications, the method includes placing the implant onto the leaflet in the first operative state.

In some applications, the method includes, while the implant is on the leaflet, causing the leaflet to follow a tortuous path between the first and third beams by transforming the implant from the first operative state toward the second operative state.

In some applications, the placing of the implant includes placing the implant such that the lip of the leaflet engages a portion of the implant, and the first, second, and third beams are substantially perpendicular to the lip of the implant.

In some applications, the placing of the implant includes placing the implant onto the leaflet in the first operative state such two of the first, second, and third beams are disposed on or adjacent one side of the leaflet, and a remaining one of the first second and third beams is disposed on or adjacent the opposing side of the leaflet.

In some applications, the implant includes a base, from which extend a plurality of beam including the first, second, and third beams, each of the plurality of beams having a terminus distal to the base, the first, second, and third beams being substantially parallel to one another. In some applications, in the first operative state, the terminus of one of the first, second, and third beams is disposed outside of a plane defined by the base and termini of the other two of the first, second, and third beams, the one of the first, second, and third beams being disposed between the other two of the first, second, and third beams. In some applications, in the second operative state, the base, as well as the termini of the first, second, and third beams, are in a single plane. In some applications, the transforming of the implant from the first operative state toward the second operative state includes moving the termini of first, second, and third beams, relative to each other, so that the termini of the first, second, and third beams are in the single plane.

In some applications, the plurality of beams includes at least five beams, a first subset of the plurality of beams includes the one of the first, second, and third beams, and a second subset of the plurality of beams includes the other two of the first, second, and third beams, the first and second subsets of the plurality of beams being mutually exclusive. In some applications, in the first operative state, the plane is defined by the termini all beams in the second subset and by the base, and the termini of all beams in the first subset are disposed outside of the plane. In some applications, in the second operative state, the base, as well as the termini of the beams in the first and second subsets, are in the single plane.

In some applications, in the first operative state, the termini of all beams in the first subset are disposed in a second plane, the second plane being angled relative to the plane.

In some applications, the first, second, and third beams are substantially perpendicular to the base, in at least one of the first and second operative states.

In some applications, the placing of the implant onto the leaflet includes sliding the implant onto the lip of the leaflet, when the implant is in the first operative state, such that the base extends along the lip of the leaflet with the one of the first, second, and third beams being on one side of the leaflet, and the other two of the first, second, and third beams being on the opposing side of the leaflet.

In some applications, the implant includes first and second U-shaped portions, each including a base and first and second beams extending substantially perpendicularly from the base, and substantially parallel to each other, and an engaging element holding together the second beam of each of the first and second U-shaped portions to form a unified beam.

In some applications, the unified beam is a central beam, disposed between the first beam of the first U-shaped portion and the first beam of the second U-shaped portion.

In some applications, the second beams of the first and second U-shaped portion are pivotable relative to the engaging element, about respective longitudinal axes of the second beams, such that the first and second U-shaped portions are pivotable relative to each other.

In some applications, the first and second U-shaped portions are identical to each other.

In some applications, the engaging element includes a longitudinally extending cylinder.

In some applications, in the first operative state, the first and second U-shaped portions are substantially in a single plane, and in the second operative state, the unified beam is disposed outside of a plane formed by the first beams of the first and second U-shaped portions.

In some applications, in the first operative state, a first distance exists between the first beams of the first and second U-shaped portions, and in the second operative state a second distance exists between the first beams of the first and second U-shaped portions, the second distance being smaller than the first distance.

In some applications, the transforming of the implant includes rotating the first and second U-shaped portions relative to each other.

In some applications, the placing of the implant includes sliding the implant onto the lip of the leaflet, when the implant is in the first operative state, such that the bases of the first and second U-shaped portions extend along the lip of the leaflet, the unified beam is disposed on or adjacent one side of the leaflet, and the first beams of the first and second U-shaped portions are disposed on or adjacent the opposing side of the leaflet.

In some applications, the implant includes a central U-shaped portion, including a pair of central beams and a connecting segment, and first and second side portions, each including at least one beam and being connected to one of the central beams by a corresponding one of first and second bends.

In some applications, the at least one beam of each of the first and second side portions is substantially parallel to at least one of the central beams. In some applications, the first and second bends facilitate motion of corresponding first and second side portions, relative to the pair of central beams, in at least two directions.

In some applications, in the first operative state, the central U-shaped portion is in a first plane, and each of the first and second side portions is in plane different from the first plane, and is disposed exteriorly to the central U-shaped portion, and in the second operative state, the central U-shaped portion and the first and second side portions are in the first plane, with the first and second side portions being disposed within the central U-shaped portion.

In some applications, the implant is formed of a single length of a flexible or resilient material.

In some applications, each of the first and second side portions includes a pair of side beams, connected to each other by an additional bend, the pair of side beams being substantially parallel to each other.

In some applications, the implant is side-to-side symmetrical about a central longitudinal axis of the central U-shaped portion.

In some applications, in the first operative state, a first distance exists between the first and second side portions, and in the second operative state a second distance exists between the first and second side portions, the second distance being smaller than the first distance.

In some applications, the implant has an intermediate operative state, in which the first and second side portions are in the first plane, and are disposed exteriorly to the central U-shaped portion. In some applications, the transforming of the implant from the first operative state toward the second operative state includes transforming the implant from the first operative state to the intermediate operative state, by pivoting the first and second side portions in a first direction relative to the central U-shaped portion, to place the first and second side portions in the first plane. In some applications, the transforming includes transforming the implant from the intermediate operative state to the second operative state by revolving the first and second side portions in a second direction relative to the central U-shaped portion, to move the first and second side portions to the interior of the central U-shaped portion.

In some applications, the first direction is about an axis perpendicular to longitudinal axes of the central beams, and the second direction is about axes parallel to the longitudinal axes of the central beams.

In some applications, the placing of the implant includes sliding the implant onto the lip of the leaflet, when the implant is in the first operative state, such that the first and second bends engage the lip of the leaflet and the connecting segment of the U-shaped portion is distal to the lip of the leaflet, with the U-shaped portion being disposed on or adjacent one side of the leaflet, and the first and second side portions being disposed on or adjacent the opposing side of the leaflet.

In some applications, the transluminally delivering includes transluminally delivering the implant to the heart chamber when the implant is in the first operative state.

In some applications, the transluminally delivering includes transluminally delivering the implant to the heart chamber when the implant is in a preparatory operative state, in which the first and second side portions are in the first plane and are exterior to the central U-shaped portion. In some applications, the method includes, prior to the placing of the implant, transitioning the implant from the preparatory operative state toward the first operative state.

In some applications, the transitioning of the implant from the preparatory state includes pivoting the first and second side portions relative to the U-shaped portion about an axis perpendicular to longitudinal axes of the central beams.

In some applications, the implant includes a tubular body arranged along a central longitudinal axis, first and second substantially U-shaped clips, each having a first elongate portion disposed on, or near, an exterior surface of the tubular body, and a second elongate portion disposed on, or near, an interior surface of the tubular body, the first and second elongate portions connected by an end portion, and an elastic element holding together the second elongate portions of the first and second clips.

In some applications, in the first operative state, the clips are disposed on a first side of the tubular body, and in the second operative state the clips are disposed on a second side of the tubular body, the second side being opposite the first side.

In some applications, the placing of the implant includes sliding the implant onto the lip of the leaflet, when the implant is in the first operative state, such that the end portions engage the lip of the leaflet, with the tubular body and the second elongate portions of the first and second clips being disposed on or adjacent one side of the leaflet, and the first elongate portions of the first and second clips being disposed on or adjacent the opposing side of the leaflet.

In some applications, the transluminally delivering includes transluminally delivering the implant to the heart chamber when the implant is in the first operative state.

In some applications, the transluminally delivering includes transluminally delivering the implant to the heart chamber when the U-shaped clips are separate from the tubular body. In some applications, the method includes, prior to or during the placing of the implant, sliding the first and second clips onto the tubular body to form the first operative state.

In some applications, the transforming of the implant includes drawing the first and second clips away from each other, about the perimeter of the tubular body and against the force of the elastic element.

The above method(s) can be performed on a living animal or on a simulation, such as on a cadaver, cadaver heart, simulator (e.g., with the body parts, heart, tissue, etc. being simulated), etc.

In accordance with some applications, there is provided a system for repairing billowing, prolapse, or flailing of a leaflet of a cardiac valve of a subject, the leaflet having a root attached to an annulus of the cardiac valve and a lip adapted to coapt with at least one other leaflet of the valve. The system includes at least one longitudinal catheter configured to be transluminally advanced toward a heart chamber of the subject adjacent the cardiac valve. In some applications, the longitudinal catheter can have a proximal part and a steerable distal part, and a longitudinal axis therebetween.

In some applications, the system includes an implant including a core and a securing element, adapted to secure tissue about the core.

In some applications, the at least one catheter adapted to transluminally deliver the implant to the heart chamber. In some applications, the at least one catheter is adapted to dispose a portion of the leaflet about the core by placing the core against a first surface of the leaflet. In some applications, the at least one catheter is adapted to, from a second, opposing, surface of the leaflet, using the securing element, secure the portion of the leaflet extending about the core, thereby deforming the region of the leaflet.

In some applications, the core and the securing element are sized and configured to cooperate with each other to retain the core and securing element in their implanted positions on the leaflet.

In some applications, the at least one catheter includes a first catheter adapted to transluminally deliver the core, and a second catheter adapted to transluminally deliver the securing element.

In some applications, the core includes a tubular core, and the securing element includes a securing clip, the securing clip having first and second longitudinal portions connected by an end portion.

In some applications, the catheter is adapted to dispose the portion of the leaflet about the tubular core when a longitudinal axis of the tubular core is substantially perpendicular to the lip of the leaflet.

In some applications, the catheter is adapted to secure the portion of the leaflet about the tubular core by the securing clip when the first and second longitudinal portions of the securing clip are substantially perpendicular to the lip of the leaflet.

In some applications, the catheter is adapted to place the securing clip such that the end portion thereof is distal to the lip of the leaflet, thereby to secure the portion of the leaflet extending about the core.

In some applications, the catheter is adapted to pierce the securing clip through tissue of the leaflet when securing the portion of the leaflet extending about the core.

In some applications, the catheter is further adapted, once the securing element is secured, to remove the core from the cardiac valve, such that the leaflet remains secured only by the securing element.

In some applications, the core includes first and second plates having a neck portion therebetween, the plates having a greater diameter than the neck portion, such that an indentation is formed between the plates, around the neck portion. In some applications, the securing element includes a panel having a groove formed therein, the groove having a plate-receiving region and a neck-receiving region, the plate-receiving region being adapted to receive one of the first and second plates of the core, and the neck receiving-region being adapted to receive the neck of the core, with the first plate extending on one side of panel, and the second plate extending on an opposing side of the panel.

In some applications, the first and second plates are substantially the same size. In some applications, the first and second plates are substantially parallel to one another.

In some applications, the plate receiving region is dimensioned to allow passage of at least one of the first and second plates therethrough, and the neck receiving region is dimensioned to limit passage of the first and second plates therethrough.

In some applications, a thickness of the panel is smaller than a height of the indentation of the core.

In some applications, the catheter is adapted to dispose a portion of the leaflet about the core by placing the first plate against the first surface of the leaflet. In some applications, the catheter is adapted to secure the portion of the leaflet extending about the core by inserting the first plate, with the portion of the leaflet disposed thereon, into the plate-receiving region of the panel, and moving the panel relative to the core, such that the neck portion of the core slides into the neck-receiving portion, while the first and second plates remain exterior to the panel on opposing sides thereof.

In accordance with some applications, there is provided a method for repairing billowing, prolapse, or flailing of a leaflet of a cardiac valve of a subject, the leaflet having a root attached to an annulus of the cardiac valve and a lip adapted to coapt with at least one other leaflet of the valve. The method includes transluminally delivering to a cardiac chamber adjacent the cardiac valve, an implant including a core and a securing element, the securing element adapted to secure tissue about the core.

In some applications, the method includes disposing a portion of the leaflet about the core by placing the core against a first surface of the leaflet.

In some applications, the method includes, from a second, opposing, surface of the leaflet, using the securing element, securing the portion of the leaflet extending about the core, thereby causing deformation of the region of the leaflet.

In some applications, the transluminally delivering includes, using a first catheter, transluminally delivering the core to a first cardiac chamber, adjacent the first surface of the leaflet, and using a second catheter, transluminally delivering the securing element to a second cardiac chamber, adjacent the second, opposing surface of the leaflet.

In some applications, the core includes a tubular core, and the securing element includes a securing clip, the securing clip having first and second longitudinal portions connected by an end portion. In some applications, the disposing includes disposing the portion of the leaflet about the tubular core when a longitudinal axis of the tubular core is substantially perpendicular to the lip of the leaflet. In some applications, the securing includes securing the portion of the leaflet about the tubular core when the first and second longitudinal portions of the securing clip are substantially perpendicular to the lip of the leaflet.

In some applications, following the securing, the end portion of the securing clip is distal to the lip of the leaflet.

In some applications, the securing includes piercing the tissue of the leaflet using the securing clip.

In some applications, the method includes, following the securing, removing the core from the cardiac valve, such that the leaflet remains secured only by the securing element.

In some applications, the core includes first and second plates having a neck portion therebetween, the plates having a greater diameter than the neck portion, such that an indentation is formed between the plates, around the neck portion. In some applications, the securing element includes a panel having a groove formed therein, the groove having a plate-receiving region and a neck-receiving region, the plate-receiving region being adapted to receive one of the first and second plates of the core, and the neck receiving-region being adapted to receive the neck of the core, with the first plate extending on one side of panel, and the second plate extending on an opposing side of the panel. In some applications, the disposing includes disposing a portion of the leaflet about the core by placing the first plate against the first surface of the leaflet.

In some applications, the securing includes inserting the first plate, with the portion of the leaflet disposed thereon, into the plate-receiving region of the panel, and moving the panel relative to the core, such that the neck portion of the core slides into the neck-receiving portion, while the first and second plates remain exterior to the panel on opposing sides thereof.

The above method(s) can be performed on a living animal or on a simulation, such as on a cadaver, cadaver heart, simulator (e.g., with the body parts, heart, tissue, etc. being simulated), etc.

In accordance with some applications, there is provided a system for repairing billowing, prolapse, or flailing of a leaflet of a cardiac valve of a subject, the leaflet having a root attached to an annulus of the cardiac valve, a lip adapted to coapt with at least one other leaflet of the valve, and an intermediate region extending between the root and the lip. The system includes at least one longitudinal catheter configured to be transluminally advanced toward a heart chamber of the subject adjacent the cardiac valve. The longitudinal catheter can have a proximal part and a steerable distal part, and a longitudinal axis therebetween.

The system can include a constraining implant.

In some applications, the at least one longitudinal catheter is adapted to transluminally deliver the constraining implant to the heart chamber and, using the constraining implant, constrain the lip so as to present the intermediate region as a substitute coaptation surface for coaptation with the at least one other leaflet.

In some applications, prior to using the constraining implant, the catheter is adapted to fold the leaflet to present the intermediate region thereof.

In some applications, the substitute coaptation surface includes at least a portion of the securing element.

In some applications, the catheter is adapted to constrain the lip of the leaflet to a downstream surface of the leaflet.

In some applications, the constraining element includes a U-shaped clip, and the catheter is adapted to constrain the lip by folding the lip toward the downstream surface of the leaflet and securing the fold using the U-shaped clip.

In some applications, the constraining element includes a plurality of U-shaped pins, and the catheter is adapted to constrain the lip by folding the lip toward the downstream surface of the leaflet and securing the fold by disposing the U-shaped pins at multiple locations along the fold.

In some applications, the catheter is adapted to constrain the lip of the leaflet to a cardiac wall, downstream of the cardiac valve.

In some applications, the constraining element includes at least one tissue anchor, and the catheter is adapted to anchor the lip of the leaflet to the cardiac wall using the at least one tissue anchor.

In some applications, the constraining element includes at least one pledget having first and second end portions connected by a longitudinal portion, the first and second end portions having a greater cross section than the longitudinal portion. In some applications, the catheter is adapted to constrain the lip of the leaflet, at the fold, by anchoring the first end of the at least one pledget to a first location in the cardiac wall, upstream of the cardiac valve and anchoring the second end of the at least one pledget to a second location in the cardiac wall downstream of the cardiac valve, through the lip of the leaflet, such that the longitudinal portion of the at least one pledget extends along a surface of the leaflet from the first location to the second location.

In some applications, the catheter is adapted to anchor the first and second ends of the at least one pledget into a coronary sinus adjacent the cardiac wall.

In some applications, the catheter is adapted to anchor first and second ends of the at least one pledget into a coronary artery within the cardiac wall.

In accordance with some applications, there is provided a method for repairing billowing, prolapse, or flailing of a leaflet of a cardiac valve of a subject, the leaflet having a root attached to an annulus of the cardiac valve, a lip adapted to coapt with at least one other leaflet of the valve, and an intermediate region extending between the root and the lip. The method includes transluminally delivering a constraining element to a heart chamber, adjacent the cardiac valve.

In some applications, the method includes presenting the intermediate region as a substitute coaptation surface for coaptation with the at least one other leaflet by constraining the lip using the constraining element.

In some applications, the substitute coaptation surface includes at least a portion of the constraining element.

In some applications, the constraining includes folding the leaflet to present the intermediate region.

In some applications, the constraining element includes a generally U-shaped clip, and the constraining includes folding the lip of the leaflet toward the downstream surface of the leaflet and securing the fold using the U-shaped clip.

In some applications, the constraining element includes a plurality of U-shaped pins, and the constraining includes folding the lip of the leaflet toward the downstream surface of the leaflet and securing the fold at multiple locations therealong using the U-shaped pins.

In some applications, the constraining includes constraining the lip of the leaflet to a cardiac wall, downstream of the cardiac valve.

In some applications, the constraining element includes at least one tissue anchor, and the constraining includes anchoring the lip of the leaflet to the cardiac wall.

In some applications, the constraining element includes at least one pledget having first and second end portions connected by a longitudinal portion, the first and second end portions having a greater cross section than the longitudinal portion. In some applications, the constraining includes anchoring the first end of the at least one pledget to a first location in the cardiac wall, upstream of the cardiac valve, and anchoring the second end of the at least one pledget to a second location in the cardiac wall downstream of the cardiac valve, through the lip of the leaflet, such that the longitudinal portion of the at least one pledget extends along an upstream surface of the leaflet from the first location to the second location.

In some applications, the anchoring of the first and second ends of the at least one pledget is into a coronary sinus adjacent the cardiac wall.

In some applications, the anchoring of the first and second ends of the at least one pledget is into a coronary artery within the cardiac wall.

The above method(s) can be performed on a living animal or on a simulation, such as on a cadaver, cadaver heart, simulator (e.g., with the body parts, heart, tissue, etc. being simulated), etc.

In accordance with some applications, there is provided a system for repairing billowing, prolapse, or flailing of a leaflet of a cardiac valve of a subject, the leaflet having a root attached to an annulus of the cardiac valve, a lip adapted to coapt with at least one other leaflet of the valve, and an intermediate region extending between the root and the lip. The system includes at least one longitudinal catheter configured to be transluminally advanced toward a heart chamber of the subject adjacent the cardiac valve. The longitudinal catheter can have a proximal part and a steerable distal part, and a longitudinal axis therebetween.

The system can include an implant including a constraining element and an artificial coaptation element.

In some applications, the at least one longitudinal catheter is adapted to transluminally deliver the constraining element and the artificial coaptation element to the heart chamber. In some applications, the at least one catheter is adapted to, using the constraining implant, constrain the lip so as to present the intermediate region. In some applications, the at least one catheter is adapted to mount the artificial coaptation element onto the intermediate region of the leaflet so as to present an artificial coaptation surface for coaptation with the at least one other leaflet.

In some applications, prior to using the constraining implant, the catheter is adapted to fold the leaflet to present the intermediate region thereof.

In some applications, the catheter is adapted to constrain the lip of the leaflet to a cardiac wall, downstream of the cardiac valve.

In some applications, the constraining element includes at least one tissue anchor, and the catheter is adapted to anchor the lip of the leaflet to the cardiac wall using the at least one tissue anchor.

In some applications, the artificial coaptation element includes a shape memory material. In some applications, the artificial coaptation element includes a wire mesh.

In some applications, the catheter is adapted to transluminally deliver the artificial coaptation element to the heart chamber in a compressed form, and to allow the artificial coaptation element to decompress following delivery or mounting thereof.

In some applications, the at least one catheter includes a first catheter adapted to transluminally deliver the constraining element, and a second catheter adapted to transluminally deliver the artificial coaptation element.

In accordance with some applications, there is provided a method for repairing billowing, prolapse, or flailing of a leaflet of a cardiac valve of a subject, the leaflet having a root attached to an annulus of the cardiac valve, a lip adapted to coapt with at least one other leaflet of the valve, and an intermediate region extending between the root and the lip. The method includes transluminally delivering a constraining element and an artificial coaptation element to a heart chamber, adjacent the cardiac valve.

The method can include presenting the intermediate region of the leaflet by constraining the lip of the leaflet using the constraining element.

In some applications, the method includes presenting an artificial coaptation surface for coaptation with the at least one other leaflet by mounting the artificial coaptation element onto the intermediate region of the leaflet.

In some applications, the method includes, prior to constraining the lip of the leaflet, folding the leaflet to present the intermediate region thereof.

In some applications, the constraining includes constraining the lip of the leaflet to a cardiac wall, downstream of the cardiac valve.

In some applications, the constraining element includes at least one tissue anchor, and the constraining includes anchoring the lip of the leaflet to the cardiac wall using the at least one tissue anchor.

The above method(s) can be performed on a living animal or on a simulation, such as on a cadaver, cadaver heart, simulator (e.g., with the body parts, heart, tissue, etc. being simulated), etc.

In accordance with some applications, there is provided a system for repairing billowing, prolapse, or flailing of a leaflet of a cardiac valve of a subject, the leaflet having a root attached to an annulus of the cardiac valve, a lip adapted to coapt with at least one other leaflet of the valve. The system includes at least one longitudinal catheter configured to be transluminally advanced toward a heart chamber of the subject adjacent the cardiac valve. The longitudinal catheter can have a proximal part and a steerable distal part, and a longitudinal axis therebetween.

The system includes a leaflet-engaging surface-implant including a flexible leaflet-engaging-surface and a tether.

In some applications, the at least one longitudinal catheter is adapted to transluminally deliver the leaflet-engaging surface-implant to the heart chamber. In some applications, the at least one catheter is adapted to attach the leaflet-engaging surface to a surface of the leaflet using the tether. In some applications, the at least one catheter is adapted to deform the leaflet-engaging-surface, when attached to the leaflet, thereby deforming or shorten the leaflet, along at least one dimension thereof.

In some applications, the leaflet-engaging-surface is non-elastic. In some applications, the catheter is adapted to deform the leaflet-engaging-surface by tensioning the tether attaching the leaflet-engaging-surface to the surface of the leaflet. In some applications, the catheter is further adapted to secure the tension in the tether to maintain the leaflet in the deformed or shortened orientation.

In some applications, the leaflet-engaging-surface is elastic. In some applications, the catheter is further adapted to stretch the leaflet-engaging-surface prior to attachment thereof to the surface of the leaflet, and to attach the leaflet-engaging-surface to the surface of the leaflet in the stretched position. In some applications, the catheter is adapted to deform the leaflet-engaging-surface by releasing the stretching thereof, following attachment to the surface of the leaflet.

In some applications, the at least one catheter includes at least two catheters adapted to hold opposing sides of the leaflet-engaging-surface for stretching thereof.

In accordance with some applications, there is provided a method for repairing billowing, prolapse, or flailing of a leaflet of a cardiac valve of a subject, the leaflet having a root attached to an annulus of the cardiac valve, a lip adapted to coapt with at least one other leaflet of the valve. The method includes transluminally delivering a leaflet-engaging surface implant including a tether and a leaflet-engaging-surface to a heart chamber, adjacent the cardiac valve.

In some applications, the method includes attaching the leaflet-engaging-surface to a surface of the leaflet using the tether.

In some applications, the method includes deforming the leaflet-engaging-surface, when attached to the leaflet, thereby deforming or shorten the leaflet, along at least one dimension thereof.

In some applications, the leaflet-engaging-surface is non-elastic. In some applications, the deforming of the leaflet-engaging-surface includes tensioning the tether attaching the leaflet-engaging-surface to the surface of the leaflet. In some applications, the method includes securing the tension in the tether to maintain the leaflet in the deformed or shortened orientation.

In some applications, the leaflet-engaging-surface is elastic. In some applications, the method includes stretching the leaflet-engaging-surface prior to attachment thereof to the surface of the leaflet. In some applications, the attaching includes attaching the leaflet-engaging-surface to the surface of the leaflet in the stretched position. In some applications, the deforming includes releasing the stretching of the leaflet-engaging-surface, following the attaching.

In some applications, the stretching includes, using two catheters, drawing opposing sides of the leaflet-engaging-surface away from each other.

The above method(s) can be performed on a living animal or on a simulation, such as on a cadaver, cadaver heart, simulator (e.g., with the body parts, heart, tissue, etc. being simulated), etc.

In accordance with some applications, there is provided a system for repairing billowing, prolapse, or flailing of a leaflet of a cardiac valve of a subject, the leaflet having a root attached to an annulus of the cardiac valve, a lip adapted to coapt with at least one other leaflet of the valve, the root of the leaflet being adjacent a coronary artery. The system includes at least one longitudinal catheter configured to be transluminally advanced toward a heart chamber of the subject adjacent the cardiac valve. The longitudinal catheter can have a proximal part and a steerable distal part, and a longitudinal axis therebetween.

The system includes an implant including a guidewire having first and second ends, and first and second pledgets adapted to attach to the respective first and second ends of the guidewire.

In some applications, the at least one longitudinal catheter is adapted to transluminally deliver the guidewire and the first and second pledgets to the heart chamber, via the coronary artery. In some applications, the at least one longitudinal catheter is adapted to pierce the first end of the guidewire through two positions in the leaflet, thereby to form a fold in the leaflet, the two positions and the fold being between the root of the leaflet and the lip of the leaflet. In some applications, the at least one longitudinal catheter is adapted to deploy the first and second pledgets at the first and second ends of the guidewire, while tensioning the guidewire, to maintain the fold in the leaflet and to reduce the length of the leaflet from the root to the lip.

In some applications, the system includes a pledget delivery system adapted to transluminally deliver the first pledget to the first end of the guidewire over the guidewire, via the at least one catheter and via the coronary artery.

In accordance with some applications, there is provided a method for repairing billowing, prolapse, or flailing of a leaflet of a cardiac valve of a subject, the leaflet having a root attached to an annulus of the cardiac valve, a lip adapted to coapt with at least one other leaflet of the valve, the root of the leaflet being adjacent a coronary artery. The method includes transluminally delivering to a heart chamber adjacent the cardiac valve, via the coronary artery, an implant including a guidewire having first and second ends and first and second pledgets adapted to attach to the respective first and second ends of the guidewire.

The method can include piercing the first end of the guidewire through two positions in the leaflet, thereby forming a fold in the leaflet, the two positions and the fold being between the root of the leaflet and the lip of the leaflet.

The method can include deploying the first and second pledgets at the first and second ends of the guidewire, while tensioning the guidewire, to maintain the fold in the leaflet and to reduce the length of the leaflet from the root to the lip.

The above method(s) can be performed on a living animal or on a simulation, such as on a cadaver, cadaver heart, simulator (e.g., with the body parts, heart, tissue, etc. being simulated), etc.

In accordance with some applications, there is provided a system for repairing billowing, prolapse, or flailing of a leaflet of a cardiac valve of a subject, the leaflet having a root attached to an annulus of the cardiac valve, a lip adapted to coapt with at least one other leaflet of the valve, the root of the leaflet being attached to a cardiac wall. The system includes at least one longitudinal catheter configured to be transluminally advanced toward a heart chamber of the subject adjacent the cardiac valve. The longitudinal catheter can have a proximal part and a steerable distal part, and a longitudinal axis therebetween.

The system can include a clip implant including having a clip portion defining a bend and a wire finger attached to the clip portion.

In some applications, the at least one longitudinal catheter is adapted to transluminally deliver the clip implant to the heart chamber. In some applications, the at least one catheter is adapted to mount the clip implant onto the leaflet such that the bend engages the lip of the leaflet, and the wire finger extends to a contact point between the root of the leaflet and the cardiac wall, such that the clip implant mechanically limits the leaflet from flailing.

In some applications, the wire portion is substantially U-shaped and includes two longitudinal portions connected by an end portion, and wherein the catheter is adapted to mount the clip implant such that the end portion is disposed at the contact point.

In some applications, the catheter is adapted to mount the clip implant such that the wire portion is disposed against, or adjacent, a downstream surface of the leaflet.

In accordance with some applications, there is provided a method for repairing billowing, prolapse, or flailing of a leaflet of a cardiac valve of a subject, the leaflet having a root attached to an annulus of the cardiac valve, a lip adapted to coapt with at least one other leaflet of the valve, the root of the leaflet being attached to a cardiac wall. The method includes transluminally delivering to a heart chamber adjacent the cardiac valve, via a clip implant including having a clip portion defining a bend and a wire finger attached to the clip portion.

The method can include mounting the clip implant onto the leaflet such that the bend engages the lip of the leaflet, and the wire finger extends to a contact point between the root of the leaflet and the cardiac wall, such that the clip implant mechanically limits the leaflet from flailing.

In some applications, the mounting includes mounting the clip implant such that the wire portion is disposed against, or adjacent, a downstream surface of the leaflet.

In accordance with some applications, there is provided a system for use with a leaflet of a cardiac valve of a subject. The system can include a catheter configured to be transluminally advanced toward a heart chamber of the subject adjacent the cardiac valve, the catheter having a proximal part and a steerable distal part, and a longitudinal axis therebetween.

In some applications, the system can include a device including first, second, and third beams and having a first operative state and a second operative state.

The catheter can be adapted to transluminally deliver the device to the heart chamber. In some applications, the catheter can be adapted to position the device at the leaflet in the first operative state, with the first and third beams on a first side of the leaflet, and the second beam on a second opposing side of the leaflet. In some applications, the catheter is configured to transform the device from the first operative state toward the second operative state while the first and third beams remain on the first side of the leaflet and the second beam remains on the second opposing side of the leaflet.

In some applications, at least two of the first, second, and third beams are substantially parallel to each other, in at least one of the first operative state and the second operative state.

In some applications, the device is an implant. In some applications, the implant is at least one of flexible and resilient. In some applications, the implant includes a resilient metal. In some applications, the implant includes a shape memory material. In some applications, the implant includes an elastic component.

In some applications, the catheter is adapted to position the implant onto the leaflet in the first operative state by positioning the implant such that the first, second, and third beams are substantially perpendicular to the lip of the leaflet.

In some applications, the implant includes a base, from which extend a plurality of beam including the first, second, and third beams, each of the plurality of beams having a terminus distal to the base, the first, second, and third beams being substantially parallel to one another. In some applications, in the first operative state, the terminus of one of the first, second, and third beams is disposed outside of a plane defined by the base and termini of the other two of the first, second, and third beams, the one of the first, second, and third beams being disposed between the other two of the first, second, and third beams. In some applications, in the second operative state, the base, as well as the termini of the first, second, and third beams, are in a single plane.

In some applications, the plurality of beams includes at least five beams. In some applications, a first subset of the plurality of beams includes the one of the first, second, and third beams, and a second subset of the plurality of beams includes the other two of the first, second, and third beams, the first and second subsets of the plurality of beams being mutually exclusive. In the first operative state, the plane is defined by the termini all beams in the second subset and by the base, and the termini of all beams in the first subset are disposed outside of the plane. In some applications, in the second operative state, the base, as well as the termini of the beams in the first and second subsets, are in the single plane.

In some applications, in the first operative state, the termini of all beams in the first subset are disposed in a second plane, the second plane being angled relative to the plane.

In some applications, the first, second, and third beams are substantially perpendicular to the base, in at least one of the first and second operative states.

In some applications, the catheter is adapted to slide the implant onto the lip of the leaflet, when the implant is in the first operative state, such that the base extends along the lip of the leaflet, thereby positioning the implant onto the leaflet.

In some applications, the implant includes first and second U-shaped portions, each including a base and first and second beams extending substantially perpendicularly from the base, and substantially parallel to each other. The implant can include an engaging element holding together the second beam of each of the first and second U-shaped portions to form a unified beam. In some applications, the unified beam is a central beam, disposed between the first beam of the first U-shaped portion and the first beam of the second U-shaped portion. The second beams of the first and second U-shaped portion are pivotable relative to the engaging element, about respective longitudinal axes of the second beams, such that the first and second U-shaped portions are pivotable relative to each other.

In some applications, the first and second U-shaped portions are identical to each other.

In some applications, the engaging element includes a longitudinally extending cylinder.

In some applications, in the first operative state, the first and second U-shaped portions are substantially in a single plane, and in the second operative state, the unified beam is disposed outside of a plane formed by the first beams of the first and second U-shaped portions.

In some applications, in the first operative state, a first distance exists between the first beams of the first and second U-shaped portions, and in the second operative state a second distance exists between the first beams of the first and second U-shaped portions, the second distance being smaller than the first distance.

In some applications, the catheter is adapted to rotate the first and second U-shaped portions relative to each other, thereby transforming the implant from the first operative state to the second operative state.

In some applications, the unified beam includes the second beam of the implant, and the first beams of the first and second U-shaped portions include the first and third beams of the implant, respectively.

In some applications, the implant includes a central U-shaped portion, including a pair of central beams and a connecting segment, and first and second side portions, each including at least one beam and being connected to one of the central beams by a corresponding one of first and second bends. In some applications, the at least one beam of each of the first and second side portions is substantially parallel to at least one of the central beams. In some applications, the first and second bends facilitate motion of corresponding first and second side portions, relative to the pair of central beams, in at least two directions.

In some applications, in the first operative state, the central U-shaped portion is in a first plane, and each of the first and second side portions is in plane different from the first plane, and is disposed exteriorly to the central U-shaped portion, and in the second operative state, the central U-shaped portion and the first and second side portions are in the first plane, with the first and second side portions being disposed within the central U-shaped portion.

In some applications, the implant is formed of a single length of a flexible or resilient material.

In some applications, each of the first and second side portions includes a pair of side beams, connected to each other by an additional bend, the pair of side beams being substantially parallel to each other.

In some applications, the implant is side-to-side symmetrical about a central longitudinal axis of the central U-shaped portion.

In some applications, in the first operative state, a first distance exists between the first and second side portions, and in the second operative state a second distance exists between the first and second side portions, the second distance being smaller than the first distance.

In some applications, the implant has an intermediate operative state, in which the first and second side portions are in the first plane, and are disposed exteriorly to the central U-shaped portion. In some applications, for transforming the implant from the first operative state to the second operative state, the catheter is adapted to transform the implant from the first operative state to the intermediate operative state, by pivoting the first and second side portions in a first direction relative to the central U-shaped portion, to place the first and second side portions in the first plane, and to further transform the implant from the intermediate operative state to the second operative state by revolving the first and second side portions in a second direction relative to the central U-shaped portion, to move the first and second side portions to the interior of the central U-shaped portion.

In some applications, the first direction is about an axis perpendicular to longitudinal axes of the central beams, and the second direction is about axes parallel to the longitudinal axes of the central beams.

In some applications, the U-shaped portion includes the second beam, and the first and second side portions include the first and third beams, respectively.

In some applications, the catheter is adapted to slide the implant onto the lip of the leaflet, when the implant is in the first operative state, such that the first and second bends engage the lip of the leaflet and the connecting segment of the U-shaped portion is distal to the lip of the leaflet, thereby to position the implant onto the leaflet.

In some applications, the catheter is adapted to transluminally deliver the implant to the heart chamber when the implant is in the first operative state.

In some applications, the catheter is adapted to transluminally deliver the implant to the heart chamber when the implant is in a preparatory operative state, in which the first and second side portions are in the first plane and are exterior to the central U-shaped portion. In some applications, the catheter is further adapted, prior to placing the implant onto the leaflet, to transition the implant from the preparatory operative state to the first operative state, by pivoting the first and second side portions relative to the U-shaped portion about an axis perpendicular to longitudinal axes of the central beams.

In some applications, the implant includes a tubular body arranged along a central longitudinal axis. In some applications, the implant includes first and second substantially U-shaped clips, each having a first elongate portion disposed on, or near, an exterior surface of the tubular body, and a second elongate portion disposed on, or near, an interior surface of the tubular body, the first and second elongate portions connected by an end portion. In some applications, the implant includes an elastic element holding together the second elongate portions of the first and second clips.

In some applications, in the first operative state, the clips are disposed on a first side of the tubular body, and in the second operative state the clips are disposed on a second side of the tubular body, the second side being opposite the first side.

In some applications, the catheter is adapted to slide the implant onto the lip of the leaflet, when the implant is in the first operative state, such that the end portions engage the lip of the leaflet, thereby placing the implant onto the leaflet. In some applications, the tubular body and the second elongate portions of the first and second clips forming the second beam of the implant and the first elongate portions of the first and second clips forming the first and second beams of the implant.

In some applications, the catheter is adapted to transluminally deliver the implant to the heart chamber when the implant is in the first operative state.

In some applications, the catheter is adapted to transluminally deliver the implant to the heart chamber when the U-shaped clips are separate from the tubular body. In some applications, the catheter is further adapted, prior to or during placing the implant onto the leaflet, to place the first and second clips onto the tubular body.

In some applications, the catheter is adapted to transform the implant from the first operative state to the second operative state, by drawing the first and second clips away from each other, about the perimeter of the tubular body and against the force of the elastic element.

In some applications, the catheter is adapted to form a bulge in the leaflet by transforming the device from the first operative state toward the second operative state while the first and third beams remain on the first side of the leaflet and the second beam remains on the second opposing side of the leaflet.

In some applications, the system includes an attaching device, transluminally deliverable by the catheter, and configured to at least temporarily secure two segments of the leaflet at the bulge.

In some applications, the catheter is further adapted to hold the two segments of the leaflet together to hold the bulge by moving the first beam and the third beam toward each other.

In some applications, the attaching device is configured to secure the two segments together when the two segments are held together.

In some applications, the attaching device includes a helical needle defining a helical lumen, the helical needle configured to be advanced distally out of the longitudinal catheter and to rotationally extend through, and secure together the two segments at the bulge.

In some applications, the system includes a suture extending through the helical lumen of the helical needle, wherein the suture is attached to a detachable needle tip of the helical needle and is configured to secure together the two segments at the bulge following removal of the helical needle from the heart chamber.

In some applications, the system includes a pushing-wire extending through the lumen of the helical needle and configured to push the detachable distal tip of the helical needle distally relative to the needle body, thereby to detach the detachable distal tip from the needle body.

In some applications, the system includes a cutting device configured to cut through tissue of the leaflet. In some applications, the at least one catheter is further adapted to transluminally deliver the cutting device to the heart chamber and operate the cutting device to cut a portion of the leaflet disposed between the two segments at the bulge, thereby to form cut edges.

In some applications, the cut edges are at least temporarily secured by the attaching device.

In some applications, the cutting device is adapted to extend distally out of the second beam of the device. In some applications, the at least one catheter is further adapted, following transforming of the device from the first operative state toward the second operative state and following the securing together of the two segments, to draw the device proximally, such that during the drawing of the device the cutting device cuts the tissue of the leaflet between the two segments at the bulge.

In some applications, the cutting device is further configured to clamp the cut tissue prior to complete cutting thereof, and the at least one catheter is further adapted to transluminally remove the cutting device, together with the cut tissue clamped by the cutting device, from the heart chamber.

In accordance with some applications, there is provided a method for repairing a leaflet of a cardiac valve of a subject. The method includes transluminally delivering to a heart chamber, adjacent the cardiac valve, a device including first, second, and third beams and having a first operative state and a second operative state.

In some applications, the method can include positioning the device at the leaflet in the first operative state, with the first and third beams on a first side of the leaflet, and the second beam on a second opposing side of the leaflet.

In some applications, the method can include, while the first and third beams remain on the first side of the leaflet, and the second beam remains on the second opposing side of the leaflet, transforming the device from the first operative state toward the second operative state.

In some applications, the device includes an implant, and wherein the transluminally delivering includes transluminally delivering the implant, the positioning includes positioning the implant, and the transforming includes transforming the implant.

In some applications, the positioning of the implant includes positioning the implant such that the first, second, and third beams are substantially perpendicular to a lip of the leaflet.

In some applications, the implant includes a base, from which extend a plurality of beam including the first, second, and third beams, each of the plurality of beams having a terminus distal to the base, the first, second, and third beams being substantially parallel to one another. In some applications, the positioning of the implant in the first operative state includes placing the implant such that the terminus of one of the first, second, and third beams is disposed outside of a plane defined by the base and termini of the other two of the first, second, and third beams, the one of the first, second, and third beams being disposed between the other two of the first, second, and third beams. In some applications, in the second operative state, the base, as well as the termini of the first, second, and third beams, are in a single plane. In some applications, the transforming of the implant from the first operative state toward the second operative state includes moving the termini of first, second, and third beams, relative to each other, so that the termini of the first, second, and third beams are in the single plane.

In some applications, the plurality of beams includes at least five beams. In some applications, a first subset of the plurality of beams includes the one of the first, second, and third beams, and a second subset of the plurality of beams includes the other two of the first, second, and third beams, the first and second subsets of the plurality of beams being mutually exclusive. In some applications, the positioning of the implant in the first operative state includes positioning the implant such that the plane is defined by the termini all beams in the second subset and by the base, and the termini of all beams in the first subset are disposed outside of the plane. In some applications, the transforming of the implant includes moving the termini of all the beams in the first subset such that, in the second operative state, the base, as well as the termini of the beams in the first and second subsets, are in the single plane.

In some applications, the positioning of the implant in the first operative state includes positioning the implant such that the termini of all beams in the first subset are disposed in a second plane, the second plane being angled relative to the plane.

In some applications, the positioning of the implant includes positioning of the implant while the first, second, and third beams are substantially perpendicular to the base.

In some applications, the positioning of the implant onto the leaflet includes sliding the implant onto the lip of the leaflet, when the implant is in the first operative state, such that the base extends along the lip of the leaflet.

In some applications, the implant includes first and second U-shaped portions, each including a base and first and second beams extending substantially perpendicularly from the base, and substantially parallel to each other. In some applications, the implant includes an engaging element holding together the second beam of each of the first and second U-shaped portions to form a unified beam. In some applications, the unified beam is a central beam, disposed between the first beam of the first U-shaped portion and the first beam of the second U-shaped portion. In some applications, the second beams of the first and second U-shaped portion are pivotable relative to the engaging element, about respective longitudinal axes of the second beams, such that the first and second U-shaped portions are pivotable relative to each other. In some applications, the positioning of the implant in the first operative state includes positioning the implant such that the first beams of the first and second U-shaped portions are at a first distance from each other.

In some applications, the positioning of the implant in the first operative state includes positioning the implant onto the leaflet while the first and second U-shaped portions are substantially in a single plane, and the transforming includes pivoting the first and second U-shaped portions relative to each other to move the unified beam to be disposed outside of a plane formed by the first beams of the first and second U-shaped portions.

In some applications, the transforming includes pivoting the first and second U-shaped portions relative to each other to reduce the distance between the first beams of the first and second U-shaped portions to a second distance exists between the first beams of the first and second U-shaped portions, the second distance being smaller than the first distance.

In some applications, the positioning of the implant includes sliding the implant onto the lip of the leaflet, when the implant is in the first operative state, such that the bases of the first and second U-shaped portions extend along the lip of the leaflet, the unified beam functions as the second beam and is on the second opposing side of the leaflet, and the first beams of the first and second U-shaped portions function as the first and third beams of the device and are disposed on the first side of the leaflet.

In some applications, the implant includes a central U-shaped portion, including a pair of central beams and a connecting segment. In some applications, the implant includes first and second side portions, each including at least one beam and being connected to one of the central beams by a corresponding one of first and second bends. In some applications, the at least one beam of each of the first and second side portions is substantially parallel to at least one of the central beams. In some applications, the first and second bends facilitate motion of corresponding first and second side portions, relative to the pair of central beams, in at least two directions. In some applications, the positioning of the implant in the first operative state, includes positioning the implant onto the leaflet while the central U-shaped portion is in a first plane, and each of the first and second side portions is in plane different from the first plane, and is disposed exteriorly to the central U-shaped portion. In some applications, the transforming includes moving the first and second side portions to be in the first plane, within the central U-shaped portion.

In some applications, the positioning of the implant in the first operative state includes positioning the implant onto the leaflet while a first distance exists between the first and second side portions, and the transforming of the implant includes reducing the distance between the first and second side portions to a second distance, smaller than the first distance.

In some applications, the implant has an intermediate operative state, in which the first and second side portions are in the first plane, and are disposed exteriorly to the central U-shaped portion. In some applications, the transforming of the implant from the first operative state toward the second operative state includes transforming the implant from the first operative state to the intermediate operative state, by pivoting the first and second side portions in a first direction relative to the central U-shaped portion, to place the first and second side portions in the first plane, and transforming the implant from the intermediate operative state to the second operative state by revolving the first and second side portions in a second direction relative to the central U-shaped portion, to move the first and second side portions to the interior of the central U-shaped portion.

In some applications, the pivoting in the first direction includes pivoting about an axis perpendicular to longitudinal axes of the central beams, and the revolving in the second direction includes revolving about axes parallel to the longitudinal axes of the central beams.

In some applications, the positioning of the implant includes sliding the implant onto the lip of the leaflet, when the implant is in the first operative state, such that the first and second bends engage the lip of the leaflet, and the connecting segment of the U-shaped portion is distal to the lip of the leaflet.

In some applications, the transluminally delivering includes transluminally delivering the implant to the heart chamber when the implant is in the first operative state.

In some applications, the transluminally delivering includes transluminally delivering the implant to the heart chamber when the implant is in a preparatory operative state, in which the first and second side portions are in the first plane and are exterior to the central U-shaped portion. In some applications, the method includes, prior to the placing of the implant, transitioning the implant from the preparatory operative state toward the first operative state.

In some applications, the transitioning of the implant from the preparatory state includes pivoting the first and second side portions relative to the U-shaped portion about an axis perpendicular to longitudinal axes of the central beams.

In some applications, the implant includes a tubular body arranged along a central longitudinal axis and first and second substantially U-shaped clips, each having a first elongate portion disposed on, or near, an exterior surface of the tubular body, and a second elongate portion disposed on, or near, an interior surface of the tubular body, the first and second elongate portions connected by an end portion. In some applications, the implant includes an elastic element holding together the second elongate portions of the first and second clips. In some applications, in the first operative state, the clips are disposed on a first side of the tubular body, and in the second operative state the clips are disposed on a second side of the tubular body, the second side being opposite the first side. In some applications, the positioning of the implant includes sliding the implant onto the lip of the leaflet, when the implant is in the first operative state, such that the end portions engage the lip of the leaflet, with the tubular body and the second elongate portions of the first and second clips functioning as the second beam, and the first elongate portions of the first and second clips functioning as the first and third beams.

In some applications, the transluminally delivering includes transluminally delivering the implant to the heart chamber when the implant is in the first operative state.

In some applications, the transluminally delivering includes transluminally delivering the implant to the heart chamber when the U-shaped clips are separate from the tubular body. In some applications, the method includes, prior to or during the positioning of the implant, sliding the first and second clips onto the tubular body to form the first operative state.

In some applications, the transforming of the implant includes drawing the first and second clips away from each other, about the perimeter of the tubular body and against the force of the elastic element.

In some applications, the transforming of the device from the first operative state toward the second operative state includes forming a bulge in the leaflet while the first and third beams remain on the first side of the leaflet and the second beam remains on the second opposing side of the leaflet. In some applications, the method includes, using an attaching device, at least temporarily securing two segments of the leaflet at the bulge.

In some applications, the method includes holding the two segments of the leaflet together to hold the bulge by moving the first beam and the third beam toward each other.

In some applications, the at least temporarily securing is carried out while the two segments of the leaflet are held together.

In some applications, the method includes transluminally delivering the attaching device to the heart chamber adjacent the cardiac valve.

In some applications, the attaching device includes a helical needle defining a helical lumen, and the at least temporarily securing includes rotationally advancing the helical needle to extend through, and secure together, the two segments at the bulge.

In some applications, a suture extends through the helical lumen of the helical needle, wherein the suture is attached to a detachable needle tip of the helical needle. In some applications, the method includes, following the rotationally advancing of the helical needle anchoring the detachable distal tip of the helical needle to tissue of the leaflet. In some applications, the method includes detaching the detachable distal tip of the helical needle from a main body of the helical needle. In some applications, the method includes helically retracting the main body of the helical needle away from the detachable distal tip while sliding the helical needle over the suture, thereby removing the helical needle from the anatomical site while leaving the suture in the anatomical site securing the two segments at the bulge.

In some applications, the detaching of the detachable distal tip includes, using a pushing-wire extending through the lumen of the helical needle, pushing the detachable distal tip of the helical needle distally relative to the needle body.

In some applications, the method includes transluminally delivering a cutting device to the heart chamber. In some applications, the method includes using the cutting device, cutting a portion of the leaflet disposed between the two segments at the bulge, thereby forming cut edges.

In some applications, the at least temporarily securing includes at least temporarily securing the cut edges.

In some applications, the transluminally delivering of the cutting device includes advancing the cutting device distally out of the second beam of the device, and the cutting includes drawing the device proximally such that during the drawing of the device the cutting device cuts the tissue of the leaflet between the two segments at the bulge.

In some applications, the method includes, prior to completion of the cutting of the tissue between the two segments, clamping the cut tissue in a clamping end of a clamping device, and removing the clamping device and the cut tissue clamped thereto from the cardiac chamber.

In some applications, the method includes, prior to clamping, advancing the clamping device distally out of the second beam of the device.

In some applications, the cutting device functions as the clamping device, and the advancing of the cutting device includes advancing of the clamping device.

In accordance with some applications, there is provided a system for use with a leaflet of a cardiac valve of a subject. The system includes a catheter configured to be transluminally advanced toward a heart chamber of the subject adjacent the cardiac valve, the catheter having a proximal part and a steerable distal part, and a longitudinal axis therebetween.

In some applications, the system includes a cutting device, advanceable distally out of the catheter, adapted to cut through tissue of a leaflet of the cardiac valve thereby to form cut tissue and cut edges at the cardiac valve.

In some applications, the system can include a helical needle defining a helical lumen, the helical needle configured to be advanced distally out of the catheter and to rotationally extend through, and secure together, two tissue segments of the leaflet.

In some applications, the system includes a suture extending through the helical lumen of the helical needle.

In some applications, the helical needle and the suture are configured to extend along the two tissue segments of the leaflet to suture the two tissue segments to each other.

In some applications, the cutting device includes a wire forming a wire loop having a closed distal end, and a tightening element slidably coupled to the wire such that distal sliding of the tightening element over the wire causes contraction of the wire loop and cutting of tissue disposed within the wire loop, thereby forming the cut tissue and the cut edges. In some applications, the helical needle and the suture are configured to secure together the cut edges.

In some applications, the helical needle includes a needle body terminating at a detachable distal tip, the detachable distal tip being configured to be anchored to tissue at the cardiac valve and to detach from the needle body.

In some applications, a distal end of the suture is attached to the detachable distal tip of the helical needle.

In some applications, following detachment of the detachable distal tip of the helical needle from the needle body, the needle body is adapted to be retracted helically away from the detachable distal tip while sliding over and along the suture.

In some applications, the system can include a pushing-wire extending through the helical lumen of the helical needle and configured to push the detachable distal tip of the helical needle distally relative to the needle body, thereby to detach the detachable distal tip from the needle body.

In some applications, the cutting device and the helical needle are adapted to be removed from the heart chamber following cutting of the tissue by the cutting device, while leaving the suture in the leaflet of the cardiac valve securing the two tissue segments.

In some applications, the system can include a clamp having a distal clamping end, the clamp being advanceable distally out of the catheter to engage the cut tissue, prior to complete detachment of the cut tissue from the leaflet.

In some applications, the clamp is adapted to be removed from the heart chamber, with the cut tissue clamped thereto during or following removal of the cutting device and the helical needle from the heart chamber.

In some applications, the clamp is configured to be advanced through the catheter alongside the helical needle.

In some applications, the cutting device functions as the clamp.

In some applications, the system can include a holding device including first, second, and third beams, and having a first operative state and a second operative state, the securing device being advanceable distally out of the catheter. In some applications, the catheter is adapted to transluminally deliver the securing device to the heart chamber. In some applications, the catheter is adapted to position the device at the leaflet in the first operative state, with the first and third beams on a first side of the leaflet, and the second beam on a second opposing side of the leaflet. In some applications, the catheter is adapted, while the first and third beams remain on the first side of the leaflet, and the second beam remains on the second opposing side of the leaflet, to transform the device from the first operative state toward the second operative state.

In some applications, the cutting device is adapted to be advanced distally out of the second beam of the holding device. In some applications, the catheter is further adapted, following transforming of the holding device from the first operative state toward the second operative state and following the securing together of the two tissue segments, to draw the holding device proximally, such that during the drawing of the holding device the cutting device cuts the tissue of the leaflet between the two segments at the bulge.

In some applications, the catheter is adapted to form a bulge in the leaflet by transforming the holding device from the first operative state toward the second operative state. In some applications, the helical needle and the suture are adapted to secure the two tissue segments of the leaflet at the bulge.

In some applications, the catheter is further adapted to hold the two segments of the leaflet together to hold the bulge by moving the first beam and the third beam toward each other.

In some applications, the helical needle is configured to secure the two tissue segments together when the two tissue segments are held together.

In accordance with some applications, there is provided a method for repairing a leaflet of a cardiac valve of a subject. In some applications, the method includes securing together two segments of tissue of the leaflet of the cardiac valve, and excising tissue from between the two segments of tissue of the leaflet, with a cutting device, to form cut edges.

In some applications, the excising occurs before the securing, and the securing includes securing the cut edges as the cut edges are formed, and prior to complete detachment of the tissue from the valve leaflet.

In some applications, the excising is carried out using a cutting wire forming a wire loop, advanceable distally out of a catheter, and includes sliding a tightening element distally over the wire to cause contraction of the wire loop and cutting of tissue disposed within the wire loop, thereby to form the cut edges.

In some applications, the securing occurs before the excising, and the excising includes excising the tissue from between the secured segments, to form exposed cut edges.

In some applications, the method includes transluminally delivering to a heart chamber, adjacent the cardiac valve, a device including first, second, and third beams and having a first operative state and a second operative state.

In some applications, the method includes positioning the device at the leaflet in the first operative state, with the first and third beams on a first side of the leaflet, and the second beam on a second opposing side of the leaflet.

In some applications, the method includes, while the first and third beams remain on the first side of the leaflet, and the second beam remains on the second opposing side of the leaflet, transforming the device from the first operative state toward the second operative state, thereby forming a bulge in the leaflet.

In some applications, the securing together of the two segments includes securing together the two segments at the bulge.

In some applications, the securing together of the two segments includes advancing a helical needle through the two segments.

In some applications, the method includes, prior to the excising, advancing the cutting device to the cardiac valve via a transluminal catheter.

In some applications, the helical needle includes a helical lumen and has a suture extending through the helical lumen, the method including removing the helical needle from the cardiac valve while leaving the suture attaching the two segments.

In accordance with some applications, there is provided a system for use with a cardiac valve of a subject, the system including a catheter configured to be transluminally advanced toward a heart chamber of the subject adjacent the cardiac valve, the catheter having a proximal part and a steerable distal part, and a longitudinal axis therebetween.

The system can include a helical implant defining a pair of turns, wherein in a first operative state of the helical implant the pair of turns has a first pitch and in a second operative state of the helical implant the pair of turns has a second pitch, the second pitch being smaller than the first pitch.

In some applications, the catheter is adapted to transluminally deliver the implant to the heart chamber. In some applications, the catheter is adapted to place the helical implant, in the first operative state, onto tissue of the cardiac valve. In some applications, the catheter is adapted, while the helical implant remains in the first operative state, to draw the tissue between the turns of the pair. In some applications, the catheter is adapted, while the tissue remains between the turns of the pair, to transform the helical implant from the first operative state toward the second operative state, thereby plicating the tissue.

In some applications, the catheter is adapted to place the helical implant, in the first operative state, onto the annulus of the cardiac valve.

In some applications, the at least one helical implant includes a plurality of helical implants. In some applications, the catheter is adapted to transluminally deliver, place, draw, and transform each of the plurality of helical implants at tissue in a different location along the annulus of the cardiac valve.

In some applications, the system includes a vacuum generator functionally associated with the catheter, and wherein the vacuum generator is configured to generate a vacuum drawing the tissue of the cardiac valve between the turns of the pair.

In some applications, the helical implant is at least one of flexible and resilient. In some applications, the helical implant includes a resilient metal. In some applications, the helical implant includes a shape memory material.

In some applications, the system includes a diaphragm disposed between a distal end of the catheter and the vacuum generator, the diaphragm adapted to prevent flow of fluids into a proximal end of the catheter during operation of the vacuum generator.

In some applications, a distal end of the catheter is partially cut away to define an implant-deployment portal, such that during placement of the helical implant, a distal portion of the implant is disposed within the implant-deployment portal and is in contact with tissue of the cardiac valve, and a proximal portion of the helical implant is separated from the tissue of the cardiac valve by the catheter.

In accordance with some applications, there is provided a method for repairing a leaflet of a cardiac valve of a subject, the method including transluminally delivering to a heart chamber, adjacent the cardiac valve, a helical implant defining a pair of turns, wherein in a first operative state of the helical implant the pair of turns has a first pitch and in a second operative state of the helical implant the pair of turns has a second pitch, the second pitch being smaller than the first pitch.

In some applications, the method can include placing the helical implant, in the first operative state, onto tissue of the cardiac valve.

In some applications, the method can include, while the implant remains in the first operative state, drawing the tissue between the turns of the pair, and while the tissue remains between the turns of the pair, transforming the helical implant from the first operative state toward the second operative state, thereby plicating the tissue.

In some applications, the placing includes placing the helical implant, in the first operative state, onto the annulus of the cardiac valve.

In some applications, the steps of delivering, placing, drawing, and transforming are carried out for each of a plurality of the helical implants, wherein the placing includes placing each of the plurality of helical implants in a different location along the annulus of the cardiac valve.

In some applications, the drawing includes generating a vacuum drawing the tissue of the cardiac valve between the turns of the pair.

In some applications, the method includes placing a diaphragm between the helical implant and a vacuum generator generating the vacuum, thereby to prevent flow of fluids from the cardiac valve into the vacuum generator during operation of the vacuum generator.

Any of the above method(s) can be performed on a living animal or on a simulation, such as on a cadaver, cadaver heart, simulator (e.g., with the body parts, heart, tissue, etc. being simulated), etc.

### BRIEF DESCRIPTION OF THE FIGURES

The foregoing discussion will be understood more readily from the following detailed description when taken in conjunction with the accompanying Figures, in which:

FIG. 1 is a schematic illustration of a mitral valve of a heart, having a prolapsed leaflet in need of repair;

FIG. 2 is a schematic side view illustration of placement of a tissue anchor in the mitral valve shown in FIG. 1, in accordance with some applications;

FIGS. 3A and 3B are, respectively, a schematic side view illustration and a schematic top view illustration of placement of a cutting wire around excess tissue of a heart valve leaflet, in accordance with some applications;

FIGS. 4A and 4B are, respectively, a schematic side view illustration and a schematic top view illustration of tightening of the cutting wire around the excess tissue for cutting thereof, while securing together cut edges of the tissue, in accordance with some applications;

FIGS. 5A and 5B are, respectively, a schematic side view illustration and a schematic top view illustration of continued tightening of the cutting wire and cutting of the excess tissue and continued securing of the cut edges, while a clamp holds the cut tissue, in accordance with some applications;

FIGS. 6A and 6B are, respectively, a schematic side view illustration and a schematic top view illustration of continued tightening of the cutting wire and cutting of the excess tissue and continued securing of the cut edges, while the clamp continues holding the cut tissue, in accordance with some applications;

FIGS. 7A and 7B are, respectively, a schematic side view illustration and a schematic top view illustration of complete detachment of the clamped excess tissue, in accordance with some applications;

FIGS. 8A, 8B, 8C, and 8D are schematic side view illustrations, and FIG. 8E, is a schematic top view illustration, of phases of suturing the secured cut edges and removal of the cutting wire, in accordance with some applications;

FIG. 9 is a schematic illustration of the mitral valve of FIG. 1 following repair thereof using the method of FIGS. 3A to 8E;

FIGS. 10A and 10B are schematic perspective view illustrations of examples of control handles for controlling a device useful for implementing the method of FIGS. 3A to 8E, in accordance with some applications;

FIGS. 11A to 11D are schematic illustrations of an implant system, and of phases of use thereof, for repairing a leaflet of the mitral valve by suturing a portion of the leaflet between the root and lip of the leaflet, in accordance with some applications;

FIGS. 12A, 12B, and 12C are schematic top view or sectional illustrations of phases of repairing a leaflet of the mitral valve by suturing a portion of the leaflet to the root of the leaflet, in accordance with some applications;

FIG. 13 is a schematic sectional illustration of repairing a leaflet of the mitral valve by suturing a portion of the leaflet to a coronary artery surrounding the mitral valve, in accordance with some applications;

FIGS. 14A to 14D are schematic illustrations of an implant, and phases of use thereof, for repairing a leaflet of a heart valve, in accordance with some applications;

FIGS. 15A, 15B, and 15C are schematic top view illustrations of phases of repairing a leaflet of the mitral valve using an implant, in accordance with some applications;

FIGS. 16A to 16D are schematic illustrations of an implant, and phases of use thereof, for repairing a leaflet of a heart valve, in accordance with some applications;

FIGS. 17A to 17D are schematic top view illustrations of phases of repairing a leaflet of the mitral valve using an implant, in accordance with some applications;

FIGS. 18A to 18C are schematic illustrations of an implant, and phases of use thereof, for repairing a leaflet of a heart valve, in accordance with some applications;

FIGS. 19A to 19C are schematic illustrations of an implant, and phases of use thereof, for repairing a leaflet of a heart valve, in accordance with some applications;

FIG. 20 is a schematic view illustration of phases of placing an implant onto a leaflet of a cardiac valve, thereby to repair the leaflet, in accordance with some applications;

FIGS. 21A to 21D are schematic illustrations of an implant, and phases of use thereof, for repairing a leaflet of a heart valve, in accordance with some applications;

FIGS. 22A to 22C are schematic illustrations of an implant, and phases of use thereof, for repairing a leaflet of a heart valve, in accordance with some applications;

FIGS. 23A to 23C are schematic illustrations of an implant, and phases of use thereof, for repairing a leaflet of a heart valve, in accordance with some applications;

FIGS. 24A to 24D are schematic illustrations of an implant, and phases of use thereof, for repairing a leaflet of a heart valve, in accordance with some applications;

FIGS. 25A to 25C are schematic illustrations of an implant, and phases of use thereof, for repairing a leaflet of a heart valve, in accordance with some applications;

FIGS. 26A to 26C are schematic illustrations of an implant, and phases of use thereof, for repairing a leaflet of a heart valve, in accordance with some applications;

FIGS. 27A to 27E are schematic sectional illustrations of phases of repairing a leaflet of the mitral valve using an anchoring implant, in accordance with some applications;

FIGS. 28A to 28D are schematic sectional illustrations and a top view illustration of phases of repairing a leaflet of the mitral valve, in accordance with some applications;

FIGS. 29A and 29B are schematic top view illustrations of phases of repairing a leaflet of the mitral valve using an implant, in accordance with some applications;

FIGS. 30A and 30B are schematic top view illustrations of phases of repairing a leaflet of the mitral valve using an implant, in accordance with some applications;

FIGS. 31A to 31E are schematic sectional illustrations of phases of repairing a leaflet of the mitral valve using an implant, in accordance with some applications;

FIGS. 32A to 32D are schematic illustrations of an implant, and phases of use thereof, for repairing a leaflet of a heart valve, in accordance with some applications;

FIG. 33 is a schematic illustration of a native valve of a heart, having a prolapsed leaflet in need of repair;

FIG. 34 is a schematic illustration of placement of a holding device in the native valve shown in FIG. 33, in accordance with some applications;

FIGS. 35A and 35B are schematic illustrations of phases of transforming the holding device of FIG. 34 from a first operative state toward a second operative state, in accordance with some applications;

FIGS. 36A and 36B are schematic illustrations of phases of securing together segments of the tissue of mitral valve using a helical needle, in accordance with some applications;

FIG. 37 is a schematic illustration showing detachment of a needle tip of the helical needle of FIGS. 36A and 36B, in accordance with some applications;

FIG. 38 is a schematic illustration of retraction of the helical needle of FIGS. 36A and 36B, while continuing to secure the segments of the tissue of the mitral valve, in accordance with some applications;

FIGS. 39A, 39B, and 39C are schematic illustrations of phases of cutting of excess tissue between the secured segments of the tissue of the mitral valve, in accordance with some applications;

FIGS. 40A and 40B are schematic illustrations of clamping and removal of the cut tissue of the mitral valve, in accordance with some applications;

FIG. 41 is a schematic illustration of the mitral valve of FIG. 33 following repair thereof using the method of FIGS. 34 to 40B;

FIG. 42 is a schematic illustration of a mitral valve and a tricuspid valve of a heart, both having leaflets that do not coapt properly and being in need of repair, and of advancement of a catheter to the mitral valve, in accordance with some applications;

FIG. 43 is a schematic illustration of insertion of the catheter of FIG. 42 into the mitral valve in need of repair, in accordance with some applications;

FIG. 44 is a schematic illustration of arrangement of a distal end of the catheter of FIGS. 42 and 43 adjacent the annulus of a native valve, in accordance with some applications;

FIGS. 45A and 45B are schematic illustrations of placing of a helical implant onto the annulus of a native valve, and drawing tissue of the native valve into spaces between turns of the helical implant, in accordance with some applications;

FIG. 46 is a schematic illustration of transforming the shape of the helical implant of FIGS. 44 and 45, thereby plicating the tissue of the annulus, in accordance with some applications; and

FIGS. 47A and 47B are schematic illustrations of the mitral valve and of the tricuspid valve, following repair thereof using multiple helical implants and using the method of FIGS. 43 to 46, in accordance with some applications.

### DETAILED DESCRIPTION

The principles of the devices and methods for repair of a leaflet of a heart valve of a subject, and specifically the devices and methods for transcatheter or transluminal removal of excess tissue from a leaflet of a heart valve of a subject, may be better understood with reference to the drawings and the following description.

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features can be omitted or simplified in order not to obscure the disclosure. Additionally, in order to avoid undue clutter from having too many reference numbers and lead lines on a particular drawing, some elements may not be explicitly identified in every drawing that contains that element.

It is to be understood that the scope of the invention is not limited in its application to the details of construction and the arrangement of the components set forth in the following description or illustrated in the drawings. Applications of the invention are capable of other implementations or of being practiced or carried out in various ways. Furthermore, it is to be understood that the phraseology and terminology employed in the disclosure is for the purpose of description and should not be regarded as limiting.

For the purposes of this application, the term “subject” relates to any mammal, particularly humans.

For the purposes of this application, the term “cardiac wall” relates to any wall of the heart.

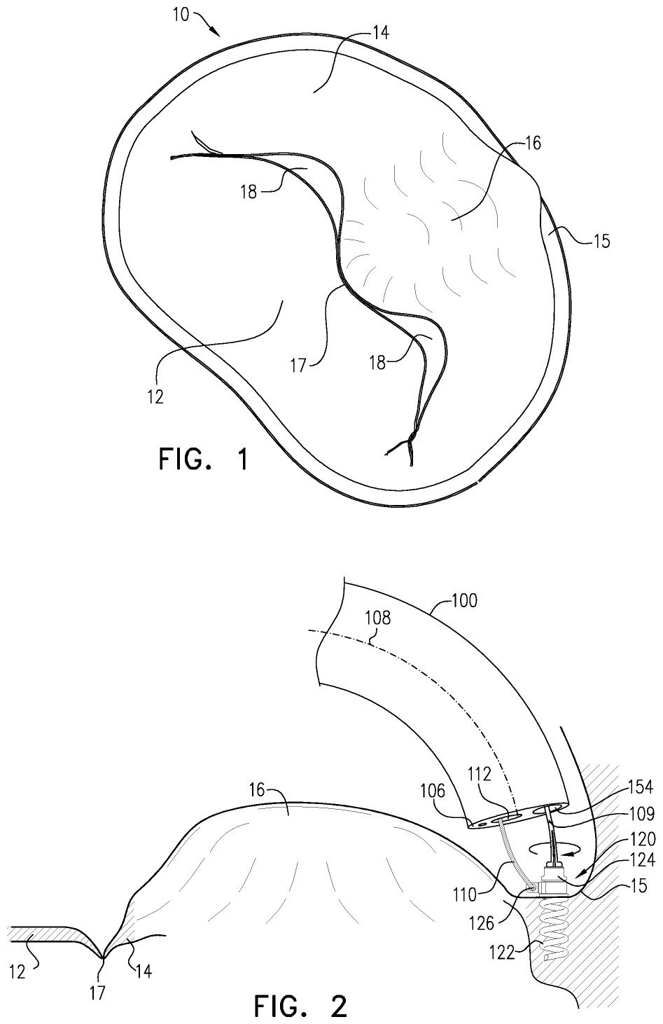

Referring now to the drawings, excess tissue in the leaflet, and improper closing thereof, are schematically shown in FIG. 1. Specifically, FIG. 1 shows a mitral valve 10 having a first leaflet 12 and a second leaflet 14, surrounded by annulus 15. Second leaflet 14 has excess tissue 16, such that the leaflets coapt at point (or edge) 17, with gaps 18 existing in the valve when it is closed.

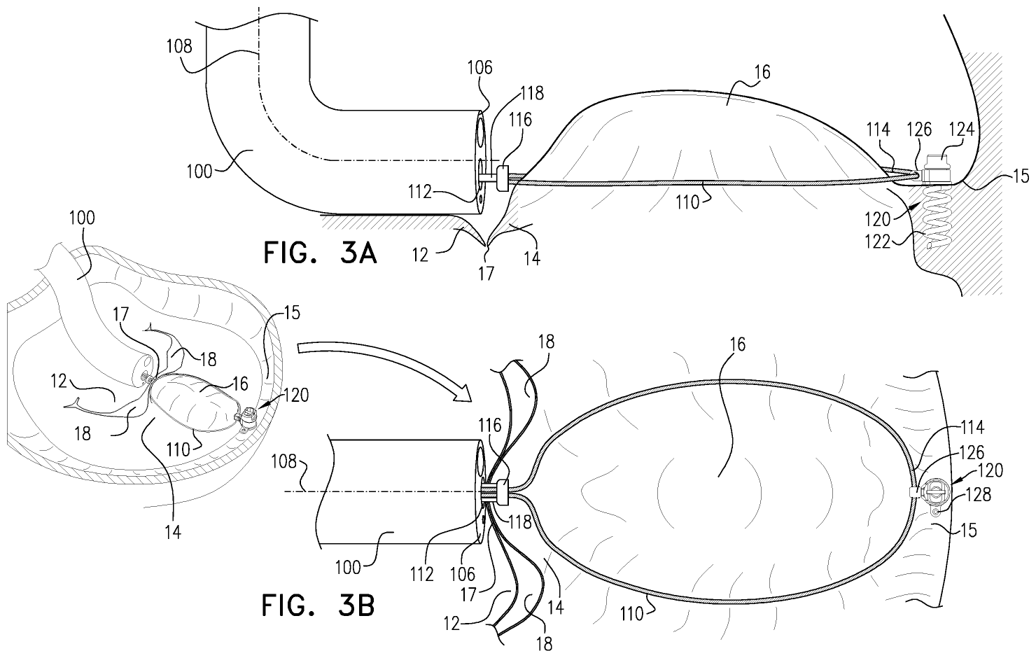

FIG. 2 is a schematic side view illustration of placement of a tissue anchor 120 in the mitral valve 10 shown in FIG. 1, in accordance with some applications.

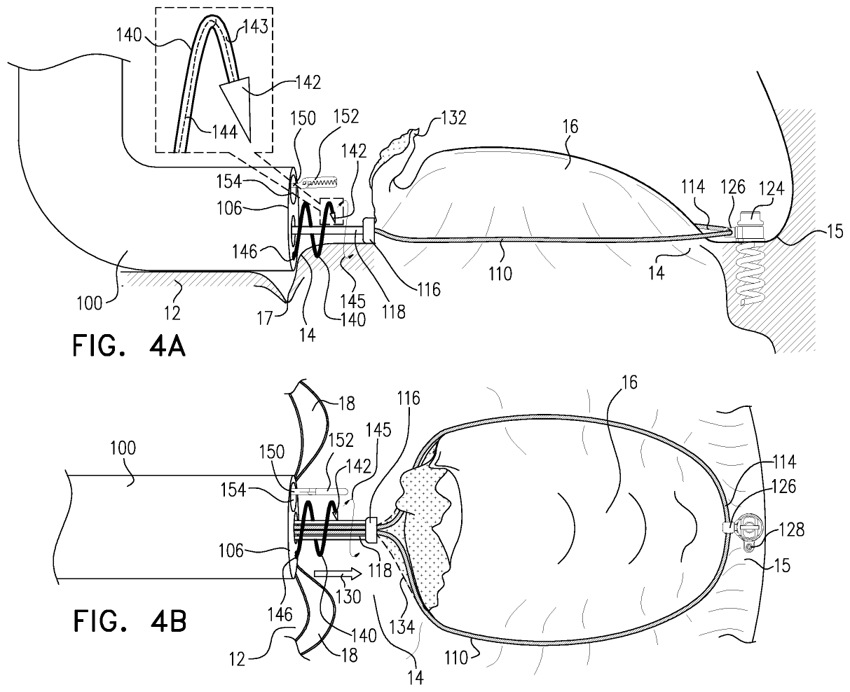

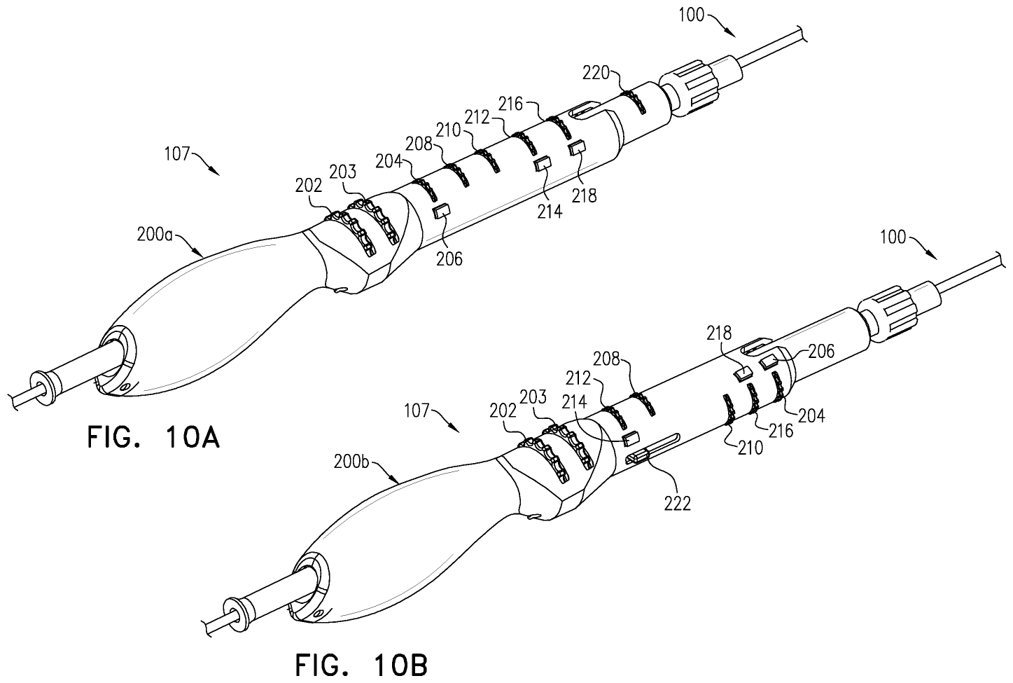

As seen in FIG. 2, a longitudinal catheter 100 is advanced toward an anatomical site of the subject. In the illustrated example, a distal part 106 of the catheter is advanced to an atrium (e.g., a left atrium) of the heart, to be positioned upstream of a heart valve (e.g., a mitral valve) having a first leaflet 12 and a second leaflet 14, surrounded by an annulus 15. Leaflet 14 has prolapsed or excess tissue 16. Gaps 18 exist between leaflets 12 and 14 at point (or edge) 17, where the leaflets should coapt (seen clearly in FIG. 3B). Longitudinal catheter 100 also has an extracorporeal proximal part 107 (e.g., as shown in FIGS. 10A-B). Distal part 106 is guidable to the anatomical site, such as by being actively steerable itself (e.g., by being operatively coupled by one or more pullwires to proximal part 107, such as to a steering controller 203 thereof shown in FIGS. 10A and 10B), or by being passively guided and/or steered (e.g., by extending over or through another steerable element, such as an actively steerable catheter). A longitudinal axis 108 extends between proximal part 107 and steerable distal part 106 of catheter 100. Catheter 100 is transluminally advanced to the anatomical site using any method known in the art, for example via the vena cava and, if necessary, via the septum separating the atria of the heart.

In some applications, and as shown, a tissue anchor 120 is advanced to the anatomical site and driven into the tissue of annulus 15, for example by a driving tool 109. As shown, driving tool 109 may extend distally from a lumen of catheter 100, here shown as lumen 154. In some applications, driving tool 109 is rotatable, such that rotation of driving tool 109 screws a portion of tissue anchor 120 into the tissue of annulus 15.

Tissue anchor 120 includes a tissue-engaging element 122, inserted into the tissue of annulus 15, and a head 124 extending above the tissue at the anatomical site. A wire 110 is coupled to a first-device anchoring point 126 of head 124, such that tissue anchor 120 anchors the wire, as explained in further detail hereinbelow. For some applications, and as shown, first-device anchoring point 126 defines an eyelet, and wire 110 is reversibly coupled to the eyelet by being threaded through the eyelet.

In some applications, and as shown, the tissue anchor is anchored on the opposite side of excess tissue 16 from the lip of the leaflet, so as to facilitate positioning of wire 110 around the excess tissue, as explained in further detail herein.

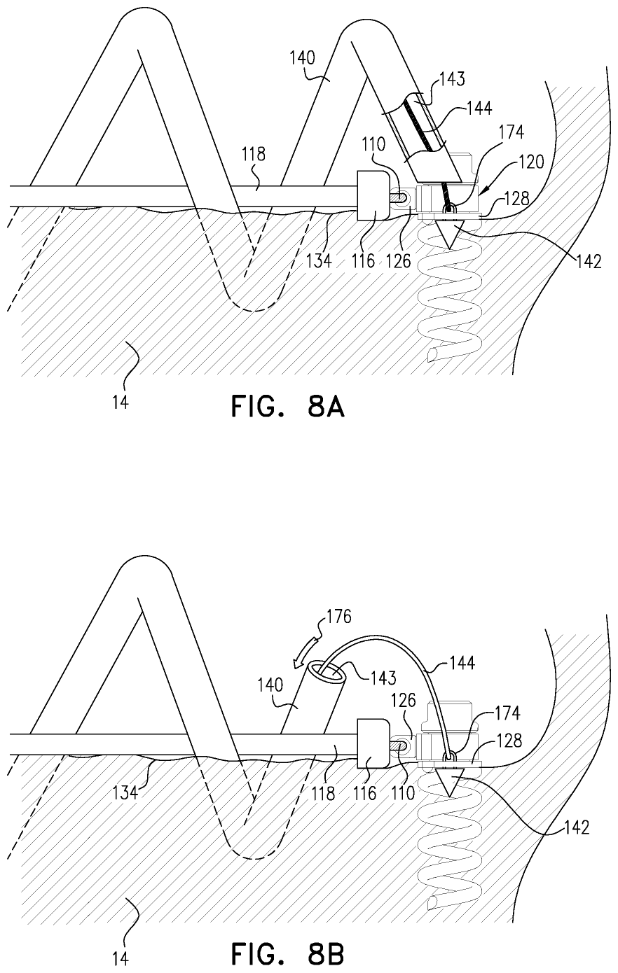

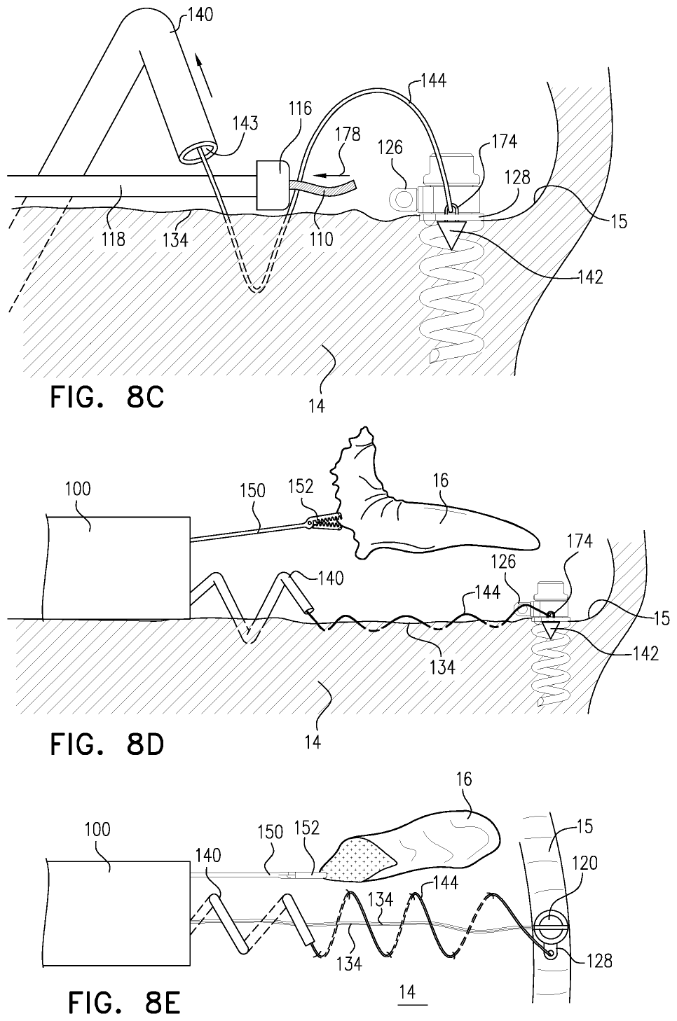

For some applications, and as shown, tissue anchor 120 includes a second-device anchoring point 128 (see FIG. 3B), which is described in further detail hereinbelow with respect to FIGS. 7A to 8E.

Following anchoring of tissue anchor 120 to annulus 15, driving tool 109 can be retracted from the anatomical site, for example via lumen 154 of catheter 100.

FIGS. 3A and 3B are, respectively, a schematic side view illustration and a schematic top view illustration of transcatheter placement of a cutting wire around excess tissue of a heart valve leaflet, in accordance with a step of some applications.

As seen in FIGS. 3A and 3B, wire 110 is advanced distally out of steerable distal part 106 of catheter 100, and forms a wire loop. Herein, the reference numeral 110 relates interchangeably to the wire and to the wire loop formed of the wire.

In some applications, such as the illustrated example, wire loop 110 can be advanced to the target anatomical site through a lumen 112 of catheter 100. In some applications, the wire loop can be advanced to the target location attached to distal part 106 of catheter 100. Wire loop 110 includes a closed distal end 114, which is anchored to first-device anchoring point 126 of tissue anchor 120, as described hereinabove.

A tightening element 116 is slidably coupled to the wire loop, such that sliding of tightening element 116 distally over wire 110 causes contraction of the wire loop and cutting of tissue that is disposed within the wire loop. Stated differently, when tightening element 116 is moved along wire 110, the area of the wire loop is reduced, which results in the wire cutting tissue that is engaged by the wire loop. As such, wire loop 110 functions as a cutting device for cutting tissue at the anatomical site.

For some applications, wire loop 110 can be replaced by a different cutting device, mutatis mutandis.

In some applications, tightening element 116 is functionally associated with a driving element 118 (e.g., for driving motion of the tightening element). In the illustrated example, driving element 118 is shown as a driving shaft, tube, or channel.

As seen in FIGS. 3A and 3B, wire loop 110 is placed around the excess tissue 16 of leaflet 14 of the heart valve.

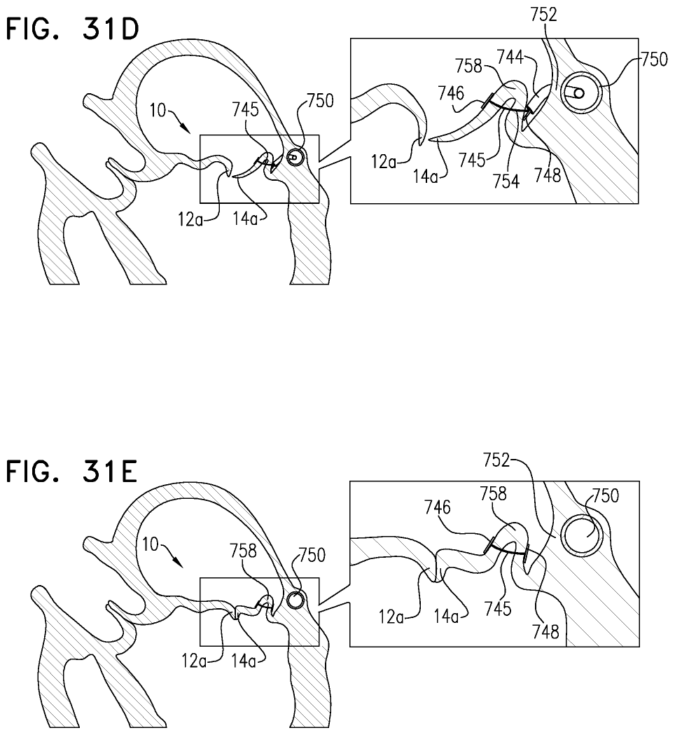

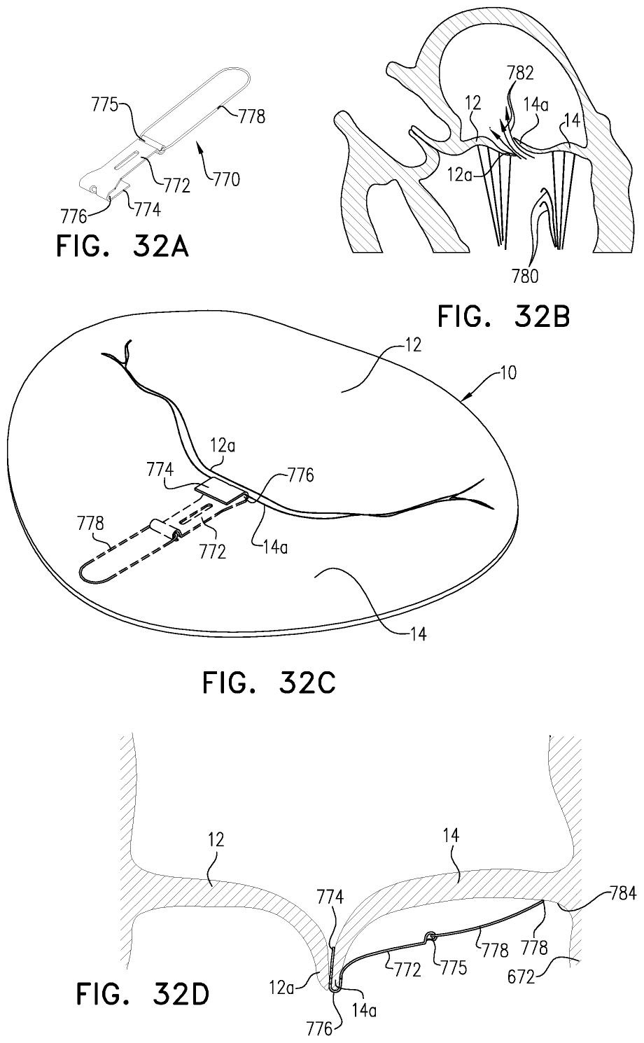

For some applications, prior to placement of wire loop 110 around excess tissue 16, the excess tissue can be formed into a bulge, for example using a holding device and/or in accordance with the method described hereinbelow with respect to FIGS. 34 to 35B.

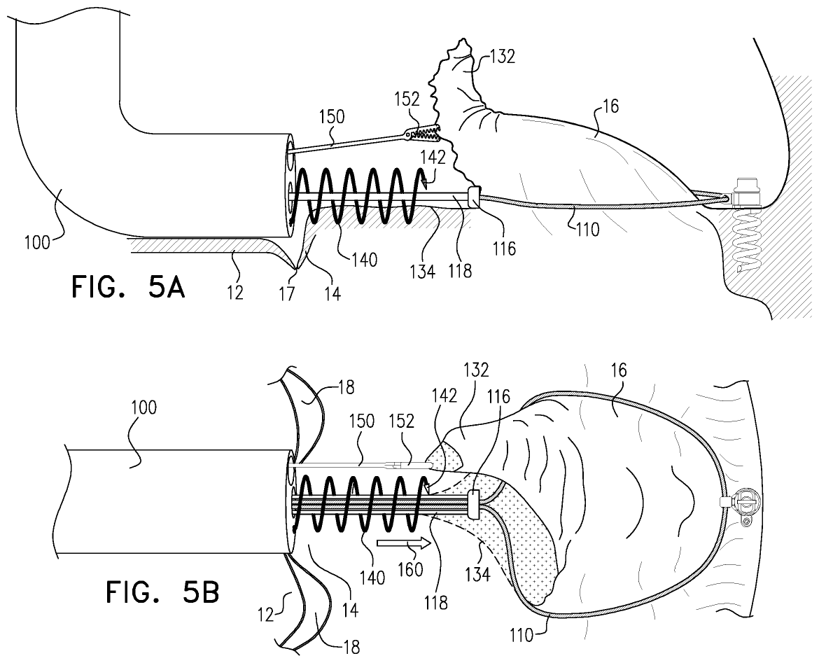

Reference is now made to FIGS. 4A and 4B, which are, respectively, a schematic side view illustration and a schematic top view illustration of initial tightening of wire loop 110 around excess tissue 16 for cutting thereof, while an edge-attaching device holds together cut edges of the tissue, in accordance with a further step of some applications.

As seen, tightening element 116 of wire loop 110 is moved forward in the direction of arrow 130, shown clearly in FIG. 4B. As a result, a portion 132 of the excess tissue 16 is cut from leaflet 14, forming cut edges 134 at the leaflet.

An edge-attaching device, here shown as a helical needle 140 having a distal needle tip 142 is rotationally extended distally out of distal part 106 of catheter 100, about the cut edges 134. As a result, cut edges 134 are secured together. Helical needle 140 includes a helical lumen 143, which has a suture 144 extending helically therethrough, as described in further detail hereinbelow. The direction of rotation of needle 140 is shown by reference numeral 145. As helical needle 140 rotates, it catches cut edges 134 from above and from below, and in a sense surrounds the cut edges, thus securing them together. Helical needle 140 can, as it rotates, draw cut edges 134 together. In some applications, helical needle can secure cut edges 134 together temporarily, and a permanent attachment element, such as suture 144, can chronically maintain the attachment of the cut edges following removal of helical needle 140, as described in further detail hereinbelow.

In some applications, needle 140 can be advanced to leaflet 14 through catheter 100. In some applications, needle 140 can be advanced through the same lumen as wire loop 110. In some applications, such as the illustrated example, needle 140 can be advanced through a dedicated lumen 146 in catheter 100. In some applications, helical needle 140 is advanced about wire 110, such that a linear portion of the wire, proximal to tightening element 116, as well as driving element 118 driving the tightening element, extend axially through the center of the helix of helical needle 140. Lumen 146 can be a helical lumen, or can be a linear lumen. In examples in which lumen 146 is linear, needle 140 automatically becomes helical upon exposure out of the lumen, for example by use of shape memory materials for the helical needle.

In some applications, helical needle 140 is operatively coupled to tightening element 116, for example as described further hereinbelow with respect to FIGS. 10A and 10B. In some applications, tip 142 of helical needle 140 remains at a fixed distance from tightening element 116, at least during cutting of the tissue and securing together of cut edges 134 as they are formed. In some applications, advancement of tightening element 116, and further cutting of excess tissue 16, is operably coupled to, or dependent on, concurrent advancement of helical needle 140, to secure together cut edges 134 as they are formed by wire loop 110, for example as described further hereinbelow with respect to FIGS. 10A and 10B.

In some applications, tightening element 116 can be advanced distally over the linear portion of wire 110 only when followed by advancement of helical needle 140. It is hypothesized that, for some applications, such operable coupling facilitates the securing together of cut edges 134 by reducing a likelihood of the cut edges moving apart from each other (e.g., by a distance that is greater than the transverse diameter of the helix of helical needle 140) before the needle has engaged (e.g., pierced) the leaflets.

Although helical needle 140 is shown as having the shape of a cylindrical helix, i.e., having a constant transverse diameter along its length, for some applications the transverse diameter of the helical needle can vary along its length, such as being wider at the distal end than at the proximal end, e.g., it can have the shape of a conical helix. It is hypothesized that, for some applications, a wider (e.g., flared) distal end can facilitate engagement of cut edges 134 and/or drawing together of the cut edges.

Although helical needle 140 is shown as having a constant pitch, for some applications, the helical needle can have a variable pitch.

For some applications, helical needle 140 can be replaced by a different edge-attaching device, mutatis mutandis. For example, alternate edge-attaching devices can include clamps, pins, hooks, tissue adhesive, and the like.

A clamp 150, having a clamping end 152, is advanced distally out of distal part 106 of catheter 100, typically alongside and laterally from helical needle 140 and wire loop 110. As will be described in further detail hereinbelow, clamp 150 is configured to engage excess tissue 16 (e.g., cut portion 132 thereof).

In some applications, and as shown, clamp 150 can be advanced to leaflet 14 through catheter 100, for example through lumen 154. In some applications, catheter 100 can include a single lumen, such that wire loop 110, helical needle 140, and clamp 150 are all advanced to leaflet 14, or to the anatomical site, through the same lumen.

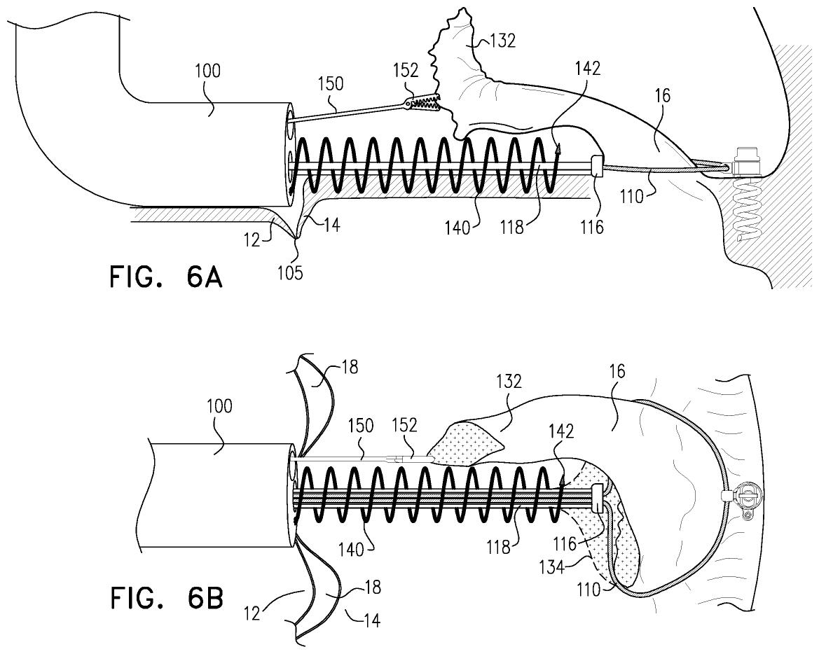

Reference is now made to FIGS. 5A and 5B, which are, respectively, a schematic side view illustration and a schematic top view illustration of continued sliding of tightening element 116 relative to wire loop 110, and therefore continued cutting of excess tissue 16 by wire loop 110, while clamp 150 holds cut portion 132 of the excess tissue, and helical needle 140 secures cut edges 134, in accordance with a further step of some applications.

As seen in FIGS. 5A and 5B, tightening element 116 continues to be advanced distally in the direction of arrow 160 to tighten the wire loop 110 thereby continuing to cut the excess tissue 16. Helical needle 140 continues to be rotationally advanced following tightening element 116, and thus holds together cut edges 134 as they are formed. As seen, in some applications, tip 142 of helical needle 140 remains at a fixed distance relative to tightening element 116, as the tightening element and the helical needle are advanced.

Clamping end 152 of clamp 150 can comprise jaws (e.g., as shown), and can be actuatable (e.g., openable and closeable) from an extracorporeal proximal end of the apparatus and/or system, e.g., as described hereinbelow with reference to FIGS. 10A-B.

While FIGS. 4A-4B show clamp 150 being advanced out of catheter 100 (e.g., with clamping end 152 in a closed and/or delivery state), FIGS. 5A-5B show clamping end 152 having been clamped onto excess tissue 16 (e.g., by the clamping end having been opened, and subsequently closed onto the excess tissue). It is to be noted that, although clamp 150 is shown as having been clamped onto excess tissue 16 after tightening of wire loop 110 (and thereby cutting of the excess tissue) has begun, for some applications, the clamp can clamp onto the excess tissue before cutting of the excess tissue has begun (e.g., before any tightening of the wire loop).

Clamping end 152 of clamp 150 continues to hold cut portion 132 of excess tissue 16 of leaflet 14 during the further tightening of wire loop 110 and the further cutting of the excess tissue. In some applications, clamp 150 holds the cut tissue aside, e.g., as shown for example in FIGS. 5A-B, to ensure that the cut tissue does not interfere with operation of wire loop 110 and of helical needle 140. In some applications, clamp 150 can be advanced further distally out of catheter 100 as cutting of the excess tissue advances, as seen by comparison of FIGS. 4A and 5A.

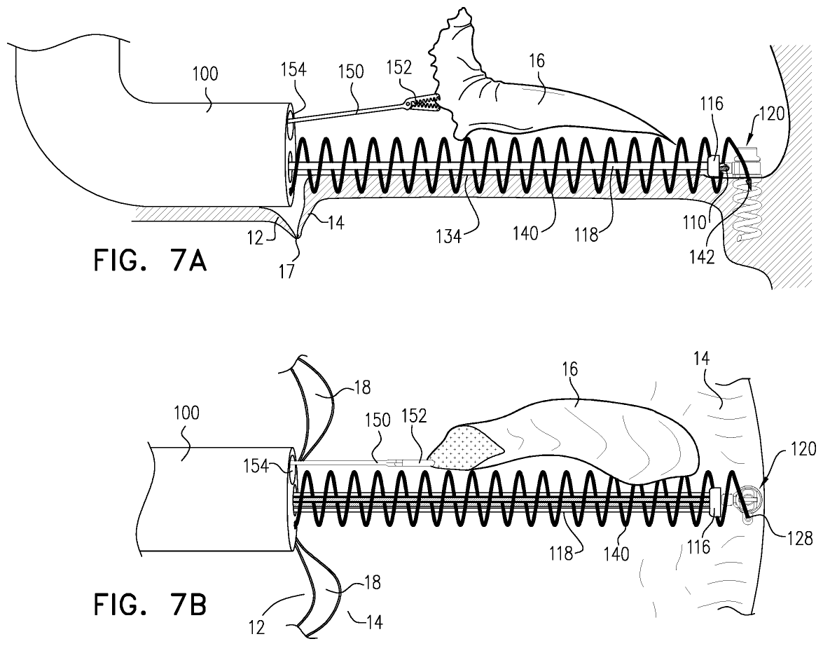

Turning now to FIGS. 6A and 6B, which shows a further step in accordance with some applications, it is seen that tightening element 116 continues tightening wire loop 110 around excess tissue 16, and cutting the excess tissue, thereby extending cut edges 134. Helical needle 140 continues to advance, in some applications with tip 142 at a fixed distance from tightening element 116, and to secure cut edges 134 together as they are formed. Concurrently, clamp 150 continues holding cut portion 132 of excess tissue 16.

Reference is now made to FIGS. 7A and 7B, which are, respectively, a schematic side view illustration and a schematic top view illustration of completion of cutting of excess tissue 16 by wire loop 110, and complete detachment of the excess tissue held by clamp 150 from leaflet 14, while helical needle 140 secures together cut edges 134 along the entire length thereof, in accordance with a further step of some applications.

As seen in FIGS. 7A and 7B, tightening element 116 of wire loop 110 has been pushed distally to an extent sufficient that excess tissue 16 has become fully detached from leaflet 14, and is now held secure only by clamping end 152 of clamp 150. For some applications, at this stage wire loop 110 has a minimal area, e.g., appearing essentially as a longitudinal wire (or two parallel longitudinal wire lengths). For some applications, at this stage tightening element 116 engages tissue anchor 120.

Following completion of cutting of excess tissue 16, helical needle 140 can continue to be rotationally advanced to the entirety of secure cut edges 134 together. For some applications, and as shown, at this stage helical needle 140 is operably decoupled from tightening element 116, such that tip 142 of the helical needle may cease to be at the same fixed distance from the tightening element, e.g., such that the tightening element remains stationary during the advancement of the helical needle. For some applications, and as shown, helical needle 140 can be rotationally advanced to extend beyond tightening element 116. Tip 142 of helical needle 140 is anchored in place, such as by being anchored to tissue anchor 120 (e.g., to second-device anchoring point 128 thereof), or by being anchored directly to the tissue of annulus 15, e.g., as described in further detail hereinbelow.

In some applications, clamp 150, together with the fully detached excess tissue 16 clamped thereto, can be removed from the anatomical site at this stage, for example via lumen 154 of catheter 100.

In some applications, clamp 150 can continue to clamp the detached excess tissue 16 at the anatomical site, until wire loop 110 and helical needle 140 are removed from the anatomical site, as described in further detail herein. For some applications, clamp 150 and excess tissue 16 can be removed via lumen 154. For some applications, clamp 150 and excess tissue 16 can be removed by withdrawing catheter 100, e.g., with the clamp and/or the excess tissue remaining exposed from the catheter.

Reference is now made to FIGS. 8A, 8B, 8C, and 8D, which are schematic side view illustrations, and to FIG. 8E, which is a schematic top view illustration, of phases of suturing secured cut edges 134 and removal of wire loop 110 and helical needle 140, in accordance with further steps of some applications.

As seen in FIG. 8A, suture 144 extends helically through helical lumen 143 of helical needle 140, and is secured to tip 142 of the helical needle at a needle-anchoring point 174. It is to be understood that, throughout this application, although the term “suture” may be applied to a component that is the same or similar in material and/or structure to a surgical suture, the scope of this term includes the use of other materials and/or structures.

Once tip 142 of helical needle 140 has been anchored in place (e.g., to tissue anchor 120, such as to second-device anchoring point 128 thereof), the tip of the helical needle is detached from the body (i.e., the remainder) of the helical needle, and helical needle 140 is rotationally retracted (e.g., unscrewed) from the tissue, e.g., about driving element 118 with wire 110 disposed within (FIG. 8B).

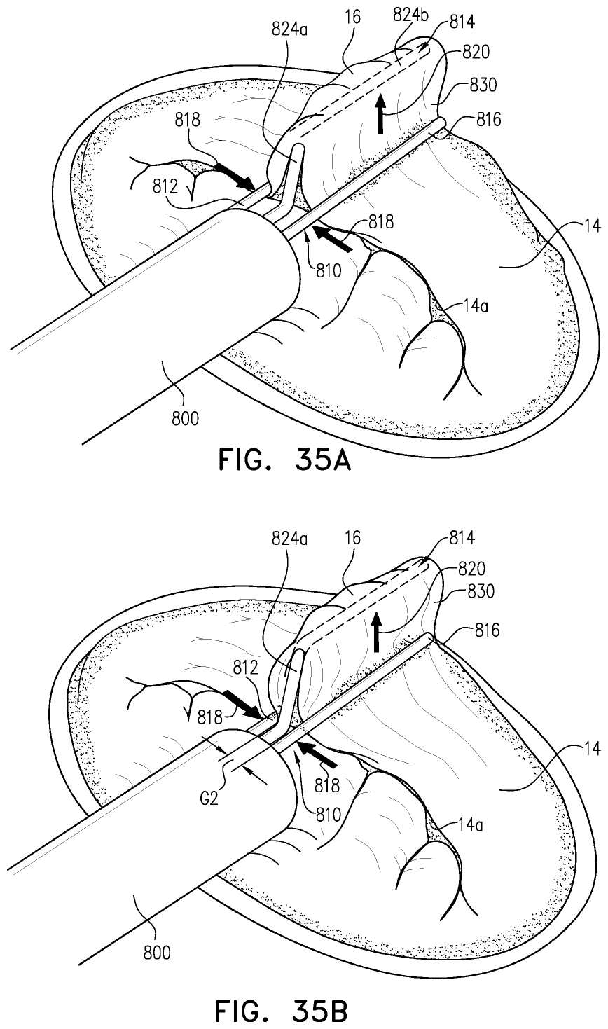

In some applications, tip 142 of helical needle 140 can be detached using a pushing-wire extending through lumen 143 of the helical needle, for example as described hereinbelow with respect to FIG. 37.

In some applications, needle tip 142 can be anchored directly to tissue of annulus 15, and not to tissue anchor 120. Such examples can be utilized, for example, when tissue anchor 120 is to be subsequently retracted from the anatomical site, as explained in further detail herein.

As seen in FIG. 8B, the body of helical needle 140 is rotationally retracted, in a direction indicated by arrow 176, leaving tip 142 of the helical needle, and suture 144 in place.

Turning to FIG. 8C, it is seen that wire loop 110 is detached from first device anchoring point 126 of tissue anchor 120, and is retracted in the direction of arrow 178, through suture 144. In some applications, wire loop 110 is retracted together with, or at a fixed distance from, helical needle 140. In some applications, wire loop 110 can be retracted independently of helical needle 140. In some applications, wire loop 110 can be fully retracted before retraction of helical needle 140 is initiated. Wire loop 110 can also be retracted subsequently to retraction of helical needle 140, provided that suture 144 remains loose enough for passage of the wire loop therethrough, during retraction of the wire loop.

For applications in which wire loop 110 is originally attached by being threaded through an eyelet defined by first-device anchoring point 126, the wire loop can be detached by being unthreaded (e.g., unlooped), e.g., by releasing one proximal end of the wire and pulling the other end of the wire proximally.

As seen in FIGS. 8D and 8E, which are corresponding side view planar and top view planar illustrations of a phase of suturing and retraction after further retraction of helical needle 140 into catheter 100, for example into the lumen through which it was advanced to the anatomical site. Similarly, the wire loop can have been retracted into the catheter, for example into the lumen through which it was advanced to the anatomical site. Following retraction of the wire loop, suture 144 can be tightened (e.g., by pulling proximally), in order to further secure cut edges 134. Retraction of needle 140 can continue until the needle is fully retracted, and subsequently catheter 100 can be removed from the anatomical site, leaving suture 144 securing cut edges 134 together.

In some applications, clamp 150 together with the cut excess tissue 16 clamped thereto (FIG. 7B), remain at the anatomical site during retraction of needle 140 and tightening of suture 144, as shown in FIGS. 8D and 8E. In some applications, clamp 150 together with cut excess tissue 16 can be removed from the anatomical site following retraction of wire loop 110 and of helical needle 140.

In some applications, tissue anchor 120 can remain anchored in annulus 15 of the heart valve, also following completion of the procedure and removal of catheter 100 from the heart valve. In some applications, following healing of leaflet 14, tissue anchor 120 can be removed from the heart valve, for example, in a separate procedure that can include decoupling anchor 120 from suture 144 (e.g., by decoupling the suture from distal needle tip 142, or by decoupling the distal needle tip from anchor 120).

In some applications, e.g., such as examples in which distal needle tip 142 is anchored directly to the tissue of annulus 15, tissue anchor 120 can be removed from the anatomical site together with wire 110, by detaching the tissue anchor from the tissue of annulus 15, while keeping the wire attached to the tissue anchor, and then retracting the wire with the tissue anchor. In some applications, distal needle tip 142, rather than tissue anchor 120, would continue to retain suture 144 securing cut edges 134, following removal of tissue anchor 120.

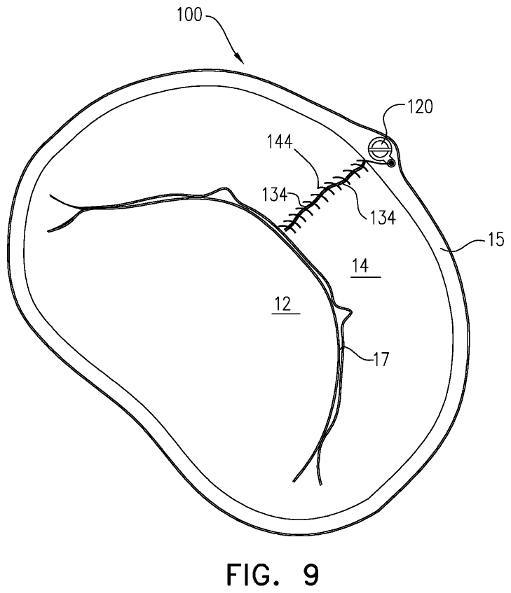

Reference is now made to FIG. 9, which is a schematic top view illustration of the mitral valve following repair thereof using the method of FIGS. 3A to 8E. As seen, the excess tissue has been removed from the valve leaflet, such that the leaflets 12 and 14 now better coapt at reference numeral 17, i.e., better closing the valve. Suture 144 extends along leaflet 14, holding together cut edges 134, and is held by tissue anchor 120 which remains in annulus 15 of the valve.

Reference is now made to FIGS. 10A and 10B, which are schematic illustrations of examples of control handles for controlling a device useful for implementing the techniques described with reference to FIGS. 3A to 8E, in accordance with some applications.

As shown, for some applications a proximal part 107 of longitudinal catheter 100 comprises a control handle such as a control handle 200a (FIG. 10A) or a control handle 200b (FIG. 10B). Although control handles 200a and 200b are shown as separate examples, one or more features of each can be substituted and/or combined with one or more features of the other. Control handles 200a and 200b are operable by a user such as a surgeon, and can be used for control and manipulation of one or more of the elements of the system discussed hereinabove, including catheter 100, wire loop 110, tightening element 116, tissue anchor 120, helical needle 140, tip 142 of the helical needle, clamp 150, and/or suture 144 (FIGS. 3A to 8E). Control handles 200a and 200b can be mechanically coupled to the system elements that they control, and can form a user interface for mechanical interaction with the system.

Alternatively or additionally, control handles 200a and 200b can comprise a user interface for one or more computerized control elements that control operation of the system elements. For the purposes of this application, the term “computerized control element”, and the equivalent term “computerized controller”, refer to a computing circuit or element for controlling operation of mechanical and/or electrical components of the system. The computerized control element includes a processing unit functionally associated with a non-tangible computer readable storage medium. The storage medium stores instructions, which, when executed by the processing unit, carry out actions which control the operation of the mechanical and/or electrical components of the system. For example, the instructions can include instructions to advance one or more components of the system, or to retract one or more components of the system. The computerized control element can be functionally associated with, or can include, a user interface for receiving of user input, which input can be used to trigger execution of specific instructions stored in the storage medium.

For the purpose of this application, the term “user interface” relates to any mechanical or electrical device with which a user interacts in order to control the system, either by direct, e.g., mechanical, control of the system or a component thereof, or by providing input to a computerized control element controlling any component of the system. As such, a user interface can include a handle, knob, or button for moving one or more mechanical components of the system, a computer interface such as a keyboard, mouse, screen, or the like, used to provide input to a computerized control element controlling one or more components of the system, or any other mechanism by which the user, such as a surgeon, operates the system.

It is to be noted that control handles 200a and 200b are extracorporeal control portions of catheter 100, and their shape and configuration can vary from that shown. For example, configurations are possible in which the extracorporeal control portion is not a handle shaped to be held.

The following description relates to unified control handles which control all the system elements. However, it will be appreciated that control of the system elements can be distributed between multiple different control handles or other user interfaces, as is suitable for the specific implementation of the system and environment in which the system is employed.

Each of control handles 200a and 200b can include a catheter controller 202 for advancing and retracting catheter 100 (FIGS. 3A to 8E), here shown as a rotatable engagement element, which, when rotated in one direction, controls advancement of the catheter, and when rotated in the opposing direction, controls retraction of the catheter. While the illustrated example shows catheter controller 202 as a wheel, it is appreciated that any other catheter controller can be used, such as a sliding controller, a touch pad controller, and the like. Alternatively or additionally, advancement and retraction of catheter 100 can be achieved by axially moving the catheter as a whole, e.g., by pushing and pulling the control handle.

Each of control handles 200a and 200b can include one or more catheter-steering controllers 203 for steering catheter 100 (FIGS. 3A to 8E), here shown as a rotatable engagement element. For example, rotation of catheter-steering controller 203 in one direction can control steering of the catheter in a first direction, and rotation in the opposing direction can controls steering of the catheter in a second, opposing, direction. For example, catheter-steering controller(s) 203 can be operatively coupled to distal part 106 of catheter 100 via one or more pullwires. While the illustrated example shows catheter-steering controller 203 as a wheel, facilitating steering of the catheter in two directions, it is appreciated that additional catheter-steering controllers can be used to steer the catheter in additional directions. It is further appreciated that any other catheter controller can be used, such as a sliding controller, a touch pad controller, a joystick controller, and the like.

Each of control handles 200a and 200b can include a wire loop controller 204, for advancing wire loop 110 (FIGS. 3A to 8C) distally out of the catheter, and optionally also for retracting the wire loop. In the illustrated example, wire loop controller 204 is shown as a rotatable engagement element, which, when rotated in one direction, controls advancement of wire 110, and when rotated in the opposing direction, controls retraction of the wire. While the illustrated example shows wire loop controller 204 as a wheel, it is appreciated that any other wire loop controller can be used, such as a sliding controller, a touch pad controller, and the like.

In some applications, wire loop controller 204 can include, or be functionally associated with, a wire detachment button 206, adapted to control detachment of wire 110 from the tissue anchor (FIG. 8C). Wire detachment button 206 can be a depressible button, or any other suitable type of button or interface with which the user can interact. In some applications, wire detachment button 206 can be implemented as part of wire loop controller 204, for example when depression of wire loop controller 204 results in detachment of wire 110 from tissue anchor 120, and rotation of the wire loop controller controls advancement and retraction of the wire.

Each of control handles 200a and 200b can include a tightening element controller 208, for sliding tightening element 116 distally over wire loop 110 (FIGS. 3A to 7B), and/or distally to the catheter or out of the catheter. In some applications, tightening element controller 208 is functionally associated with driving element 118 (FIGS. 3A to 7B), and controls advancement of tightening element 116 by controlling advancement of the driving element. In the illustrated example, tightening element controller 208 is shown as a rotatable engagement element which, when rotated in one direction, induces advancement of tightening element 116, and in some applications, when rotated in the opposing direction, can induce retraction of the tightening element. While the illustrated example shows tightening element controller 208 as a rotatable engagement element, it is appreciated that any other tightening element controller can be used, such as a sliding controller, a touch pad controller, and the like.

Each of control handles 200a and 200b can include a tissue anchor controller 210, which can be functionally coupled to anchor driving tool 109, for driving tissue anchor 120 (FIGS. 3A to 8E) to the anatomical site and anchoring the tissue anchor in annulus 15 (FIGS. 3A to 8E). In the illustrated example, tissue anchor controller 210 is shown as a rotatable engagement element which, when rotated in one direction, rotates anchoring tool 109 in a first direction (e.g., to screw tissue anchor 120 into the anatomical site), and when rotated in the opposing direction, rotates anchoring tool 109 in a second, opposing, direction (e.g., to release tissue anchor 120 from the anatomical site). However, any other type of tissue anchor and/or tissue anchor driver can be used, such as dart-like tissue anchors that are inserted axially into the tissue.

In some applications, each of control handles 200a and 200b can include a needle controller 212, for advancing helical needle 140 (FIGS. 3A to 8E) distally to the catheter, or out of the catheter, and for retracting the helical needle. In the illustrated example, needle controller 212 is shown as a rotatable engagement element, which, when rotated in one direction, controls advancement of needle 140, and when rotated in the opposing direction, controls retraction of the needle. While the illustrated example shows needle controller 212 as a wheel, it is appreciated that any other needle controller can be used, such as a sliding controller, a touch pad controller, and the like.

In some applications, needle controller 212 can include, or can be functionally associated with, a tip detachment button 214, adapted to control detachment of needle tip 142 (FIGS. 3A to 8E) from the remainder of needle 140. Tip detachment button 214 can be a depressible button, or any other suitable type of button or interface with which the user can interact. In some applications, tip detachment button 214 can be implemented as part of needle controller 212, for example when rotation of the needle controller controls advancement and retraction of needle 140, and depression of needle controller 212 results in detachment of needle tip 142 from the remainder of the needle.

In some applications, control handles 200a and 200b can include a clamp controller 216, for advancing clamp 150 (FIGS. 4A to 7B) distally to the catheter, or out of the catheter, and for retracting the clamp. In the illustrated example, clamp controller 216 is shown as a rotatable engagement element, which, when rotated in one direction, controls advancement of clamp 150, and when rotated in the opposing direction, controls retraction of the clamp. While the illustrated example shows clamp controller 216 as a wheel, it is appreciated that any other clamp controller can be used, such as a sliding controller, a touch pad controller, and the like.

In some applications, clamp controller 216 can include, or can be functionally associated with, a clamp actuator 218, adapted to control opening and closing of clamping end 152 (FIGS. 4A to 7B). Clamp actuator 218 can be a depressible button, or any other suitable type of button, lever, or interface with which the user can interact. In some applications, clamp actuator 218 can be implemented as part of clamp controller 216, for example when rotation of the clamp controller controls advancement and retraction of the clamp, depression of clamp controller 216 results in opening of clamping end 152, and release of clamp controller 216 results in closing of clamping end 152.

In some applications, each of control handles 200a and 200b can include a unified engagement element that, when actuated, controls motion of clamp controller 216 and of helical needle 140 in unison, for example at a fixed distance from one another.

In the example shown in FIG. 10A, control handle 200a includes the unified engagement element in the form of a rotatable engagement element 220, which is distinct from tightening element controller 208 and needle controller 212. When rotatable engagement element 220 is rotated in one direction, it controls advancement of tightening element 116 and of helical needle 140, in unison, for example at a fixed distance from one another. In some applications, in which it is desired for tightening element 116 and helical needle 140 to be retracted in unison, when rotatable engagement element 220 is rotated in the opposing direction, it can control retraction of the tightening element and the helical needle in unison.

In the example shown in FIG. 10B, control handle 200b includes a coupling element 222, functionally associated with tightening element controller 208 and with needle controller 212. In a first operative state of coupling element 222, for example when slid to the distal part of the slot in which it is located, tightening element controller 208 and needle controller 212 become operatively coupled (e.g., locked) to each other to form the unified engagement element, such that rotation of either one of the tightening element controller or the needle controller controls motion of both tightening element 116 and of helical needle 140 in unison, and/or at a fixed distance from one another. In a second operative state of coupling element 222, for example when slid to the proximal part of the slot in which it is located, tightening element controller 208 and needle controller 212 become decoupled. In this second operative state, actuation of tightening element controller 208 controls motion only of tightening element 116, and actuation of needle controller 212 controls motion only of helical needle 140.

The following description relates to additional implants and implantation methods for repairing a flailing leaflet of a heart valve. The methods and implants discussed herein are all shown with respect to repair of a leaflet of the mitral valve. However, they are equally applicable and useful for treatment of leaflets of the tricuspid valve.

When referring to anatomical structures or mechanical structures mentioned with respect to FIGS. 1-10B, the same reference numerals will be used hereinbelow.

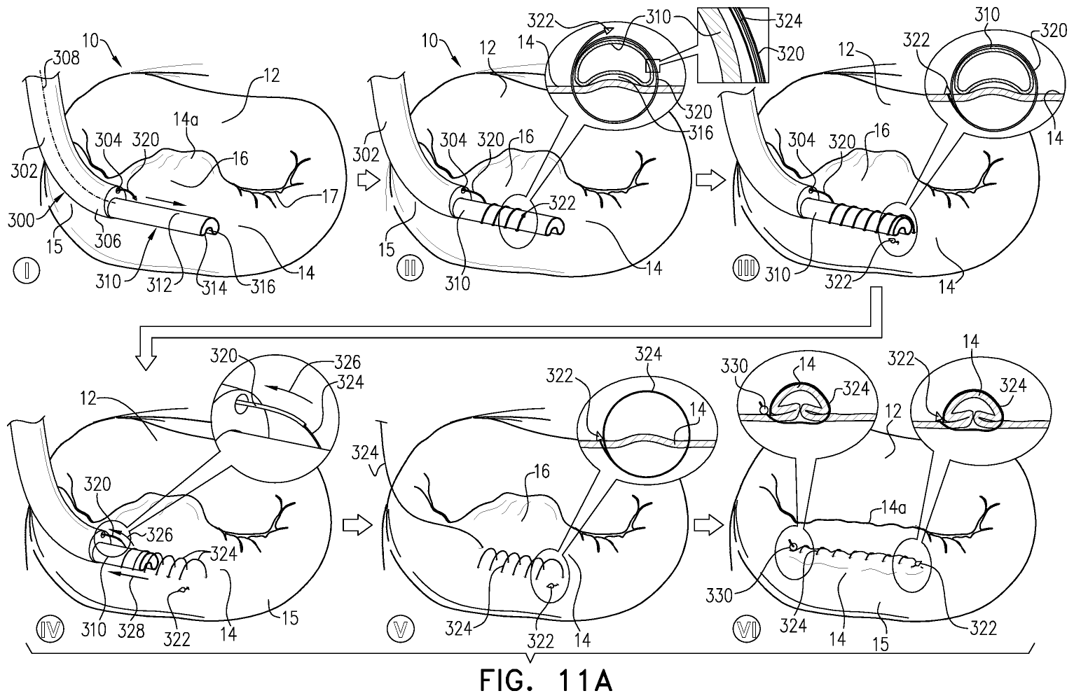

Reference is now made to FIGS. 11A-11D, which are schematic illustrations of phases of use of a system 300 for repairing second leaflet 14 of mitral valve 10 by suturing a portion of the leaflet between the root and lip of the leaflet, in accordance with some applications. FIG. 11A is a top view illustration showing at least some phases of a technique of using system 300 to repair the leaflet. In the example illustrated in FIG. 11A, second leaflet 14 includes excess tissue 16, e.g., causing prolapse and/or suboptimal coaptation with first leaflet 12 (e.g., along a portion of edge 17).

As seen at phase I, a longitudinal catheter 302 is advanced toward an anatomical site of the subject. Longitudinal catheter 302 is substantially similar to longitudinal catheter 100 described hereinabove with respect to FIGS. 2-10B. In the illustrated example, a distal part 306 of the catheter is advanced to an atrium (e.g., a left atrium) of the heart, to be positioned upstream of a heart valve (e.g., mitral valve 10). Longitudinal catheter 302 also has an extracorporeal proximal part (e.g., as shown in FIGS. 10A-B). Distal part 306 is guidable to the anatomical site, such as by being actively steerable itself (e.g., by being operatively coupled by one or more pullwires to the proximal part, such as to a steering controller 203 thereof shown in FIGS. 10A and 10B), or by being passively guided and/or steered (e.g., by extending over or through another steerable element, such as an actively steerable catheter). A longitudinal axis 308 extends between the proximal part and steerable distal part 306 of catheter 302. Catheter 302 is transluminally advanced to the anatomical site using any method known in the art, for example via the vena cava and, if necessary, via the septum separating the atria of the heart.

A mount 310 is advanced to the heart of the subject, e.g., to an atrium upstream of the valve to be treated. In some applications, such as the illustrated example, mount 310 can be advanced to the target anatomical site through a lumen of catheter 302, and distally out of steerable distal part 306 of catheter 302. In some applications, the mount can be advanced to the target location attached to distal part 306 of catheter 302.

In some applications, such as the example illustrated in FIG. 11A, mount 310 has a crescent-shaped cross section, perpendicular to a longitudinal axis thereof. The crescent shape defines a leaflet-facing surface 314 that is concave, thereby defining a cavity 316 extending longitudinally along at least part of mount 310. Mount 310 can have an opposite surface 312, oriented to face away from the leaflet. Surface 312 can be convex (e.g., its shape being defined by the crescent shape).

It is to be noted that mount 310 can optionally have a different cross-sectional shape. In some applications, however, in each case, opposite surface 312 is shaped substantially as an arc 317 (e.g., a circular arc) that, if extrapolated into a full circle (indicated by dashed line 318;

FIGS. 11B to 11D), would extend around leaflet-facing surface 314 with a space 319 between the arc and the leaflet-facing surface. This is illustrated in FIG. 11B for crescent-shaped mount 310 in which leaflet-facing surface 314 is concave, in FIG. 11C for a mount 310′ (i.e., a variant of mount 310) having a cross-sectional shape is that of a circular segment (e.g., a semicircle) in which leaflet-facing surface 314 is substantially flat, and in FIG. 11D for a mount 310″ (i.e., a variant of mount 310) having a cross-sectional shape is substantially that of a gibbous moon, in which leaflet-facing surface 314 is convex.

Mount 310 is positioned with surface 314 facing leaflet 14, e.g., in contact with the leaflet (phase I). It is to be understood that, in such a position, a portion of leaflet 14 can be disposed within space 319, and can be disposed within cavity 316 of the mount. As seen in phases I and II, a helical needle 320 having a distal needle tip 322 is then rotationally advanced distally out of distal part 306 of catheter 302, helically about mount 310. Helical needle 320 can be similar to, or identical to, helical needle 140 described hereinabove with respect to FIGS. 4A and 4B. Rotation of helical needle 320 can be performed while mount 310 remains stationary with respect to leaflet 14. Helical needle 320 includes a helical lumen, which has a suture 324 extending helically therethrough, as described in further detail hereinbelow. As seen clearly in the enlarged portion of phase II, as helical needle 320 rotates, a portion of the tissue of second leaflet 14 (e.g., the portion that is disposed within space 319), between annulus 15 and lip 14a of the leaflet, is caught by the helical needle.

In some applications, during rotation of helical needle 320, the helical needle is stabilized by or guided by mount 310. More specifically, during rotation of helical needle 320, the helical needle engages mount 310 along arc 317. For some applications, mount 310 is structured such that an angle corresponding to arc 317 of opposite surface 312 is greater than 180 degrees, and as such the arc contacts needle 320 through more than half of each rotation. This further stabilizes helical needle 320 (e.g., the needle cannot be pushed closer to surface 314 than to surface 312).

It is to be noted that a mount, similar to mount 310, can be used in a similar manner to stabilize or guide helical needle 140 during carrying out of the method described hereinabove with respect to FIGS. 4A to 9.

In the example illustrated in FIG. 11A, mount 310 and helical needle 320 are advanced along the width of leaflet 14, substantially parallel to the annulus and/or to the lip of the leaflet. However, in some applications, the mount and helical needle can be advanced in other directions or orientations, mutatis mutandis.

In some applications, needle 320 can be advanced through the same lumen as mount 310. In some applications, such as the illustrated example, needle 320 can be advanced through a dedicated lumen 304 in catheter 302. Lumen 304 can be a helical lumen, or can be a linear lumen. In examples in which lumen 304 is linear, needle 320 can automatically become helical upon exposure out of the lumen, for example by use of shape memory materials for the helical needle.

Although helical needle 320 is shown as having the shape of a cylindrical helix, i.e., having a constant transverse diameter along its length, for some applications the transverse diameter of the helical needle can vary along its length, such as being wider at the distal end than at the proximal end, e.g., it can have the shape of a conical helix.

Although helical needle 320 is shown as having a constant pitch, for some applications, the helical needle can have a variable pitch.

As seen at phase III, helical needle 320 continues to be rotationally advanced toward the distal end of mount 310. At this stage, tip 322 of helical needle 320 is detached from the body (i.e., the remainder) of helical needle 320, but remains attached to suture 324, stopping sliding of the suture back out of the tissue.

Turning to phase IV, it is seen that helical needle 320 is rotationally retracted (e.g., unscrewed) from the tissue, e.g., about mount 310, in a direction indicated by arrow 326, leaving suture 324 in place. Mount 310 is linearly retracted from the tissue, in a direction indicated by arrow 328. In some applications, mount 310 is retracted together with, or at a fixed distance from, helical needle 320. In some applications, mount 310 can be retracted subsequently to retraction of helical needle 320 provided that suture 324 remains loose enough for passage of the mount therethrough, during retraction of the mount. In some applications, mount 310 can be retracted prior to retraction of helical needle 320.

Retraction of helical needle 320 and mount 310 can be, for example, into catheter 302, e.g., into the respective lumens from which they were advanced to the anatomical site.

Phase V illustrates the suture 324 threaded through leaflet 14, following complete removal of helical needle 320, mount 310, and catheter 302.

Following complete retraction of the mount 310 and helical needle 320, suture 324 can be tensioned (e.g., by pulling proximally), as shown at phase VI. Following tensioning of suture 324, a second locking element 330 can be locked to the suture to secure the suture in its tensioned state, and a proximal end of the suture can be cut.

As seen in the enlarged portion of phase VI, following tensioning of suture 324, tissue of second leaflet 14 which was caught by helical needle 320 is plicated. As a result, the length of second leaflet 14 from annulus 15 to lip 14a is shortened relative to the initial length shown at phase I, thereby improving coaptation between leaflets 14 and 12.

For some applications, suture 324 may be sufficiently tensioned so as to plicate or shorten the tissue along the longitudinal direction of the suture. For some such applications, helical needle 320 is selected to have a relatively large pitch, which creates a greater distance between locations at which the suture passes through the tissue, and therefore enhances plicating of the tissue between such locations upon subsequent tensioning of the suture. That is, a relatively small pitch may be selected to favor contraction in a direction transverse to the axis of the helical needle, whereas a relatively large pitch may be selected to favor contraction along, or in a direction parallel with, the axis of the helical needle. For some applications, such a technique can be used, mutatis mutandis, to contract or plicate the annulus.

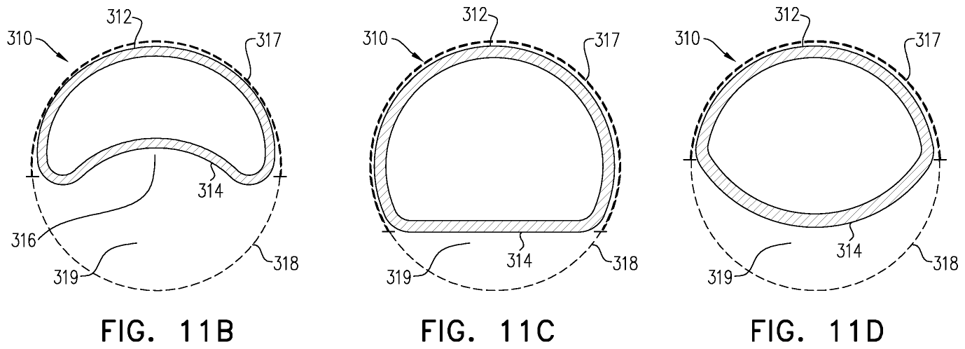

Reference is now made to FIGS. 12A, 12B, and 12C, which are schematic top view or sectional illustrations of phases of repairing second leaflet 14 of mitral valve 10 by suturing a portion of the leaflet to the root of the leaflet, in accordance with some applications. The method shown in FIGS. 12A, 12B, and 12C is similar to that shown in FIG. 11A, with FIGS. 12A, 12B, and 12C corresponding approximately to phases III, IV, and VI of FIG. 11A.

As seen in FIG. 12A, longitudinal catheter 302 has been advanced toward the anatomical site, at the root of second leaflet 14, substantially as described hereinabove with respect to phase I of FIG. 11A. Mount 310 has been advanced distally out of catheter 302, and helical needle 320 has been rotationally extended distally out of catheter 302, about mount 310, in the direction of arrow 332, substantially as described hereinabove with respect to phases I, II, and III of FIG. 11A. However, in the example shown in FIG. 12A, mount 310 has been positioned along root 333 of leaflet 14 such that helical needle 320 engages both leaflet 14 and atrial wall 334 of the atrium upstream of valve 10. For example, and as shown, as needle 320 is helically advanced, it can enter atrial wall 334 and exit leaflet 14. As such, the helical needle has captured therein tissue of the leaflet, at root 333 of the leaflet, adjoining atrial wall 334.

As seen in FIG. 12B, following rotational advancement of needle 320 to the edge of mount 310, tip 322 of helical needle 320 is detached from the body (i.e., the remainder) of helical needle 320, but remains attached to suture 324, stopping sliding of the suture back out of the tissue.

Helical needle 320 is then rotationally retracted (e.g., unscrewed) from the tissue, e.g., about mount 310, leaving suture 324 in place, e.g., as described with reference to phase IV of FIG. 11, mutatis mutandis. Mount 310 can be retracted linearly from the tissue, in a direction indicated by arrow 336. In some applications, mount 310 is retracted together with, or at a fixed distance from, helical needle 320. In some applications, mount 310 can be retracted subsequently to retraction of helical needle 320 provided that suture 324 remains loose enough for passage of the mount therethrough, during retraction of the mount. In some applications, mount 310 can be retracted prior to retraction of helical needle 320.

Retraction of helical needle 320 and mount 310 can be, for example, into catheter 302, e.g., into the respective lumens from which they were advanced to the anatomical site.

FIG. 12B shows suture 324 remaining threaded through the root of leaflet 14, following withdrawal of needle 320 and mount 310. FIG. 12C shows suture 324 having been tensioned (e.g., by pulling proximally), the tension having been locked into the suture using a second locking element 330 locked onto the suture, and the proximal part of the suture having been cut.

As seen in the FIG. 12C, following tensioning of suture 324, tissue of second leaflet 14 adjacent the cardiac wall, which tissue was caught by helical needle 320, is plicated. As a result, second leaflet 14 is effectively shortened (along a root-to-tip axis) relative to the initial length shown in the enlarged portion of FIG. 12B, and consequently lip 14a of second leaflet 14 properly coapts with lip 12a of first leaflet 12.

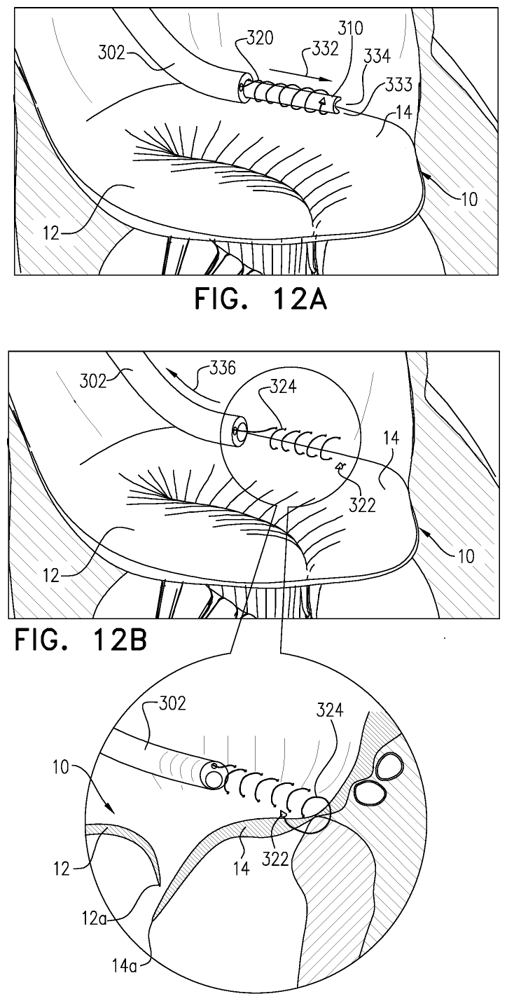

Reference is now additionally made to FIG. 13, which is a schematic sectional illustration of repairing leaflet 14 of mitral valve 10 by suturing a portion of the leaflet to a coronary artery 340 surrounding the mitral valve, in accordance with some applications.

As seen in FIG. 13, the process of FIGS. 12A to 12C can be carried out with the catheter introduced to the anatomical site via coronary artery 340, which surrounds the mitral valve. In some applications, the helical needle and suture 324 would extend through entry points in a wall of coronary artery 340, through leaflet 14 adjacent the root, and back into the coronary artery via exit points in the wall of the coronary artery. Following tensioning of the suture 324, a portion of leaflet 14 adjacent the root thereof is bound to the cardiac wall through which coronary artery 340 extends, as shown in FIG. 13. As a result, leaflet 14 is effectively shortened (along a root-to-tip axis), and consequently lip 14a of second leaflet 14 properly coapts with lip 12a of first leaflet 12. For some applications, mount 310 (or a similar mount) is used to facilitate such a technique. For some applications, such techniques are performed without the use of a mount.

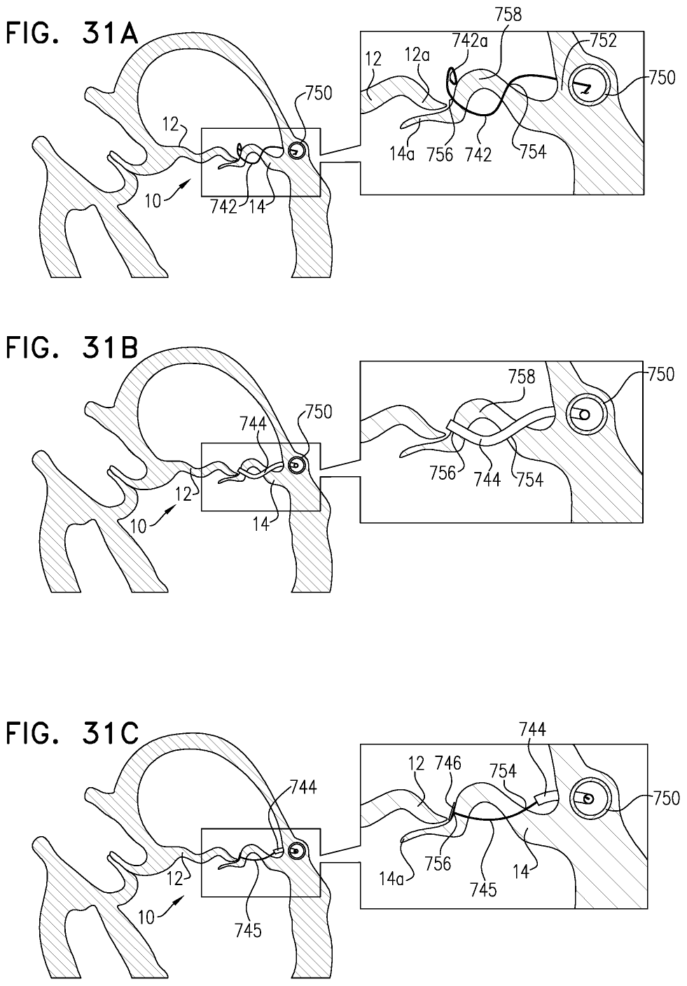

Reference is now made to FIG. 33, which is another schematic illustration of excess tissue in the leaflet of a native valve, and improper closing thereof. The reference numerals used in FIG. 33 correspond to those used in FIG. 1, and throughout the specification. FIG. 33 shows native valve 10 (depicted here as a mitral valve, but can apply to other valves) having first leaflet 12 and second leaflet 14, surrounded by annulus 15. Second leaflet 14 has excess tissue 16, such that gaps 18 exist in the valve when it is closed. The length of second leaflet 14, extending from lip 14a to annulus 15, is indicated by reference numeral 801, while the width of the leaflet, in a direction perpendicular to length 801, is indicated by reference numeral 802.

FIG. 34 is a schematic illustration of placement of a holding device 810 in the native valve 10 shown in FIG. 33, in accordance with some applications.

As seen in FIG. 34, a longitudinal catheter 800 is advanced toward an anatomical site of the subject. Longitudinal catheter 800 is substantially similar to longitudinal catheter 100 described hereinabove with respect to FIGS. 2-10B. In the illustrated example, a distal part 806 of the catheter is advanced to an atrium (e.g., a left atrium) of the heart, to be positioned upstream of a heart valve (e.g., mitral valve 10). Longitudinal catheter 800 also has an extracorporeal proximal part (e.g., as shown in FIGS. 10A-B). Distal part 806 is guidable to the anatomical site, such as by being actively steerable itself (e.g., by being operatively coupled by one or more pullwires to the proximal part, such as to a steering controller 203 thereof shown in FIGS. 10A and 10B), or by being passively guided and/or steered (e.g., by extending over or through another steerable element, such as an actively steerable catheter). A longitudinal axis 808 extends between the proximal part and steerable distal part 806 of catheter 800. Catheter 800 is transluminally advanced to the anatomical site using any method known in the art, for example via the vena cava and, if necessary, via the septum separating the atria of the heart.

In some applications, and as shown, a holding device 810, including a first beam 812, a second beam 814, and a third beam 816, is advanced distally out of steerable distal part 806 of catheter 800. Catheter 800 positions holding device 10 onto second leaflet 14 of mitral valve 10, such that first beam 812 and third beam 816 are disposed on one side of the leaflet, here shown as the upstream side of the leaflet, and second beam 814 is disposed on the opposing side of the leaflet, here shown as the downstream side of the leaflet. In some applications, the catheter positions first beam 812 and third beam 816 at lateral edges of excess tissue 16 of leaflet 14, such that the entirety of the excess tissue is disposed between beams 812 and 816.

For some applications, catheter 800 positions the holding device 810 onto second leaflet 14 when the holding device in a first operative state, to define a first distance G1 between beams 412 and 416. In this first operative state, beams 812, 814, and 816, can be substantially in a single plane.

For some applications, beams 812, 814, and 816 can be fixedly attached to each other and move as a unit relative to catheter 800. As such, the beams can be advanced out of catheter 800 as a single unit.

For some other applications, beams 812, 814, and 816 can be moved independently of one another relative to catheter 800, and can each be advanced separately out of catheter 800.

For some applications, beams 812, 814, and 816 can all be advanced out of a single lumen in catheter 800. For some applications, at least two of beams 812, 814, and 816 can be advanced out of two different lumens in catheter 800, or each of the beams can be advanced out of a dedicated lumen in the catheter.

For some applications, holding device 810, and specifically beams 812, 814, and/or 816, can be flexible and/or resilient. For example, in some applications, holding device 810 and/or the beams thereof can be formed of a resilient metal, or of a shape memory material.

Reference is now made to FIGS. 35A and 35B, which are schematic illustrations of phases of transforming the holding device 810 from the first operative state toward a second operative state, in accordance with some applications.

In some applications, and as shown, catheter 800 transforms holding device 810 from the first operative state shown in FIG. 34 toward the second operative state shown in FIG. 35B by moving first beam 812 and third beam 816 toward each other in the direction of arrows 818, while moving second beam 814 away from, and for some applications perpendicular to, the plane formed by beams 812 and 816, here shown as in an upward direction, indicated by arrow 820.

In some applications, second beam 814 is bent, or deformed, during the transforming of holding device 810 from the first operative state toward the second operative state. The bent beam 814 includes a transition portion 824a which turns, in some applications at an obtuse angle, to the remainder of the beam 824b. In some such applications, remainder 824b is substantially parallel to beams 812 and 816.

In some applications, in the second operative state of holding device 810, a second distance G2 exists between first beam 812 and second beam 816, the distance G2 being smaller than the distance G1. In some applications, distance G2 is no more than twice as great as the width of second beam 814. For example, distance G2 can be substantially equal to the width of the second beam, or can be smaller than the width of the second beam.

In some applications, in the second operative state of holding device 810, second beam 814 is disposed outside of a plane defined by first beam 812 and third beam 816.

As seen in FIG. 35B, following transformation of holding device 810 to the second operative state, excess tissue 16 forms a bulge 830, which bulge is held between first beam 812 and third beam 816, and is supported, from the downstream side of the leaflet, by second beam 814. In the illustrated embodiment, bulge 830 extends toward the upstream side of leaflet 14, and into the atrium.

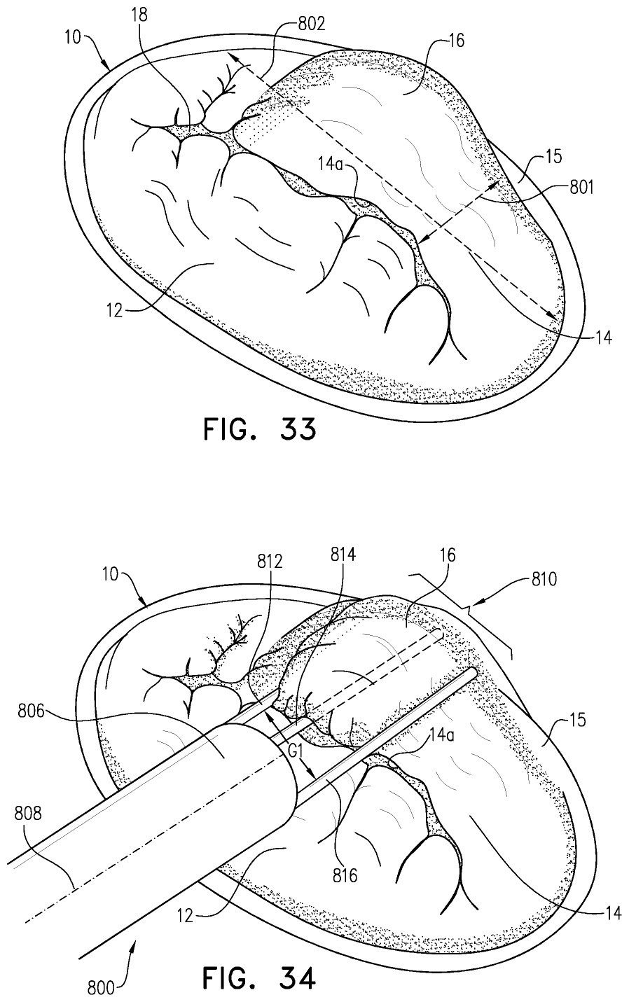

Reference is now made to FIGS. 36A and 36B, which are schematic illustrations of phases of securing together segments of the tissue of mitral valve 10 using a helical needle 840, in accordance with some applications.

As seen, an attaching device, here shown as a helical needle 840 having a distal needle tip 842, is rotationally extended distally out of distal part 806 of catheter 800. Helical needle 840 can be similar to, or identical to, helical needle 140 described hereinabove with respect to FIGS. 4A and 4B. Helical needle 840 includes a helical lumen 843, which has a suture 844 extending helically therethrough, as described in further detail hereinbelow.

For some applications, helical needle 840 rotates while advancing along length 801 toward annulus 15, substantially perpendicular to the lip of the leaflet. However, in some applications, the mount and helical needle can be advanced in other directions or orientations, mutatis mutandis.

For some applications, helical needle 840 rotates about first beam 812 and third beam 816, and beneath second beam 814, to attach two segments of leaflet. A first of the two segments can include a portion of the base of bulge 830 above first beam 812, a portion of leaflet 14 to the side of the first beam, and tissue of the leaflet which is engaged by the first beam. Similarly, a second of the two segments can include a portion of the base of bulge 830 above third beam 816, a portion of leaflet 14 to the side of the third beam, and tissue of the leaflet which is engaged by the third beam. The direction of rotation of needle 840 is shown by reference numeral 845.

In some applications, helical needle 840 can secure the two segments of leaflet 14 together temporarily, and a permanent attachment element, such as suture 844, can subsequently chronically maintain the attachment of the two segments following removal of helical needle 840, as described in further detail hereinbelow.

In some applications, needle 840 can be advanced to leaflet 14 through catheter 800. In some applications, needle 840 can be advanced through the same lumen as holding device 810. In some applications, needle 140 can be advanced through a dedicated lumen in catheter 800, similar to lumen 146 of FIGS. 4A and 4B. The dedicated lumen can be a helical lumen, or can be a linear lumen. In examples in which the dedicated lumen is linear, needle 840 automatically becomes helical upon exposure out of the lumen, for example by use of shape memory materials for the helical needle.

Although helical needle 840 is shown as having the shape of a cylindrical helix, i.e., having a constant transverse diameter along its length, for some applications the transverse diameter of the helical needle can vary along its length, such as being wider at the distal end than at the proximal end, e.g., it can have the shape of a conical helix.

Although helical needle 840 is shown as having a constant pitch, for some applications, the helical needle can have a variable pitch.

For some applications, helical needle 840 can be replaced by a different edge-attaching device, mutatis mutandis. For example, alternate attaching devices for attaching the two segments of the tissue can include clamps, pins, hooks, tissue adhesive, and the like.

For some applications, during rotation thereof, helical needle 840 can be guided by or stabilized by a mount, similar to mount 310 of FIGS. 11A to 11D, and in a similar manner to that described with respect to FIGS. 11A to 11D.

Reference is now made to FIG. 37, which is a schematic illustration of detachment of needle tip 842 from helical needle 840, in accordance with some applications. As seen in FIG. 37, following rotation of needle 840 to secure the desired segments of leaflet 14, here shown as segments extending from lip 14a to annulus 15, tip 842 of the helical needle is detached from the body (i.e., the remainder) of helical needle 840, but remains attached to suture 844, preventing sliding of the suture back out of the tissue.

For some applications, detachment of needle tip 842 from the remainder of helical needle 840 is carried out by a pushing-wire 846. Pushing-wire 846 extends through lumen 843 of helical needle 840, and is configured to push the detachable needle tip 842 distally relative to the remainder of helical needle 840, thereby detaching the needle tip from the needle body.

It is to be noted that a pushing-wire, similar to pushing-wire 846, can be used in a similar manner to detach needle tip 142 from the remainder of helical needle 140 during carrying out of the method described hereinabove with respect to FIGS. 4A to 9, or to detach needle tip 322 from the remainder of helical needle 320 during carrying out of the methods described hereinabove with respect to FIGS. 11A to 13.

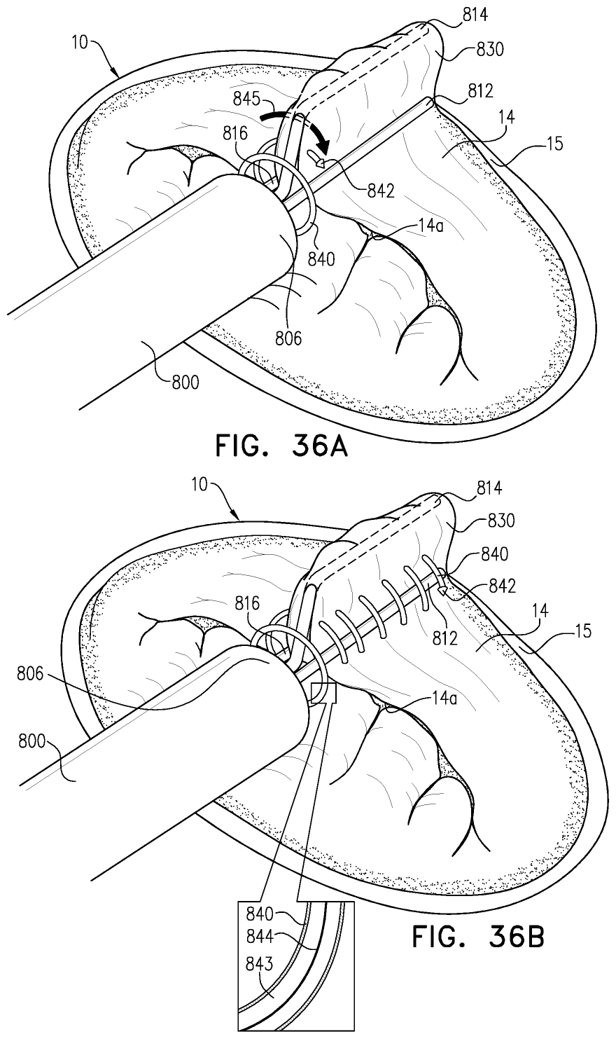

Turning now to FIG. 38, the Figure is a schematic illustration of retraction of helical needle 840, while continuing to secure the segments of leaflet 14, in accordance with some applications. It is seen that helical needle 840 is rotationally retracted (e.g., unscrewed) from the tissue, in a direction indicated by arrow 848, leaving suture 844 in place, and needle tip 842 anchoring the suture directly to tissue of bulge 830.

For some applications, and as shown, first beam 812 and/or third beam 816 can be proximally retracted from bulge 830 prior to retraction of helical needle 840. For some such applications, second beam 814 remains extended distally out of catheter 800, and supports bulge 830, as shown in FIG. 38.

For some applications, beams 812 and 816 can be linearly retracted from the tissue, in a direction indicated by arrow 850. In some applications, beams 812 and 816 are retracted concurrently with, and/or at a fixed distance from, helical needle 840. In some applications, beams 812 and 816 can be retracted subsequently to retraction of helical needle 840 provided that suture 844 remains sufficiently loose for passage of the beams therethrough, during retraction of the beams. In some applications, beams 812 and 816 can be retracted prior to retraction of helical needle 840.

Retraction of helical needle 840 and beams 812 and 816 can be, for example, into catheter 800, e.g., into the respective lumens from which they were advanced to the cardiac valve.

Following complete retraction of helical needle 840 and of beams 812 and 816, suture 844 can be tensioned (e.g., by pulling proximally). Following tensioning of suture 844, a second locking element 882 can be locked to the suture to secure the suture in its tensioned state, and a proximal end of the suture can be cut, substantially as described hereinabove with respect to FIG. 11A.

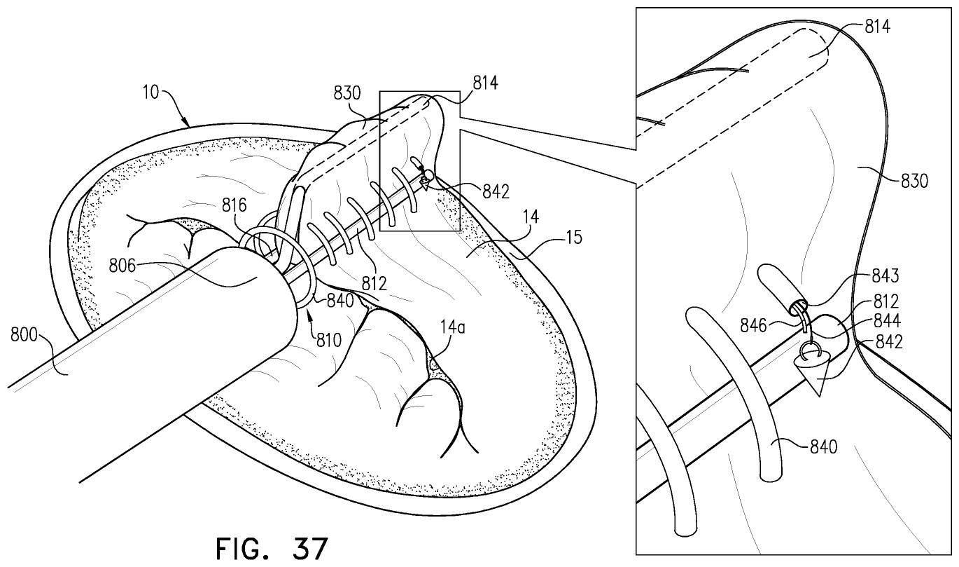

Upon tensioning of suture 844, tissue of second leaflet 14 which was caught by helical needle 840 is plicated. As a result, the effective width of second leaflet 14 (indicated by 802 in FIG. 33), is shortened relative to the initial width, thereby improving coaptation between leaflets 14 and 12. However, for some applications, it is desirable to reduce the amount residual tissue remaining in bulge 830, for example by cutting away some tissue from the bulge, as described herein with respect to FIGS. 39A to 41.

Reference is now made to FIGS. 39A, 39B, and 39C, which are schematic illustrations of phases of cutting of excess tissue 16 between the secured segments of bulge 830 of leaflet 14, in accordance with some applications.

As seen, in some applications, following complete retraction of helical needle 840, and in some applications also following retraction of beams 812 and 816, a cutting device 860 is advanced distally to an end of bulge 830, between the two secured segments of tissue.

In the application shown in FIGS. 39A to 39C, the cutting device 860 comprises a longitudinal stem 862 having one or more (e.g., one, a pair of, three of, etc.) blades 864 including sharp edges 865 attached to a distal end thereof. Blades 864 are pivotable relative to a longitudinal axis of stem 862 about an axis 866, between a first operative state suitable for storage and delivery, and a second operative state suitable for cutting. In the first, storage, operative state, shown in block I of FIG. 39A, blades 864 are disposed substantially parallel to the longitudinal axis of stem 862, and in the second, cutting, operative state, shown in block II of FIG. 39A, the blades are substantially perpendicular to the longitudinal axis of the stem, with sharp edges 865 pointing in a proximal direction. Cutting device 860 can be actuatable (e.g., transitionable between operative states) from an extracorporeal proximal end of the apparatus and/or system, e.g., as described hereinabove with reference to FIGS. 10A-10B.

For some applications, cutting device 860 can extend distally out of a distal end 870 of second beam 814. In such applications, second beam 814 includes a lumen 872 which accommodates cutting device 860 therein, with the cutting device is in the first operative state, until the cutting device is required. Upon retraction of helical needle 840, second beam 814 can be further advanced distally out of catheter 800, as seen by comparison of FIGS. 38 and 39A, so that distal end 870 extends distally out of bulge 830. At this stage, cutting device 860 can be advanced distally out of second beam 814, and can be transitioned from the storage operative state to the cutting operative state, such that sharp edges 865 of blades 864 are directed toward bulge 830.

However, it is to be appreciated that cutting device 860 can be any suitable cutting device, and can be advanced to the vicinity of bulge 830 independently of beam 814 or of holding device 810. For example, the cutting device can comprise a cutting wire or a wire loop, substantially as described hereinabove with respect to FIGS. 4A to 9, which can be advanced to the mitral valve via catheter 800, e.g., via a dedicated lumen or via the lumen through which helical needle 840 was advanced.

Turning to FIG. 39B, it is seen that second beam 814, with cutting device 860 extending distally therefrom, is retracted proximally into catheter 800 in the direction of arrow 876. During retraction of second beam 814, sharp edges 865 of blades 864 cut through tissue of bulge 830, between the segments of the bulge which are secured by suture 844. As a result, cut tissue 878 extends above cutting device 860, and cut edges 880 are formed in bulge 830, between the sutured segments.

Turning to FIG. 39C, it is seen that cutting device 860 and second beam 814 are moved further proximally, resulting in further detachment of cut tissue 878 from bulge 830, and extension of cut edges 880. Proximal motion of second beam 814 and cutting device 860 can be caused by further retraction of beam 814 into catheter 800, and/or by proximal retraction of catheter 800, together with beam 814 and cutting device 860, away from leaflet 14. Both types of motion are shown in FIG. 39C. As seen in FIG. 39C, the cut tissue 878 is almost completely detached from bulge 830.

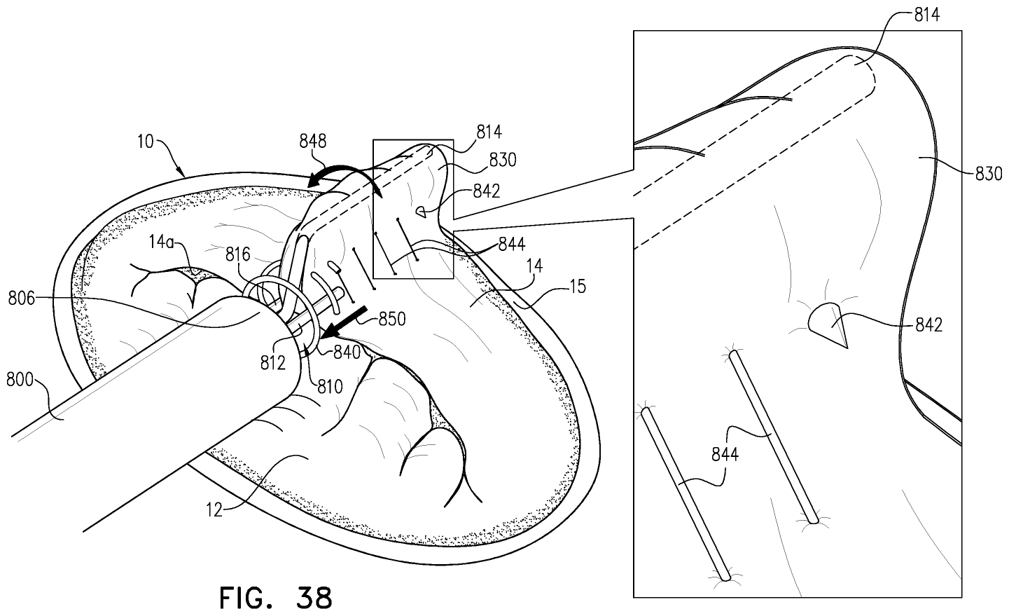

Reference is now made to FIGS. 40A and 40B, which are schematic illustrations of clamping and removal of cut tissue 878 from the mitral valve 10, in accordance with some applications. As seen in FIG. 40A, just prior to, or just following, complete detachment of cut tissue 878 from the remainder of bulge 830, a clamp having a distal clamping end is advanced distally out of catheter 800, and engages the cut tissue. In the illustrated embodiment, cutting device 860 functions as the clamp, by transitioning at least partway back toward the storage operative orientation, such that blades 864 clamp part (e.g., an end) of cut tissue 878.

However, any other type of clamp can be used, such as clamp 150 described hereinabove with respect to FIGS. 4A to 9. The clamp can be advanced distally out of catheter 800 as described with respect to FIGS. 4A to 9, e.g., through a dedicated lumen or through the same lumen as holding device 810 or as helical needle 840. The clamp can be advanced distally out of the catheter in a closed and/or delivery state, and can be operated to clamp cut tissue 878, substantially as described. The clamping end of the clamp can comprise jaws, and can be actuatable (e.g., openable and closeable) from an extracorporeal proximal end of the apparatus and/or system, e.g., as described hereinbelow with reference to FIGS. 10A-10B.

As seen in FIG. 40B, second beam 814, having cut tissue 878 clamped thereto, is removed from the cardiac chamber adjacent mitral valve 10. In some applications, in which the clamp is separate from second beam 814, the second beam, cutting device, and clamp are removed from the mitral valve.

For some applications, beam 814 including cutting device 860 and cut tissue 878 clamped thereto (or the second beam, cutting device, and another clamp having the cut tissue clamped thereto) can be removed by retraction thereof into one or more lumens of catheter 800. For some applications, beam 814 and cut tissues 878 can be removed by withdrawing catheter 800, e.g., with beam 814 and/or cut tissue 878 remaining exposed from the catheter.

It is to be appreciated that for some applications (e.g., applications in which the cutting device and clamp are independent from second beam 814), second beam 814 can be retracted from mitral valve 10 together with the first and third beams, e.g., during retraction of helical needle 840.

It is to be appreciated that for some applications in which beams 812, 814, and 816 cannot be moved longitudinally relative to each other, and/or cannot be retracted from the mitral valve separately from each other (e.g., are axially fixed with respect to each other), beams 812 and 816 can be retracted together with beam 814, during cutting of the excess tissue from bulge 830. For such applications, tensioning of suture 844 can be carried out following cutting of the excess tissue, so that the suture remains slack enough to allow retraction of beams 812 and 816 during the cutting.

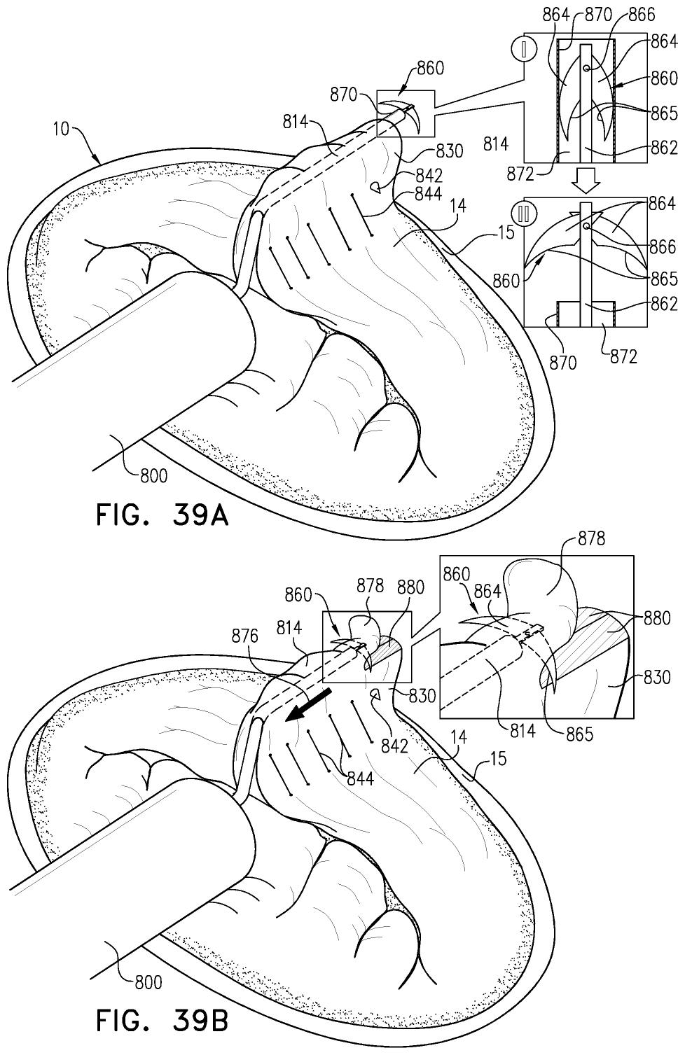

Reference is now made to FIG. 41, which is a schematic top view illustration of mitral valve 10 following repair thereof using the method of FIGS. 34 to 40B. As seen, the excess tissue has been removed from bulge formed of the valve leaflet, such that the leaflets 12 and 14 now better coapt at reference numeral 17, i.e., better closing the valve. Suture 844 extends along leaflet 14, from lip 14a toward annulus 15, substantially perpendicular to the lip, and holds together cut edges 880. The suture is held by needle tip 842, which remains adjacent the tissue at one of the attached segments of the bulge, and by a locking element 882, placed onto the end of suture 844 substantially as described hereinabove with respect to FIGS. 11A to 13.

It is to be appreciated that the method described hereinabove with respect to FIGS. 33 to 41 is substantially similar to the method described hereinabove with respect to FIGS. 1 to 9, in that both methods include a step of cutting tissue away from the leaflet, and securing the cut edges of tissue. In the method of FIGS. 1 to 9, the tissue is cut to form the cut edges, which are then secured. By contrast, in the method of FIGS. 33 to 41, the tissue is initially secured, and then cut edges (which are already secured to one another) are formed by cutting away of the tissue.

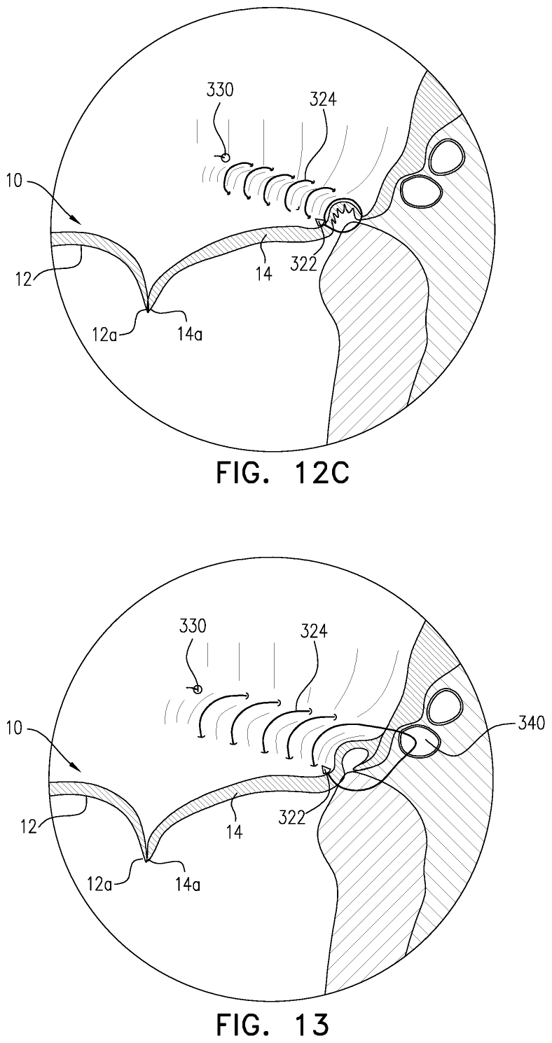

Reference is now made to FIGS. 14A to 14D, which are schematic illustrations of an implant 350, and phases of use thereof, for repairing a leaflet of a heart valve, in accordance with some applications.

Implant 350 comprises a frame 351 that includes a first leaflet-engaging end 352a and a second leaflet-engaging end 352b. First and second leaflet-engaging ends 352a and 352b are connected to each other by an intermediate portion of the frame. The intermediate portion includes a generally U-shaped central portion 354, having first and second arm portions 356a and 356b extending from respective ends of central portion 354 to respective leaflet-engaging ends 352a and 352b of the arm portions.

Implant 350 further comprises at least one tether 360 that extends away from leaflet-engaging ends 352a and 352b. In some applications, one tether 360 extends away from each of the leaflet-engaging ends, e.g., each tether is fixedly attached to a corresponding one of the leaflet-engaging ends. In some applications, such as the illustrated example, a hollow lumen 358 extends through the entire length of frame 351, from first leaflet-engaging end 352a, via first arm portion 356a, central portion 354, and second arm portion 356b, to second leaflet-engaging end 352b. In such examples, a single tether 360 can extend through the entirety of lumen 358, and out of leaflet-engaging ends 352a and 352b, e.g., as shown.

In some applications, the length of frame 351 is formed as a unitary structure.

Frame 351 can be a flexible and/or resilient device. For example, in some applications, frame 351 can be formed of a resilient metal, or of a shape memory material.

In some applications, frame 351 is generally planar. Optionally, and as shown, arm portions 356 can be curved out of plane, e.g., to curve around the leaflet.

In some applications, in a first operative state of implant 350, which is a rest state devoid of force applied to the implant, arm portions 356a and 356b extend outwardly from ends of central portion 354. In the rest state, a distance d1 between ends of arm portions 356a and 356b which connect to leaflet-engaging ends 352a and 352b, respectively, is greater than a distance d2 between ends of the arm portions which engage central portion 354. In the rest state, shown in FIG. 14A, leaflet-engaging ends 352a and 352b are angled inwardly with respect to arm portions 356a and 356b, such that a distance d3, smaller than d1, extends between ends of leaflet-engaging ends 352a and 352b.

When a force is applied to leaflet-engaging ends 352a and 352b, pushing them towards each other, arm portions 356a and 356b pivot relative to central portion 354, to decrease the distance d2, and to bring leaflet-engaging ends 352a and 352b closer to each other, as explained in further detail hereinbelow.

Turning to FIG. 14B, first leaflet 12 has excess tissue 16b, such that a gap 18b is formed between lips 12a and 14a of leaflets 12 and 14.

As seen in FIG. 14B, implant 350 is transluminally delivered to the heart chamber adjacent mitral valve 10, e.g., to the heart chamber upstream of the mitral valve, when the implant is in the rest state. In some applications, the implant is delivered by at least one longitudinal catheter, similar to catheter 100 described hereinabove, the catheter being configured to be transluminally advanced toward the heart chamber. As described hereinabove, the longitudinal catheter has a proximal part and a steerable distal part, and a longitudinal axis therebetween.

As seen in FIG. 14C, the catheter is adapted to be steerable so as to engage first leaflet-engaging end 352a with lip 12a of leaflet 12 at a first location along the lip, and to engage second leaflet-engaging end 352b with lip 12a at a second location along the lip. In this arrangement, central portion 354 and arm portions 356a and 356b are disposed on a downstream side of mitral valve 10, with excess tissue 16b resting on, and/or between, the central portion and the arm portions. Leaflet-engaging ends 352a and 352b extend past and around lip 12a, and can engage an upstream surface of leaflet 12.

In some applications, such as in the illustrated example, implant 350 engages leaflet 12 such that arm segments of U-shaped central portion 354 are substantially perpendicular to lip 12a.

Turning to FIG. 14D, it is seen that tensioning of tether 360 applies a force to leaflet-engaging ends 352a and 352b, drawing them, as well as arm portions 356a and 356b, toward each other. As a result, implant 350 transitions to a second operative state. Motion of arm portions 356a and 356b toward each other causes excess tissue 16b to become trapped between the arms, e.g., forming a bulge 361 in the downstream surface of leaflet 12. The tension in tether 360 is secured by a locking element 362 which is slid onto both ends of the tether, to secure the tether in its tensioned state. Tensioning of tether 360, and locking of the tension therein can be carried out by the catheter, for example in response to human operation of a control handle, e.g., similarly to that described hereinabove with respect to catheter 100 and control handles thereof, mutatis mutandis.

As seen in FIG. 14D, as a result of the formation of the bulge in leaflet 12, lip 12a of the leaflet has been shortened, such that leaflets 12 and 14 properly coapt along the entire length thereof, and gap 18b has been closed.

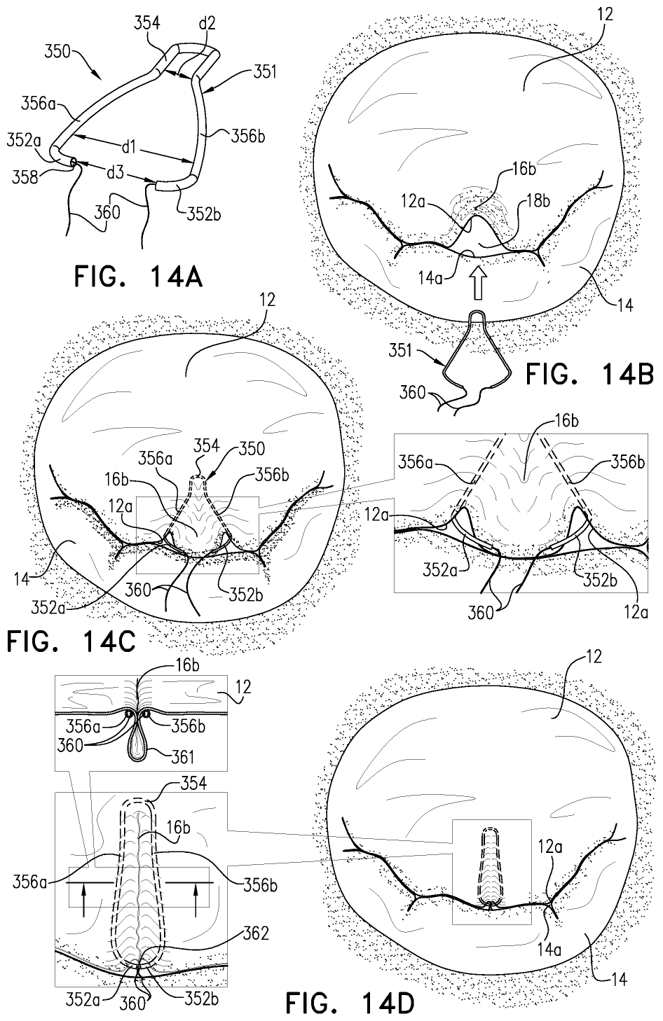

Reference is now made to FIGS. 15A, 15B, and 15C, which are schematic top view illustrations of phases of repairing a leaflet of a heart valve using another implant 370, in accordance with some applications.

Implant 370 includes a first leaflet-engaging clip 372a and a second leaflet-engaging clip 372b. Each of clips 372a and 372b includes a first surface 374, adapted to engage an upstream surface of the lip of a valve leaflet, and a second surface 376, adapted to engage a downstream surface of the lip of a valve-leaflet, the first and second surfaces being connected by a hinge portion 378. First and second leaflet-engaging clips 372a and 372b are connected to each other by one or more tethers 380, extending between first surfaces 374. In the example shown, implant 370 comprises a pair of tethers 380. Each of clips 372a and 372b has a first operative state, which is an open state, in which surfaces 374 and 376 are angled relative to each other, about hinge 378, and a second operative state, which is a closed state, in which surfaces 374 and 376 are substantially parallel to one another.

In the illustrated example, implant 370 is used to repair first leaflet 12 of mitral valve 10. As seen in FIG. 15A, first leaflet 12 has excess tissue 16b, such that a gap 18b is formed between lips 12a and 14a of leaflets 12 and 14.

Implant 370 is transluminally delivered to the heart chamber adjacent mitral valve 10, e.g., to the heart chamber upstream of the mitral valve. In some applications, the implant is delivered by at least one elongate catheter 390, the catheter being configured to be transluminally advanced toward the heart chamber. As described hereinabove with respect to catheter 100, longitudinal catheter 390 has a proximal part and a steerable distal part, and a longitudinal axis therebetween. At least one clip-mounting tool 392, to which at least one of clips 372a and 372b is connected, is advanced distally out of catheter 390, with the connected clip being in the open state.

Catheter 390 and/or tool 392 is adapted to be steerable so as to bring clip-mounting tool 392 to lip 12a of the leaflet, for the clip-mounting tool to clamp the clip mounted thereon onto the lip of the leaflet, by transitioning the from the open state to the closed state. In FIG. 15A, first leaflet-engaging clip 372a has already been clamped onto a lip 12a at a first location, adjacent an end of excess tissue 16b. As seen, surface 374 of the clip presses against the upstream surface of leaflet 12, while surface 376 presses against the downstream surface of the leaflet. Clip-mounting tool 392 is prepared to clamp clip 372b, which is mounted thereon in the open position, onto lip 12a.

Turning to FIG. 15B, it is seen that catheter 390 and clip-mounting tool 392 have been used to clamp second leaflet-engaging clip 372b onto the lip 12a of leaflet 12 at a second location along the lip, such that excess tissue 16b extends between the first and second clips. In this arrangement, tethers 380 extend over excess tissue 16b, as seen clearly in FIG. 15B.

Following attachment of second clip 372b to the lip of the leaflet, clip-mounting tool 392 can be retracted into catheter 390, and optionally out of the subject, as seen in FIG. 15B. Tethers 380 continue to extend through catheter 390, e.g., to a control handle thereof or outside of the catheter, for access by a human operator.

In some applications, a single clip-mounting tool 392 can be used to deliver and place both clips 372a and 372b, for example one after the other. In some applications, a separate clip-mounting tool is used for delivery and placement of each of the clips 372a and 372b.

Turning to FIG. 15C, it is seen that tensioning of tethers 380 draws leaflet-engaging clips 372a and 372b toward each other. Motion of clips 372a and 372b toward each other causes excess tissue 16b to plicate a region 394 of lip 12a of leaflet 12 disposed between the clips. In some applications, the tension in tethers 380 can be secured by locking elements 396 which are slid onto tethers 380 in order to secure the tethers in their tensioned state. Although FIG. 15C shows each tether 380 having a corresponding locking element 396, for some applications a single locking element can be used to lock multiple (e.g., all) tethers 380.

Tensioning of tethers 380, and locking of the tension therein can be carried out by the catheter, for example in response to human operation of a control handle, e.g., similarly to that described hereinabove with respect to catheter 100 and to control handles thereof, mutatis mutandis.

As seen in FIG. 15C, as a result of plication of region 394 of leaflet 12, lip 12a of the leaflet has been shortened, such that leaflets 12 and 14 properly coapt along the entire length thereof, and gap 18b has been closed.

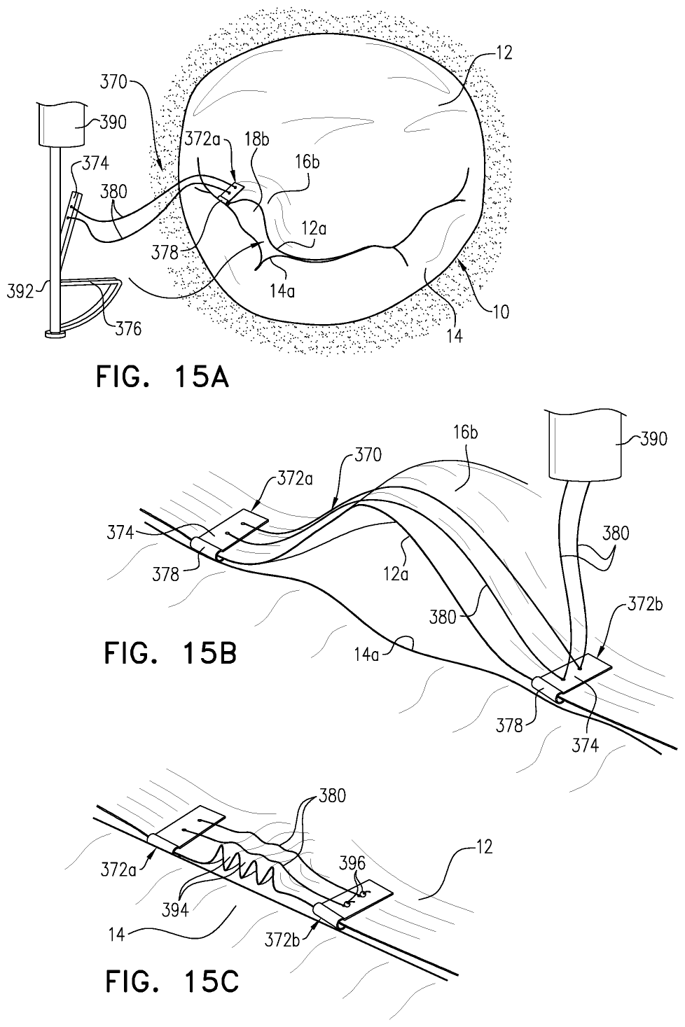

Reference is now made to FIGS. 16A to 16D, which are schematic illustrations of an implant, and phases of use thereof, for repairing a leaflet of a heart valve, in accordance with some applications.

FIG. 16A is a schematic perspective view illustration of an implant 400. As seen, implant 400 includes a piercing element 402 and a securing element 404. Piercing element 402 includes a base 406 from which extends a piercing tip 408. In some applications, such as the illustrated example, securing element 404 includes a housing 410 adapted to receive piercing tip 408, and to secure the piercing tip therein. As shown, securing element 404 can define a hollow 414 that is adapted to receive piercing tip 408, can be substantially cylindrical, and/or can terminate at a wall portion 412. However, in some applications, the securing element can have a different structure. The securing element can be formed from a biocompatible elastomer adapted to receive and grip the piercing tip 408 when implanted, as explained herein.

Turning to FIG. 16B, first leaflet 12 has excess tissue 16b, such that a gap 18b is formed between lips 12a and 14a of leaflets 12 and 14.

As seen in FIG. 16B, implant 400 can transluminally delivered to the heart chamber adjacent mitral valve 10 (e.g., to the heart chamber upstream of the mitral valve) while piercing element 402 is separate from securing element 404. Optionally, in some applications, implant 400 can be delivered while piercing tip 408 is secured within securing element 404. In such examples, the piercing element and the securing element are separated and rejoined during implantation of implant 400.

In some applications, implant 400 is delivered by at least one longitudinal catheter, e.g., similar to catheter 100 described hereinabove, the catheter being configured to be transluminally advanced toward the heart chamber. As described hereinabove, the longitudinal catheter has a proximal part and a steerable distal part, and a longitudinal axis therebetween.

As seen in FIG. 16C, the catheter is adapted to be steerable so as to create a folded (or plicated) region 416 of leaflet 12, by folding the leaflet. The catheter is then configured to insert piercing tip 408 of piercing element 402 through at least two layers of the leaflet at folded region 416. The catheter is further configured to secure at least onefold of the folded region is secured by connecting securing element 404 to piercing tip 408, so as to secure the piercing tip within the hollow of securing element 404. When securing piercing tip 408 with securing element 404, both piercing element 402 and securing element 404 are on the same side of leaflet 12, shown in FIG. 16C as the upstream side of the leaflet.

Folding of the leaflet, piercing of the folded region using piercing element 402, and securing of piercing tip 408 of the piercing element, can be carried out by the catheter, for example in response to human operation of a control handle, e.g., similarly to that described hereinabove with respect to catheter 100 and to control handles thereof, mutatis mutandis.

As a result of folding and securing the leaflet, at least two layers of tissue are disposed between piercing element 402 and securing element 404, the two or more layers of tissue forming at least one bulge along lip 12a. Consequently, lip 12a is shortened, such that leaflets 12 and 14 properly coapt along the entire length thereof, and gap 18b has been closed.

In some applications, such as that shown in FIG. 16C, folded region 416, as well as piercing element 402 and securing element 404, are oriented toward the upstream side of the valve. However, in some applications, such as that shown in FIG. 16D, folded region 416, piercing element 402, and securing element 404, are oriented toward the downstream side of the valve.

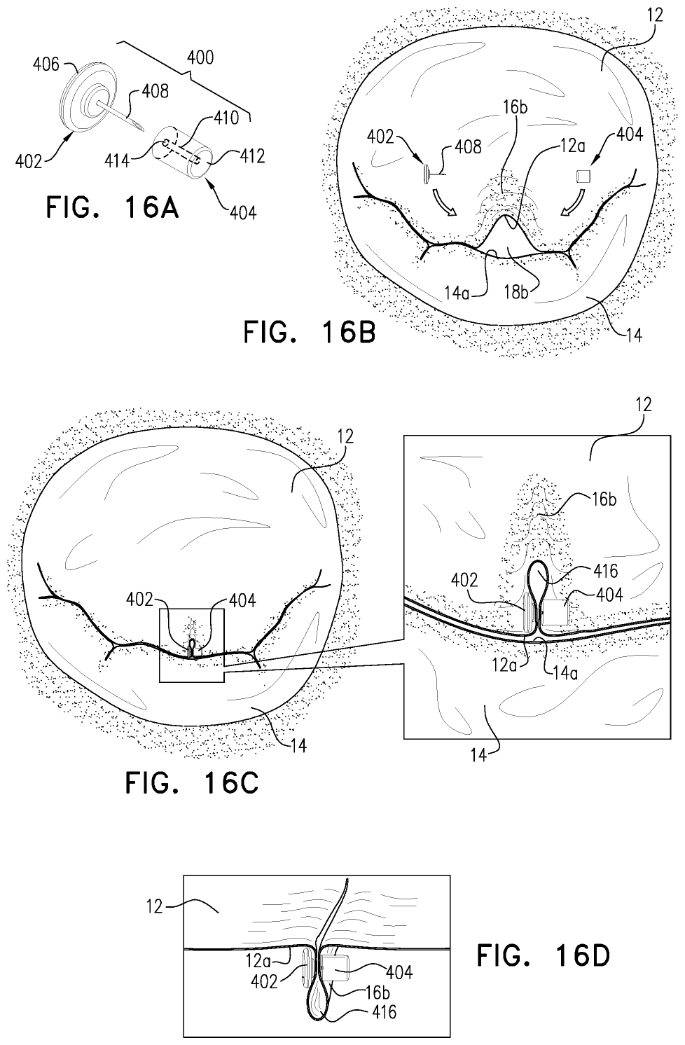

Reference is now made to FIGS. 17A, 17B, 17C, and 17D, which are schematic top view illustrations of phases of repairing a leaflet of the mitral valve using a tether implant which includes one or more tethers 420, in accordance with some applications.

In the illustrated example, the tether implant is used to repair second leaflet 14 of mitral valve 10. As seen in FIG. 17A, a prolapse 16 exists in second scallop (i.e., P2) of leaflet 14, the second scallop being delineated by separations 422a and 422b between the second scallop and the adjacent scallops (i.e., P1 and P3).

Tether(s) 420 of the tether implant is (are) transluminally delivered to the heart chamber adjacent mitral valve 10, e.g., to the heart chamber upstream of the mitral valve. In some applications, the implant is delivered by at least one longitudinal catheter 424, the catheter being configured to be transluminally advanced toward the heart chamber. As described hereinabove with respect to catheter 100, longitudinal catheter 424 has a proximal part and a steerable distal part, and a longitudinal axis therebetween.

Catheter 424 is adapted to be steerable so as to guide tether(s) 420 of the tether implant to leaflet 14, and to thread a distal end 421a of tether 420 through separation 422a into the ventricle, alongside a downstream surface of leaflet 14, and back through separation 422b into the atrium (FIG. 17A).

Turning to FIG. 17B, a gripping tool 426, such as a snare, is advanced distally out of catheter 424, and is adapted to grip distal end 421a of tether 420 of the tether implant. As seen in FIG. 17C, gripping tool 426 has pulled distal end 421a of tether 420 back to catheter 424. As a result, tether 420 of the tether implant is now wrapped around the central scallop of second leaflet 14.

Turning to FIG. 17D, subsequent tensioning of tether 420 causes prolapse 16 to be compressed. The tension in tether 420 is then secured by a locking element 428 which are slid onto ends 421a and 421b of the tether, to secure the tether in its tensioned state.

Tensioning of tether 420, and locking of the tension therein can be carried out by catheter 422, for example in response to human operation of a control handle, e.g., similarly to that described hereinabove with respect to catheter 100 and to control handles thereof, mutatis mutandis.

As seen in FIG. 17D, as a result of the formation of folds in leaflet 14, lip 14a of the leaflet has been shortened, such that leaflets 12 and 14 properly coapt along the entire length thereof.

In some applications, the tether implant can include more than one tether 420. In such examples, each tether 420 of the tether implant is threaded through separations 422b and 422a and tensioned as described herein, e.g., such that the tethers 420 of the tether implant extend parallel to one another and parallel to lip 14a of the second leaflet.

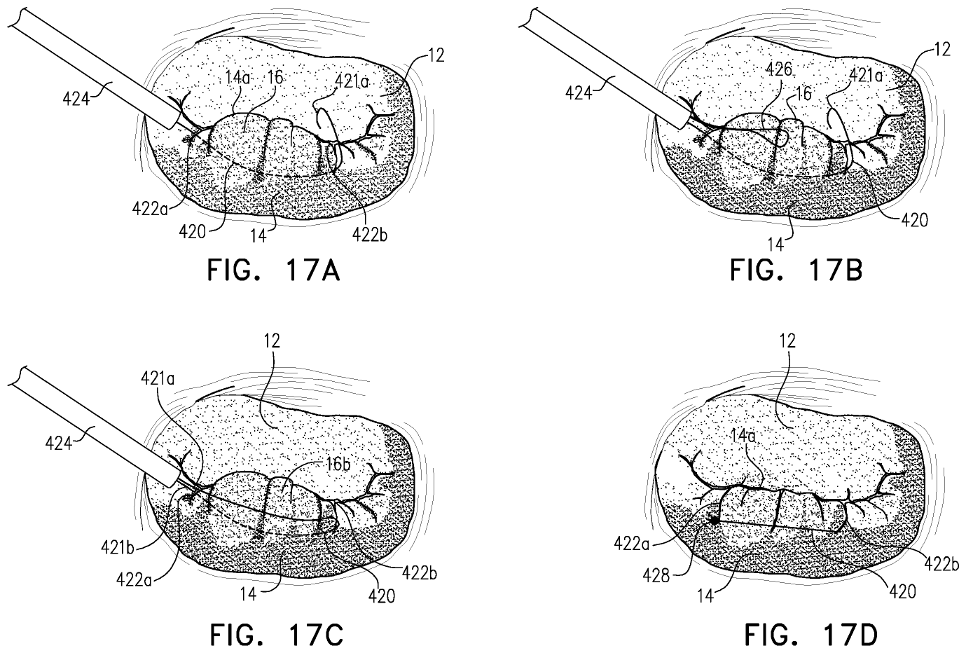

Reference is now made to FIGS. 18A to 18C, which are schematic illustrations of an implant 450, and phases of use thereof, for repairing a leaflet of a heart valve, in accordance with some applications.

As seen in FIG. 18A, implant 450 includes a base portion 452, from which extend a plurality of beams, here shown as five beams 454a, 454b, 454c, 454d, and 454e, each of the beams having a terminus 456 distal to base portion 452. Beams 454a, 454b, 454c, 454d, and 454e can be substantially parallel to one another, and/or can be substantially perpendicular to base portion 452. Although five beams are shown, implant 450 can include any number of beams, and often include at least three beams.

Implant 450 can be a flexible and/or resilient device. For example, in some applications, implant 450 can be formed of a resilient or shape memory material (e.g., nitinol, spring steel, or cobalt-chrome).

In some applications, in a first operative state of implant 450, which is an open state, termini 456 of a first subset of the plurality of beams are not in the same plane as termini 456 of a second subset of the plurality of beams. In some applications, the beams in the first subset alternate with the beams in the second subset. For example, in the example illustrated in FIG. 18A, the first subset includes second beam 454b and fourth beam 454d, whose termini 456 are disposed in a lower plane than termini of the first, third, and fifth beams 454a, 454c, and 454e.

In a second operative state of implant 450, which is a closed state, base portion 452 as well as termini 456 of all of beams 454a, 454b, 454c, 454d, and 454e can be disposed in a single plane (e.g., as shown). In some applications, during the transition from the open state toward the close state, termini of beams 454a, 454b, 454c, 454d, and 454e move toward the single plane. In the illustrated examples, the termini reach the single plane, and in some applications the termini can even pass each other, and continue to move past the single plane, relative to each other.

Turning now to FIG. 18B, first leaflet 12 of mitral valve 10 has excess tissue 16b, such that a gap 18b is formed between lips 12a and 14a of leaflets 12 and 14.

As seen in FIG. 18B, implant 450 is transluminally delivered to the heart chamber adjacent mitral valve 10, e.g., to the heart chamber upstream of the mitral valve, in the first, open, operative state.

In some applications, implant 450 is delivered by at least one longitudinal catheter, similar to catheter 100 described hereinabove, the catheter being configured to be transluminally advanced toward the heart chamber. As described hereinabove, the longitudinal catheter has a proximal part and a steerable distal part, and a longitudinal axis therebetween.

As seen in FIG. 18C, the catheter is adapted to be steerable so as to slide implant 450 onto leaflet 12. This can be done, for example, in a manner in which beams 454a, 454b, 454c, 454d, and 454e are substantially perpendicular to lip 12a of the leaflet, and base portion 452 extends along the lip of the leaflet, e.g., as shown. Implant 450 is placed onto leaflet 12 in the open state, such that the beams of one of the subsets are disposed upstream of the leaflet, and beams of the other of the subsets are disposed downstream of the leaflet. In the illustrated example, beams 454a, 454c, and 454e of the second subset are disposed upstream leaflet 12, and beams 454b and 454d of the second subset are disposed downstream of the leaflet. Because of the finger-like nature of beams 454, they can slide between chordae tendineae without becoming ensnared.

The catheter is then configured to transform implant 450 to the second, closed operative state, in which beams 454 move toward a single plane, e.g., by removing a restraint and thereby allowing the implant to responsively (e.g., elastically or resiliently) move into its closed operative state. As seen in the enlarged sectional portion of FIG. 18C, when implant 450 is in the second state, leaflet 12 follows a tortuous path between beams 454a and 454e, going under and over the alternating beams.

Placing the implant onto the leaflet and transforming the implant from the first operative state to the second operative state can be carried out by the catheter, for example in response to human operation of a control handle, e.g., similarly to that described hereinabove with respect to catheter 100 and to control handles thereof, mutatis mutandis.

In some applications, the catheter can be configured to modify the arrangement of implant 450, e.g., when transitioning from the open state to the closed state, by applying force to implant 450 or to a portion thereof. The applied force actively changes, or deforms, the shape of the implant.

In some applications, the catheter can be configured to modify the arrangement of implant 450 by removing a constraint, constraining implant 450 in a specific state. Removal of constraints is particularly useful when the implant is formed of an elastic or resilient material, such as a shape memory material.

As a result of leaflet 12 following a tortuous path over and under beams of implant 450, excess tissue 16b is consumed by the tortuous path, and the effective length of lip 12a of leaflet 12 is shortened, thereby reducing prolapse and/or regurgitation.

Although the illustrated example shows that in the open state the beams alternate between the first subset and the second subset (i.e., neither subset includes two adjacent beams), in some applications the beams can be divided into the two subsets in other ways.

It is to be noted that while the illustrated example shows the lateral-most beams 454a and 454e disposed on the upstream surface of leaflet 12, the arrangement of implant 450 can also be reversed, such that the lateral-most beams are disposed against the downstream surface of the leaflet.

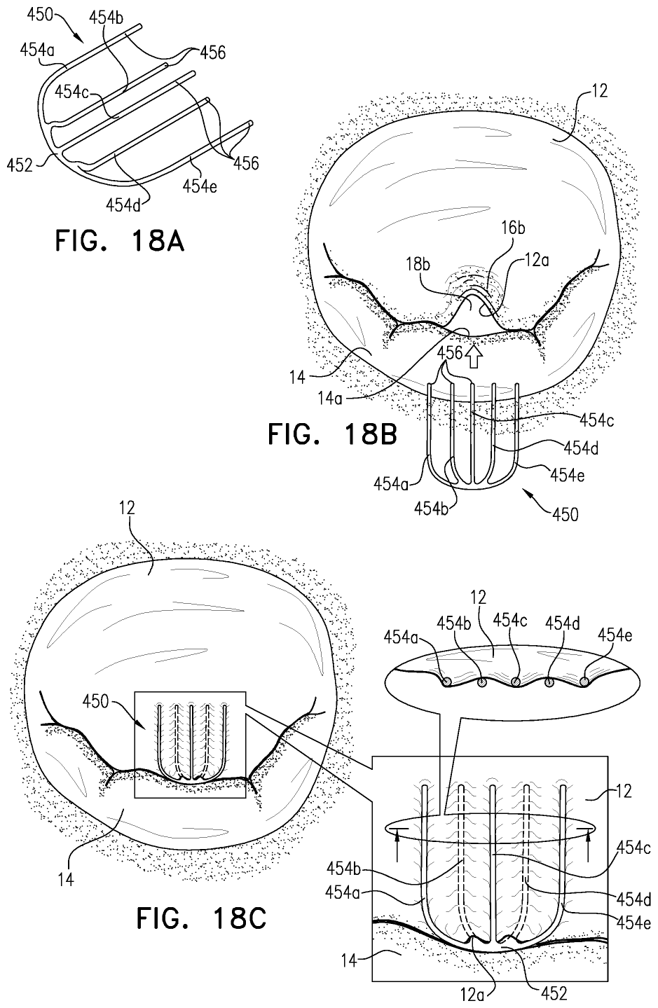

Reference is now made to FIGS. 19A to 19C, which are schematic illustrations of an implant 460, and phases of use thereof, for repairing a leaflet of a heart valve, in accordance with some applications.

Turning to FIG. 19A, implant 460 includes a first U-shaped portion 462, including a base portion 464 from which extend two beams 466 and 468. Implant 460 includes a second U-shaped portion 472, including a base portion 474 from which extend two beams 476 and 478. Beams 466 and 468 can be substantially perpendicular to base portion 464, and/or substantially parallel to each other. Similarly, beams 476 and 478 are substantially perpendicular to base portion 474, and substantially parallel to each other. In some applications, U-shaped portions 462 and 472 can be substantially identical to one another.

Beams 468 and 476 are engaged with one another, and are held together within a longitudinally extending cylindrical engagement element 480, to form a unified central beam. Beams 468 and 476 are pivotable within cylindrical engagement element 480, about longitudinal axes of the beams. Consequently, first U-shaped portion 462 and second U-shaped portion 472 are rotatable, or pivotable, relative to each other.

Implant 460 can be a flexible and/or resilient device. For example, in some applications, implant 460 can be formed of a resilient metal, or of a shape memory material.

In some applications, in a first operative state of implant 460, shown in FIG. 19A, U-shaped portions 462 and 472 are disposed such that a first distance, indicated by H1 in FIG. 19B, exists between beams 466 and 478. In this state, U-shaped portions 462 and 472 can be disposed substantially in a single plane.

In a second operative state of implant 460, U-shaped portions 462 and 472 are rotated toward each other, such that the unified central beam is disposed in a different plane than edge beams 466 and 478. In the second operative state, a second distance, indicated by H2 in FIG. 19C, exists between beams 466 and 478 thereof. Distance H2 can be smaller than distance H1.

As seen in FIG. 19B, first leaflet 12 has excess tissue 16b, such that a gap 18b is formed between lips 12a and 14a of leaflets 12 and 14. Implant 460 is transluminally delivered to a heart chamber adjacent mitral valve 10, e.g., to the heart chamber upstream of the mitral valve, in the first, planar, operative state.

In some applications, implant 460 is delivered by at least one longitudinal catheter, similar to catheter 100 described hereinabove, the catheter being configured to be transluminally advanced toward the heart chamber. As described hereinabove, the longitudinal catheter has a proximal part and a steerable distal part, and a longitudinal axis therebetween.

As seen in FIG. 19C, the catheter is adapted to be steerable so as to slide implant 460 onto leaflet 12, e.g., such that beams 466 and 478, as well as the unified central beam, are substantially perpendicular to lip 12a of the leaflet, and base portions 464 and 474 extend along the lip of the leaflet. Implant 460 is placed onto leaflet 12 such that edge beams 466 and 478 engage, or are disposed adjacent to, one surface of the leaflet, and the central unified beam, including central beams 468 and 476, wrapped in cylindrical engagement element 480, engages, or is disposed adjacent to, an opposing surface of the leaflet. In the illustrated example, the edge beams are disposed adjacent, or on, the upstream surface of leaflet 12, and the central unified beam is disposed adjacent, or on, the downstream surface of the leaflet.

The catheter is then configured to transform implant 460 to the second, angled operative state, in which U-shaped portions 462 and 472 are angled relative to one another. As seen in the enlarged portion of FIG. 19C, in this orientation, edge beams 466 and 478 are disposed in a different plane than the unified central beam, here shown as a lower plane. In this orientation, leaflet 12 follows a tortuous path between beams 466 and 478, going over the central unified beam.

Placing the implant onto the leaflet and transforming the implant from the first operative state to the second operative state can be carried out by the catheter, for example in response to human operation of a control handle, e.g., similarly to that described hereinabove with respect to catheter 100 and to control handles thereof, mutatis mutandis.

In some applications, the catheter can be configured to modify the arrangement of implant 460, e.g., when transitioning from the first state to the second state, by applying force to implant 460 or to a portion thereof. The applied force actively changes, or deforms, the shape of the implant.

In some applications, the catheter can be configured to modify the arrangement of implant 460 by removing a constraint, constraining implant 460 in a specific state. Removal of constraints is particularly useful when the implant is formed of an elastic or resilient material, such as a shape memory material.

As a result of leaflet 12 following a tortuous path over and under beams of implant 460, excess tissue 16b is utilized for completion of the tortuous path, and the effective length of lip 12a of leaflet 12 is shortened. Consequently, leaflets 12 and 14 properly coapt during ventricular systole. As seen in the enlarged portion of FIG. 19C, for some applications implant 460 (e.g., base portion 464 and 474 thereof) can abut leaflet 14, during ventricular systole.

It is to be noted that while the illustrated example shows edge beams 466 and 478 disposed on the upstream surface of leaflet 12, and the central beam is disposed against the downstream surface of the leaflet, the arrangement of implant 460 can also be reversed, such that the edge beams are disposed against the downstream surface of the leaflet and the central beam is disposed against the upstream surface of the leaflet.

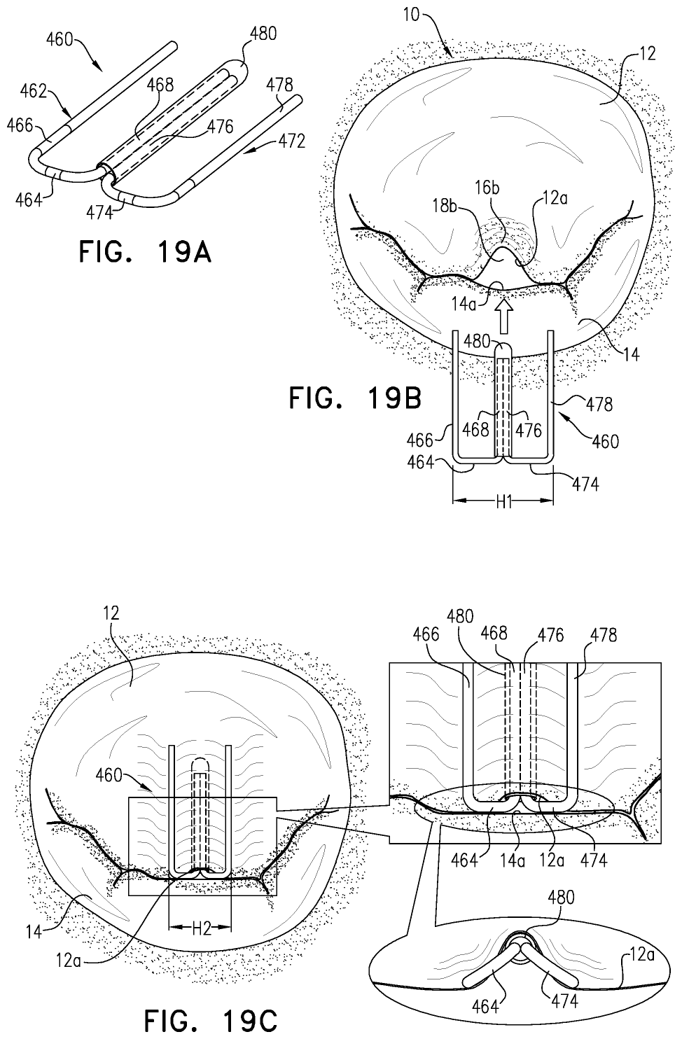

Reference is now made to FIG. 20, which is a schematic view illustration of phases of placing an implant 500 onto a leaflet of a cardiac valve, thereby to repair the leaflet, in accordance with some applications.

Implant 500 is formed of a length of a flexible and/or resilient material. For example, in some applications, implant 500 can be formed of a resilient metal, or of a shape memory material. In some applications, implant 500 is unitarily formed, e.g., from a single piece of stock material.

In some applications, implant 500 is substantially reflectionally symmetrical about an axis of symmetry 501, and operation thereof can be carried out symmetrically on both sides thereof, as explained hereinbelow.

Implant 500 is shaped to define a first beam 502, terminating at one end thereof in a free end 503, and at the other end thereof in a first bend 504. A second beam 506 extends from bend 504, to a second bend 508. A generally U-shaped portion 510 extends from second bend 508 to a third bend 512. U-shaped portion 510 can include a pair of longitudinal beams 510a, separated by a curved base portion 510b. A third beam 514 extends from third bend 512 to a fourth bend 516, and a fourth beam 518 extends from fourth bend 516 to a second free end 519.

As seen in phase I of FIG. 20, in a first operative state of implant 500, the implant can be planar. In this operative state, each of bends 504, 508, 512, and 516, can be substantially a 180-degree bend (subject to constraints of the material), such that beams 502, 506, 514, and 518, as well as beams 510a of U-shaped portion 510, can be substantially parallel to one another. In this arrangement, bends 504, 508, 512, and 516, as well as base portion 510b of U-shaped portion 510, can be in a single plane.

Turning to phase II, it is seen that beams 506 and 514 pivot upward about bends 508 and 512, respectively, as indicated by arrows 520. Such pivoting causes bends 504 and 516 to be in a different plane than bends 508 and 512, resulting in a second operative state of implant 500. In this operative state, beams 502, 506, 514, and 518 are in a different plane than U-shaped portion 510, or in multiple different planes (e.g., when beams 506 and 514 do not pivot to the same angular extent relative to U-shaped portion 510). The second operative state can also be considered an open operative state.

As seen at phase III, implant 500 is transluminally delivered to the heart chamber adjacent a heart valve, such as the mitral valve, e.g., to the heart chamber upstream of the mitral valve. As shown in phase III, implant 500 is delivered in the second, open, operative state. However, in some applications, the order of phases II and III can be reversed, such that implant 500 is delivered to the heart chamber in the first planar operative state shown at phase I, and is transitioned to the second operative state, as shown at phase II, when in the heart chamber.

Implant 500 is adapted to be placed onto a leaflet 522 of a heart valve, e.g., a leaflet that has excess tissue 526. For example, the heart valve can be a mitral valve, as described hereinabove, or can be a tricuspid valve. Leaflet 522 can be any leaflet of the heart valve, such as the anterior or posterior leaflet of the mitral valve, or any one of the leaflets of the tricuspid valve.

In some applications, implant 500 is delivered by at least one longitudinal catheter, similar to catheter 100 described hereinabove, the catheter being configured to be transluminally advanced toward the heart chamber. As described hereinabove, the longitudinal catheter has a proximal part and a steerable distal part, and a longitudinal axis therebetween.

The catheter is adapted to be steerable so as to slide implant 500 onto leaflet 522, in the direction of arrow 528, e.g., such that beams 502, 506, 514, and 518, as well as longitudinal beams 510a of U-shaped portion 510 are substantially perpendicular to lip 522a of the leaflet. In this arrangement, bends 508 and 512 are adjacent lip 522a, and base portion 510b of U-shaped portion 510 is distal to the lip. In some applications, the implant is placed in the vicinity of excess tissue 526.

Implant 500 is placed onto leaflet 522 in the second, open, operative state, such that U-shaped portion 510 engages, or is disposed adjacent to, a downstream surface of the leaflet, and beams 502, 506, 514, and 518 are disposed above an upstream surface of the leaflet.

Turning to phase IV, it is seen that the catheter is configured to transform implant 500 back to the first, planar operative state, by pivoting beams 506 and 514 downward about bends 508 and 512, respectively, as indicated by arrows 530. The pivoting of beams 506 and 514 at phase IV is in an opposite direction to the pivoting at phase II. Following pivoting of beams 506 and 514, beams 502, 506, 514, and 518 engage, or are disposed adjacent to, the upstream surface of leaflet 522, as shown at phase V. As shown, leaflet 522 can be sandwiched between beam 506 and one of beams 510a, and between beam 514 and the other of beams 510a.

At phase VI, the catheter is configured to pivot beam 506 about bend 508 and beam 514 about bend 512, such that beams 506 and 514 revolve around beams 510a, e.g., without substantially deflecting with respect to beams 510a. As seen in phases VI and VII, this causes wrapping of the tissue of leaflet 522 about beams 510a, 502, 506, 514, and 518. As such, when implant 500 is in the third, fully folded, operative state, leaflet 522 follows a tortuous path between beams 502 and 518, as shown in the sectional portion of phase VII.

For some applications, and as shown, the revolution of beams 506 and 514 about beams 510a can be by as much as 180 degrees. As such, following their revolution about beams 510a, beams 502, 506, 514, and 518 can lie in substantially the same plane as U-shaped portion 510, and are disposed within the footprint of the U-shaped portion, i.e., medially with respect to beams 510a.

In phase VII, implant 500 can be considered to be in a third, operative state, which can also be called a fully folded operative state. For clarity, the third operative state of implant 500 is also shown in phase VIII of FIG. 20.

Placing the implant onto the leaflet and transforming the implant between operative states thereof can be carried out by the catheter, for example in response to human operation of a control handle, e.g., similarly to that described hereinabove with respect to catheter 100 and to control handles thereof, mutatis mutandis.

In some applications, the catheter can be configured to modify the arrangement of implant 500, e.g., when transitioning from one phase of FIG. 20 to another, by applying force to implant 500 or to a portion thereof. The applied force actively changes, or deforms, the shape of the implant.

In some applications, the catheter can be configured to modify the arrangement of implant 500 by removing a constraint, constraining implant 500 in a specific phase, or arrangement. Removal of constraints is particularly useful when the implant is formed of an elastic or resilient material, such as a shape memory material.

As a result of leaflet 522 following a tortuous path over and under beams of implant 500, excess tissue 526 is utilized for completion of the tortuous path, and the effective length of lip 522a of leaflet 522 is shortened.

It is to be noted that, in some applications, beams 502 and 518 and bends 504 and 516 can be omitted, and instead the free ends of the implant are at the ends of beams 506 and 514.

It is to be noted that while the illustrated example shows U-shaped portion 510 being placed onto the downstream surface of leaflet 522 at phase III, with beams 502, 506, 514, and 518 being disposed above the upstream surface of the leaflet, the arrangement of implant 500 can also be reversed, such that the beams are disposed against the downstream surface of the leaflet and the U-shaped portion above the upstream surface of the leaflet. In such examples, at phase IV, the U-shaped portion would be pivoted relative to the beams, to reach the first operative state of the implant, when on the leaflet.

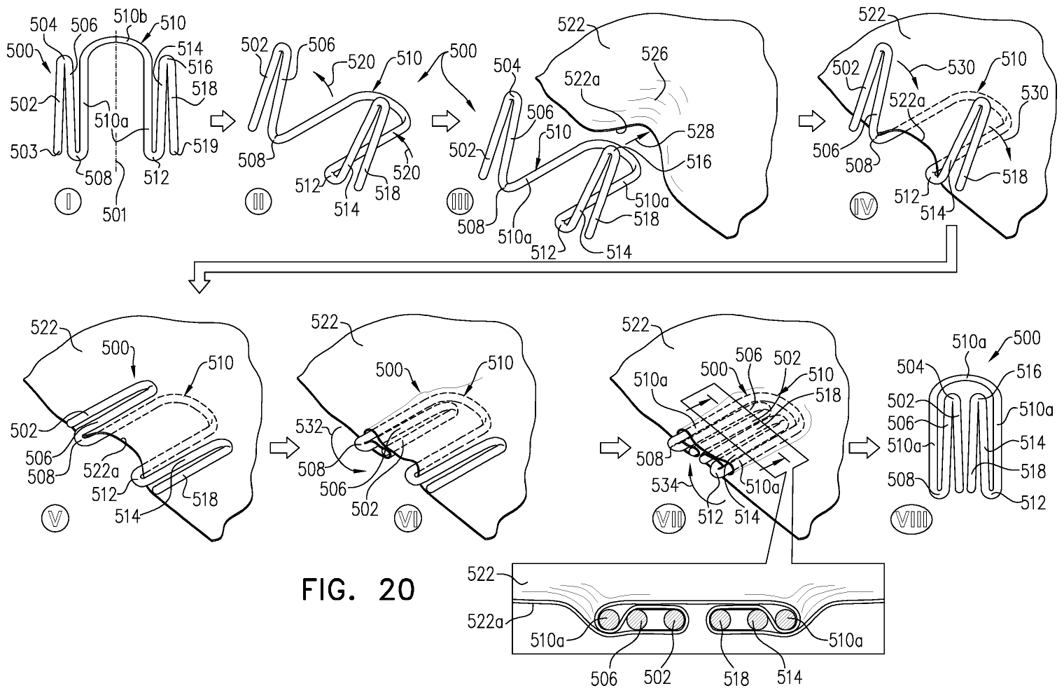

Reference is now made to FIGS. 21A to 21D, which are schematic illustrations of an implant 540, and phases of use thereof, for repairing a leaflet of a heart valve, in accordance with some applications.

As seen in FIG. 21A, implant 540 comprises a tubular body 542, having a pair of clips 544 mounted thereon. In some applications, each of clips 544 is substantially U-shaped and has a first elongate portion 546 disposed on, or near, an exterior surface of tubular body 542, and a second elongate portion 547 disposed on, or near, an interior surface of tubular body 542. Within each clip 544, elongate portions 546 and 547 are connected to each other by an end portion 548, typically such that portions 546 and 547 are substantially parallel with each other, e.g., such that the clip generally resembles a bobby pin used for styling hair. An elastic element 550 (e.g., an elastic band) couples clips 544 to each other (e.g., couples the second elongate portion 547 of one clip to the second elongate portion of the other clip) in a manner that draws the clips toward each other. Elastic element 550 can be disposed inside tubular body 542, e.g., as shown. In some applications, elastic element 550 is disposed adjacent to end portions 548 of clips 544, e.g., as shown.

As seen in FIG. 21B, in a first operative state of implant 540, clips 544 are disposed on a first side of tubular body 542, shown in FIG. 21B as an upper side of the tubular body. Clips 544 can be close to one another at the center of the upper side of the tubular body, or can be slightly separated from each other.

Implant 540 is used to repair leaflet 12 of mitral valve 10, in accordance with some applications. As seen in FIG. 21B, first leaflet 12 has excess tissue 16b, such that a gap 18b is formed between lips 12a and 14a of leaflets 12 and 14.

Implant 540 is transluminally delivered to the heart chamber adjacent mitral valve 10, e.g., to the heart chamber upstream of the mitral valve. In some applications, implant 540 can be delivered in the first operative state, e.g., as shown in FIG. 21B.

In some applications, implant 540 is delivered by at least one longitudinal catheter, similar to catheter 100 described hereinabove, the catheter being configured to be transluminally advanced toward the heart chamber. As described hereinabove, the longitudinal catheter has a proximal part and a steerable distal part, and a longitudinal axis therebetween.

The catheter is adapted to be steerable so as to slide implant 540 onto leaflet 12, in the direction of arrow 552, e.g., such that tubular body 542, as well as first and second elongate portions 546 and 547 of clips 544, are substantially perpendicular to lip 12a of the leaflet. In some applications, the implant is placed in the vicinity of excess tissue 16b.

Implant 540 is placed onto leaflet 12 in the first operative state, such that tubular body 542 engages, or is disposed adjacent to, a downstream surface of the leaflet, and first (external) elongate portions 546 of clips 544 are disposed above an upstream surface of the leaflet. For some applications, leaflet 12 is held by implant 540, such that lip 12a of the leaflet is adjacent, or engages, base portions 548 of clips 544, as shown in the inset of FIG. 21C. In this arrangement, three beams of implant 540 engage leaflet 12—tubular body 542 serving as one beam, and elongate portions 546 of two clips 544 serving as the other two beams.

The catheter is then configured to rotate clips 544 about tubular body 542, against force applied by elastic element 550, in the direction of arrows 554 in the inset of FIG. 21C. Such rotation transforms implant 540 to a second operative state, in which clips 544 engage an opposing side of tubular body 542 than in the first operative state, shown as the lower side of tubular body in the upper inset of FIG. 21D. Rotation of clips 544 about tubular body 542 causes wrapping of tissue of leaflet 12 about the tubular body. As such, when implant 540 is in the second operative state, leaflet 12 follows a tortuous path between clips 544, about tubular body 542, as shown in the upper inset of FIG. 21D.

Placing the implant onto the leaflet and transforming the implant between operative states thereof can be carried out by the catheter, for example in response to human operation of a control handle, e.g., similarly to that described hereinabove with respect to catheter 100 and to control handles thereof, mutatis mutandis.

In some applications, the catheter can be configured to modify the arrangement of implant 540, e.g., when transitioning from the first state to the second state, by applying force to implant 540 or to a portion thereof. The applied force actively changes, or deforms, the shape of the implant.

In some applications, the catheter can be configured to modify the arrangement of implant 540 by removing a constraint, constraining implant 540 in a specific state.

As a result of leaflet 12 following a tortuous path about tubular body 542 of implant 540, excess tissue 16b is utilized for completion of the tortuous path, and the effective length of lip 12a of leaflet 12 is shortened.

As seen in the lower inset of FIG. 21D, in some applications, following repair of the leaflet 12, tubular body 542 of implant 540 can be removed from the mitral valve. In such examples, a bulge 558 on the upstream side of the leaflet, formed by tissue of leaflet 12 wrapping tubular body 542, is maintained by clips 544, even when the tubular body is removed.

It is to be noted that, while the illustrated example shows tubular body 542 being placed onto the downstream surface of leaflet 12, with beams 546 of clips 544 being disposed above the upstream surface of the leaflet, the arrangement of implant 540 can also be reversed. In the reversed arrangement, the implant 540 would be placed onto leaflet 12 in the second operative state thereof, such that clips 544 are disposed against a bottom side of tubular body 542, elongate portions 546 of clips 544 are disposed against the downstream surface of the leaflet, and tubular body 542 is disposed above the upstream surface of the leaflet. In such examples, the catheter is adapted to rotate clips 544 upwards relative to tubular body 542, transitioning the implant from the second operative state to the first operative state. In such examples, bulge 558 would be formed on a downstream side of the leaflet.

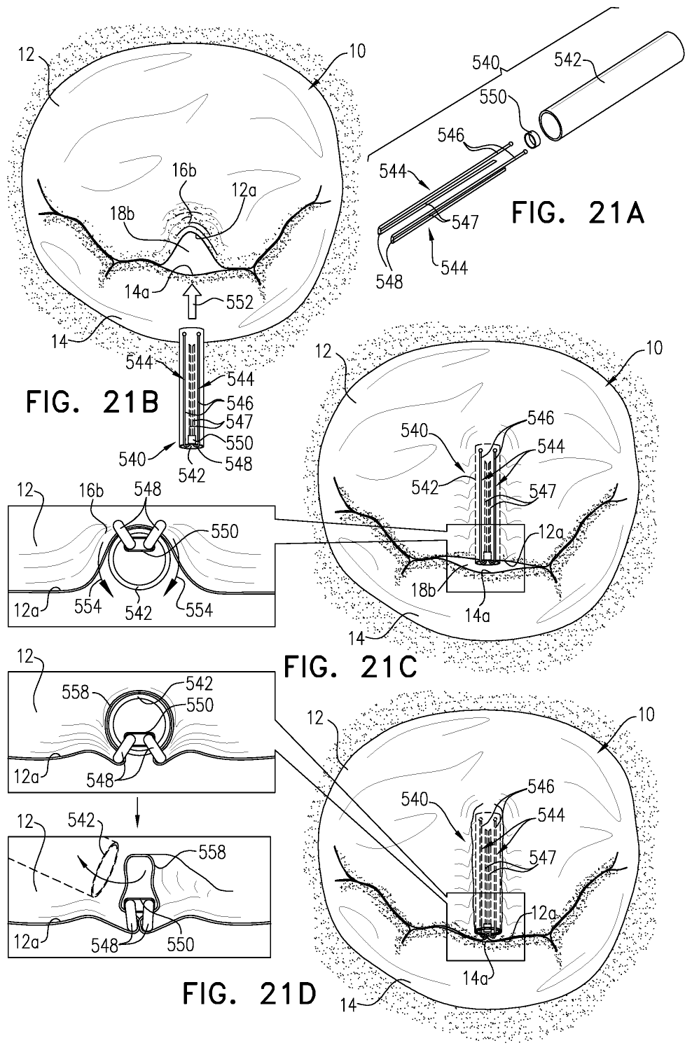

Reference is now made to FIGS. 22A to 22C, which are schematic illustrations of an implant 560, and phases of use thereof, for repairing a leaflet of a heart valve, in accordance with some applications.

Turning to FIG. 22A, implant 560 comprises a tubular core 562, e.g., a mandrel, and a securing clip 564. in some applications, securing clip 564 is substantially U-shaped and has first and second elongate portion 566, connected to each other by an end portion 568, typically such that elongate portions 566 are substantially parallel with each other, e.g., such that the clip generally resembles a bobby pin used for styling hair. Turning to FIG. 21B, in some applications, implant 560 is used to repair leaflet 12 of mitral valve 10. As seen, first leaflet 12 has excess tissue 16b, such that a gap 18b is formed between lips 12a and 14a of leaflets 12 and 14.

Tubular core 562 and securing clip 564 of implant 560 are transluminally delivered to the heart chamber adjacent mitral valve 10, e.g., to the heart chamber upstream of the mitral valve. In some applications, components of implant 560 are delivered by at least one longitudinal catheter, similar to catheter 100 described hereinabove, the catheter being configured to be transluminally advanced toward the heart chamber. As described hereinabove, the longitudinal catheter has a proximal part and a steerable distal part, and a longitudinal axis therebetween. In some applications, a first catheter is used to deliver tubular core 562, and a second catheter is used to deliver securing clip 564.

The catheter manipulating tubular core 562 is adapted to be steerable so as to slide the tubular core beneath leaflet 12, e.g., from the lip toward the root of the leaflet in the direction of arrow 572, e.g., such that tubular core 562 is substantially perpendicular to lip 12a of the leaflet. In some applications, the implant is placed in the vicinity of excess tissue 16b. As a result, excess tissue 16b rests on tubular core 562, as shown in inset I of FIG. 22C. The catheter manipulating securing clip 564 (be it the same catheter as the one manipulating core 562, or a different catheter), or another catheter or tool, is then configured to cause tissue of leaflet 12 to wrap about tubular core 562, such that the downstream surface of the leaflet engages an exterior surface of the core. The catheter manipulating securing clip 564 then pushes the clip, in a direction indicated by arrow 574—from the root of the leaflet toward the lip thereof—such that elongate portions 566 of clip 564 engage the upstream surface of leaflet 12, with end portion 568 being close to an end of the tubular core, distal to lip 12a. Thus, securing clip 564 secures the tissue about tubular core 562, and tissue of leaflet 12 forms a bulge 578 about the core, shown in inset II of FIG. 22C. The dimensions and mechanical structure of core 562, and of clip 564, are designed and configured to ensure cooperation therebetween, which cooperation ensures that core 562 is held in place, wrapped with tissue, to form bulge 578.

Placing the tubular core and securing clip onto the leaflet can be carried out by the catheter(s), for example in response to human operation of a control handle, e.g., similarly to that described hereinabove with respect to catheter 100 and to control handles thereof, mutatis mutandis.

As a result of leaflet 12 being secured around tubular core 562 of implant 560, excess tissue 16b is utilized for formation of bulge 578, and the effective length of lip 12a of leaflet 12 is shortened.

As seen in inset III of FIG. 22C, in some applications, following repair of the leaflet 12, tubular core 562 of implant 560 can be removed from the mitral valve. In such examples, bulge 578 on the upstream side of the leaflet, formed by tissue of leaflet 12 wrapping tubular core 562, is maintained by securing clip 564, even when the tubular core has been removed.

It is to be noted that while the illustrated example shows tubular core 562 being placed onto the downstream surface of leaflet 12 at inset I, with securing clip 564 engaging the upstream surface of leaflet 12 to hold tissue of the leaflet around the core, the arrangement of implant 560 can also be reversed. In the reversed arrangement, the tubular core 562 would be placed onto the upstream surface of leaflet 12, with the tissue wrapped around the core from the lower side, such that the upstream surface of the leaflet engages the exterior of the tubular core. Securing clip 564 is then placed above tubular core 562, in engagement with a downstream surface of the leaflet. In such examples, bulge 578 would be formed on a downstream side of the leaflet.

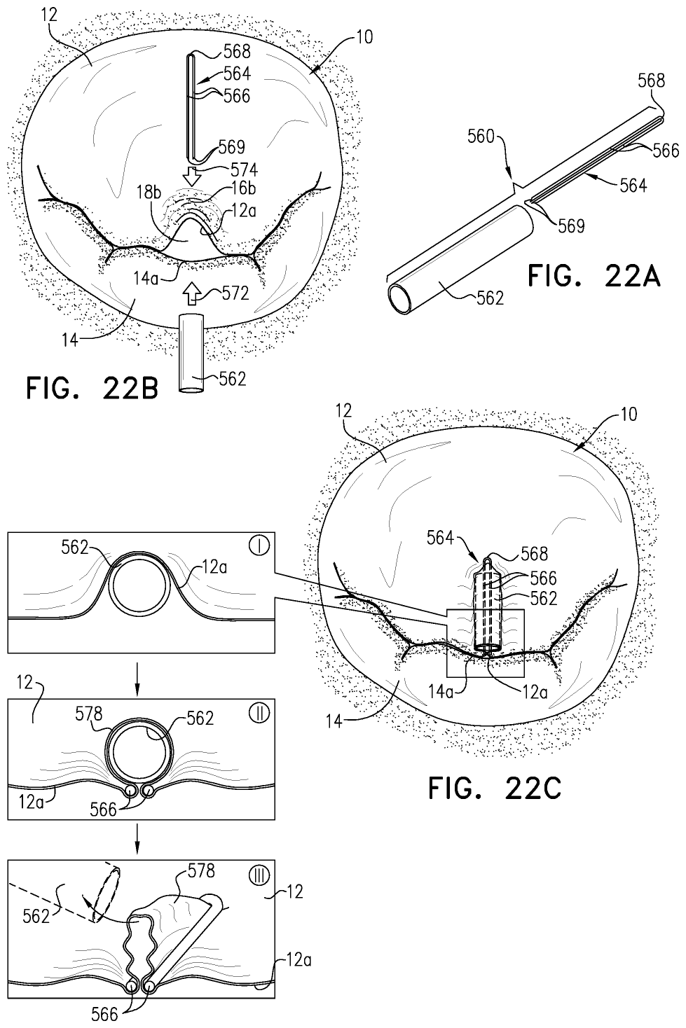

Reference is now made to FIGS. 23A to 23C are schematic illustrations of an implant 560, and phases of use thereof, for repairing a leaflet of a heart valve, in accordance with some applications.

In the example of FIGS. 23A to 23C, tips 569 of elongate portions 566 of securing clip 564 can be sharpened, so as to be able to penetrate tissue, as explained hereinbelow.

As discussed hereinabove with respect to FIGS. 22A to 22C, tubular core 562 and securing clip 564 of implant 560 are transluminally delivered to the heart chamber adjacent mitral valve 10, e.g., to the heart chamber upstream of the mitral valve. In some applications in which tips 569 are sharpened, the tips can be secured by suitable end elements, e.g., elastomeric end elements, during delivery of securing clip 564 to the heart chamber.

The catheter manipulating tubular core 562 is adapted to be steerable so as to place the tubular core against an upstream surface of leaflet 12, at an area including excess tissue 16b. In some applications, an end of tubular core 562 engages, or is adjacent, lip 12a of leaflet 12, and the core is substantially perpendicular to the lip.

A catheter (e.g., the catheter manipulating core 562, a catheter manipulating, or adapted to manipulate, securing clip 564, or another catheter), is configured to cause tissue of leaflet 12 to wrap about tubular core 562, such that the upstream surface of the leaflet engages an exterior surface of the core, as seen in inset I of FIG. 23C. A catheter manipulating securing clip 564 then pushes the securing clip, in a direction indicated by arrow 574—from the root of the leaflet toward the lip thereof—such that tips 569 penetrate tissue of leaflet 12. As a result, end portion 568 of clip 564 is disposed against an upstream side of the leaflet, close to an end of tubular core distal to lip 12a, and elongate portions 566 engage the downstream surface of leaflet 12. Thus, elongate portions 566 of securing clip 564 secures the tissue about tubular core 562, and tissue of leaflet 12 forms a bulge 578 about the core, shown in inset II of FIG. 23C. The dimensions and mechanical structure of tubular core 562, and of securing clip 564, are designed and configured to ensure cooperation therebetween, which cooperation ensures that core 562 is held in place, wrapped with tissue, to form bulge 578.

Placing the tubular core and securing clip onto the leaflet can be carried out by the catheter(s), for example in response to human operation of a control handle, e.g., similarly to that described hereinabove with respect to catheter 100 and to control handles thereof, mutatis mutandis.

As a result of leaflet 12 being secured around tubular core 562 of implant 560, excess tissue 16b is utilized for formation of bulge 578, and the effective length of lip 12a of leaflet 12 is shortened.

In some applications, the catheter can be configured to secure tips 569 of securing clip 564 following penetration of the sharpened tips through the tissue of leaflet 12, for example using elastomeric end elements.

As seen in inset III of FIG. 23C, in some applications, following repair of the leaflet 12, tubular core 562 of implant 560 can be removed from the mitral valve. In such examples, bulge 578 on the downstream side of the leaflet, formed by tissue of leaflet 12 wrapping tubular core 562, is maintained by securing clip 564, even when the tubular core has been removed.

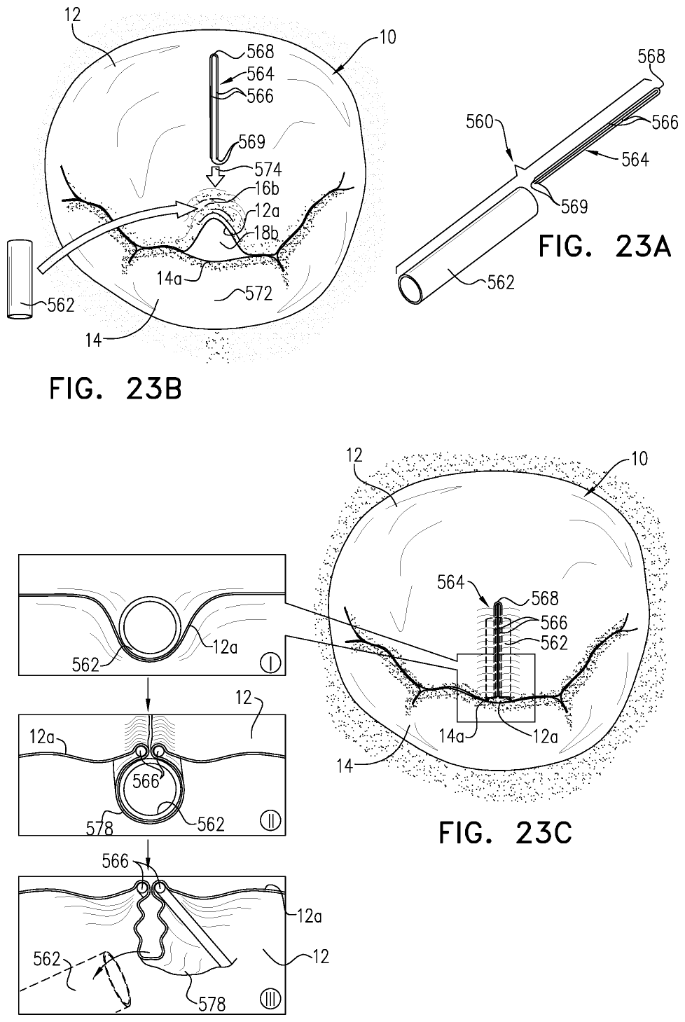

Reference is now made to FIGS. 24A to 24D, which are schematic illustrations of an implant 600, and phases of use thereof, for repairing a leaflet of a heart valve, in accordance with some applications.

Implant 600 includes a core element 602, comprising a first plate 604a and a second plate 604b, connected by a neck portion 606. Plates 604a and 604b have a greater diameter, or a greater cross section, than neck portion 606, such that an indentation 608 is formed between plates 604a and 604b, around neck portion 606.

In the illustrated example, first plate 604a and second plate 604b are substantially the same size. However, in some applications, the first and second plates need not be the same size, provided that both plates have a greater cross section than neck portion 606.

In the illustrated example, first plate 604a and second plate 604b are substantially parallel to one another. However, in some applications, the two plates can be angled relative to each other.

In the illustrated example, first plate 604a and second plate 604b are circular, and neck portion 606 is cylindrical and has a circular cross section. However, the plates and the neck portion can have other shapes or other cross sections. Additionally, plates 604 need not have the same shaped cross section as neck portion 606, or as one another.

Implant 600 includes a securing element 612, which comprises a panel 614 having a groove, or hollow, formed therein. The groove includes a generally circular region 616 and a linear region 618.

Securing element 612 is dimensioned to receive core element 602 therein. As such, a thickness of securing element 612 is smaller than a height of indentation 608, such that the panel 614 can be received within indentation 608. Additionally, a cross section of circular region 616 is greater than a diameter of at least one of first plate 604a and second plate 604b, to allow at least one of the plates to pass through circular region 616 of the groove. Furthermore, a width WLR of linear region 618 of the groove is greater than a diameter of neck portion 606.

In FIG. 24A, implant 600 is shown in a first operative state, in which core element 602 is separate from securing element 612.

As explained in further detail hereinbelow, in a second operative state of implant 600, first plate 604a is disposed on one side of panel 614, second plate 604b is disposed on a second, opposing side of panel 614, and neck portion 606 extends through linear region 618 of securing element 612. In a sense, the second operative state of implant 600 is similar to a button, or rivet, held within a button loop.

The transition from the first operative state of implant 600, to the second operative state, is accomplished by two steps. First, first plate 604a or second plate 604b is inserted into circular region 616 of securing element 612, such that panel 614 is vertically aligned with, and surrounds, neck portion 606, and neck portion 606 is substantially centered within circular region 616. Subsequently, core element 602 is moved relative to securing element 612, such that neck portion 606 slides into linear region 618, while plates 604a and 604b are exterior to panel 614 and are disposed on opposing sides thereof.

It is to be noted that while circular region 616 is shown as having a circular shape, in examples in which plates 604a and 604b have a different shape, or cross section, the shape of circular region 616 can be modified to match that of plates 604a and/or 604b. The main requirement is that at least one of plates 604a and 604b be able to extend through the region 616, and that neither of plates 604a and 604b be able to extend through linear region 618, when the plates are parallel to panel 614.

Turning to FIG. 24B, first leaflet 12 has excess tissue 16b, such that a gap 18b is formed between lips 12a and 14a of leaflets 12 and 14.

Core element 602 and securing element 612 of implant 600 are transluminally delivered to the heart chamber adjacent mitral valve 10, e.g., to the heart chamber upstream of the mitral valve. In some applications, core element 602 and securing element 612 of implant 600 are delivered by at least one longitudinal catheter, similar to catheter 100 described hereinabove, the catheter being configured to be transluminally advanced toward the heart chamber. As described hereinabove, the longitudinal catheter has a proximal part and a steerable distal part, and a longitudinal axis therebetween. In some applications, a first catheter is used to deliver core element 602, and a second catheter is used to deliver securing element 612.

The catheter manipulating core element 602 is adapted to be steerable so as to slide the core element beneath leaflet 12, from the lip toward the root of the leaflet, in the direction of arrow 622. As a result, excess tissue 16b rests on core element 602. In some applications, core element 602 is held beneath leaflet 12 such that one plate thereof, e.g., first plate 604a, engages a downstream surface of the leaflet.

The catheter manipulating securing element 612, is then configured to place securing element 612 against an upstream surface of leaflet 12, such that first plate 604a of core element 602 is aligned with, and extends into, circular region 616 of the groove of the securing element. Because tissue of leaflet 12 is disposed between core element 602 and securing element 612, when first plate 604a extends into circular region 616, at least some of excess tissue 16b, disposed above first plate 604 is wrapped around the first plate and is also pushed into circular region 616, as seen in the enlarged portion of FIG. 24C.

As seen in FIG. 24D, the catheter manipulating core element 602 pushes the core element relative to securing element 612, away from lip 12a of leaflet 12, in a direction indicated by reference numeral 624. In some applications, core element 602 is pushed until tissue surrounding neck portion 606 engages an end of linear region 618, distal to circular region 616. Motion of core element 602 relative to securing element 612 causes neck portion 606 to slide along linear region 618 of securing element 612, wrapped in excess tissue 16b of leaflet 12. Simultaneously, plates 604a and 604b extend above and beneath panel 614 surrounding linear region 618, and ensure that core element 602 remain secured to securing element 612. Thus, tissue of leaflet 12 forms a bulge about plate 604a and neck portion 606 of core element 602.

As mentioned hereinabove, the dimensions and mechanical structure of core element 602 and of securing element 612, are designed and configured to ensure cooperation therebetween, which cooperation secures core element 602, wrapped with tissue, within securing element 612.

Placing core element 602 and securing element 612 onto the leaflet, and relative motion therebetween, can be carried out by the catheter(s), for example in response to human operation of a control handle, e.g., similarly to that described hereinabove with respect to catheter 100 and to control handles thereof, mutatis mutandis.

As a result of tissue of leaflet 12 being secured around core element 602 of implant 600, excess tissue 16b is utilized for formation of a bulge, and the effective length of lip 12a of leaflet 12 is shortened.

It is to be noted that while the illustrated example shows motion of core element 602 away from lip 12a of the leaflet, for the core element to engage linear region 618 of securing element 612, the mechanical locking of the core element to the securing element can be accomplished by pushing of the securing element relative to the core element, toward lip 12a, in a direction inverse to arrow 624. In such examples, core element 602 would initially be placed in the location desired to be its final location under the leaflet.

It is to be noted that while the illustrated example shows core element 602 being placed onto the downstream surface of leaflet 12, and securing element 612 engaging the upstream surface of the leaflet to hold tissue of the leaflet around the core, the arrangement of implant 600 can also be reversed. In the reversed arrangement, core element 602 is placed onto the upstream surface of leaflet 12, with the tissue wrapped around second plate 604b the core element, such that the upstream surface of the leaflet engages the second plate. Securing element 612 is then placed beneath core element 602, in engagement with a downstream surface of the leaflet. In such examples, a bulge would be formed on a downstream side of the leaflet.

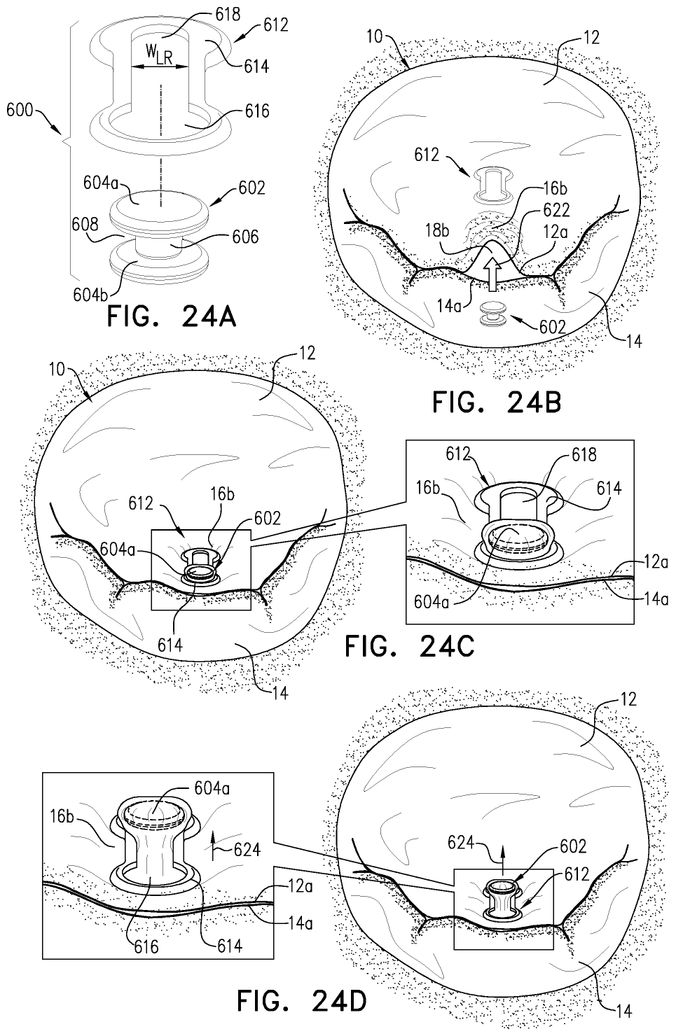

Reference is now made to FIGS. 25A to 25C, which are schematic illustrations of a clip implant 630, and phases of use thereof, for repairing a leaflet of a heart valve, in accordance with some applications.

Clip implant 630 includes a central portion 632, including a strip of a flexible material, formed substantially in a U-shape. Longitudinal bars 634 extend from corners of one end of central portion 632, and are connected to one another by a horizontal bar 635. Similarly, longitudinal bars 636 extend from corners of an opposing end of central portion 632, and are connected to one another by a horizontal bar 637, substantially parallel to horizontal bar 635. Longitudinal bars 634 together with horizontal bar 635 form a first frame, and longitudinal bars 636 together with horizontal bar 637 form a second frame. In some applications, cushioning elements 638 are disposed on horizontal bars 635 and 637.

Clip implant 630, or at least the frames thereof, is (are) resilient, and has a predefined preload, which pushes to maintain horizontal bars 635 and 637 at a predetermined distance D from one another. When horizontal bars 635 and 637 are pulled apart from each other, for example by application of force thereto, and are then released, the preload in clip implant pushes horizontal bars 635 and 637 back toward each other, to regain the predetermined distance between the two bars.

As seen in FIG. 25B, second leaflet 14 has prolapsed, or has flail, such that lip 14a of second leaflet 14 prolapses into the atrium, and does not properly coapt with lip 12a of the first leaflet. Second leaflet 14 includes an intermediate region 640, extending between lip 14a and a root of leaflet 14, or the annulus 15 adjacent leaflet 14.

Clip implant 630 is transluminally delivered to the heart chamber adjacent mitral valve 10, e.g., to the heart chamber upstream of the mitral valve. In some applications, implant 630 is delivered by at least one longitudinal catheter, similar to catheter 100 described hereinabove, the catheter being configured to be transluminally advanced toward the heart chamber. As described hereinabove, the longitudinal catheter has a proximal part and a steerable distal part, and a longitudinal axis therebetween.

A catheter, e.g., the catheter used to deliver implant 630, or another catheter, is configured to fold second leaflet 14 downward, such that lip 14a of leaflet 14 engages a downstream surface of the leaflet. The intermediate region 640 is then presented at the fold of the leaflet.

The catheter is then configured to place clip implant 630 onto the fold in leaflet 14, as shown in FIG. 25C, to constrain the lip of the leaflet in the folded position. In some applications, clip implant 630 is placed such that longitudinal bars 634 and 636 are disposed substantially perpendicular to lip 14a, horizontal bars 635 and 637 are disposed substantially parallel to the lip, and U-shaped portion 632 engages an upstream surface of leaflet 14, at the fold thereof. Cushioning element 638 are adapted to ensure that horizontal bars 635 and 637 do not rupture the tissue of leaflet 14, when the leaflet is held within clip implant 630.

In some applications, in order to mount clip implant 630 onto the folded leaflet, the catheter must push apart horizontal bars 635 and 637 to insert the tissue into the clip, and then release the horizontal bars for the preload of the clip implant to close the gap between the horizontal bars, and to secure the tissue therewithin. In some applications, the distance D, which exists between horizontal bars 635 and 637 in a rest state of clip implant, e.g., when no force is applied thereto, is select to be substantially equal to the thickness of two layers of leaflet 14, or slightly smaller, to ensure that the horizontal bars 635 and 637 hold the leaflet in the folded position.

As seen in FIG. 25C, particularly in the sectional portion thereof, folding of leaflet 14 and constraining lip 14a in the folded position, presents intermediate region 640 of leaflet 14 as a substitute coaptation surface for coaptation of lip 12a of first leaflet 12. Thus, as seen in FIG. 25C, following folding and constraining of lip 14a, lip 12a of leaflet 12 can properly coapt against intermediate region 640, thus repairing, or improving function of, mitral valve 10. In some applications, the material of strip 632 can form part of the substitute coaptation surface.

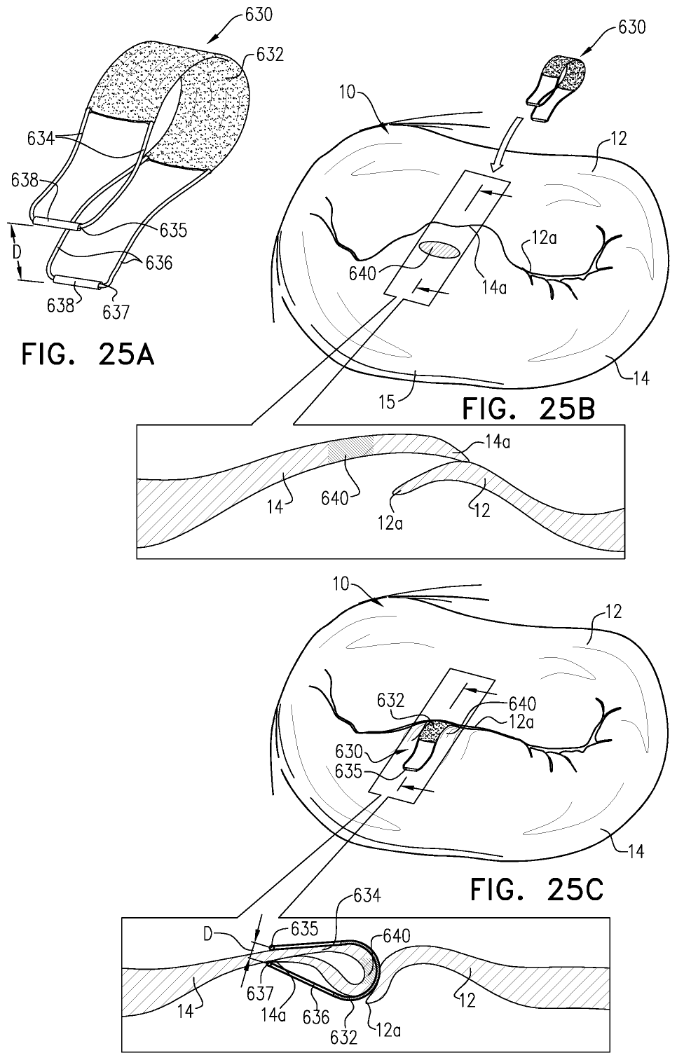

Reference is now made to FIGS. 26A to 26C, which are schematic illustrations of a pin implant 650, and phases of use thereof, for repairing a leaflet of a heart valve, in accordance with some applications.

Pin implant 650 is formed of a generally planar, U-shaped piece of a resilient material, such as a resilient metal. The U-shaped piece includes a base portion 652, and a pair of arm portions 654 extending from the base portion. At, or near, base portion 652, there is a base distance Db between arm portions 654. Extending from an end of each of arm portion 654 are end portion 656, which turn inwardly toward each other, such that an end distance De, smaller than base distance Db, exists between end portions 656.

Pin implant 650 is formed of a resilient material, having a predefined preload, which, in the rest state of the pin implant, maintains end portions 656 at end distance De from one another. When end portions 656 are pulled apart from each other, for example by application of force thereto, and are then released, the preload in the pin implant pushes end portions 656 back toward each other, to regain end distance De.

In FIG. 26B, the condition of mitral valve 10 is similar to that described with respect to FIG. 25B, with second leaflet 14 flailing, such that lip 14a of second leaflet 14 prolapses into the atrium, and does not properly coapt with lip 12a of the first leaflet. Similarly, second leaflet 14 includes an intermediate region 640, extending between lip 14a and a root of leaflet 14, or the annulus 15 adjacent leaflet 14.

Pin implants 650 are used to repair leaflet 14 in a similar manner to that described hereinabove with respect to clip implant 630 of FIG. 25A to 25C. The pin implants are also adapted to secure leaflet 14 in a folded position, such that intermediate region 640 is presented to lip 12a of first leaflet 12, for coaptation therewith.

As seen, pin implants 650 are transluminally delivered to the heart chamber adjacent mitral valve 10, e.g., to the heart chamber upstream of the mitral valve. In some applications, pin implants 650 are delivered by at least one longitudinal catheter, similar to catheter 100 described hereinabove, the catheter being configured to be transluminally advanced toward the heart chamber. As described hereinabove, the longitudinal catheter has a proximal part and a steerable distal part, and a longitudinal axis therebetween.

A catheter, e.g., the catheter used to deliver pin implant(s) 650, or another catheter, is configured to fold second leaflet 14 downward, such that lip 14a of leaflet 14 engages a downstream surface of the leaflet. The intermediate region 640 is then presented at the fold of the leaflet.

The catheter is then configured to place pin implant 650 onto the fold in leaflet 14, as shown in FIG. 25C, to constrain the lip of the leaflet in the folded position. In some applications, pin implant 650 is placed such that arms 654 are disposed substantially perpendicular to lip 14a, and U-shaped portion 652 engages an upstream surface of leaflet 14, at the fold thereof. End portions 656 are adapted to penetrate into tissue of leaflet 14, to secure pin implant 650 on the leaflet.

In some applications, in order to mount pin implant 650 onto the folded leaflet, the catheter must push apart arms 654 to insert the tissue into the pin implant, and then release the arms for the preload of the pin implant to close the gap between end portions 656, and to secure the tissue therewithin. In some applications, the end distance De, which exists in the rest state of pin implant 650, e.g., when no force is applied thereto, is selected to be substantially equal to the thickness of two layers of leaflet 14, or slightly smaller, to ensure that end portion 656 hold the leaflet in the folded position.

As seen in FIG. 26C, a plurality of pin implants 650, here shown as three pin implants 650a, 650b, and 650c, are used to constrain lip 14a of second leaflet 14. In some applications, the pin implants 650a, 650b, and 650c can be delivered to the mitral valve, and placed onto folded leaflet 14 in sequence. Stated differently, a catheter can be used to deliver and place pin implant 650a, subsequently the same catheter can be used to deliver and place pin implant 650b, and finally the same catheter can be used to deliver and place pin implant 650c. In some applications, multiple catheters can be used to deliver pin implants 650a, 650b, and 650c to the mitral valve substantially at the same time, and to place the pin implants onto the folded leaflet at substantially the same time.

As seen in FIG. 26C, particularly in the sectional portion thereof, folding of leaflet 14 and constraining lip 14a in the folded position, presents intermediate region 640 of leaflet 14 as a substitute coaptation surface for coaptation of lip 12a of first leaflet 12. Thus, as seen in FIG. 26C, following folding and constraining of lip 14a, lip 12a of leaflet 12 can properly coapt against intermediate region 640, thus repairing, or improving function of, mitral valve 10.

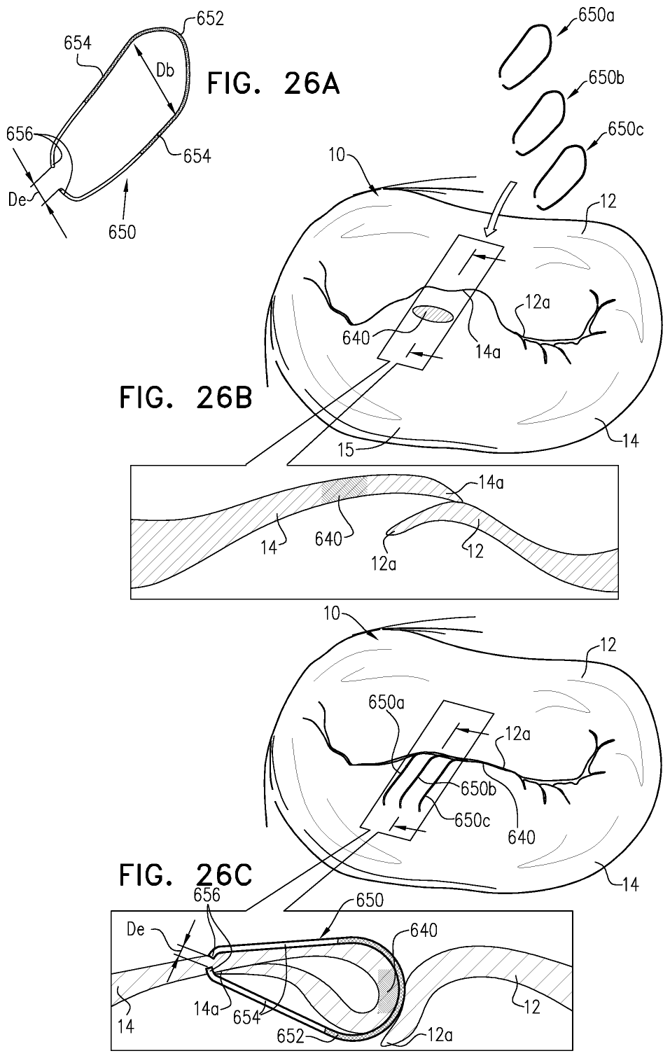

Reference is now made to FIGS. 27A to 27E, which are schematic sectional illustrations of phases of repairing second leaflet 14 of mitral valve 10 using a constraining implant 660, in accordance with some applications.

As seen in FIG. 27A, first leaflet 12 or second leaflet 14 has prolapsed, or has flail, such that lip 14a of second leaflet 14 prolapses into the atrium, and does not properly coapt with lip 12a of the first leaflet. Second leaflet 14 includes intermediate region 640, extending between lip 14a and a root of leaflet 14, or the annulus 15 adjacent leaflet 14.

Turning to FIG. 27B, it is seen that constraining implant 660 comprises a tissue anchor including a tissue engaging element 662 and a head 664. Constraining implant 660 is transluminally delivered to the heart chamber adjacent mitral valve 10, e.g., to the heart chamber upstream of the mitral valve. As seen, implant 660 is delivered by a longitudinal catheter 680, similar to catheter 100 described hereinabove, the catheter being configured to be transluminally advanced toward the heart chamber. As described hereinabove, the longitudinal catheter has a proximal part and a steerable distal part, and a longitudinal axis therebetween. In some applications, catheter 680 engages head 664 of implant 660, during delivery thereof.

A catheter, e.g., catheter 680 used to deliver implant 660, or another catheter, is configured to fold second leaflet 14 downward, such that lip 14a of leaflet 14 engages a cardiac wall 672 of the heart chamber downstream of the mitral valve. The intermediate region 640 is then presented at the fold of the leaflet.

Catheter 680 is then configured to anchor lip 14a of second leaflet 14 to cardiac wall 672 using constraining implant 660, as shown in FIG. 27C, to constrain the lip of the leaflet in the folded position. In some applications, catheter 680 can include a driving tool, which may extend distally from a lumen of catheter 680. In some applications, the driving tool is rotatable, such that rotation of the driving tool screws a portion of implant 660 into the tissue of cardiac wall 672.

As seen in FIG. 27D, particularly in the sectional portion thereof, folding of leaflet 14 and constraining lip 14a in the folded position, presents intermediate region 640 of leaflet 14 as a substitute coaptation surface for coaptation of lip 12a of first leaflet 12. Thus, as seen in FIG. 27D, following folding and constraining of lip 14a, first leaflet 12 can properly coapt against intermediate region 640, thus repairing, or improving function of, mitral valve 10.

Turning to FIG. 27E, in some applications, following constraining of lip 14a to cardiac wall 672, a secondary implant 690, defining an artificial coaptation surface 692, can be mounted over the constrained leaflet 14. Artificial coaptation surface 692 forms a substitute coaptation surface for coaptation of lip 12a of first leaflet 12. Thus, as seen in FIG. 27D, following folding and constraining of lip 14a, leaflet 12 can properly coapt against intermediate region 640, thus repairing, or improving function of, mitral valve 10.

In some applications, secondary implant 690 can be transluminally delivered to the cardiac chamber, downstream of mitral valve 10, by catheter 680 (e.g., following delivery and implantation of implant 660), or by another catheter. In some applications, secondary implant 690 can be formed of a shape memory material or of a wire mesh. In some applications, secondary implant 690 can be delivered to the cardiac chamber in a compressed form. In such examples, secondary implant 690 decompresses, or inflates, following delivery thereof and/or following implantation thereof over the constrained leaflet.

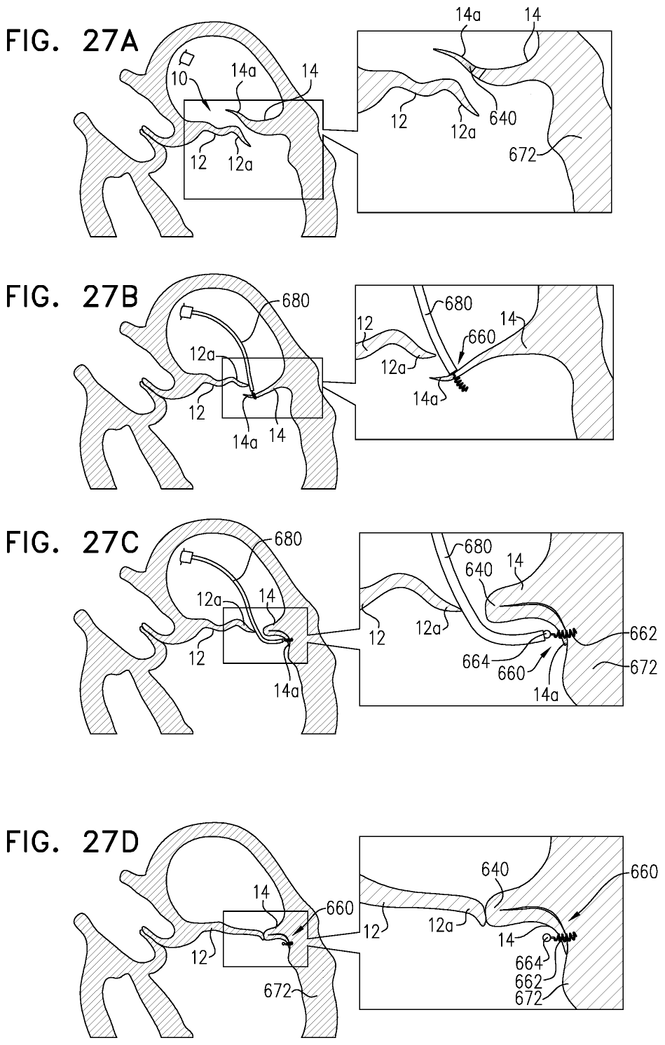

Reference is now made to FIGS. 28A, 28B, 28C, and 28D, which are schematic sectional illustrations and a top view illustration of phases of repairing a leaflet 14 of mitral valve 10 using a plurality of constraining pledget implants 700 constraining the lip 14a of the leaflet, in accordance with some applications.

As seen in FIG. 28A, first leaflet 12 or second leaflet 14 has prolapsed, or has flail, such that lip 14a of second leaflet 14 prolapses into the atrium, and does not properly coapt with lip 12a of the first leaflet. Second leaflet 14 includes intermediate region 640, extending between lip 14a and a root of leaflet 14, or the annulus 15 adjacent leaflet 14.

A constraining pledget implant 700 comprises a flexible longitudinal portion 702, and first and second end portions 704a and 704b, respectively. End portions 704a and 704b are, or can be, arranged in a direction perpendicular to a longitudinal axis of longitudinal portion 702.

Constraining pledget implant 700 is transluminally delivered to the heart chamber adjacent mitral valve 10, e.g., to the heart chamber upstream of the mitral valve. As seen, implant 700 is delivered by longitudinal catheter 680, similar to catheter 100 described hereinabove, the catheter being configured to be transluminally advanced toward the heart chamber. As described hereinabove, the longitudinal catheter has a proximal part and a steerable distal part, and a longitudinal axis therebetween. In some applications, catheter 680 constraining pledget implant 700 can be disposed within a lumen of catheter 680, during delivery to the cardiac chamber.

As seen in FIG. 28A, catheter 680 begins implantation of constraining pledget implant 700 by inserting the implant into and through cardiac wall 672, at a first position, upstream of mitral valve 10. As such, first end portion 704a engages an external surface 672a of the cardiac wall, upstream of mitral valve 10, and a part of flexible longitudinal portion 702, adjacent first end portion 704a, extends through cardiac wall 672, into the heart chamber. Following insertion of the implant into the cardiac wall, end portion 704a is arranged at an angle relative to longitudinal portion 702, e.g., perpendicularly, to prevent the end portion 704a from slipping through the tissue of cardiac wall 672, into the heart chamber.

A catheter, e.g., catheter 680 used to deliver implant 700, or another catheter, is configured to fold second leaflet 14 downward, such that lip 14a of leaflet 14 engages cardiac wall 672 of the heart chamber downstream of the mitral valve. Intermediate region 640 of the leaflet is then presented at the fold of the leaflet.

Catheter 680 is then configured to anchor lip 14a of second leaflet 14 to cardiac wall 672, by inserting second end portion 704b of constraining pledget implant 700, into and through lip 14a and cardiac wall 672, at a second position, downstream of mitral valve 10, as shown in FIG. 28B. This constrains lip 14a of leaflet 14 in the folded position. In this arrangement, flexible longitudinal portion 702 of constraining pledget implant 700 extends against, and engages, an upstream surface of leaflet 14, and specifically engages intermediate region 640. Additionally, second end portion 704b is arranged at an angle relative to longitudinal portion 702, e.g., perpendicularly, to prevent the second end portion from slipping through the tissue of cardiac wall 672, into the heart chamber. Catheter 680 can then be removed from the cardiac chamber.

As seen in FIGS. 28C and 28D, folding of leaflet 14 and constraining lip 14a in the folded position, presents intermediate region 640 of leaflet 14 as a substitute coaptation surface for coaptation of lip 12a of second leaflet 12. As shown in FIG. 28D, because each implant 700 is relatively narrow, in some applications, multiple constraining pledget implants 700 are implanted at different positions along leaflet 14, constraining the lip at multiple position.

Following folding and constraining of lip 14a, leaflet 12 can properly coapt against intermediate region 640 and longitudinal portions 702, thus repairing, or improving function of, mitral valve 10, as seen clearly in FIG. 28D.

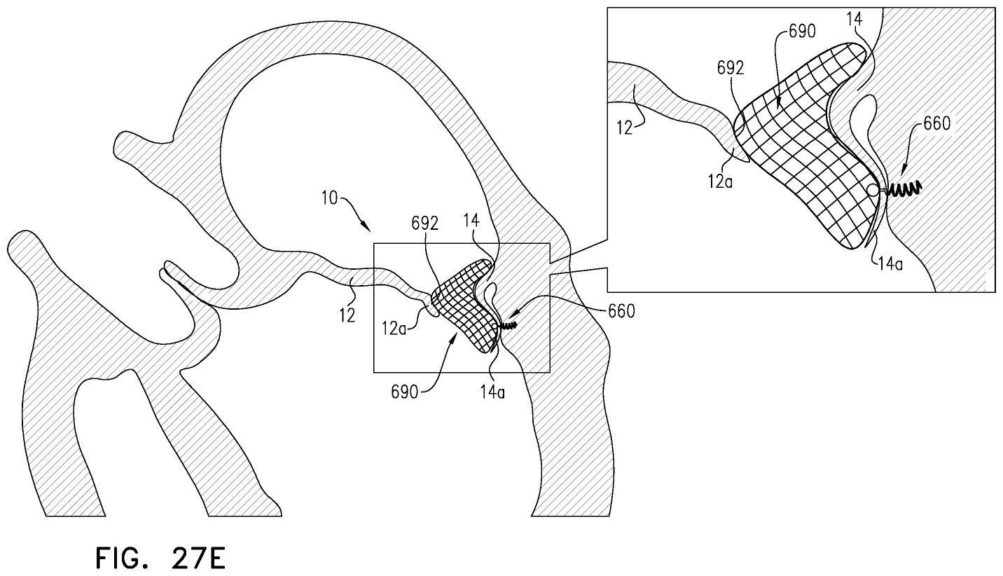

Reference is now made to FIGS. 29A and 29B, which are schematic top view illustrations of phases of repairing leaflet 14 of mitral valve 10 using a leaflet-engaging-surface implant 710, in accordance with some applications.

As seen in FIG. 29A, second leaflet 14 has prolapsed, or has flail, such that lip 14a of second leaflet 14 prolapses into the atrium, and does not properly coapt with lip 12a of the first leaflet.

A leaflet-engaging-surface implant 710, comprises a flexible leaflet-engaging-surface 712, and a tether 714. Flexible leaflet-engaging-surface 712 can be formed of a fabric, silicone, polymer, biological tissue such as pericardium, and/or any other biocompatible material. In some applications, flexible leaflet-engaging-surface 712 can be substantially non-elastic, or not elastic as perceived by the human eye. Leaflet-engaging-surface implant 710 is transluminally delivered to the heart chamber adjacent mitral valve 10, e.g., to the heart chamber upstream of the mitral valve. As seen, implant 710 is delivered by longitudinal catheter 720, similar to catheter 100 described hereinabove, the catheter being configured to be transluminally advanced toward the heart chamber. As described hereinabove, the longitudinal catheter has a proximal part and a steerable distal part, and a longitudinal axis therebetween. In some applications, tether 714 is sufficiently long so that ends of the tether extend out of the proximal end of catheter 720, and are manipulable by a medical provider implanting implant 710.

As seen in FIG. 29A, leaflet-engaging-surface 712 is attached to a surface of second leaflet 14 using tether 714, for example by suturing of the leaflet-engaging-surface to the leaflet. In some applications, in which it is desirable to shorten a length of the leaflet in order to repair it, at least one length of the tether extends along the desirable direction of shortening. For example, in the illustrated example, leaflet 14 would be repaired by shortening the distance between lip 14a and the root of the leaflet. As such, in the illustrated example, two lengths of the tether extend in the desired shortening direction, between the root and the lip of the leaflet, with a connecting length of the tether extending therebetween, substantially along the lip of the leaflet.

As seen in FIG. 29B, tether 714 is tensioned along the direction of shortening (e.g., between the lip and root of leaflet 14), e.g., by pulling both ends of tether 714 proximally, thereby to deform the leaflet, at least along the dimension between the root and the lip thereof. Following tensioning of tether 714, ends of the tether can be cut, and locking elements 716 are slid onto the cut ends of the tether, to secure the tether in its tensioned state.

As seen in FIG. 29B, following shortening of leaflet 14, leaflet 12 can properly coapt with leaflet 14. As such, the shortening of the leaflet repairs, or improves function of, mitral valve 10.

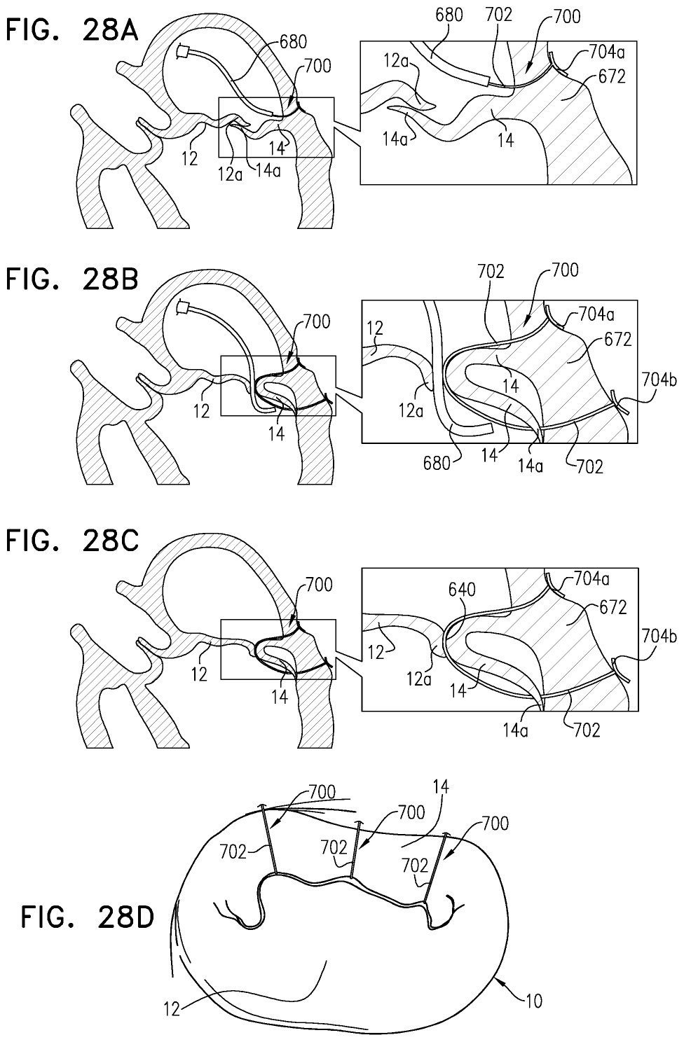

Reference is now made to FIGS. 30A and 30B, which are schematic top view illustrations of phases of repairing second leaflet 14 of mitral valve 10 using a leaflet-engaging-surface implant 730, in accordance with some applications.

As seen in FIG. 30A, second leaflet 14 has prolapsed, or has flail, such that lip 14a of second leaflet 14 prolapses into the atrium, and does not properly coapt with lip 12a of the first leaflet.

A leaflet-engaging-surface implant 730 comprises an elastic leaflet-engaging-surface 732, and a tether 734. Elastic leaflet-engaging-surface 732 can be formed of elastic cloth, elastic polymers, or another biocompatible material. Leaflet-engaging-surface implant 730 is transluminally delivered to the heart chamber adjacent mitral valve 10, substantially as described hereinabove with respect to FIG. 29A.

As seen in FIG. 29A, elastic leaflet-engaging-surface 732 is stretched in at least one dimension thereof. In the illustrated example, the stretch direction of elastic leaflet-engaging-surface 732 is indicated by arrow 736. In some applications, more than one catheter, advanced to the cardiac chamber, can be used to stretch elastic leaflet-engaging-surface 732 within the cardiac chamber.

While stretched, elastic leaflet-engaging-surface 732 is attached to a surface of second leaflet 14 using tether 734, for example by suturing of the leaflet-engaging-surface to the leaflet. In some applications, stretched elastic leaflet-engaging-surface 732 is attached to the leaflet from all sides of the surface. In some applications, when attaching elastic leaflet-engaging-surface 732 to leaflet 14, the stretched dimension of the surface is arranged along a dimension of the leaflet which should be shortened. For example, in the illustrated example, leaflet 14 is too long in the direction extending between the root and the lip of the leaflet, such that shortening of the leaflet can repair its function. As such, elastic leaflet-engaging-surface 732 is placed on the leaflet such that a stretched dimension thereof, indicated by arrow 736, extends between the root and lip of leaflet 14.

As seen in FIG. 30B, following attachment of elastic leaflet-engaging-surface 732 to leaflet 14 by tether 734, the stretching of the leaflet-engaging-surface is released. As a result, elastic leaflet-engaging-surface 732 contracts in the directions indicated by arrows 738, the leaflet attached to the surface is deformed, at least along the dimension between the root and the lip thereof.

As seen in FIG. 30B, following shortening of leaflet 14, leaflet 12 can properly coapt with leaflet 14. As such, the shortening of the leaflet repairs, or improves function of, mitral valve 10.

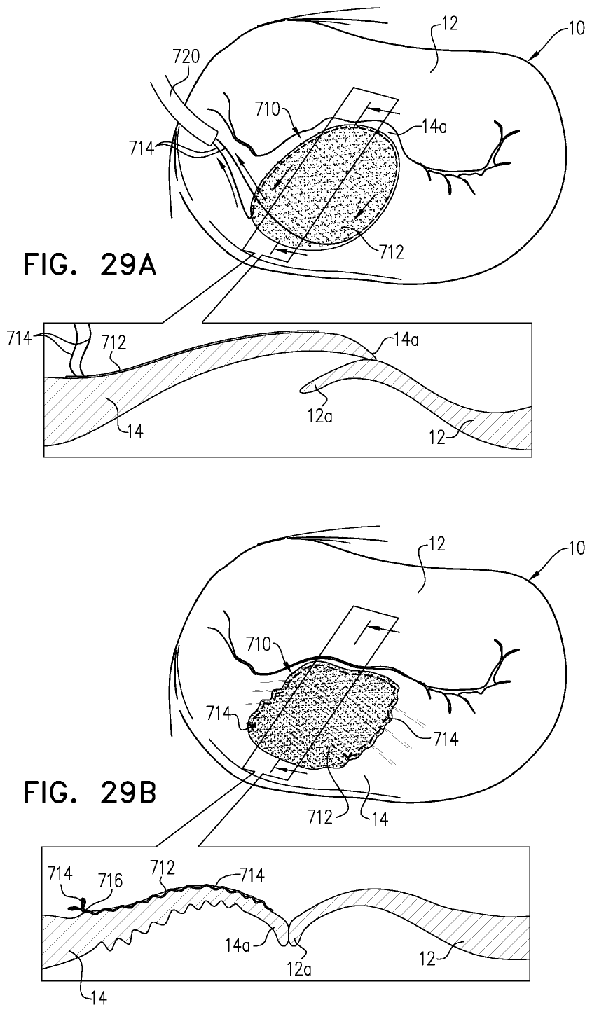

Reference is now made to FIGS. 31A, 31B, 31C, 31D, and 31E, which are schematic sectional illustrations of phases of repairing second leaflet 14 of mitral valve 10 using a pledget implant delivered through a coronary artery 750 adjacent the leaflet, in accordance with some applications.

As seen in FIG. 31A, first leaflet 12 or second leaflet 14 has prolapsed, or has flail, such that lip 14a of second leaflet 14 prolapses into the atrium, and does not properly coapt with lip 12a of the first leaflet.

A guidewire 742 is transluminally delivered, via coronary artery 750 and a wall 752 of the coronary artery, into to the heart chamber adjacent mitral valve 10, e.g., to the heart chamber upstream of the mitral valve. Guidewire 742 can be delivered through the coronary artery and into the heart chamber by a longitudinal catheter, similar to catheter 100 described hereinabove, the catheter being configured to be transluminally advanced toward the heart chamber via the coronary artery. As described hereinabove, the longitudinal catheter has a proximal part and a steerable distal part, and a longitudinal axis therebetween.

As seen, an end 742a of guidewire 742 is threaded through leaflet 14 at a first position 754 thereof, close to the root of the leaflet, from an upstream side of the leaflet to the downstream side thereof. Subsequently, end 742a of guidewire 742 is threaded through leaflet 14 at a second position 756 thereof, between first position 754 and lip 14a, from the downstream side of the leaflet to the upstream side. As a result, a section of leaflet 14, indicated by reference numeral 758, is flanked by guidewire 742. Threading of guidewire into and through the leaflet can be carried out by the catheter.

Turning to FIG. 31B, a pledget delivery system 744 is transluminally advanced, e.g., by the catheter and via coronary artery 750, over guidewire 742. Once pledget delivery system 744 is positioned within the leaflet, guidewire 742 is removed from the leaflet and from coronary artery 750, for example by the catheter, leaving the pledget delivery system in position for delivery of a pledget.

Turning to FIG. 31C, a pledget including a longitudinal portion 745 and two end portions 746 and 748 is transluminally advanced, via pledget delivery system 744, e.g., by the catheter. First end portion 746 and second end portion 748 are generally parallel to longitudinal portion 745 during delivery of the pledget, and generally perpendicular to longitudinal portion 745 following implantation of the pledget, as seen in FIGS. 31C and 31D.

In FIG. 31C, the pledget is advanced until first end 746 extends out of the leaflet at second position 756, and rests against the upstream surface of leaflet 14. Pledget delivery system 744 is retracted into the catheter, until a distal end of the pledget delivery system is disposed adjacent first position 754, at an upstream side of leaflet 14. Consequently, the pledget, and specifically longitudinal portion 745 thereof, remains the only object extending through tissue of leaflet 14.

Turning to FIG. 31D, pledget delivery system 744 is further retracted, exposing second end portion 748, such that the second end portion engages the upstream surface of leaflet 14 near first position 754. The longitudinal portion 745 can be tensioned between end portions 746 and 748. Tensioning of the longitudinal portion 745 reduces the distance between first position 754 and second position 756, thereby plicating, and effectively shortening, leaflet 14, at section 758 between the root and lip of the leaflet.

When the pledget is implanted in the leaflet, second end portion 748 is generally perpendicular to longitudinal portion 745, and is sufficiently wide to prevent retraction of the pledget from leaflet 14 toward second position 756. The two end portions 746 and 748 maintain the tension of longitudinal portion 745, thereby maintaining the reduced length between the root and lip of leaflet 14.

As seen in FIG. 31E, following complete implantation of the pledget, pledget delivery system 744 is retracted from wall 752 and from coronary artery 750, e.g., by the catheter. As a result of shortening of leaflet 14 by formation of a bulge at section 758, lip 12a of leaflet 12 can properly coapt against lip 14a of leaflet 14, thus repairing, or improving function of, mitral valve 10, as seen clearly in FIG. 31E.

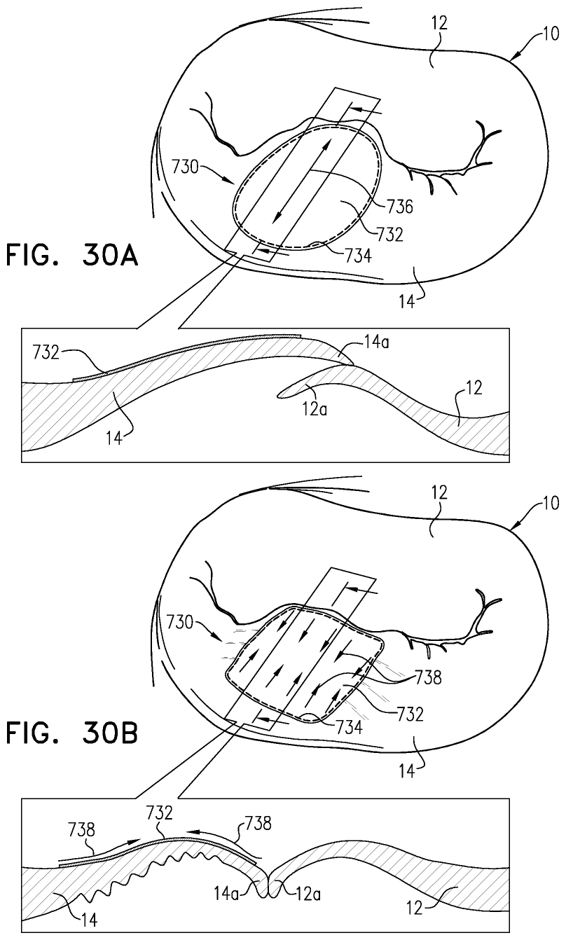

Reference is now made to FIGS. 32A to 32D, which are schematic illustrations of a flail-reducing clip implant 770, and phases of use thereof, for repairing a leaflet of a heart valve, in accordance with some applications.

Clip implant 770 includes a clip portion 772, having an integrally formed bent end 774, and a second end 775. Bent end 774 lies substantially parallel to the rest of clip portion 772. A wire finger 778 is attached to second end 745.

As seen in FIG. 32B, some of the chordae 780 holding second leaflet 14 have broken or snapped, and as a result, second leaflet 14 has flail, such that lip 14a of second leaflet 14 prolapses into the atrium. Consequently, there can be regurgitation out of the left ventricle into the left atrium, as indicated by arrows 782.

As seen in FIGS. 32C and 32D, implant 770 is transluminally delivered to the heart chamber adjacent mitral valve 10, e.g., to the heart chamber upstream of the mitral valve, in the first, planar, operative state.

In some applications, implant 770 is delivered by at least one longitudinal catheter, similar to catheter 100 described hereinabove, the catheter being configured to be transluminally advanced toward the heart chamber. As described hereinabove, the longitudinal catheter has a proximal part and a steerable distal part, and a longitudinal axis therebetween.

As seen, the catheter is adapted to be steerable so as to slide implant 770 onto leaflet 14, such that lip 14a is disposed within bend 776, with bent end 774 of the implant engaging an upstream surface of the leaflet, and clip portion 772 engaging a downstream surface of the leaflet. The catheter is further adapted to arrange wire finger 778 of implant 770 such that a distal end thereof is disposed at a point 784, at which the root of leaflet 14 engages, or turns into, cardiac wall 672. It is to be noted that the length of wire finger 778, and of the entirety of clip implant 770, is sized and configured to facilitate the positioning of the clip implant as described herein.

Placement of implant 770 as shown in FIGS. 32C and 32D in this manner provides a physical limitation to movement of the leaflet, preventing it from flailing. Thus, as seen in FIG. 32C, lip 14a of leaflet 14, having implant 770 thereon, properly coapts with lip 12a of leaflet 12. Additionally, because wire portion 778 can pivot relative to clip portion 772 at end 775, flow of blood from the atrium into the ventricle via mitral valve 10 is not disrupted.

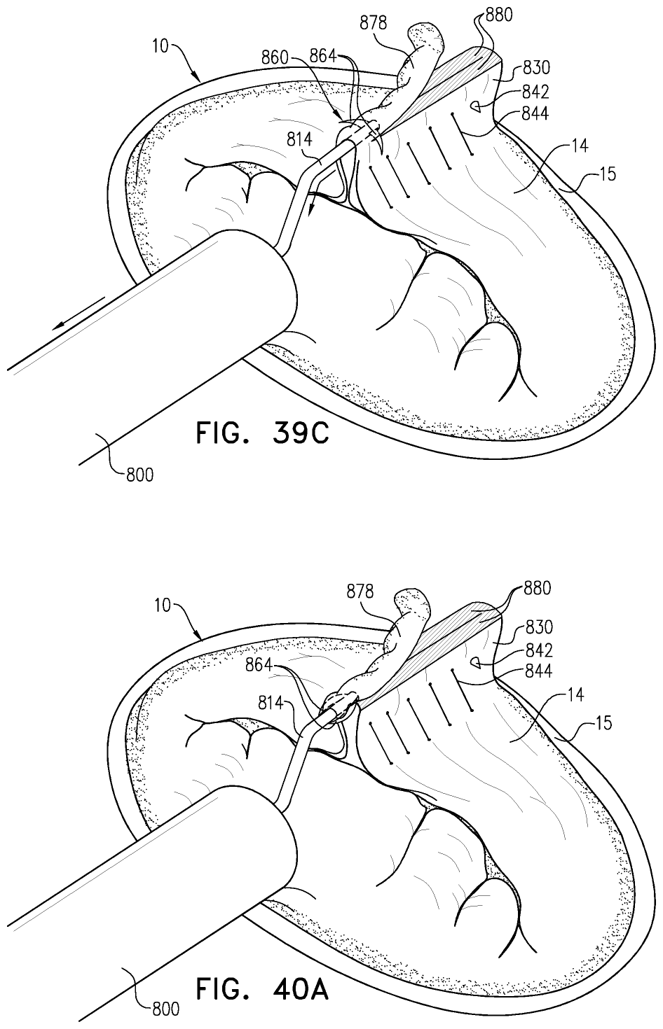

Reference is now made to FIGS. 42, 43, 44, 45A-B, 46, and 47A-B, which are schematic illustrations of a system for use with a valve of a heart of a subject, in accordance with some applications. FIG. 42 shows a mitral valve 10 and a tricuspid valve 20 of a heart, both having leaflets that do not coapt properly and being in need of repair, and of advancement of a catheter 900 to the mitral valve in accordance with some applications. The reference numerals used in FIG. 42 correspond to those used in FIG. 1, and throughout the specification. FIG. 42 shows mitral valve 10 having first leaflet 12 and second leaflet 14, surrounded by annulus 15. The first and second leaflets do not coapt properly, such that gap 18 exists in the valve when it is closed.

For some applications, tricuspid valve 20, within the same heart as mitral valve 10, may also be in need of repair in a similar manner. In the application shown in FIG. 42, tricuspid valve 20 includes leaflets 22, 23, and 24, surrounded by annulus 25. The leaflets of tricuspid valve 20 do not coapt properly with one another, such that one or more gaps 28 exist in the tricuspid valve when it is closed.

As seen in FIG. 42, a longitudinal catheter 900 is advanced toward an anatomical site of the subject. Longitudinal catheter 900 is substantially similar to longitudinal catheter 100 described hereinabove with respect to FIGS. 2-10B. In the illustrated example, a distal part 906 of the catheter is advanced to an atrium (e.g., a left atrium) of the heart, to be positioned upstream of a heart valve (e.g., mitral valve 10). Longitudinal catheter 900 also has an extracorporeal proximal part (e.g., as shown in FIGS. 10A-B). Distal part 906 is guidable to the anatomical site, such as by being actively steerable itself (e.g., by being operatively coupled by one or more pullwires to the proximal part, such as to a steering controller thereof, such as steering controller 203 shown in FIGS. 10A and 10B, or similar), or by being passively guided and/or steered (e.g., by extending over or through another steerable element, such as an actively steerable catheter). A longitudinal axis 908 extends between the proximal part and steerable distal part 906 of catheter 900. Catheter 900 is transluminally advanced to the anatomical site using any method known in the art, for example via the vena cava and, if necessary, via the septum separating the atria of the heart.

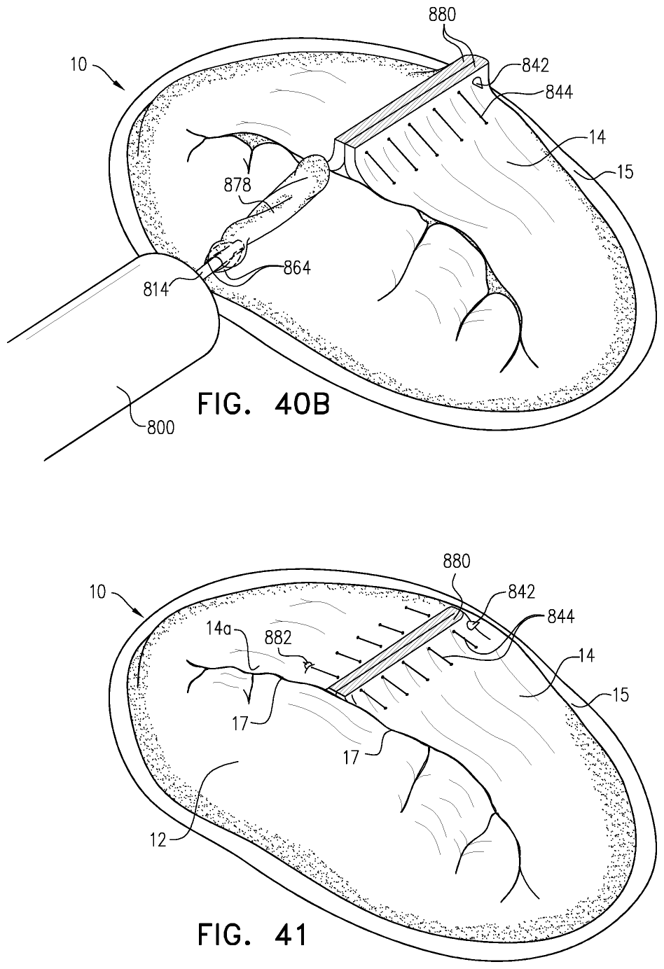

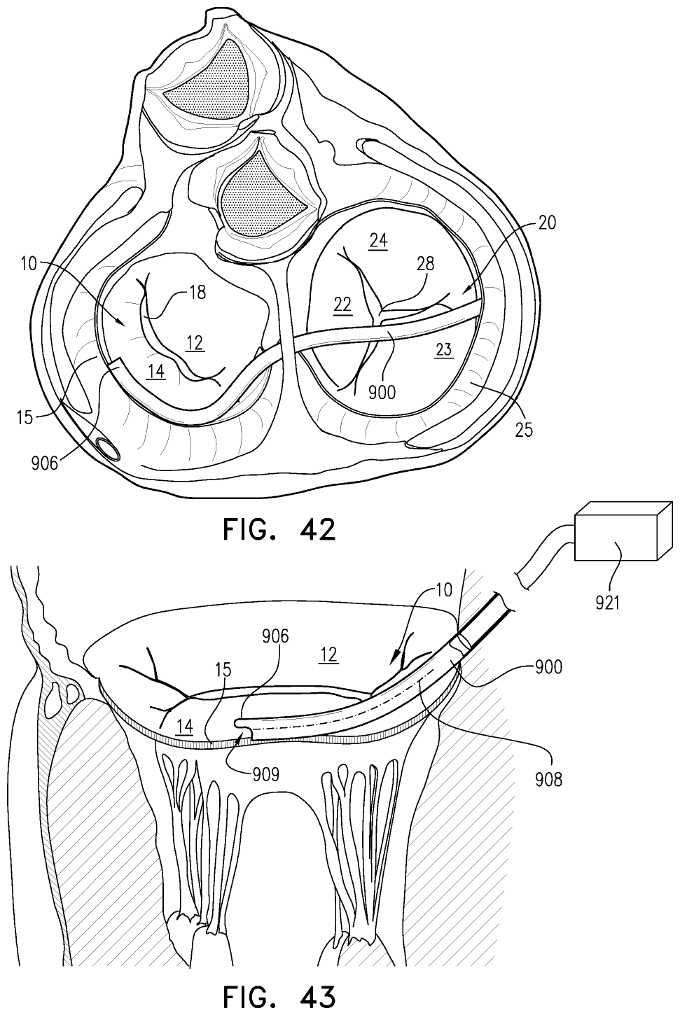

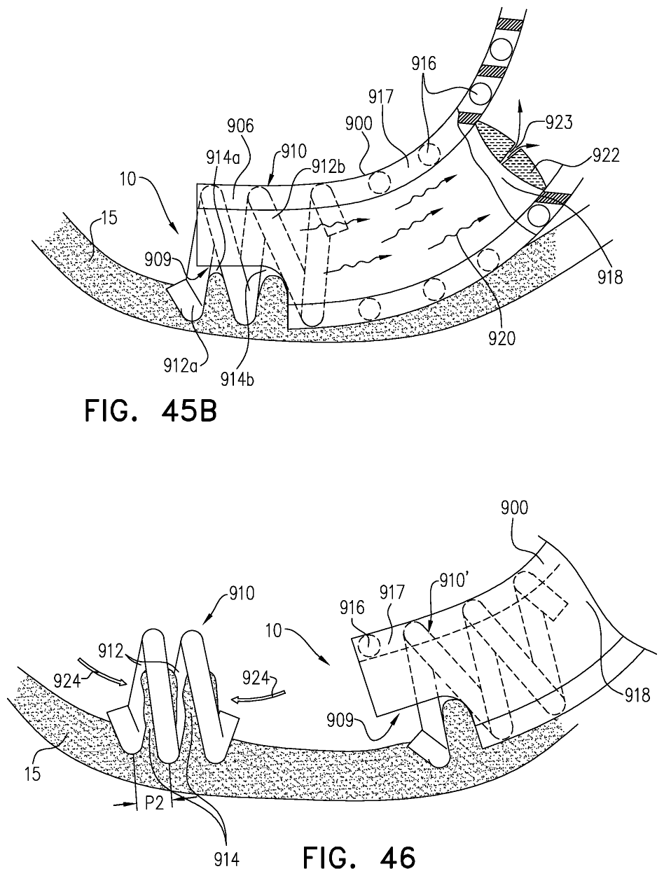

Turning now additionally to FIGS. 43 and 44, it is seen that distal end 906 of catheter 900 is partially cut-away, to define an implant-deployment portal 909. A helical implant 910 is disposed within catheter 900, near distal end 906 thereof. Helical implant 910 defines a pair of turns, labeled by reference numerals 912, each turn defining a corresponding space 914, where the dimensions of spaces 914 depend on the pitch of turns 912 in the helical implant. In a first operative state of helical implant 910, shown in FIG. 44, the turns 912 have a first pitch P1. As explained in further detail hereinbelow with respect to FIG. 46, in a second operative state of helical implant 910 the pair of turns has a second pitch P2, the second pitch being smaller than the first pitch.

Helical implant 912 is advanced to distal end 906 of catheter 900 in any suitable manner. For some applications, helical implant 910 can be rotationally advanced (e.g., screwed, etc.) through the catheter.

For some applications, catheter 900 can include a helical channel 916, extending through a wall 917 of the catheter, in addition to a central lumen 918, as illustrated in FIG. 44. For such applications, helical implant 910 can be rotationally advanced through helical channel 916 to distal end 906. For some applications, a pitch of helical channel 916 is equal to first pitch P1, and the structure of the helical channel may assist in maintaining the pitch of helical implant 910 during advancing of the helical implant.

For some applications in which multiple helical implants 910 are to be used, as explained in further detail hereinbelow, each helical implant 910 can be advanced through helical channel 916 separately (e.g., one at a time), for example by a pushing wire or other pushing tool.

For other applications, multiple helical implants 910 can be placed in helical channel 916 one behind the other. A pushing force can be applied to the most proximal of the helical implants, for example by a pushing wire or tool. That proximal implant pushes distally on the next implant, and each other implant pushes distally on the next implant, such that the pushing force applied to the proximal implant pushes forward all the implants in channel 916, one after the other.

For some applications, helical implant 910 can be pushed linearly along the longitudinal axis of the catheter, to distal end 906.

For some applications, helical implant 910 is flexible and/or resilient. For some applications, helical implant 910 comprises a resilient metal. For some applications, helical implant 910 comprises a shape memory material.

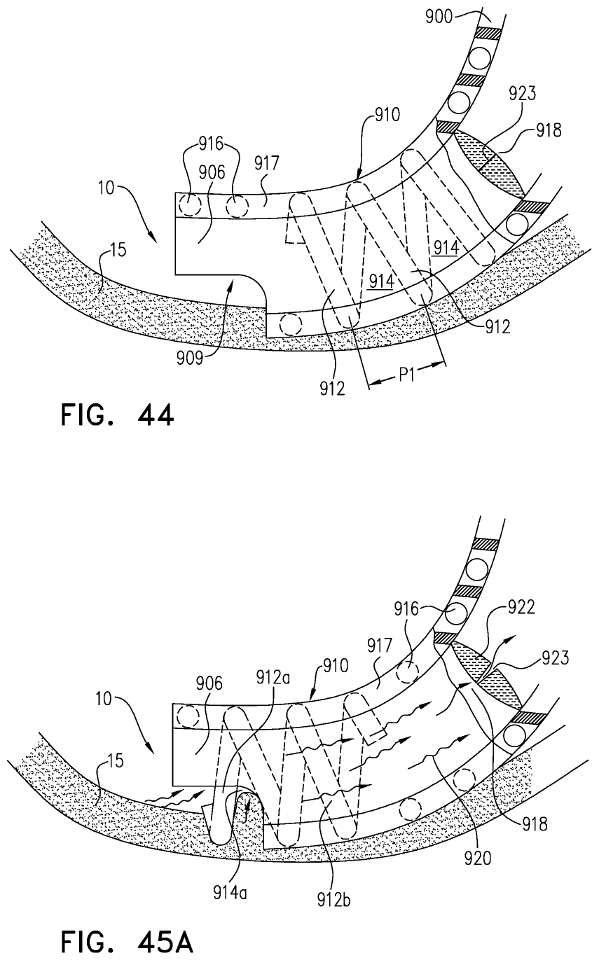

FIGS. 45A and 45B show helical implant 910 being placed onto annulus 15 of mitral valve 10, and tissue of the mitral valve (e.g., its annulus) being drawn into spaces 914 between turns 912 of the helical implant, in accordance with some applications.

As seen in FIG. 45A, while implant 910 is in the first operative state, the implant is advanced to distal end 906 of catheter 900, such that a first turn 912a of the helical implant is in implant-deployment portal 909. In this state, the turn 912a engages tissue of annulus 15 of mitral valve 10. The tissue of the annulus is drawn into space 914a of turn 912a, e.g., by drawing the tissue toward and/or into portal 909. This can be achieved, for example, by applying suction 920 (e.g., vacuum) through catheter 900, for example by activation of a pump 921, functionally associated with the proximal part of catheter 900.

Turning to FIG. 45B, it is seen that catheter 900 has been retracted in a proximal direction, while helical implant 910 remains attached to annulus 15, such that the second turn 912b is disposed in implant-deployment portal 909. In this state, turn 912b engages tissue of annulus 15 of mitral valve 10. The tissue of the annulus is drawn into space 914b of turn 912b, for example by use of vacuum pump 921. For some applications, a diaphragm 922 is disposed within catheter 900, between implant-deployment portal 909 and pump 921, so as to inhibit or limit flow of fluids from the distal end of the catheter to the proximal end thereof, at least in the absence of suction. Diaphragm 922 can include a slit 923 that is normally closed, preventing blood from flowing proximally into catheter 900 when that is not desired. When suction is applied by pump 921, the additional force increases the pressure gradient across diaphragm 922 causing the slit to open, and allowing flow in a proximal direction through the catheter. It is to be understood that other arrangements of diaphragm 922 (with or without a slit) or another type of valve member can also be used to achieve such behavior.

FIG. 46 shows the shape of helical implant 910 being changed from the first operative state to the second operative state, thereby plicating the tissue of annulus 15, in accordance with some applications.

In some applications, and as shown, catheter 900 transforms helical implant 910 from the first operative state shown in FIGS. 45A and 45B toward the second operative state shown in FIG. 46 by moving turns 912 toward each other in the direction of arrows 924. As a result, tissue of annulus 15, disposed within spaces 914, is plicated therein. For some applications, this is achieved by implant 910 being biased to assume the second operative state (e.g., due to elasticity and/or shape memory). For some applications, this is achieved by implant 910 being constrained in the first operative state during drawing of the tissue into spaces 914 (and optionally for the preceding advancement of the implant), for example by the implant being disposed within channel 916 which has pitch P1, and being subsequently released to responsively move toward the second operative state.

For some applications, following placement of helical implant 910, catheter 900 can be moved to another location along annulus 15, for placement of a second helical implant 910′, as shown in FIG. 46.

Following placement of a desired number of helical implants 910, catheter 900 can be retracted from the cardiac chamber or from the anatomical site, for example by pulling it in a proximal direction, or using any other method known in the art.

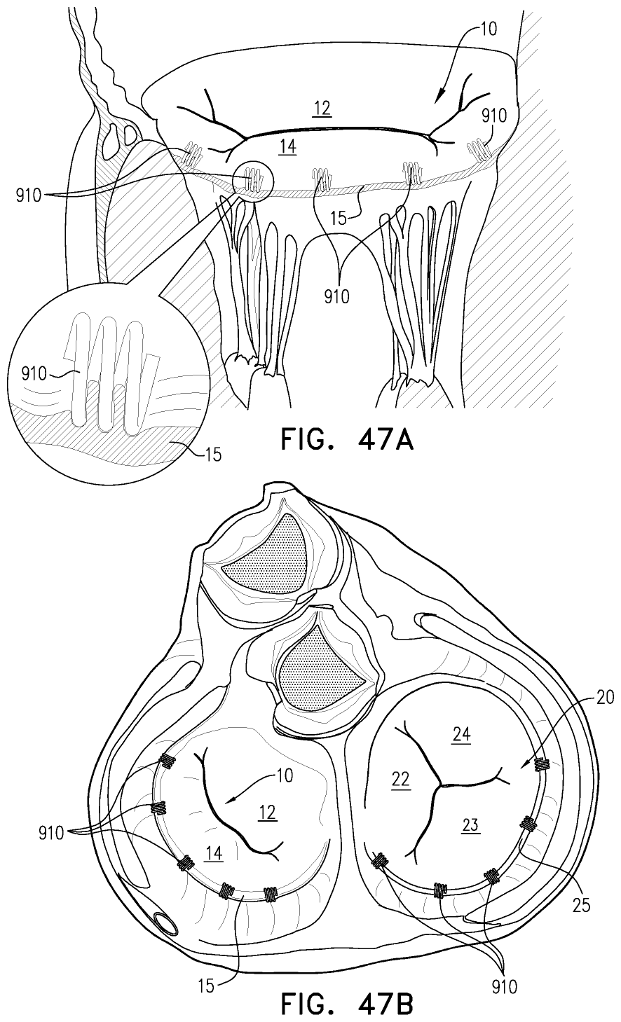

Turning now to FIGS. 47A and 47B, mitral valve 10 is shown following repair thereof using multiple helical implants 910, each positioned on, and plicating, the annulus, in accordance with the steps of FIGS. 44 to 46. As seen in FIG. 47A, four helical implants 910 were placed by the catheter. Following placement of the helical catheter, the gap between leaflets 12 and 14 has been obviated and the leaflets coapt properly.

FIG. 47B shows the repaired mitral valve 10, as well as tricuspid valve 20, the tricuspid valve having been repaired using five helical implants 910. Each of helical implants 910 was applied to annulus 25 of tricuspid valve 20 using the steps described herein with respect to FIGS. 44 to 46.

For some applications, helical implants 910 may be replaced by at least one helical needle implant, which is driven through the tissue of the annulus, e.g., similar to the helical needles described herein, except that it remains implanted in the heart. After being driven through the tissue, the helical needle implant axially contracts (e.g., due to shape memory), and thereby pinches or plicates the tissue in a similar manner to that described herein.

It is to be noted that, although various examples herein are shown in use in mitral valve 10, and only on one leaflet thereof, the implants and methods disclosed herein similarly be used to repair the other leaflet of the mitral valve, as well as to repair other valves (e.g., the tricuspid valve) and leaflets thereof.

In accordance with some applications, the teachings herein include system for repairing billowing, prolapse, or flailing of a leaflet of a cardiac valve of a subject, the leaflet having a root attached to an annulus of the cardiac valve and a lip adapted to coapt with at least one other leaflet of the valve, the system including at least one longitudinal catheter configured to be transluminally advanced toward a heart chamber of the subject adjacent the cardiac valve, the longitudinal catheter having a proximal part and a steerable distal part, and a longitudinal axis therebetween. In some applications, the system includes a longitudinal mount, advanceable distally out of the longitudinal catheter, the mount having a first curved surface and a second curved surface.

In some applications, the system includes a helical needle defining a helical lumen, the helical needle configured to be advanced distally out of the longitudinal catheter and to axially and rotationally extend about the first surface of the longitudinal mount and through tissue of the leaflet, drawing the tissue against the second surface of the longitudinal mount.

In some applications, the system includes a suture extending through the helical lumen of the helical needle.

In some applications, the at least one longitudinal catheter is adapted to: (i) transluminally advance the mount adjacent tissue of the leaflet in an advancement direction; (ii) transluminally advance the helical needle and the suture about the mount along and through the leaflet, thereby drawing tissue of the leaflet against the second surface of the mount; (iii) secure a distal end of the suture; (iv) remove the mount and the helical needle from the cardiac valve, while maintaining the suture threaded helically through the tissue of the leaflet; and (v) tension the suture, thereby to deform the tissue of the leaflet disposed within the suture.

Some such examples are illustrated, for example, in FIGS. 11 to 13 described in detail hereinabove.

In accordance with some applications, the teachings herein include system for repairing billowing, prolapse, or flailing of a leaflet of a cardiac valve of a subject, the leaflet having a root attached to an annulus of the cardiac valve and a lip adapted to coapt with at least one other leaflet of the valve, the system including at least one longitudinal catheter configured to be transluminally advanced toward a heart chamber of the subject adjacent the cardiac valve, the longitudinal catheter having a proximal part and a steerable distal part, and a longitudinal axis therebetween. The system can also include an implant including first and second leaflet engagement portions adapted to engage the lip of the leaflet and at least one tether extending between the first and second leaflet engagement portions.

In some applications, the at least one catheter is adapted to: (i) transluminally deliver the implant to the heart chamber adjacent the cardiac valve; (ii) engage the first leaflet engagement portion to a first location on the lip of the leaflet; (iii) engage the second leaflet engagement portion to a second location on the lip of the leaflet; and (iv) draw the first and second leaflet engagement portions toward each other by tensioning the at least one tether between the first and second leaflet engagement portions.

An example implementation is illustrated in FIGS. 14, in which the leaflet-engaging portions are ends of implant 350, and the tether extends through the implant. Another example implementation is illustrated in FIGS. 15, in which the leaflet-engaging portions are leaflet-engaging clips, connected by multiple tethers.

In accordance with some applications, the teachings herein include a system for repairing billowing, prolapse, or flailing of a leaflet of a cardiac valve of a subject, the leaflet having a root attached to an annulus of the cardiac valve and a lip adapted to coapt with at least one other leaflet of the valve, the system including at least one longitudinal catheter configured to be transluminally advanced toward a heart chamber of the subject adjacent the cardiac valve, the longitudinal catheter having a proximal part and a steerable distal part, and a longitudinal axis therebetween and a piercing implant

In some applications, the piercing implant includes (i) a piercing element including a base from which extends a piercing tip and (ii) a securing element, adapted to secure the piercing tip of the piercing element.

In some applications, the at least one catheter is adapted to: (i) transluminally deliver the piercing implant to the heart chamber; (ii) create a folded region of the leaflet by folding the leaflet; (iii) insert the piercing tip of the piercing element through at least two layers of the leaflet at the folded region; and (iv) secure the at least one fold at the folded region by connecting the securing element to the piercing tip of the piercing element, such that the base of the piercing element and the securing element are on the same side of the leaflet, with the at least two layers disposed therebetween.

One example implementation of such a system is illustrated in FIG. 16.

In accordance with some applications, the teachings herein include a system for repairing billowing, prolapse, or flailing of a leaflet of a cardiac valve of a subject, the leaflet having a root attached to an annulus of the cardiac valve and a lip adapted to coapt with at least one other leaflet of the valve, the leaflet including a first indentation and a second indentation, the system including at least one longitudinal catheter configured to be transluminally advanced toward a heart chamber of the subject adjacent the cardiac valve, the longitudinal catheter having a proximal part and a steerable distal part, and a longitudinal axis therebetween and a tether implant.

In some applications, the tether implant includes (i) a tether having a proximal end and a distal end and (ii) at least one locking element, adapted to secure at least one end of the tether.

In some applications, the at least one catheter is adapted to: (i) transluminally deliver the tether to the heart chamber; (ii) thread the distal end of the tether through the first indentation in the leaflet, from a first side of the leaflet to a second side of the leaflet; (iii) thread the distal end of the tether through the second indentation in the leaflet, from the second side of the leaflet to the first side of the leaflet; (iv) tension the tether threaded through the first and second indentations, to draw the first and second indentations toward each other; and (v) secure the proximal and distal ends of the tether, using the at least one locking element, to maintain the tension in the tether.

One example implementation of such a system is illustrated in FIG. 17.

In accordance with some applications, the teachings herein include a system for repairing billowing, prolapse, or flailing of a leaflet of a cardiac valve of a subject, the leaflet having a root attached to an annulus of the cardiac valve and a lip adapted to coapt with at least one other leaflet of the valve, the system including at least one longitudinal catheter configured to be transluminally advanced toward a heart chamber of the subject adjacent the cardiac valve, the longitudinal catheter having a proximal part and a steerable distal part, and a longitudinal axis therebetween; and an implant.

In some applications, the implant includes first, second, and third beams and having a first operative state and a second operative state.

In some applications, the at least one catheter is adapted to: (i) transluminally deliver the implant to the heart chamber; (ii) place the implant onto the leaflet in the first operative state; and (iii) transform the implant from the first operative state toward the second operative state, thereby to cause the leaflet to follow a tortuous path between the first and third beams.

One example implementation of such a system is illustrated in FIGS. 18, in which the first, second, and third beams are implemented as three of beams 454. An example implementation is also illustrated in FIGS. 19, in which the first, second, and third beams are implemented as beam 466, the central unified beam, and beam 478, respectively. An example implementation is also illustrated in FIG. 20, in which the first beam is implemented as segments 502 and/or 506, the second beam is implemented as beams 510a, and third beam is implemented as segments 514 and/or 518. An example implementation is also illustrated in FIGS. 21, in which the first and third beams are implemented by elongate portions 546 of clips 544, and the second beam is implemented by tubular body 542.

In accordance with some applications, the teachings herein include a system for repairing billowing, prolapse, or flailing of a leaflet of a cardiac valve of a subject, the leaflet having a root attached to an annulus of the cardiac valve and a lip adapted to coapt with at least one other leaflet of the valve, the system including at least one longitudinal catheter configured to be transluminally advanced toward a heart chamber of the subject adjacent the cardiac valve, the longitudinal catheter having a proximal part and a steerable distal part, and a longitudinal axis therebetween and an implant.

In some applications, the implant includes (i) a core and (ii) a securing element, adapted to secure tissue about the core.

In some applications, the at least one catheter adapted to: (i) transluminally deliver the implant to the heart chamber; (ii) dispose a portion of the leaflet about the core by placing the core against a first surface of the leaflet; and (iii) from a second, opposing, surface of the leaflet, using the securing element, secure the portion of the leaflet extending about the core, thereby deforming the region of the leaflet.

One example implementation is illustrated in FIGS. 22 and 23, in which the core is a mandrel, or tubular core 562, and the securing element is clip 564. An example implementation is also illustrated in FIGS. 24, in which the core is a rivet, or core element 602, and the securing element is panel 612.

In accordance with some applications, the teachings herein include a system for repairing billowing, prolapse, or flailing of a leaflet of a cardiac valve of a subject, the leaflet having a root attached to an annulus of the cardiac valve, a lip adapted to coapt with at least one other leaflet of the valve, and an intermediate region extending between the root and the lip, the system including at least one longitudinal catheter configured to be transluminally advanced toward a heart chamber of the subject adjacent the cardiac valve, the longitudinal catheter having a proximal part and a steerable distal part, and a longitudinal axis therebetween and a constraining implant.

In some applications, the at least one longitudinal catheter is adapted to: (i) transluminally deliver the constraining implant to the heart chamber; and (ii) using the constraining implant, constrain the lip so as to present the intermediate region as a substitute coaptation surface for coaptation with the at least one other leaflet.

Example implementations of such systems are illustrated in FIGS. 25, 26, 27A-27D, and 28.

In accordance with some applications, the teachings herein include a system for repairing billowing, prolapse, or flailing of a leaflet of a cardiac valve of a subject, the leaflet having a root attached to an annulus of the cardiac valve, a lip adapted to coapt with at least one other leaflet of the valve, and an intermediate region extending between the root and the lip, the system including at least one longitudinal catheter configured to be transluminally advanced toward a heart chamber of the subject adjacent the cardiac valve, the longitudinal catheter having a proximal part and a steerable distal part, and a longitudinal axis therebetween and an implant.

In some applications, the implant includes (i) a constraining element; and (ii) an artificial coaptation element.

In some applications, the at least one longitudinal catheter is adapted to: (i) transluminally deliver the constraining element and the artificial coaptation element to the heart chamber; (ii) using the constraining implant, constrain the lip so as to present the intermediate region; and (iii) mount the artificial coaptation element onto the intermediate region of the leaflet so as to present an artificial coaptation surface for coaptation with the at least one other leaflet.

An example implementation is illustrated in FIG. 27E.

In accordance with some applications, the teachings herein include a system for repairing billowing, prolapse, or flailing of a leaflet of a cardiac valve of a subject, the leaflet having a root attached to an annulus of the cardiac valve, a lip adapted to coapt with at least one other leaflet of the valve, the system including at least one longitudinal catheter configured to be transluminally advanced toward a heart chamber of the subject adjacent the cardiac valve, the longitudinal catheter having a proximal part and a steerable distal part, and a longitudinal axis therebetween and a leaflet-engaging surface-implant.

In some applications, the leaflet-engaging surface-implant includes (i) a flexible leaflet-engaging-surface and (ii) a tether.

In some applications, the at least one longitudinal catheter is adapted to: (i) transluminally deliver the leaflet-engaging surface-implant to the heart chamber; (ii) attach the leaflet-engaging surface to a surface of the leaflet using the tether; and (iii) deform the leaflet-engaging-surface, when attached to the leaflet, thereby to deform or shorten the leaflet, along at least one dimension thereof.

Example implementations are illustrated in FIG. 29 and in FIG. 30.

In accordance with some applications, the teachings herein include a system for repairing billowing, prolapse, or flailing of a leaflet of a cardiac valve of a subject, the leaflet having a root attached to an annulus of the cardiac valve, a lip adapted to coapt with at least one other leaflet of the valve, the root of the leaflet being adjacent a coronary artery, the system including at least one longitudinal catheter configured to be transluminally advanced toward a heart chamber of the subject adjacent the cardiac valve, the longitudinal catheter having a proximal part and a steerable distal part, and a longitudinal axis therebetween; and an implant.

In some applications, the implant includes (i) a guidewire having first and second ends; and (ii) first and second pledgets adapted to attach to the respective first and second ends of the guidewire.

In some applications, the at least one longitudinal catheter is adapted to: (i) transluminally deliver the guidewire and the first and second pledgets to the heart chamber, via the coronary artery; (ii) pierce the first end of the guidewire through two positions in the leaflet, thereby to form a fold in the leaflet, the two positions and the fold being between the root of the leaflet and the lip of the leaflet; and (iii) deploy the first and second pledgets at the first and second ends of the guidewire, while tensioning the guidewire, to maintain the fold in the leaflet and to reduce the length of the leaflet from the root to the lip.

An example implementation is shown in FIG. 31.

In accordance with some applications, the teachings herein include a system for repairing billowing, prolapse, or flailing of a leaflet of a cardiac valve of a subject, the leaflet having a root attached to an annulus of the cardiac valve, a lip adapted to coapt with at least one other leaflet of the valve, the root of the leaflet being attached to a cardiac wall, the system including at least one longitudinal catheter configured to be transluminally advanced toward a heart chamber of the subject adjacent the cardiac valve, the longitudinal catheter having a proximal part and a steerable distal part, and a longitudinal axis therebetween; and a clip implant.

In some applications, the clip implant includes a clip portion defining a bend and a wire finger attached to the clip portion.

In some applications, the at least one longitudinal catheter is adapted to: (i) transluminally deliver the clip implant to the heart chamber; (ii) mount the clip implant onto the leaflet such that the bend engages the lip of the leaflet, and the wire finger extends to a contact point between the root of the leaflet and the cardiac wall, such that the clip implant mechanically limits the leaflet from flailing.

An example implementation of such is shown in FIG. 32.

For the purposes of this specification and the claims that follow, the term “substantially” is defined as “at least 95%” of the related quantity. For example, “substantially perpendicular” means at least 95% perpendicular, or having an angle in the range of 85° to 95°.

It should be understood that the use of “and/or” is defined inclusively such that the term “a and/or b” should be read to include the sets: “a and b,” “a or b,” “a,” “b.”

The present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Further, the techniques, methods, operations, steps, etc. described or suggested herein can be performed on a living animal or on a non-living simulation, such as on a cadaver, cadaver heart, simulator (e.g., with the body parts, tissue, etc. being simulated), etc.

Although the operations of some of the disclosed examples are described in a particular, sequential order for convenient presentation, it should be understood that this manner of description encompasses rearrangement, unless a particular ordering is required by specific language set forth above. For example, operations or steps described sequentially can in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms can vary depending on the particular implementation and are discernible by one of ordinary skill in the art.

Example Applications (some non-limiting examples of the concepts herein are recited below):

Example 1. A system for use with a subject, the system comprising: (A) a longitudinal catheter configured to be transluminally advanced toward an anatomical site of the subject, the longitudinal catheter having a proximal part and a steerable distal part, and a longitudinal axis therebetween; (B) a wire, advanceable distally out of the longitudinal catheter, the wire forming a wire loop having a closed distal end, and a tightening element slidably coupled to the wire such that distal sliding of the tightening element over the wire causes contraction of the wire loop and cutting of tissue disposed within the wire loop, thereby forming cut tissue and cut edges at the anatomical site; (C) a helical needle defining a helical lumen, the helical needle configured to be advanced distally out of the longitudinal catheter and to rotationally extend through, and secure together, the cut edges at the anatomical site; and (D) a suture extending through the helical lumen of the helical needle, wherein the helical needle and the suture are configured to extend along the cut edges of the anatomical site to suture the cut edges during distal sliding of the tightening element.

Example 2. The system according to example 1, further including a tissue anchor, configured to be anchored to tissue at the anatomical site, wherein at least one of the wire loop and the helical needle is attachable to the tissue anchor.

Example 3. The system according to example 2, wherein the helical needle includes a needle body terminating at a detachable distal tip, the detachable distal tip being configured to attach to the tissue anchor and to detach from the needle body.

Example 4. The system according to example 3, wherein a distal end of the suture is attached to the detachable distal tip of the helical needle.

Example 5. The system according to example 4, wherein, following detachment of the detachable distal tip of the helical needle from the needle body, the needle body is retractable helically away from the detachable distal tip while sliding over and along the suture.

Example 6. The system according to example 3, further comprising a pushing-wire extending through the helical needle and configured to push the detachable distal tip of the helical needle distally relative to the needle body, thereby to detach the detachable distal tip from the needle body.

Example 7. The system according to any one of examples 1 to 6, wherein the wire and the helical needle are adapted to be removed from the anatomical site following cutting of the tissue disposed within the wire loop, while leaving the suture in the anatomical site for suturing of the cut edges.

Example 8. The system according to any one of examples 1 to 7, further including a clamp having a distal clamping end, the clamp being advanceable distally out of the longitudinal catheter to engage the cut tissue, prior to complete detachment of the cut tissue from the anatomical site.

Example 9. The system according to example 8, wherein the clamp is adapted to be removed from the anatomical site, with the cut tissue clamped thereto following removal of the wire and the helical needle from the anatomical site.

Example 10. The system according to example 8 or example 9, wherein the clamp is configured to be advanced through the longitudinal catheter alongside the helical needle.

Example 11. The system according to any one of examples 1 to 10, wherein the helical needle follows the tightening element at a fixed distance therefrom, at least during distal sliding of the tightening element.

Example 12. The system according to any one of examples 1 to 11, wherein the anatomical site comprises a cardiac valve, and the cut tissue comprises a portion of a leaflet of the cardiac valve.

Example 13. The system according to any one of examples 1 to 12, wherein the helical needle and the suture are configured to rotationally extend through the cut edges at the anatomical site as the cut edges are formed.

Example 14. The system according to any one of examples 1 to 13, wherein, in at least a first state of the system, the tightening element is operably coupled to the helical needle such that the tightening element can be advanced distally over the wire only when followed by advancement of the helical needle.

Example 15. The system according to example 14, further comprising a user interface enabling a user to control the system, the user interface including a unified engagement element that, when actuated, controls motion of the tightening element and of the helical needle in unison.

Example 16. The system according to example 15, wherein, the unified engagement element, when actuated, controls motion of the tightening element and of the helical needle at a fixed distance from one another.

Example 17. The system according to example 15 or of example 16, wherein the user interface comprises: (i) a first engagement element that, when actuated, control motion of the tightening element; and (ii) a second engagement element that, when actuated, controls motion of the helical needle.

Example 18. The system according to example 17, wherein the unified engagement element comprises a third engagement element, distinct from the first and second engagement elements.

Example 19. The system according to example 17, wherein: (i) the user interface further comprises a coupling element, functionally associated with the first and second engagement elements; (ii) in a first operative state of the coupling element, the first and second engagement elements are coupled to form the unified engagement element, such that actuation of one of the first and second engagement elements controls motion of the tightening element and of the helical needle in unison or at a fixed distance from one another; and (iii) in a second operative state of the coupling element, the first and second engagement elements are decoupled, such that actuation of the first engagement element controls motion only of the tightening element, and actuation of the second engagement element controls motion only of the helical needle.

Example 20. The system according to any one of examples 1 to 19, wherein at least one of the wire and the helical needle is adapted to be (i) advanced through the longitudinal catheter to the anatomical site, or (ii) removed from the anatomical site via the longitudinal catheter.

Example 21. The system according to any one of examples 1 to 20, further comprising a longitudinal mount, adapted to be advanced distally out of the longitudinal catheter, such that the helical needle is adapted to rotate about, and be stabilized or guided by the longitudinal mount during rotation of the helical needle.

Example 22. The system according to any one of examples 1 to 21, further comprising a tissue-engaging tool including first, second, and third beams, advanceable distally out of the longitudinal catheter, the tissue-engaging tool adapted to (i) position the first and third beams on a first side of the tissue and the second beam on an opposing side of the tissue; and (ii) form a bulge in the tissue by movement of the second beam with respect to the first beam and the third beam, wherein the wire loop is adapted to circumscribe the bulge on the first side of the tissue.

Example 23. A system for use with a subject, the system comprising: (A) a cutting device, advanceable and steerable toward an anatomical site of the subject, wherein motion of the cutting device at the anatomical site causes cutting of tissue engaged by the cutting device, thereby forming cut edges at the anatomical site; and (B) an edge-attaching device configured to at least temporarily secure the cut edges at the anatomical site, wherein, in at least a first state of the system, the edge-attaching device is operably coupled to the cutting device such that the cutting device can be advanced distally at the anatomical site only when followed by advancement of the edge-attaching device, to secure the cut edges to one another as the cut edges are formed.

Example 24. The system according to example 23, wherein, in the first state of the system, the edge-attaching device is operably coupled to the cutting device such that the cutting device can be advanced distally only when followed by advancement of the edge-attaching device at a fixed distance from the cutting device.

Example 25. The system according to example 23 or example 24, further comprising a user interface enabling a user to control the system, the user interface including a unified engagement element that, when actuated, controls motion of the cutting device and of the edge-attaching device in unison.

Example 26. The system according to example 25, wherein, the unified engagement element, when actuated, controls motion of the cutting device and of the edge-attaching device at a fixed distance from one another.

Example 27. The system according to example 25 or example 26, wherein the user interface comprises: (A) a first engagement element that, when actuated, control motion of the cutting device; and (B) a second engagement element that, when actuated, controls motion of the edge-attaching device.

Example 28. The system according to example 27, wherein the unified engagement element comprises a third engagement element, distinct from the first and second engagement elements.

Example 29. The system according to example 27, wherein: (i) the user interface further comprises a coupling element, functionally associated with the first and second engagement elements; (ii) in a first operative state of the coupling element, the first and second engagement elements are coupled to form the unified engagement element, such that actuation of one of the first and second engagement elements controls motion of the cutting device and of the edge-attaching device in unison; and (iii) in a second operative state of the coupling element, the first and second engagement elements are decoupled, such that actuation of the first engagement element controls motion only of the cutting device, and actuation of the second engagement element controls motion only of the edge-attaching device.

Example 30. A system for use with a subject, the system comprising: (A) a cutting device, advanceable and steerable toward an anatomical site of the subject, wherein motion of the cutting device at the anatomical site causes cutting of tissue engaged by the cutting device, thereby forming cut edges at the anatomical site; (B) an edge-attaching device configured to at least temporarily secure the cut edges at the anatomical site as the cut edges are formed; and (C) a user interface enabling a user to control the system, the user interface including a unified engagement element that, when actuated, controls motion of the cutting device and of the edge-attaching device in unison.

Example 31. The system according to example 30, wherein the unified engagement element, when actuated, controls motion of the cutting device and of the edge-attaching device at a fixed distance from one another.

Example 32. The system according to example 30 or example 31, wherein the user interface comprises: (A) a first engagement element that, when actuated, control motion of the cutting device; and (B) a second engagement element that, when actuated, controls motion of the edge-attaching device.

Example 33. The system according to example 32, wherein the unified engagement element comprises a third engagement element, distinct from the first and second engagement elements.

Example 34. The system according to example 32, wherein: (i) the user interface further comprises a coupling element, functionally associated with the first and second engagement elements; (ii) in a first operative state of the coupling element, the first and second engagement elements are coupled to form the unified engagement element, such that actuation of one of the first and second engagement elements controls motion of the cutting device and of the edge-attaching device in unison; and (iii) in a second operative state of the coupling element, the first and second engagement elements are decoupled, such that actuation of the first engagement element controls motion only of the cutting device, and actuation of the second engagement element controls motion only of the edge-attaching device.

Example 35. The system according to any one of examples 23 to 34, further comprising a longitudinal catheter configured to be transluminally advanced toward the anatomical site of the subject, the longitudinal catheter having a proximal part and a steerable distal part, wherein the cutting device and the edge-attaching device are adapted to be advanceable and steerable to the anatomical site distally out of the longitudinal catheter.

Example 36. The system according to example 35, wherein the cutting device and the edge-attaching device are adapted to be advanced to the anatomical site through the longitudinal catheter.

Example 37. The system according to any one of examples 23 to 36, wherein the cutting device and the edge-attaching device are adapted to be removed from the anatomical site following cutting of the tissue and securing together of the cut edges.

Example 38. The system according to any one of examples 23 to 37, further comprising a permanent edge-attaching device adapted to permanently attach the cut edges at the anatomical site.

Example 39. The system according to example 38, wherein the edge-attaching device is adapted to temporarily secure the cut edges at the anatomical site, and the permanent edge-attaching device is adapted to permanently attach the cut edges.

Example 40. The system according to example 39, wherein the edge-attaching device comprises a needle, and the permanent edge-attaching device comprises a suture, extending through a lumen of the needle.

Example 41. The system according to example 40, wherein the needle is a helical needle and the lumen is a helical lumen.

Example 42. The system according to any one of examples 23 to 41, wherein the cutting device comprises a wire forming a wire loop having a closed distal end and a tightening element slidably coupled to the wire, such that distal sliding of the tightening element over the wire causes contraction of the wire loop and cutting of the tissue disposed within the wire loop.

Example 43. The system according to any one of examples 23 to 42, further comprising a clamp having a distal clamping end, the clamp being advanceable to the anatomical site to engage cut tissue cut by the cutting device, prior to complete detachment of the cut tissue from the anatomical site.

Example 44. The system according to example 43, wherein the clamp is adapted to be removed from the anatomical site, with the cut tissue clamped thereto, following removal of at least one of the cutting device and the edge-attaching device from the anatomical site.

Example 45. The system according to any one of examples 23 to 44, further comprising a tissue anchor, configured to be anchored to tissue at the anatomical site for anchoring of at least one of the cutting device and the edge-attaching device.

Example 46. The system according to any one of examples 23 to 45, wherein the anatomical site comprises a cardiac valve, and the tissue engaged by the cutting device and cut thereby comprises a portion of a leaflet of the cardiac valve.

Example 47. A method of removing tissue from an anatomical site of a subject, the method comprising: (A) transluminally advancing a longitudinal catheter toward the anatomical site, the longitudinal catheter having a proximal part and a steerable distal part; (B) advancing a wire loop distally out of the longitudinal catheter, the wire loop having a closed distal end, a proximal end, and a tightening element slidably coupled to the wire loop; (C) advancing a helical needle distally out of the longitudinal catheter, the helical needle including a helical lumen and a suture extending through the helical lumen; (D) placing the wire loop about tissue at the anatomical site; (E) distally sliding the tightening element relative to the wire loop, thereby cutting the tissue disposed within the wire loop and forming cut tissue and cut edges at the anatomical site; and (F) during the distally sliding of the tightening element, advancing the helical needle and the suture along the cut edges to secure the cut edges to one another.

Example 48. The method according to example 47, further comprising advancing a tissue anchor through the longitudinal catheter to the anatomical site and anchoring the tissue anchor to tissue at the anatomical site.

Example 49. The method according to example 48, further comprising, following the advancing of the helical needle: (i) anchoring a detachable distal tip of the helical needle to the tissue anchor, the detachable distal tip having a distal end of the suture attached thereto; (ii) detaching the detachable distal tip of the helical needle from a main body of the helical needle; and (iii) helically retracting the main body of the helical needle away from the detachable distal tip while sliding over the suture, thereby removing the helical needle from the anatomical site while leaving the suture in the anatomical site securing the cut edges.

Example 50. The method according to example 49, wherein the detaching comprises using a pushing-wire extending through the helical needle, pushing the detachable distal tip of the helical needle distally relative to the needle body, thereby to detach the detachable distal tip from the needle body.

Example 51. The method according to any one of examples 47 to 50, further comprising, following the cutting of the tissue, removing the wire loop from the anatomical site.

Example 52. The method according to any one of examples 47 to 51, further comprising: (i) prior to completion of the cutting of the tissue disposed within the wire loop, advancing a clamp having a distal clamping end to the anatomical site and clamping the cut tissue in the distal clamping end of the clamp; and (ii) removing the clamp and the cut clamped tissue from the anatomical site.

Example 53. The method according to example 52, wherein the advancing of the clamp is through the longitudinal catheter, and wherein during the advancing of the clamp, the clamp is alongside the helical needle.

Example 54. The method according to any one of examples 47 to 53, wherein during the sliding of the tightening element and the advancing of the helical needle, the helical needle remains at a fixed distance from the tightening element.

Example 55. The method according to any one of examples 47 to 54, wherein the anatomical site comprises a cardiac valve, and the tissue disposed within the wire loop comprises a portion of a leaflet of the cardiac valve.

Example 56. The method according to any one of examples 47 to 55, wherein the advancing of the helical needle and the suture is carried out as the cut edges are formed.

Example 57. The method according to any one of examples 47 to 56, wherein the distally sliding of the tightening element relative to the wire loop is operably coupled to the advancing of the helical needle, such that the tightening element can be advanced distally relative to the wire loop only when followed by advancement of the helical needle.

Example 58. The method according to any one of examples 47 to 57, further comprising controlling (i) of the sliding of the tightening element relative to the wire loop and (ii) of the advancing of the helical needle by a user actuating a unified engagement element of a user interface, to control motion of the tightening element and of the helical needle in unison.

Example 59. The method according to example 58, wherein the controlling of the sliding of the tightening element relative to the wire loop and of the advancing of the helical needle by the user actuating the unified engagement element comprises controlling motion of the tightening element and of the helical needle at a fixed distance from one another.

Example 60. The method according to example 58 or example 59, wherein the user interface comprises: (A) a first engagement element that, when actuated, controls motion of the tightening element; (B) a second engagement element that, when actuated, controls motion of the helical needle; and (C) a coupling element, functionally associated with the first and second engagement elements, such that (i) in a first operative state of the coupling element, the first and second engagement elements are coupled to form the unified engagement element, such that actuation of one of the first and second engagement elements controls motion of the tightening element and of the helical needle in unison, and (ii) in a second operative state of the coupling element, the first and second engagement elements are decoupled, such that the actuation of the first engagement element controls motion only of the tightening element, and actuation of the second engagement element controls motion only of the helical needle, wherein (1) the method further includes, prior to the distally sliding of the tightening element relative to the wire loop and prior to the advancing of the helical needle, ensuring that the coupling element of the user interface is in the first operative state.

Example 61. The method according to any one of examples 47 to 60, wherein at least one of the advancing of the wire loop and the advancing of the helical needle is through the longitudinal catheter.

Example 62. A method of removing tissue from a valve leaflet of a heart valve of a subject, the method comprising: (A) excising tissue from the valve leaflet, with a wire loop, to form cut edges; and (B) securing together the cut edges as the cut edges are formed, and prior to complete detachment of the tissue from the valve leaflet.

Example 63. The method according to example 62, wherein the securing together of the cut edges comprises advancing a helical needle through the cut edges.

Example 64. The method according to example 62 or example 63, further comprising, prior to the excising, advancing the wire loop to the heart valve via a transluminal longitudinal catheter.

Example 65. The method according to example 63, wherein the advancing of the helical needle comprises advancing the helical needle at a fixed distance with relationship to a tightening element of the wire loop.

Example 66. The method according to any one of examples 63 to 65, wherein the helical needle includes a helical lumen and has a suture extending through the helical lumen, the method further comprising removing the helical needle from the heart valve while leaving the suture attaching the cut edges.

Example 67. A method of removing tissue from an anatomical site of a subject, the method comprising: (A) using a cutting device disposed at the anatomical site, cutting tissue engaged by the cutting device at the anatomical site, thereby forming cut tissue and cut edges; and (B) at least temporarily securing together the cut edges with an edge-attaching device as the cut edges are formed and prior to complete detachment of the cut tissue from the anatomical site, wherein (i) the cutting is operably coupled to the securing together of the cut edges, such that the cutting device can cut tissue at the anatomical site only when followed by advancement of the edge-attaching device, to secure the cut edges to one another during forming of the cut edges by the cutting device.

Example 68. The method according to example 67, wherein the cutting is operably coupled to the securing together of the cut edges such that the cutting device can cut tissue at the anatomical site only when followed by advancement of the edge-attaching device at a fixed distance from the cutting device.

Example 69. The method according to example 67 or example 68, further comprising controlling of the cutting and of the securing together, by a user actuating a unified engagement element of a user interface, to control motion of the cutting device and of the edge-attaching device, in unison.

Example 70. The method according to example 69, wherein the controlling of the cutting and of the securing together by the user actuating the unified engagement element comprises controlling motion of the cutting device and of the edge-attaching device at a fixed distance from one another.

Example 71. The method according to example 69 or example 70, wherein the user interface comprises: (A) a first engagement element that, when actuated, controls motion of the cutting device; (B) a second engagement element that, when actuated, controls motion of the edge-attaching device; and (C) a coupling element, functionally associated with the first and second engagement elements, such that (i) in a first operative state of the coupling element, the first and second engagement elements are coupled to form the unified engagement element, such that actuation of one of the first and second engagement elements controls motion of the cutting device and of the edge-attaching device in unison, and (ii) in a second operative state of the coupling element, the first and second engagement elements are decoupled, such that actuation of the first engagement element controls motion only of the cutting device, and actuation of the second engagement element controls motion only of the edge-attaching device, wherein (1) the method further comprises, prior to the cutting and prior to the securing together, ensuring that the coupling element of the user interface is in the first operative state.

Example 72. A method of removing tissue from an anatomical site of a subject, the method comprising: (A) using a cutting device disposed at the anatomical site, cutting tissue engaged by the cutting device at the anatomical site, thereby forming cut tissue and cut edges; (B) at least temporarily securing together the cut edges with an edge-attaching device as the cut edges are formed and prior to complete detachment of the cut tissue from the anatomical site; and (C) controlling the cutting and the securing together, by a user actuating a unified engagement element of a user interface, to control motion of the cutting device and of the edge-attaching device, in unison.

Example 73. The method according to example 72, wherein the controlling of the cutting and of the securing together comprises actuating the unified engagement element to control motion of the cutting device and of the edge-attaching device at a fixed distance from one another.

Example 74. The method according to example 72 or example 73, wherein the user interface comprises: (A) a first engagement element that, when actuated, controls motion of the cutting device; (B) a second engagement element that, when actuated, controls motion of the edge-attaching device; and (C) a coupling element, functionally associated with the first and second engagement elements, such that (i) in a first operative state of the coupling element, the first and second engagement elements are coupled to form the unified engagement element, such that actuation of one of the first and second engagement elements controls motion of the cutting device and of the edge-attaching device in unison, and (ii) in a second operative state of the coupling element, the first and second engagement elements are decoupled, such that actuation of the first engagement element controls motion only of the cutting device, and actuation of the second engagement element controls motion only of the edge-attaching device, wherein (1) the method further comprises, prior to the cutting and prior to the securing together, ensuring that the coupling element of the user interface is in the first operative state.

Example 75. The method according to any one of examples 67 to 74, further comprising: (i) transluminally advancing a longitudinal catheter toward the anatomical site, the longitudinal catheter having a proximal part and a steerable distal part; and (ii) advancing and steering the cutting device and the edge-attaching device to the anatomical site distally out of the longitudinal catheter.

Example 76. The method according to example 75, wherein the advancing and steering of the cutting device and of the edge-attaching device are through the longitudinal catheter.

Example 77. The method according to any one of examples 67 to 76, further comprising removing the cutting device and the edge-attaching device from the anatomical site following the cutting and the securing together.

Example 78. The method according to any one of examples 67 to 77, wherein the securing together comprises temporarily securing together the cut edges at the anatomical site by the edge-attaching device, the method further comprising permanently attaching the cut edges at the anatomical site.

Example 79. The method according to example 78, wherein the temporarily securing together comprises advancing a needle through the cut edges to hold them together, and the permanently attaching comprises extending a suture through the needle.

Example 80. The method according to example 79, wherein the needle is a helical needle.

Example 81. The method according to any one of examples 67 to 80, wherein the cutting device comprises a wire loop having a closed distal end and a tightening element slidably coupled to the wire loop, wherein the cutting of the tissue comprises sliding of the tightening element distally relative to the wire loop contracting the wire loop and cutting tissue disposed within the wire loop.

Example 82. The method according to any one of examples 67 to 81, further comprising using a clamp having a distal clamping end advanced to the anatomical site, engaging the cut tissue, prior to complete detachment of the cut tissue from the anatomical site.

Example 83. The method according to example 82, further comprising removing the clamp from the anatomical site, with the cut tissue clamped thereto, following removal of at least one of the cutting device and the edge-attaching device from the anatomical site.

Example 84. The method according to any one of examples 67 to 83, further comprising anchoring a tissue anchor to tissue at the anatomical site, and anchoring at least one of the cutting device and the edge-attaching device thereto.

Example 85. The method according to any one of examples 67 to 84, wherein the anatomical site comprises a cardiac valve, and the cut tissue comprises a portion of a leaflet of the cardiac valve.

Example 86. A system for use with a leaflet of a cardiac valve of a subject, the system comprising: (A) at least one longitudinal catheter configured to be transluminally advanced toward a heart chamber of the subject adjacent the cardiac valve, the longitudinal catheter having a proximal part and a steerable distal part, and a longitudinal axis therebetween; (B) a longitudinal mount, advanceable distally out of the longitudinal catheter, the mount having a first curved surface and a second surface; (C) a helical needle defining a helical lumen, the helical needle configured to be advanced distally out of the longitudinal catheter and to axially and rotationally extend about the first curved surface of the longitudinal mount and through tissue of the leaflet, drawing the tissue against the second surface of the longitudinal mount; and (D) a suture extending through the helical lumen of the helical needle, wherein the at least one longitudinal catheter is adapted to: (i) transluminally advance the mount-adjacent tissue of the leaflet in an advancement direction; (ii) transluminally advance the helical needle and the suture about the mount along and through the leaflet, thereby drawing tissue of the leaflet against the second surface of the mount; (iii) secure a distal end of the suture; (iv) remove the mount and the helical needle from the cardiac valve, while maintaining the suture threaded helically through the tissue of the leaflet; and (v) tension the suture, thereby to deform the tissue of the leaflet disposed within the suture.

Example 87. The system according to example 86, further including at least one locking element, configured to be applied to the suture by the at least one longitudinal catheter, following tensioning of the suture, to secure the suture in its tensioned state.

Example 88. The system according to example 87, wherein the helical needle includes a needle body terminating at a detachable distal tip, and wherein the distal end of the suture is attached to the detachable distal tip of the helical needle.

Example 89. The system according to example 88, further comprising a pushing-wire extending through the helical needle and configured to push the detachable distal tip of the helical needle distally relative to the needle body, thereby to detach the detachable distal tip from the needle body.

Example 90. The system according to example 88, wherein the longitudinal catheter is adapted to remove the helical needle, following detachment of the detachable distal tip of the helical needle from the needle body, by retracting the needle body helically away from the detachable distal tip while sliding over and along the suture.

Example 91. The system according to any one of examples 86 to 90, further comprising a user interface enabling a user to control motion of the longitudinal catheter and operation thereof.

Example 92. The system according to any one of examples 86 to 91, wherein at least one of the mount and the helical needle is adapted to be advanced through the longitudinal catheter to the heart chamber, or removed from the heart chamber via the longitudinal catheter.

Example 93. The system according to example 92, wherein the mount and the helical needle are adapted to be advanced through a single lumen of the longitudinal catheter.

Example 94. The system according to example 92, wherein the longitudinal catheter includes at least two lumens, and each of the mount and the helical needle is adapted to be advanced through a different one of the at least two lumens of the longitudinal catheter.

Example 95. The system according to any one of examples 86 to 94, wherein: (i) the mount has a crescent-shaped cross section in a direction perpendicular to a longitudinal axis of the mount, the crescent-shape defining an exterior curved surface as the first curved surface, and an interior curved surface defining a cavity as the second curved surface, and (ii) the helical needle is adapted, during rotation thereof, to draw tissue into the cavity.

Example 96. The system according to any one of examples 86 to 94, wherein: (i) the first curved surface is an arc of a first circle having a first radius, and the second curved surface is an arc of a second circle having a second radius, (ii) the second radius is not smaller than the first radius, and (iii) when the mount is circumscribed by the helical needle, there is a gap between the mount and the helical needle along at least one portion of the mount.

Example 97. The system according to any one of examples 86 to 96, wherein the helical needle includes a shape memory material.

Example 98. A method of deforming a leaflet of a cardiac valve of a subject, the leaflet extending between a root attached to a cardiac wall and a lip adapted to coapt with at least one other leaflet, the method comprising: (A) transluminally advancing a longitudinal catheter toward the cardiac valve, the longitudinal catheter having a proximal part and a steerable distal part; (B) advancing a longitudinal mount distally out of the longitudinal catheter, the longitudinal mount having a first curved surface and a second curved surface; (C) advancing a helical needle distally out of the longitudinal catheter about the first curved surface of the mount, along and through the leaflet, thereby drawing tissue of the leaflet against the second surface of the mount, the helical needle including a helical lumen and a suture extending through the helical lumen; (D) following the advancing of the helical needle, securing a distal end of the suture adjacent the leaflet; (E) removing the mount and the helical needle from the cardiac valve, while maintaining the suture threaded helically through the tissue of the leaflet; and (F) tensioning the suture, thereby to deform the tissue disposed within the suture.

Example 99. The method according to example 98, wherein the securing of the distal end of the suture comprises sliding a locking element onto the distal end of the suture to secure the distal end of the suture.

Example 100. The method according to example 98 or example 99, further comprising, following the tensioning of the suture, sliding another locking element onto the suture to secure the suture in its tensioned state.

Example 101. The method according to any one of examples 98 to 100, further comprising, following the advancing of the helical needle, detaching a detachable distal tip of the helical needle from a main body of the helical needle, wherein the removing of the helical needle includes helically retracting the main body of the helical needle away from the detachable distal tip while sliding over the suture.

Example 102. The method according to any one of examples 98 to 101, wherein at least one of the advancing of the longitudinal mount, the advancing of the helical needle, and the removing of the longitudinal mount and the helical needle is carried out via the longitudinal catheter.

Example 103. The method according to example 102, wherein the advancing of the longitudinal mount and the advancing of the helical needle are carried out via a single lumen of the longitudinal catheter.

Example 104. The method according to example 102, wherein the advancing of the mount is carried out via a first lumen of the longitudinal catheter, and the advancing of the helical needle is carried out via a second lumen of the longitudinal catheter, the first lumen being different from the second lumen.

Example 105. The method according to any one of examples 98 to 104, wherein: (i) the mount has a crescent-shaped cross section in a direction perpendicular to a longitudinal axis of the mount, the crescent-shape defining an exterior curved surface as the first curved surface, and an interior curved surface defining a cavity as the second curved surface, and (ii) the advancing of the helical needle comprises drawing tissue of the leaflet into the cavity, during rotation of the helical needle.

Example 106. The method according to any one of examples 98 to 104, wherein: (i) the first curved surface is an arc of a first circle having a first radius, and the second curved surface is an arc of a second circle having a second radius, the second radius being not smaller than the first radius, and (ii) the advancing of the helical needle comprises drawing tissue of the leaflet into a gap between the mount and the helical needle, during rotation of the helical needle.

Example 107. The method according to any one of examples 98 to 106, wherein the advancing of the mount includes advancing the mount along the leaflet in a direction substantially perpendicular to the lip of the leaflet, thereby deforming the tissue of the leaflet disposed within the suture to shorten a width of the leaflet.

Example 108. The method according to any one of examples 98 to 106, wherein the advancing of the mount includes advancing the mount along the leaflet in a direction substantially parallel to the lip of the leaflet or to the root of the leaflet, thereby deforming the tissue of the leaflet disposed within the suture to shorten a length of the leaflet between the root and the lip.

Example 109. The method according to any one of examples 98 to 106, wherein the advancing of the mount includes advancing the mount along the root of the leaflet, wherein the advancing of the helical needle about the mount draws tissue of the root of the leaflet, adjoining the cardiac wall surrounding the cardiac valve, such that deforming the tissue disposed within the suture to shorten a length of the leaflet along the root of the leaflet.

Example 110. The method according to any one of examples 98 to 106, wherein the advancing of the longitudinal catheter is via a coronary artery surrounding the leaflet, wherein the advancing of the helical needle about the mount includes advancing the helical needle through a wall of the coronary artery and through the leaflet adjacent the root of the leaflet, such that deforming the tissue disposed within the suture to shorten a length of the leaflet along the root of the leaflet.

Example 111. A system for use with a leaflet of a cardiac valve of a subject, the system comprising: (A) at least one longitudinal catheter configured to be transluminally advanced toward a heart chamber of the subject adjacent the cardiac valve, the longitudinal catheter having a proximal part and a steerable distal part, and a longitudinal axis therebetween; and (B) an implant comprising: (i) first and second leaflet engagement portions adapted to engage the lip of the leaflet; and (ii) at least one tether extending between the first and second leaflet engagement portions; wherein, the at least one longitudinal catheter is adapted to: (1) transluminally deliver the implant to the heart chamber adjacent the cardiac valve; (2) engage the first leaflet engagement portion to a first location on the lip of the leaflet; (3) engage the second leaflet engagement portion to a second location on the lip of the leaflet; and (4) draw the first and second leaflet engagement portions toward each other by tensioning the at least one tether between the first and second leaflet engagement portions.

Example 112. The system according to example 111, further comprising at least one locking element, wherein the catheter is further adapted to slide the at least one locking element onto at least one end of the at least one tether, following tensioning thereof, to maintain the tension in the at least one tether.

Example 113. The system according to example 111 or example 112, wherein the implant includes a unitary frame, having the first and second leaflet engagement portions as ends thereof, the unitary frame further including (i) a central portion, and (ii) first and second arm portions connecting the central portion to each of the first and second leaflet engagement portions, respectively, wherein (1) the at least one tether comprises a single tether, and (2) a hollow lumen extends through the unitary frame, and the single tether extends through the hollow lumen, and out of the first and second leaflet engaging portions.

Example 114. The system according to example 113, wherein the unitary frame has a rest state, in which: (i) a first distance between ends of the first and second arm portions which connect to the first and second leaflet engaging portions, respectively, is greater than a second distance between ends of the first and second arm portions which engage the central portion; and (ii) a third distance between edges of the first and second leaflet engaging portions is smaller than the first distance.

Example 115. The system according to example 114, wherein, when a force is applied to the leaflet engaging portions pushing them toward each other while the unitary frame is in the rest state, the first and second arm portions are adapted to pivot relative to the central portion, to decrease the second and third distances.

Example 116. The system according to any one of examples 113 to 115, wherein the unitary frame is substantially planar.

Example 117. The system according to any one of examples 113 to 116, wherein the unitary frame is at least one of flexible and resilient.

Example 118. The system according to any one of examples 113 to 117, wherein the unitary frame is formed of a shape memory material.

Example 119. The system according to any one of examples 114 to 118, wherein (i) the longitudinal catheter is adapted to engage the first and second leaflet engagement portions to the lip of the leaflet when the unitary frame is in the rest state of the unitary frame, and (ii) tensioning of the tether causes the unitary frame to transition from the rest state to a second operative state, in which edges of the first and second leaflet engagement portions are drawn toward each other relative to their positions in the rest state.

Example 120. The system according to any one of examples 113 to 119, wherein the catheter is adapted to engage the first and second leaflet engagement portions to the leaflet by placing the first and second arm portions, and the central portion, against a downstream surface of the leaflet, such that the first and second leaflet engagement portions extend over the lip of the leaflet to an upstream surface thereof.

Example 121. The system according to example 120, wherein the catheter is adapted, by tensioning of the tether, to cause a portion of the lip of the leaflet, between the first and second leaflet engagement portions, to deform and create a bulge in the downstream surface of the leaflet.

Example 122. The system according to example 121, wherein, following creation of the bulge, the first and second arm portions are adapted to be substantially perpendicular to the lip of the leaflet.

Example 123. The system according to example 111 or example 112, wherein the first and second leaflet engagement portions comprise first and second clips, and the catheter is adapted to engage the first and second leaflet engagement portions to the lip of the leaflet by clipping the first and second clips to the lip of the leaflet, at the first and second positions along the lip, respectively.

Example 124. The system according to example 123, wherein the catheter is adapted to engagement the first and second leaflet engagement portions to the lip in a direction substantially perpendicular to the lip of the leaflet.

Example 125. The system according to example 123 or example 124, wherein the at least one tether comprises multiple tethers, each connecting the first and second leaflet engagement portions to each other, and wherein the catheter is adapted to tension each of the multiple tethers.

Example 126. A method for repairing billowing, prolapse, or flailing of a leaflet of a cardiac valve of a subject, the leaflet having a root attached to an annulus of the cardiac valve and a lip adapted to coapt with at least one other leaflet of the cardiac valve, the method comprising: (i) transluminally delivering to a heart chamber, adjacent the cardiac valve, an implant including a first leaflet engagement portion, a second leaflet engagement portion, and at least one tether extending between the first and second leaflet engagement portions; (ii) engaging the first leaflet engagement portion with a first location on the lip of the leaflet; (iii) engaging the second leaflet engagement portion with a second location on the lip of the leaflet; and (iv) drawing the first and second leaflet engagement portions toward each other by tensioning the at least one tether between the first and second leaflet engagement portions.

Example 127. The method according to example 126, further comprising securing the at least one tether following tensioning thereof, to maintain the first and second leaflet engagement portions drawn toward each other.

Example 128. The method according to example 126 or example 127, wherein the implant includes a unitary frame, having the first and second leaflet engagement portions as ends thereof, the unitary frame further including (A) a central portion, and (B) first and second arm portions connecting the central portion to each of the first and second leaflet engagement portions, respectively, (i) wherein the at least one tether comprises a single tether, (ii) a hollow lumen extends through the unitary frame, and the single tether extends through the hollow lumen, and out of the first and second leaflet engaging portions, and (iii) tensioning of the tether reduces a distance between ends of the first and second leaflet engagement portions, thereby to deform the leaflet and create a bulge in a downstream surface of the leaflet.

Example 129. The method according to example 128, wherein the engaging of the first and second leaflet engagement portion comprises placing the first and second arm portions, and the central portion, against the downstream surface of the leaflet, such that the first and second leaflet engagement portions extend over the lip of the leaflet to an upstream surface thereof.

Example 130. The method according to example 126 or example 127, wherein the first and second leaflet engagement portions comprise first and second clips, the engaging of the first leaflet engagement portion comprises clipping the first clip onto the lip of the leaflet at a first position, and clipping the second clip onto the lip at a second position.

Example 131. The method according to example 130, wherein the clipping of the first and second clips is in a direction substantially perpendicular to the lip of the leaflet.

Example 132. The method according to example 130 or example 131, wherein the at least one tether comprises multiple tethers, each connecting the first and second leaflet engagement portions to each other, and wherein the tensioning comprises tensioning each of the multiple tethers.

Example 133. A system for use with a leaflet of a cardiac valve of a subject, the system comprising: (A) at least one longitudinal catheter configured to be transluminally advanced toward a heart chamber of the subject adjacent the cardiac valve, the longitudinal catheter having a proximal part and a steerable distal part, and a longitudinal axis therebetween; and (B) a piercing implant including: (i) a piercing element including a base from which extends a piercing tip; and (ii) a securing element, adapted to secure the piercing tip of the piercing element; the at least one catheter adapted to: (1) transluminally deliver the piercing implant to the heart chamber; (2) create a folded region of the leaflet by folding the leaflet; (3) insert the piercing tip of the piercing element through at least two layers of the leaflet at the folded region; and (4) secure at least one fold at the folded region by connecting the securing element to the piercing tip of the piercing element, such that the base of the piercing element and the securing element are on the same side of the leaflet, with the at least two layers disposed therebetween.

Example 134. The system according to example 133, wherein the securing element comprises a cylindrical housing defining a hollow, the hollow being adapted to receive the piercing tip.

Example 135. The system according to example 133, wherein the securing element comprises an elastomer.

Example 136. The system according to any one of examples 133 to 135, wherein the at least one longitudinal catheter is adapted (i) to transluminally delivery the piercing implant to the heart chamber while the piercing tip is secured by the securing element, and (ii) prior to inserting the piercing tip of the piercing element, to separate the piercing element or the piercing tip from the securing element.

Example 137. The system according to any one of examples 133 to 135, wherein the at least one longitudinal catheter is adapted to transluminally delivery the piercing implant to the heart chamber while the piercing element is separated from the securing element.

Example 138. A method for repairing billowing, prolapse, or flailing of a leaflet of a cardiac valve of a subject, the leaflet having a root attached to an annulus of the cardiac valve and a lip adapted to coapt with at least one other leaflet of the cardiac valve, the method comprising: (A) transluminally delivering to a heart chamber, adjacent the cardiac valve, a piercing implant including a piercing element having a base from which extends a piercing tip, and a securing element, the securing element adapted to secure the piercing tip of the piercing element; (B) creating a folded region of the leaflet by folding the leaflet into at least one fold; (C) inserting the piercing tip of the piercing element through at least two layers of the leaflet at the folded region; and (D) securing the at least one fold at the folded region by connecting the securing element to the piercing tip of the piercing element, wherein, following the securing, the base of the piercing element and the securing element are on the same side of the leaflet, with the at least two layers disposed therebetween.

Example 139. The method according to example 138, wherein the securing element comprises a cylindrical housing defining a hollow, and wherein the securing comprises inserting the piercing tip into the hollow.

Example 140. The method according to example 138, wherein the securing element comprises an elastomer, and wherein the securing comprises piercing the piercing tip into the elastomer.

Example 141. The method according to any one of examples 138 to 140, wherein: (i) the transluminally delivering comprises transluminally delivering the piercing implant to the heart chamber while the piercing tip is secured by the securing element, and (ii) the method further comprises, following the transluminally delivering and prior to the inserting, separating the piercing element or the piercing tip from the securing element.

Example 142. The method according to any one of examples 138 to 140, wherein the transluminally delivering comprises transluminally delivery the piercing implant to the heart chamber while the piercing element is separated from the securing element.

Example 143. The method according to any one of examples 138 to 142, wherein the creating the folded region comprises creating a folded region disposed at an upstream side of the cardiac valve, and wherein, following the securing, the base of the piercing element and the securing element are on an upstream side of the leaflet.

Example 144. The method according to any one of examples 138 to 142, wherein the creating a folded region comprises creating the folded region disposed at a downstream side of the cardiac valve, and wherein, following the securing, the base of the piercing element and the securing element are on a downstream side of the leaflet.

Example 145. A system for use with a leaflet of a cardiac valve of a subject, the system comprising: (A) at least one longitudinal catheter configured to be transluminally advanced toward a heart chamber of the subject adjacent the cardiac valve, the longitudinal catheter having a proximal part and a steerable distal part, and a longitudinal axis therebetween; and (B) a tether implant including: (i) a tether having a proximal end and a distal end; and (ii) at least one locking element, adapted to secure at least one end of the tether; the at least one longitudinal catheter adapted to: (1) transluminally deliver the tether to the heart chamber; (2) wrap the tether about the flailing or billowing portion by advancing the distal end of the tether along a first surface of the leaflet to a transition point, at the transition point, transitioning the distal end of the tether to a second, surface of the leaflet, the second surface opposing the first surface, and advancing the distal end of the tether along the second surface of the leaflet to form the tether into a closed loop around the flailing or billowing portion of the leaflet; (3) tension the tether about the flailing or billowing portion; and (4) secure the proximal and distal ends of the tether, using the at least one locking element, to maintain the tension in the tether.

Example 146. The system according to example 145, further comprising a gripping tool extending distally out of the at least one longitudinal catheter, and adapted to grip the distal end of the tether during threading of the distal end by the catheter.

Example 147. The system according to example 145 or example 146, wherein the tether implant comprises multiple tethers, each having a corresponding proximal end and a corresponding distal end, and multiple locking elements adapted to secure at least one end of each of the multiple tethers, and wherein the catheter is adapted to deliver, wrap, tension, and secure, each of the multiple tethers.

Example 148. A method for repairing billowing, prolapse, or flailing of a leaflet of a cardiac valve of a subject, the leaflet having a root attached to an annulus of the cardiac valve, a lip adapted to coapt with at least one other leaflet of the cardiac valve, and a flailing or billowing portion, the method comprising: (A) transluminally delivering to a heart chamber, adjacent the cardiac valve, a tether having a proximal end and a distal end; (B) wrapping the tether about the flailing or billowing portion by: (i) advancing the distal end of the tether along a first surface of the leaflet to a transition point; (ii) at the transition point, transitioning the distal end of the tether to a second, surface of the leaflet, the second surface opposing the first surface; (iii) advancing the distal end of the tether along the second surface of the leaflet to form the tether into a closed loop around the flailing or billowing portion of the leaflet; (iv) tensioning the tether wrapped around the flailing or billowing portion; and (v) following the tensioning, securing the proximal and distal ends of the tether, using at least one locking element, to maintain the tension in the tether.

Example 149. The method according to example 148, wherein the wrapping comprises gripping the distal end of the tether using a gripping tool extending distally out of the at least one longitudinal catheter.

Example 150. The method according to example 148 or example 149, wherein the steps of transluminally delivering, wrapping, tensioning, and securing, are carried out for each of multiple tethers.

Example 151. A system for use with a leaflet of a cardiac valve of a subject, the system comprising: (A) at least one longitudinal catheter configured to be transluminally advanced toward a heart chamber of the subject adjacent the cardiac valve, the longitudinal catheter having a proximal part and a steerable distal part, and a longitudinal axis therebetween; and (B) an implant including first, second, and third beams and having a first operative state and a second operative state, wherein the at least one longitudinal catheter is adapted to: (i) transluminally deliver the implant to the heart chamber; (ii) place the implant onto the leaflet in the first operative state; and (iii) transform the implant from the first operative state toward the second operative state, thereby to cause the leaflet to follow a tortuous path between the first and third beams.

Example 152. The system according to example 151, wherein at least two of the first, second, and third beams are substantially parallel to each other, in at least one of the first operative state and the second operative state.

Example 153. The system according to example 151 or example 152, wherein the implant is at least one of flexible and resilient.

Example 154. The system according to any one of examples 151 to 153, wherein the implant comprises a resilient metal.

Example 155. The system according to any one of examples 151 to 153, wherein the implant comprises a shape memory material.

Example 156. The system according to any one of examples 151 to 153, wherein the implant comprises an elastic component.

Example 157. The system according to any one of examples 151 to 156, wherein the longitudinal catheter is adapted to place the implant onto the leaflet in the first operative state by placing the implant such that the lip of the leaflet engages a portion of the implant, and the first, second, and third beams are substantially perpendicular to the lip of the leaflet.

Example 158. The system according to any one of examples 151 to 157, wherein the longitudinal catheter is adapted to place the implant onto the leaflet, in the first operative state, such two of the first, second, and third beams are disposed on or adjacent one side of the leaflet, and a remaining one of the first second and third beams is disposed on or adjacent the opposing side of the leaflet.

Example 159. The system according to any one of examples 151 to 158, wherein: (i) the implant comprises a base, from which extend a plurality of beam including the first, second, and third beams, each of the plurality of beams having a terminus distal to the base, the first, second, and third beams being substantially parallel to one another; (ii) in the first operative state, the terminus of one of the first, second, and third beams is disposed outside of a plane defined by the base and termini of the other two of the first, second, and third beams, the one of the first, second, and third beams being disposed between the other two of the first, second, and third beams; and (iii) in the second operative state, the base, as well as the termini of the first, second, and third beams, are in a single plane.

Example 160. The system according to example 159, wherein: (i) the plurality of beams includes at least five beams; (ii) a first subset of the plurality of beams includes the one of the first, second, and third beams, and a second subset of the plurality of beams includes the other two of the first, second, and third beams, the first and second subsets of the plurality of beams being mutually exclusive; (iii) in the first operative state, the plane is defined by the termini of all beams in the second subset and by the base, and the termini of all beams in the first subset are disposed outside of the plane; and (iv) in the second operative state, the base, as well as the termini of the beams in the first and second subsets, are in the single plane.

Example 161. The system according to example 160, wherein, in the first operative state, the termini of all beams in the first subset are disposed in a second plane, the second plane being angled relative to the plane.

Example 162. The system according to any one of examples 159 to 161, wherein the first, second, and third beams are substantially perpendicular to the base, in at least one of the first and second operative states.

Example 163. The system according to any one of examples 159 to 162, wherein the longitudinal catheter is adapted to slide the implant onto the lip of the leaflet, when the implant is in the first operative state, such that the base extends along the lip of the leaflet, thereby to place the implant onto the leaflet.

Example 164. The system according to any one of examples 151 to 158, wherein the implant comprises: (A) first and second U-shaped portions, each including a base and first and second beams extending substantially perpendicularly from the base, and substantially parallel to each other; and (B) an engaging element holding together the second beam of each of the first and second U-shaped portions to form a unified beam, wherein (i) the unified beam is a central beam, disposed between the first beam of the first U-shaped portion and the first beam of the second U-shaped portion, (ii) the second beams of the first and second U-shaped portion are pivotable relative to the engaging element, about respective longitudinal axes of the second beams, such that the first and second U-shaped portions are pivotable relative to each other.

Example 165. The system according to example 164, wherein the first and second U-shaped portions are identical to each other.

Example 166. The system according to example 164 or of example 165, wherein the engaging element comprises a longitudinally extending cylinder.

Example 167. The system according to any one of examples 164 to 166, wherein, in the first operative state, the first and second U-shaped portions are substantially in a single plane, and in the second operative state, the unified beam is disposed outside of a plane formed by the first beams of the first and second U-shaped portions.

Example 168. The system according to any one of examples 164 to 167, wherein, in the first operative state, a first distance exists between the first beams of the first and second U-shaped portions, and in the second operative state a second distance exists between the first beams of the first and second U-shaped portions, the second distance being smaller than the first distance.

Example 169. The system according to any one of examples 164 to 168, wherein the longitudinal catheter is adapted to rotate the first and second U-shaped portions relative to each other, thereby to transform the implant from the first operative state to the second operative state.

Example 170. The system according to any one of examples 164 to 169, wherein the longitudinal catheter is adapted to slide the implant onto the lip of the leaflet, when the implant is in the first operative state, such that (i) the bases of the first and second U-shaped portions extend along the lip of the leaflet, (ii) the unified beam is disposed on or adjacent one side of the leaflet, and (iii) the first beams of the first and second U-shaped portions are disposed on or adjacent the opposing side of the leaflet, thereby to place the implant onto the leaflet.

Example 171. The system according to any one of examples 151 to 158, wherein the implant comprises: (A) a central U-shaped portion, including a pair of central beams and a connecting segment; and (B) first and second side portions, each including at least one beam and being connected to one of the central beams by a corresponding one of first and second bends, wherein (i) the at least one beam of each of the first and second side portions is substantially parallel to at least one of the central beams, and (ii) the first and second bends facilitate motion of the corresponding first and second side portions, relative to the pair of central beams, in at least two directions.

Example 172. The system according to example 171, wherein, (i) in the first operative state, the central U-shaped portion is in a first plane, and each of the first and second side portions is in a plane different from the first plane, and is disposed exteriorly to the central U-shaped portion, and (ii) in the second operative state, the central U-shaped portion and the first and second side portions are in the first plane, with the first and second side portions being disposed within the central U-shaped portion.

Example 173. The system according to example 171 or example 172, wherein the implant is formed of a single length of a flexible or resilient material.

Example 174. The system according to any one of examples 171 to 173, wherein each of the first and second side portions includes a pair of side beams, connected to each other by an additional bend, the pair of side beams being substantially parallel to each other.

Example 175. The system according to any one of examples 171 to 174, wherein the implant is side-to-side symmetrical about a central longitudinal axis of the central U-shaped portion.

Example 176. The system according to any one of examples 171 to 175, wherein, (i) in the first operative state, a first distance exists between the first and second side portions, and (ii) in the second operative state a second distance exists between the first and second side portions, the second distance being smaller than the first distance.

Example 177. The system according to any one of examples 172 to 176, wherein: (i) the implant has an intermediate operative state, in which the first and second side portions are in the first plane, and are disposed exteriorly to the central U-shaped portion, and (ii) for transforming the implant from the first operative state to the second operative state, the catheter is adapted to (1) transform the implant from the first operative state to the intermediate operative state, by pivoting the first and second side portions in a first direction relative to the central U-shaped portion, to place the first and second side portions in the first plane, and (2) further transform the implant from the intermediate operative state to the second operative state by revolving the first and second side portions in a second direction relative to the central U-shaped portion, to move the first and second side portions to an interior of the central U-shaped portion.

Example 178. The system according to example 177, wherein the first direction is about an axis perpendicular to longitudinal axes of the central beams, and the second direction is about axes parallel to the longitudinal axes of the central beams.

Example 179. The system according to any one of examples 171 to 178, wherein the longitudinal catheter is adapted to slide the implant onto the lip of the leaflet, when the implant is in the first operative state, such that (i) the first and second bends engage the lip of the leaflet and the connecting segment of the U-shaped portion is distal to the lip of the leaflet, with the U-shaped portion being disposed on or adjacent one side of the leaflet, and (ii) the first and second side portions being disposed on or adjacent the opposing side of the leaflet, thereby to place the implant onto the leaflet.

Example 180. The system according to any one of examples 171 to 179, wherein the longitudinal catheter is adapted to transluminally deliver the implant to the heart chamber when the implant is in the first operative state.

Example 181. The system according to any one of examples 172 to 179, wherein (i) the longitudinal catheter is adapted to transluminally deliver the implant to the heart chamber when the implant is in a preparatory operative state, in which the first and second side portions are in the first plane and are exterior to the central U-shaped portion, and (ii) the longitudinal catheter is further adapted, prior to placing the implant onto the leaflet, to transition the implant from the preparatory operative state to the first operative state, by pivoting the first and second side portions relative to the U-shaped portion about an axis perpendicular to longitudinal axes of the central beams.

Example 182. The system according to any one of examples 151 to 158, wherein the implant comprises: (A) a tubular body arranged along a central longitudinal axis; (B) first and second substantially U-shaped clips, each having a first elongate portion disposed on, or near, an exterior surface of the tubular body, and a second elongate portion disposed on, or near, an interior surface of the tubular body, the first and second elongate portions connected by an end portion; and (C) an elastic element holding together the second elongate portions of the first and second clips, wherein, (i) in the first operative state, the clips are disposed on a first side of the tubular body, and (ii) in the second operative state the clips are disposed on a second side of the tubular body, the second side being opposite the first side.

Example 183. The system according to example 182, wherein the longitudinal catheter is adapted to slide the implant onto the lip of the leaflet, when the implant is in the first operative state, such that the (i) end portions engage the lip of the leaflet, with the tubular body and the second elongate portions of the first and second clips being disposed on or adjacent one side of the leaflet, and (ii) the first elongate portions of the first and second clips being disposed on or adjacent the opposing side of the leaflet, thereby to place the implant onto the leaflet.

Example 184. The system according to example 182 or example 183, wherein the longitudinal catheter is adapted to transluminally deliver the implant to the heart chamber when the implant is in the first operative state.

Example 185. The system according to example 182 or example 183, wherein (i) the longitudinal catheter is adapted to transluminally deliver the implant to the heart chamber when the U-shaped clips are separate from the tubular body, and (ii) the longitudinal catheter is further adapted, prior to or during placing the implant onto the leaflet, to place the first and second clips onto the tubular body.

Example 186. The system according to any one of examples 182 to 185, wherein, the longitudinal catheter is adapted to transform the implant from the first operative state to the second operative state, by drawing the first and second clips away from each other, about a perimeter of the tubular body and against a force of the elastic element.

Example 187. A method for repairing billowing, prolapse, or flailing of a leaflet of a cardiac valve of a subject, the leaflet having a root attached to an annulus of the cardiac valve and a lip adapted to coapt with at least one other leaflet of the cardiac valve, the method comprising: (i) transluminally delivering to a heart chamber, adjacent the cardiac valve, an implant having a first operative state and a second operative state, the implant including first, second, and third beams; (ii) placing the implant onto the leaflet, in the first operative state; and (iii) while the implant is on the leaflet, causing the leaflet to follow a tortuous path between the first and third beams by transforming the implant from the first operative state toward the second operative state.

Example 188. The method according to example 187, wherein the placing of the implant comprises placing the implant such that the lip of the leaflet engages a portion of the implant, and the first, second, and third beams are substantially perpendicular to the lip of the leaflet.

Example 189. The method according to example 187 or example 188, wherein the placing of the implant comprises placing the implant onto the leaflet in the first operative state such that (i) two of the first, second, and third beams are disposed on or adjacent one side of the leaflet, and (ii) a remaining one of the first second and third beams is disposed on or adjacent the opposing side of the leaflet.

Example 190. The method according to any one of examples 187 to 189, wherein: (i) the implant comprises a base, from which extend a plurality of beam including the first, second, and third beams, each of the plurality of beams having a terminus distal to the base, the first, second, and third beams being substantially parallel to one another; (ii) the placing of the implant in the first operative state comprises placing the implant such that the terminus of one of the first, second, and third beams is disposed outside of a plane defined by the base and termini of the other two of the first, second, and third beams, the one of the first, second, and third beams being disposed between the other two of the first, second, and third beams; (iii) in the second operative state, the base, as well as the termini of the first, second, and third beams, are in a single plane; and (iv) the transforming of the implant from the first operative state toward the second operative state comprises moving the termini of the first, second, and third beams, relative to each other, so that the termini of the first, second, and third beams are in the single plane.

Example 191. The method according to example 190, wherein: (i) the plurality of beams includes at least five beams; (ii) a first subset of the plurality of beams includes the one of the first, second, and third beams, and a second subset of the plurality of beams includes the other two of the first, second, and third beams, the first and second subsets of the plurality of beams being mutually exclusive; (iii) the placing of the implant in the first operative state comprises placing the implant such that the plane is defined by the termini of all beams in the second subset and by the base, and the termini of all beams in the first subset are disposed outside of the plane; and (iv) the transforming of the implant comprises moving the termini of all the beams in the first subset such that, in the second operative state, the base, as well as the termini of the beams in the first and second subsets, are in the single plane.

Example 192. The method according to example 191, wherein, the placing of the implant in the first operative state comprises placing the implant such that the termini of all beams in the first subset are disposed in a second plane, the second plane being angled relative to the plane.

Example 193. The method according to any one of examples 190 to 192, wherein the placing of the implant comprises placing of the implant while the first, second, and third beams are substantially perpendicular to the base.

Example 194. The method according to any one of examples 190 to 193, wherein the placing of the implant onto the leaflet comprises sliding the implant onto the lip of the leaflet, when the implant is in the first operative state, such that the base extends along the lip of the leaflet with the one of the first, second, and third beams being on one side of the leaflet, and the other two of the first, second, and third beams being on the opposing side of the leaflet.

Example 195. The method according to any one of examples 187 to 189, wherein the implant comprises: (A) first and second U-shaped portions, each including a base and first and second beams extending substantially perpendicularly from the base, and substantially parallel to each other; and (B) an engaging element holding together the second beam of each of the first and second U-shaped portions to form a unified beam, wherein (i) the unified beam is a central beam, disposed between the first beam of the first U-shaped portion and the first beam of the second U-shaped portion, (ii) the second beams of the first and second U-shaped portion are pivotable relative to the engaging element, about respective longitudinal axes of the second beams, such that the first and second U-shaped portions are pivotable relative to each other, and (iii) the placing of the implant in the first operative state comprises placing the implant such that the first beams of the first and second U-shaped portions are at a first distance from each other.

Example 196. The method according to any example 195, wherein (i) the placing of the implant in the first operative state comprises placing the implant onto the leaflet while the first and second U-shaped portions are substantially in a single plane, and (ii) the transforming comprises pivoting the first and second U-shaped portions relative to each other to move the unified beam to be disposed outside of a plane formed by the first beams of the first and second U-shaped portions.

Example 197. The method according to any one of examples 195 to 196, wherein, the transforming comprises pivoting the first and second U-shaped portions relative to each other to form a second distance between the first beams of the first and second U-shaped portions, the second distance being smaller than the first distance.

Example 198. The method according to any one of examples 195 to 197, wherein the placing of the implant comprises sliding the implant onto the lip of the leaflet, when the implant is in the first operative state, such that (i) the bases of the first and second U-shaped portions extend along the lip of the leaflet, (ii) the unified beam is disposed on or adjacent one side of the leaflet, and (iii) the first beams of the first and second U-shaped portions are disposed on or adjacent the opposing side of the leaflet.

Example 199. The method according to any one of examples 187 to 189, wherein the implant comprises: (A) a central U-shaped portion, including a pair of central beams and a connecting segment; and (B) first and second side portions, each including at least one beam and being connected to one of the central beams by a corresponding one of first and second bends, wherein (i) the at least one beam of each of the first and second side portions is substantially parallel to at least one of the central beams, (ii) the first and second bends facilitate motion of the corresponding first and second side portions, relative to the pair of central beams, in at least two directions, (iii) the placing of the implant in the first operative state, comprises placing the implant onto the leaflet while the central U-shaped portion is in a first plane, and each of the first and second side portions is in a plane different from the first plane, and is disposed exteriorly to the central U-shaped portion, and (iv) wherein the transforming comprises moving the first and second side portions to be in the first plane, within the central U-shaped portion.

Example 200. The method according to example 199, wherein (i) the placing of the implant in the first operative state comprises placing the implant onto the leaflet while a first distance exists between the first and second side portions, and (ii) the transforming of the implant comprises reducing the distance between the first and second side portions to a second distance, smaller than the first distance.

Example 201. The method according to any one of examples 199 to 200, wherein (i) the implant has an intermediate operative state, in which the first and second side portions are in the first plane, and are disposed exteriorly to the central U-shaped portion, and (ii) wherein the transforming of the implant from the first operative state toward the second operative state includes: (A) transforming the implant from the first operative state to the intermediate operative state, by pivoting the first and second side portions in a first direction relative to the central U-shaped portion, to place the first and second side portions in the first plane; and (B) transforming the implant from the intermediate operative state to the second operative state by revolving the first and second side portions in a second direction relative to the central U-shaped portion, to move the first and second side portions to an interior of the central U-shaped portion.

Example 202. The method according to example 201, wherein (i) the pivoting in the first direction comprises pivoting about an axis perpendicular to longitudinal axes of the central beams, and (ii) the revolving in the second direction comprises revolving about axes parallel to the longitudinal axes of the central beams.

Example 203. The method according to any one of examples 199 to 202, wherein the placing of the implant comprises sliding the implant onto the lip of the leaflet, when the implant is in the first operative state, such that (i) the first and second bends engage the lip of the leaflet and (ii) the connecting segment of the U-shaped portion is distal to the lip of the leaflet, with (a) the U-shaped portion being disposed on or adjacent one side of the leaflet, and (b) the first and second side portions being disposed on or adjacent the opposing side of the leaflet.

Example 204. The method according to any one of examples 199 to 203, wherein the transluminally delivering comprises transluminally delivering the implant to the heart chamber when the implant is in the first operative state.

Example 205. The method according to any one of examples 199 to 203, wherein (i) the transluminally delivering comprises transluminally delivering the implant to the heart chamber when the implant is in a preparatory operative state, in which the first and second side portions are in the first plane and are exterior to the central U-shaped portion, and (ii) the method further comprising, prior to the placing of the implant, transitioning the implant from the preparatory operative state toward the first operative state.

Example 206. The method according to example 205, wherein the transitioning of the implant from the preparatory state comprises pivoting the first and second side portions relative to the U-shaped portion about an axis perpendicular to longitudinal axes of the central beams.

Example 207. The method according to any one of examples 187 to 189, wherein the implant comprises: (A) a tubular body arranged along a central longitudinal axis; (B) first and second substantially U-shaped clips, each having (i) a first elongate portion disposed on, or near, an exterior surface of the tubular body, and (ii) a second elongate portion disposed on, or near, an interior surface of the tubular body, the first and second elongate portions connected by an end portion; and (C) an elastic element holding together the second elongate portions of the first and second clips, wherein, (1) in the first operative state, the clips are disposed on a first side of the tubular body, and in the second operative state the clips are disposed on a second side of the tubular body, the second side being opposite the first side, and (2) the placing of the implant comprises sliding the implant onto the lip of the leaflet, when the implant is in the first operative state, such that the end portions engage the lip of the leaflet, with the tubular body and the second elongate portions of the first and second clips being disposed on or adjacent one side of the leaflet, and the first elongate portions of the first and second clips being disposed on or adjacent the opposing side of the leaflet.

Example 208. The method according to example 207, wherein the transluminally delivering comprises transluminally delivering the implant to the heart chamber when the implant is in the first operative state.

Example 209. The method according to example 207, wherein: (i) the transluminally delivering comprises transluminally delivering the implant to the heart chamber when the U-shaped clips are separate from the tubular body, and (ii) the method further comprises, prior to or during the placing of the implant, sliding the first and second clips onto the tubular body to form the first operative state.

Example 210. The method according to any one of examples 207 to 209, wherein the transforming of the implant comprises drawing the first and second clips away from each other, about a perimeter of the tubular body and against a force of the elastic element.

Example 211. A system for use with a leaflet of a cardiac valve of a subject, the system comprising: (A) at least one longitudinal catheter configured to be transluminally advanced toward a heart chamber of the subject adjacent the cardiac valve, the longitudinal catheter having a proximal part and a steerable distal part, and a longitudinal axis therebetween; and (B) an implant including: (i) a core; and (ii) a securing element, adapted to secure tissue about the core; the at least one longitudinal catheter adapted to: (1) transluminally deliver the implant to the heart chamber; (2) dispose a portion of the leaflet about the core by placing the core against a first surface of the leaflet; and (3) from a second, opposing, surface of the leaflet, using the securing element, secure the portion of the leaflet extending about the core, thereby deforming a region of the leaflet.

Example 212. The system according to example 211, wherein the core and the securing element are sized and configured to cooperate with each other to retain the core and the securing element in their implanted positions on the leaflet.

Example 213. The system according to any one of examples 211 to 212, wherein the at least one longitudinal catheter comprises (A) a first catheter adapted to transluminally deliver the core, and (B) a second catheter adapted to transluminally deliver the securing element.

Example 214. The system according to example 211 or example 213, wherein (i) the core comprises a tubular core, and (ii) the securing element comprises a securing clip, the securing clip having first and second longitudinal portions connected by an end portion.

Example 215. The system according to example 214, wherein the longitudinal catheter is adapted to dispose the portion of the leaflet about the tubular core when a longitudinal axis of the tubular core is substantially perpendicular to the lip of the leaflet.

Example 216. The system according to example 214 or example 215, wherein the catheter is adapted to secure the portion of the leaflet about the tubular core by the securing clip when the first and second longitudinal portions of the securing clip are substantially perpendicular to the lip of the leaflet.

Example 217. The system according to any one of examples 214 to 216, wherein, the catheter is adapted to place the securing clip such that the end portion thereof is distal to the lip of the leaflet, thereby to secure the portion of the leaflet extending about the core.

Example 218. The system according to any one of examples 214 to 217, wherein, the catheter is adapted to pierce the securing clip through tissue of the leaflet when securing the portion of the leaflet extending about the core.

Example 219. The system according to any one of examples 214 to 218, wherein the catheter is further adapted, once the securing element is secured, to remove the core from the cardiac valve, such that the leaflet remains secured only by the securing element.

Example 220. The system according to any one of examples 211 to 213, wherein: (i) the core comprises first and second plates having a neck portion therebetween, the plates having a greater diameter than the neck portion, such that an indentation is formed between the plates, around the neck portion; and (ii) the securing element comprises a panel having a groove formed therein, the groove having a plate-receiving region and a neck-receiving region, the plate-receiving region being adapted to receive one of the first and second plates of the core, and the neck receiving-region being adapted to receive the neck of the core, with the first plate extending on one side of panel, and the second plate extending on an opposing side of the panel.

Example 221. The system according to example 220, wherein the first and second plates are substantially the same size.

Example 222. The system according to example 220 or example 221, wherein the first and second plates are substantially parallel to one another.

Example 223. The system according to any one of examples 220 to 222, wherein (i) the plate receiving region is dimensioned to allow passage of at least one of the first and second plates therethrough, and (ii) the neck receiving region is dimensioned to limit passage of the first and second plates therethrough.

Example 224. The system according to any one of examples 220 to 223, wherein a thickness of the panel is smaller than a height of the indentation of the core.

Example 225. The system according to any one of examples 220 to 224, wherein (i) the catheter is adapted to dispose the portion of the leaflet about the core by placing the first plate against the first surface of the leaflet, and (ii) the catheter is adapted to secure the portion of the leaflet extending about the core by: (a) inserting the first plate, with the portion of the leaflet disposed thereon, into the plate-receiving region of the panel, and (b) moving the panel relative to the core, such that the neck portion of the core slides into the neck-receiving portion, while the first and second plates remain exterior to the panel on opposing sides thereof.

Example 226. A method for repairing billowing, prolapse, or flailing of a leaflet of a cardiac valve of a subject, the leaflet having a root attached to an annulus of the cardiac valve and a lip adapted to coapt with at least one other leaflet of the cardiac valve, the method comprising: (A) transluminally delivering to a cardiac chamber adjacent the cardiac valve, an implant including a core and a securing element, the securing element adapted to secure tissue about the core; (B) disposing a portion of the leaflet about the core by placing the core against a first surface of the leaflet; and (C) from a second, opposing, surface of the leaflet, using the securing element, securing the portion of the leaflet extending about the core, thereby causing deformation of the portion of the leaflet.

Example 227. The method according to example 226, wherein the transluminally delivering comprises, (i) using a first catheter, transluminally delivering the core to a first cardiac chamber, adjacent the first surface of the leaflet, and (ii) using a second catheter, transluminally delivering the securing element to a second cardiac chamber, adjacent the second, opposing surface of the leaflet.

Example 228. The method according to example 226 or example 227, wherein (i) the core comprises a tubular core, (ii) the securing element comprises a securing clip, the securing clip having first and second longitudinal portions connected by an end portion, (iii) the disposing comprises disposing the portion of the leaflet about the tubular core when a longitudinal axis of the tubular core is substantially perpendicular to the lip of the leaflet, and (iv) the securing comprises securing the portion of the leaflet about the tubular core when the first and second longitudinal portions of the securing clip are substantially perpendicular to the lip of the leaflet.

Example 229. The method according to example 228, wherein, following the securing, the end portion of the securing clip is distal to the lip of the leaflet.

Example 230. The method according to any one of examples 228 to 229, wherein, the securing comprises piercing the tissue of the leaflet using the securing clip.

Example 231. The method according to any one of examples 228 to 230, further comprising, following the securing, removing the core from the cardiac valve, such that the leaflet remains secured only by the securing element.

Example 232. The method according to example 226 or example 227, wherein: (i) the core comprises first and second plates having a neck portion therebetween, the plates having a greater diameter than the neck portion, such that an indentation is formed between the plates, around the neck portion; (ii) the securing element comprises a panel having a groove formed therein, the groove having a plate-receiving region and a neck-receiving region, the plate-receiving region being adapted to receive one of the first and second plates of the core, and the neck receiving-region being adapted to receive the neck portion of the core, with the first plate extending on one side of the panel, and the second plate extending on an opposing side of the panel; and (iii) the disposing comprises disposing a portion of the leaflet about the core by placing the first plate against the first surface of the leaflet.

Example 233. The method according to example 232, wherein the securing comprises: (i) inserting the first plate, with the portion of the leaflet disposed thereon, into the plate-receiving region of the panel, and (ii) moving the panel relative to the core, such that the neck portion of the core slides into the neck-receiving portion, while the first and second plates remain exterior to the panel on opposing sides thereof.

Example 234. A system for use with a leaflet of a cardiac valve of a subject, the leaflet having a root attached to an annulus of the cardiac valve, a lip adapted to coapt with at least one other leaflet of the cardiac valve, and an intermediate region extending between the root and the lip, the system comprising: (A) at least one longitudinal catheter configured to be transluminally advanced toward a heart chamber of the subject adjacent the cardiac valve, the longitudinal catheter having a proximal part and a steerable distal part, and a longitudinal axis therebetween; and (B) a constraining implant, wherein, the at least one longitudinal catheter is adapted to: (i) transluminally deliver the constraining implant to the heart chamber; and (ii) using the constraining implant, constrain the lip so as to present the intermediate region as a substitute coaptation surface for coaptation with the at least one other leaflet.

Example 235. The system according to example 234, wherein, prior to using the constraining implant, the catheter is adapted to fold the leaflet to present the intermediate region thereof.

Example 236. The system according to example 234 or example 235, wherein the substitute coaptation surface includes at least a portion of a securing element.

Example 237. The system according to any one of examples 234 to 235, wherein the catheter is adapted to constrain the lip of the leaflet to a downstream surface of the leaflet.

Example 238. The system according to any one of examples 234 to 237, wherein (i) the constraining element comprises a U-shaped clip, and (ii) the catheter is adapted to constrain the lip by folding the lip toward the downstream surface of the leaflet and securing the fold using the U-shaped clip.

Example 239. The system according to any one of examples 234 to 237, wherein (i) the constraining element comprises a plurality of U-shaped pins, and (ii) the catheter is adapted to constrain the lip by folding the lip toward the downstream surface of the leaflet and securing the fold by disposing the U-shaped pins at multiple locations along the fold.

Example 240. The system according to any one of examples 234 to 236, wherein the catheter is adapted to constrain the lip of the leaflet to a cardiac wall, downstream of the cardiac valve.

Example 241. The system according to example 240, wherein the constraining element comprises at least one tissue anchor, and the catheter is adapted to anchor the lip of the leaflet to the cardiac wall using the at least one tissue anchor.

Example 242. The system according to example 240, wherein (i) the constraining element comprises at least one pledget having first and second end portions connected by a longitudinal portion, the first and second end portions having a greater cross section than the longitudinal portion, and (ii) the catheter is adapted to constrain the lip of the leaflet, at the fold, by anchoring the first end of the at least one pledget to a first location in the cardiac wall, upstream of the cardiac valve and anchoring the second end of the at least one pledget to a second location in the cardiac wall downstream of the cardiac valve, through the lip of the leaflet, (iii) such that the longitudinal portion of the at least one pledget extends along a surface of the leaflet from the first location to the second location.

Example 243. The system of example 242, wherein the catheter is adapted to anchor the first and second ends of the at least one pledget into a coronary sinus adjacent the cardiac wall.

Example 244. The system according to example 242, wherein the catheter is adapted to anchor the first and second ends of the at least one pledget into a coronary artery within the cardiac wall.

Example 245. A method for repairing billowing, prolapse, or flailing of a leaflet of a cardiac valve of a subject, the leaflet having a root attached to an annulus of the cardiac valve, a lip adapted to coapt with at least one other leaflet of the valve, and an intermediate region extending between the root and the lip, the method comprising: (A) transluminally delivering a constraining element to a heart chamber, adjacent the cardiac valve; and (B) presenting the intermediate region as a substitute coaptation surface for coaptation with the at least one other leaflet by constraining the lip using the constraining element.

Example 246. The method according to example 245, wherein the presenting of the intermediate region as the substitute coaptation surface comprises presenting at least a portion of the constraining element as the substitute coaptation surface.

Example 247. The method according to any one of examples 245 to 246, wherein the constraining comprises folding the leaflet to present the intermediate region.

Example 248. The method according to any one of examples 245 to 247, wherein (i) the constraining element comprises a generally U-shaped clip, and (ii) the constraining comprises folding the lip of the leaflet toward a downstream surface of the leaflet and securing a fold using the U-shaped clip.

Example 249. The method according to any one of examples 245 to 247, wherein (i) the constraining element comprises a plurality of U-shaped pins, and (ii) the constraining comprises folding the lip of the leaflet toward the downstream surface of the leaflet and securing the fold at multiple locations therealong using the U-shaped pins.

Example 250. The method according to any one of examples 245 to 247, wherein the constraining comprises constraining the lip of the leaflet to a cardiac wall, downstream of the cardiac valve.

Example 251. The method according to example 250, wherein the constraining element comprises at least one tissue anchor, and the constraining comprises anchoring the lip of the leaflet to the cardiac wall.

Example 252. The method according to example 250, wherein: (i) the constraining element comprises at least one pledget having first and second end portions connected by a longitudinal portion, the first and second end portions having a greater cross section than the longitudinal portion; and (ii) the constraining comprises: (a) anchoring the first end of the at least one pledget to a first location in the cardiac wall, upstream of the cardiac valve; and (b) anchoring the second end of the at least one pledget to a second location in the cardiac wall downstream of the cardiac valve, through the lip of the leaflet, such that the longitudinal portion of the at least one pledget extends along an upstream surface of the leaflet from the first location to the second location.

Example 253. The method according to example 252, wherein the anchoring of the first and second ends of the at least one pledget is into a coronary sinus adjacent the cardiac wall.

Example 254. The method according to example 252, wherein the anchoring of the first and second ends of the at least one pledget is into a coronary artery within the cardiac wall.

Example 255. A system for use with a leaflet of a cardiac valve of a subject, the leaflet having a root attached to an annulus of the cardiac valve, a lip adapted to coapt with at least one other leaflet of the valve, and an intermediate region extending between the root and the lip, the system comprising: (A) at least one longitudinal catheter configured to be transluminally advanced toward a heart chamber of the subject adjacent the cardiac valve, the longitudinal catheter having a proximal part and a steerable distal part, and a longitudinal axis therebetween; and (B) an implant comprising: (i) a constraining element; and (ii) an artificial coaptation element, wherein, the at least one longitudinal catheter is adapted to: (1) transluminally deliver the constraining element and the artificial coaptation element to the heart chamber; (2) using the constraining implant, constrain the lip so as to present the intermediate region; and (3) mount the artificial coaptation element onto the intermediate region of the leaflet so as to present an artificial coaptation surface for coaptation with the at least one other leaflet.

Example 256. The system according to example 255, wherein, prior to using the constraining implant, the catheter is adapted to fold the leaflet to present the intermediate region thereof.

Example 257. The system according to any one of examples 255 to 256, wherein the catheter is adapted to constrain the lip of the leaflet to a cardiac wall, downstream of the cardiac valve.

Example 258. The system according to any one of examples 255 to 257, wherein the constraining element comprises at least one tissue anchor, and the catheter is adapted to anchor the lip of the leaflet to the cardiac wall using the at least one tissue anchor.

Example 259. The system according to any one of examples 255 to 258, wherein the artificial coaptation element comprises a shape memory material.

Example 260. The system according to any one of examples 255 to 259, wherein the artificial coaptation element comprises a wire mesh.

Example 261. The system according to any one of examples 255 to 260, wherein the catheter is adapted (i) to transluminally deliver the artificial coaptation element to the heart chamber in a compressed form, and (ii) to allow the artificial coaptation element to decompress following delivery or mounting thereof.

Example 262. The system according to any one of examples 255 to 261, wherein the at least one catheter comprises (i) a first catheter adapted to transluminally deliver the constraining element, and (ii) a second catheter adapted to transluminally deliver the artificial coaptation element.

Example 263. A method for repairing billowing, prolapse, or flailing of a leaflet of a cardiac valve of a subject, the leaflet having a root attached to an annulus of the cardiac valve, a lip adapted to coapt with at least one other leaflet of the valve, and an intermediate region extending between the root and the lip, the method comprising: (A) transluminally delivering a constraining element and an artificial coaptation element to a heart chamber, adjacent the cardiac valve; (B) presenting the intermediate region of the leaflet by constraining the lip of the leaflet using the constraining element; and (C) presenting an artificial coaptation surface for coaptation with the at least one other leaflet by mounting the artificial coaptation element onto the intermediate region of the leaflet.

Example 264. The method according to example 263, further comprising, prior to constraining the lip of the leaflet, folding the leaflet to present the intermediate region thereof.

Example 265. The method according to any one of examples 263 to 264, wherein the constraining comprises constraining the lip of the leaflet to a cardiac wall, downstream of the cardiac valve.

Example 266. The method according to any one of examples 263 to 265, wherein (i) the constraining element comprises at least one tissue anchor, and (ii) the constraining comprises anchoring the lip of the leaflet to the cardiac wall using the at least one tissue anchor.

Example 267. A system for use with a leaflet of a cardiac valve of a subject, the system comprising: (A) at least one longitudinal catheter configured to be transluminally advanced toward a heart chamber of the subject adjacent the cardiac valve, the longitudinal catheter having a proximal part and a steerable distal part, and a longitudinal axis therebetween; and (B) a leaflet-engaging surface-implant including: (i) a flexible leaflet-engaging-surface; and (ii) a tether; wherein, the at least one longitudinal catheter is adapted to: (1) transluminally deliver the leaflet-engaging surface-implant to the heart chamber; (2) attach the leaflet-engaging surface to a surface of the leaflet using the tether; and (3) deform the leaflet-engaging-surface, when attached to the leaflet, thereby to deform or shorten the leaflet, along at least one dimension thereof.

Example 268. The system according to example 267, wherein (i) the leaflet-engaging-surface is non-elastic, (ii) the catheter is adapted to deform the leaflet-engaging-surface by tensioning the tether attaching the leaflet-engaging-surface to the surface of the leaflet, and (iii) the catheter is further adapted to secure the tension in the tether to maintain the leaflet in the deformed or shortened orientation.

Example 269. The system according to example 267, wherein (i) the leaflet-engaging-surface is elastic, (ii) the catheter is further adapted to stretch the leaflet-engaging-surface prior to attachment thereof to the surface of the leaflet, and to attach the leaflet-engaging-surface to the surface of the leaflet in the stretched position, and (iii) the catheter is adapted to deform the leaflet-engaging-surface by releasing the stretching thereof, following attachment to the surface of the leaflet.

Example 270. The system according to example 269, wherein the at least one catheter comprises at least two catheters adapted to hold opposing sides of the leaflet-engaging-surface for stretching thereof.

Example 271. A method for repairing billowing, prolapse, or flailing of a leaflet of a cardiac valve of a subject, the leaflet having a root attached to an annulus of the cardiac valve, a lip adapted to coapt with at least one other leaflet of the valve, the method comprising: (A) transluminally delivering a leaflet-engaging surface implant including a tether and a leaflet-engaging-surface to a heart chamber, adjacent the cardiac valve; (B) attaching the leaflet-engaging-surface to a surface of the leaflet using the tether; and (C) deforming the leaflet-engaging-surface, when attached to the leaflet, thereby to deform or shorten the leaflet, along at least one dimension thereof.

Example 272. The method according to example 271, wherein: (i) the leaflet-engaging-surface is non-elastic, (ii) the deforming of the leaflet-engaging-surface comprises tensioning the tether attaching the leaflet-engaging-surface to the surface of the leaflet, and (iii) the method further comprises securing the tension in the tether to maintain the leaflet in the deformed or shortened orientation.

Example 273. The method according to example 271, wherein (i) the leaflet-engaging-surface is elastic, (ii) the method further comprising stretching the leaflet-engaging-surface prior to attachment thereof to the surface of the leaflet, (iii) the attaching comprises attaching the leaflet-engaging-surface to the surface of the leaflet in the stretched position, and (iv) the deforming comprises releasing the stretching of the leaflet-engaging-surface, following the attaching.

Example 274. The method according to example 273, wherein the stretching comprises, using two catheters, drawing opposing sides of the leaflet-engaging-surface away from each other.

Example 275. A system for use with a leaflet of a cardiac valve of a subject, a root of the leaflet being adjacent a coronary artery, the system comprising: (A) at least one longitudinal catheter configured to be transluminally advanced toward a heart chamber of the subject adjacent the cardiac valve, the longitudinal catheter having a proximal part and a steerable distal part, and a longitudinal axis therebetween; and (B) an implant including a pledget including a longitudinal portion, terminating at two ends thereof in first and second end portions; wherein, the at least one longitudinal catheter is adapted to: (i) transluminally deliver the pledget to the heart chamber, via the coronary artery; (ii) pierce through two positions in the leaflet, thereby to form a fold in the leaflet, the two positions and the fold being between the root of the leaflet and the lip of the leaflet; and (iii) deploy the pledget, wherein, following deploying of the pledget, the first and second ends portions are perpendicular to the longitudinal portion and engage a surface of the leaflet, thereby to maintain the tension in the longitudinal portion and the fold in the leaflet, and to reduce the length of the leaflet from the root to the lip.

Example 276. The system according to example 275, further comprising: (A) a guidewire having first and second ends; and (B) a pledget delivery system, wherein the catheter is further adapted to: (i) transluminally deliver the guidewire and the pledget delivery system to the heart chamber, via the coronary artery; (ii) use the guidewire to pierce through the two positions in the leaflet; (iii) advance the pledget delivery system, over the guidewire, to extend between the two positions; and (iv) deploy the pledget via the pledget delivery system.

Example 277. A method for repairing billowing, prolapse, or flailing of a leaflet of a cardiac valve of a subject, the leaflet having a root attached to an annulus of the cardiac valve, a lip adapted to coapt with at least one other leaflet of the valve, the root of the leaflet being adjacent a coronary artery, the method comprising: (A) transluminally delivering to a heart chamber adjacent the cardiac valve, via the coronary artery, a guidewire having first and second ends, a pledget delivery system, and a pledget including a longitudinal portion and first and second end portions; (B) piercing the first end of the guidewire through two positions in the leaflet, thereby to form a fold in the leaflet, the two positions and the fold being between the root of the leaflet and the lip of the leaflet; (C) advancing the pledget delivery system over the guidewire, through the two positions in the leaflet; and (D) deploying the pledget such that the first and second end portions engage the surface of the leaflet at the two positions, and the longitudinal portion extends between the two positions, and tensioning the longitudinal portion, to maintain the fold in the leaflet and to reduce the length of the leaflet from the root to the lip.

Example 278. A system for use with a leaflet of a cardiac valve of a subject, a root of the leaflet being attached to a cardiac wall, the system comprising: (A) at least one longitudinal catheter configured to be transluminally advanced toward a heart chamber of the subject adjacent the cardiac valve, the longitudinal catheter having a proximal part and a steerable distal part, and a longitudinal axis therebetween; and (B) a clip implant including having a clip portion defining a bend and a wire finger attached to the clip portion, wherein, the at least one longitudinal catheter is adapted to: (i) transluminally deliver the clip implant to the heart chamber; and (ii) mount the clip implant onto the leaflet such that the bend engages the lip of the leaflet, and the wire finger extends to a contact point between the root of the leaflet and the cardiac wall, such that the clip implant mechanically limits the leaflet from flailing.

Example 279. The system according to example 278, wherein (i) the wire finger is substantially U-shaped and includes two longitudinal portions connected by an end portion, and (ii) the catheter is adapted to mount the clip implant such that the end portion is disposed at the contact point.

Example 280. The system according to example 278 or example 279, wherein the catheter is adapted to mount the clip implant such that the wire portion is disposed against, or adjacent, a downstream surface of the leaflet.

Example 281. A method for repairing billowing, prolapse, or flailing of a leaflet of a cardiac valve of a subject, the leaflet having a root attached to an annulus of the cardiac valve, a lip adapted to coapt with at least one other leaflet of the valve, the root of the leaflet being attached to a cardiac wall, the method comprising: (A) transluminally delivering to a heart chamber adjacent the cardiac valve, via a clip implant including having a clip portion defining a bend and a wire finger attached to the clip portion; and (B) mounting the clip implant onto the leaflet such that the bend engages the lip of the leaflet, and the wire finger extends to a contact point between the root of the leaflet and the cardiac wall, such that the clip implant mechanically limits the leaflet from flailing.

Example 282. The method according to example 281, wherein the mounting comprises mounting the clip implant such that the wire portion is disposed against, or adjacent, a downstream surface of the leaflet.

Example 283. A system for use with a leaflet of a cardiac valve of a subject, the system comprising: (A) a catheter configured to be transluminally advanced toward a heart chamber of the subject adjacent the cardiac valve, the catheter having a proximal part and a steerable distal part, and a longitudinal axis therebetween; and (B) a device including first, second, and third beams and having a first operative state and a second operative state, wherein the catheter is adapted to: (i) transluminally deliver the device to the heart chamber; (ii) position the device at the leaflet in the first operative state, with the first and third beams on a first side of the leaflet, and the second beam on a second opposing side of the leaflet; and (iii) while the first and third beams remain on the first side of the leaflet, and the second beam remains on the second opposing side of the leaflet, transform the device from the first operative state toward the second operative state.

Example 284. The system of example 283, wherein the device is an implant.

Example 285. The system according to example 284, wherein at least two of the first, second, and third beams are substantially parallel to each other, in at least one of the first operative state and the second operative state.

Example 286. The system according to example 284 or example 285, wherein the implant is at least one of flexible and resilient.

Example 287. The system according to any one of examples 284 to 286, wherein the implant comprises a resilient metal.

Example 288. The system according to any one of examples 284 to 286, wherein the implant comprises a shape memory material.

Example 289. The system according to any one of examples 284 to 286, wherein the implant comprises an elastic component.

Example 290. The system according to any one of examples 284 to 289, wherein the catheter is adapted to position the implant onto the leaflet in the first operative state by positioning the implant such that the first, second, and third beams are substantially perpendicular to a lip of the leaflet.

Example 291. The system according to any one of examples 284 to 290, wherein: (i) the implant comprises a base, from which extend a plurality of beam including the first, second, and third beams, each of the plurality of beams having a terminus distal to the base, the first, second, and third beams being substantially parallel to one another; (ii) in the first operative state, the terminus of one of the first, second, and third beams is disposed outside of a plane defined by the base and termini of the other two of the first, second, and third beams, the one of the first, second, and third beams being disposed between the other two of the first, second, and third beams; and (iii) in the second operative state, the base, as well as the termini of the first, second, and third beams, are in a single plane.

Example 292. The system according to example 291, wherein: (i) the plurality of beams includes at least five beams; (ii) a first subset of the plurality of beams includes the one of the first, second, and third beams, and a second subset of the plurality of beams includes the other two of the first, second, and third beams, the first and second subsets of the plurality of beams being mutually exclusive; (iii) in the first operative state, the plane is defined by the termini all beams in the second subset and by the base, and the termini of all beams in the first subset are disposed outside of the plane; and (iv) in the second operative state, the base, as well as the termini of the beams in the first and second subsets, are in the single plane.

Example 293. The system according to example 292, wherein, in the first operative state, the termini of all beams in the first subset are disposed in a second plane, the second plane being angled relative to the plane.

Example 294. The system according to any one of examples 291 to 293, wherein the first, second, and third beams are substantially perpendicular to the base, in at least one of the first and second operative states.

Example 295. The system according to any one of examples 291 to 294, wherein the catheter is adapted to slide the implant onto a lip of the leaflet, when the implant is in the first operative state, such that the base extends along the lip of the leaflet, thereby to position the implant onto the leaflet.

Example 296. The system according to any one of examples 284 to 290, wherein the implant comprises: (A) first and second U-shaped portions, each including a base and first and second beams extending substantially perpendicularly from the base, and substantially parallel to each other; and (B) an engaging element holding together the second beam of each of the first and second U-shaped portions to form a unified beam, wherein (i) the unified beam is a central beam, disposed between the first beam of the first U-shaped portion and the first beam of the second U-shaped portion, and (ii) the second beams of the first and second U-shaped portion are pivotable relative to the engaging element, about respective longitudinal axes of the second beams, such that the first and second U-shaped portions are pivotable relative to each other.

Example 297. The system according to example 296, wherein the first and second U-shaped portions are identical to each other.

Example 298. The system according to example 296 or of example 297, wherein the engaging element comprises a longitudinally extending cylinder.

Example 299. The system according to any one of examples 296 to 298, wherein, (i) in the first operative state, the first and second U-shaped portions are substantially in a single plane, and (ii) in the second operative state, the unified beam is disposed outside of a plane formed by the first beams of the first and second U-shaped portions.

Example 300. The system according to any one of examples 296 to 299, wherein, (i) in the first operative state, a first distance exists between the first beams of the first and second U-shaped portions, and (ii) in the second operative state a second distance exists between the first beams of the first and second U-shaped portions, the second distance being smaller than the first distance.

Example 301. The system according to any one of examples 296 to 300, wherein the catheter is adapted to rotate the first and second U-shaped portions relative to each other, thereby transforming the implant from the first operative state to the second operative state.

Example 302. The system according to any one of examples 296 to 301, wherein (i) the unified beam comprises the second beam of the implant, and (ii) the first beams of the first and second U-shaped portions comprise the first and third beams of the implant, respectively.

Example 303. The system according to any one of examples 284 to 290, wherein the implant comprises: (A) a central U-shaped portion, including a pair of central beams and a connecting segment; and (B) first and second side portions, each including at least one beam and being connected to one of the central beams by a corresponding one of first and second bends, wherein (i) the at least one beam of each of the first and second side portions is substantially parallel to at least one of the central beams, and (ii) the first and second bends facilitate motion of the corresponding first and second side portions, relative to the pair of central beams, in at least two directions.

Example 304. The system according to example 303, wherein, (i) in the first operative state, the central U-shaped portion is in a first plane, and each of the first and second side portions is in a plane different from the first plane, and is disposed exteriorly to the central U-shaped portion, and (ii) in the second operative state, the central U-shaped portion and the first and second side portions are in the first plane, with the first and second side portions being disposed within the central U-shaped portion.

Example 305. The system according to example 303 or example 304, wherein the implant is formed of a single length of a flexible or resilient material.

Example 306. The system according to any one of examples 303 to 305, wherein each of the first and second side portions includes a pair of side beams, connected to each other by an additional bend, the pair of side beams being substantially parallel to each other.

Example 307. The system according to any one of examples 303 to 306, wherein the implant is side-to-side symmetrical about a central longitudinal axis of the central U-shaped portion.

Example 308. The system according to any one of examples 303 to 307, wherein, (i) in the first operative state, a first distance exists between the first and second side portions, and (ii) in the second operative state a second distance exists between the first and second side portions, the second distance being smaller than the first distance.

Example 309. The system according to any one of examples 304 to 308, wherein (i) the implant has an intermediate operative state, in which the first and second side portions are in the first plane, and are disposed exteriorly to the central U-shaped portion, and (ii) for transforming the implant from the first operative state to the second operative state, the catheter is adapted to (a) transform the implant from the first operative state to the intermediate operative state, by pivoting the first and second side portions in a first direction relative to the central U-shaped portion, to place the first and second side portions in the first plane, and to (b) further transform the implant from the intermediate operative state to the second operative state by revolving the first and second side portions in a second direction relative to the central U-shaped portion, to move the first and second side portions to the interior of the central U-shaped portion.

Example 310. The system according to example 309, wherein the first direction is about an axis perpendicular to longitudinal axes of the central beams, and the second direction is about axes parallel to the longitudinal axes of the central beams.

Example 311. The system according to any one of examples 303 to 310, wherein the U-shaped portion comprises the second beam, and the first and second side portions comprise the first and third beams, respectively.

Example 312. The system according to any one of examples 303 to 311, wherein the catheter is adapted to slide the implant onto a lip of the leaflet, when the implant is in the first operative state, such that the first and second bends engage the lip of the leaflet and the connecting segment of the U-shaped portion is distal to the lip of the leaflet, thereby to position the implant onto the leaflet.

Example 313. The system according to any one of examples 303 to 312, wherein the catheter is adapted to transluminally deliver the implant to the heart chamber when the implant is in the first operative state.

Example 314. The system according to any one of examples 304 to 312, wherein (i) the catheter is adapted to transluminally deliver the implant to the heart chamber when the implant is in a preparatory operative state, in which the first and second side portions are in the first plane and are exterior to the central U-shaped portion, and (ii) the catheter is further adapted, prior to placing the implant onto the leaflet, to transition the implant from the preparatory operative state to the first operative state, by pivoting the first and second side portions relative to the central U-shaped portion about the axis perpendicular to longitudinal axes of the central beams.

Example 315. The system according to any one of examples 284 to 290, wherein the implant comprises: (A) a tubular body arranged along a central longitudinal axis; (B) first and second substantially U-shaped clips, each having a first elongate portion disposed on, or near, an exterior surface of the tubular body, and a second elongate portion disposed on, or near, an interior surface of the tubular body, the first and second elongate portions connected by an end portion; and (C) an elastic element holding together the second elongate portions of the first and second clips, wherein, (i) in the first operative state, the clips are disposed on a first side of the tubular body, and (ii) in the second operative state the clips are disposed on a second side of the tubular body, the second side being opposite the first side.

Example 316. The system according to example 315, wherein (i) the catheter is adapted to slide the implant onto a lip of the leaflet, when the implant is in the first operative state, such that the end portions engage the lip of the leaflet, thereby to place the implant onto the leaflet, and (ii) the tubular body and the second elongate portions of the first and second clips forming the second beam of the implant and the first elongate portions of the first and second clips forming the first and second beams of the implant.

Example 317. The system according to example 315 or example 316, wherein the catheter is adapted to transluminally deliver the implant to the heart chamber when the implant is in the first operative state.

Example 318. The system according to example 315 or example 316, wherein (i) the catheter is adapted to transluminally deliver the implant to the heart chamber when the U-shaped clips are separate from the tubular body, and (ii) wherein the catheter is further adapted, prior to or during placing the implant onto the leaflet, to place the first and second clips onto the tubular body.

Example 319. The system according to any one of examples 315 to 318, wherein, the catheter is adapted to transform the implant from the first operative state to the second operative state, by drawing the first and second clips away from each other, about a perimeter of the tubular body and against a force of the elastic element.

Example 320. The system according to example 283, wherein: (i) the catheter is adapted to form a bulge in the leaflet by transforming the device from the first operative state toward the second operative state while the first and third beams remain on the first side of the leaflet and the second beam remains on the second opposing side of the leaflet, and (ii) the system further comprises an attaching device, transluminally deliverable by the catheter, and configured to at least temporarily secure two segments of the leaflet at the bulge.

Example 321. The system of example 320, wherein the catheter is further adapted to hold the two segments of the leaflet together to hold the bulge by moving the first beam and the third beam toward each other.

Example 322. The system of example 320 or of example 321, wherein the attaching device is configured to secure the two segments together when the two segments are held together.

Example 323. The system according to any one of examples 320 to example 322, wherein the attaching device comprises a helical needle defining a helical lumen, the helical needle configured to be advanced distally out of the catheter and to rotationally extend through, and secure together the two segments at the bulge.

Example 324. The system according to example 323, further comprising a suture extending through the helical lumen of the helical needle, wherein the suture is attached to a detachable needle tip of the helical needle and is configured to secure together the two segments at the bulge following removal of the helical needle from the heart chamber.

Example 325. The system according to example 324, further comprising a pushing-wire extending through the lumen of the helical needle and configured to push the detachable needle tip of the helical needle distally relative to a needle body, thereby to detach the detachable needle tip from the needle body.

Example 326. The system according to any one of examples 320 to 325, further comprising a cutting device configured to cut through tissue of the leaflet, wherein the catheter is further adapted to: (i) transluminally deliver the cutting device to the heart chamber; and (ii) operate the cutting device to cut a portion of the leaflet disposed between the two segments at the bulge, thereby to form cut edges.

Example 327. The system according to example 326, wherein the cut edges are at least temporarily secured by the attaching device.

Example 328. The system according to example 326 or example 327, wherein (i) the cutting device is adapted to extend distally out of the second beam of the device, and (ii) the catheter is further adapted, following transforming of the device from the first operative state toward the second operative state and following the securing together of the two segments, to draw the device proximally, such that during the drawing of the device the cutting device cuts the tissue of the leaflet between the two segments at the bulge.

Example 329. The system according to any one of examples 326 to 328, wherein (i) the cutting device is further configured to clamp the cut tissue prior to complete cutting thereof, and (ii) the catheter is further adapted to transluminally remove the cutting device, together with the cut tissue clamped by the cutting device, from the heart chamber.

Example 330. A method for repairing a leaflet of a cardiac valve of a subject, the method comprising: (i) transluminally delivering to a heart chamber, adjacent the cardiac valve, a device including first, second, and third beams and having a first operative state and a second operative state; (ii) positioning the device at the leaflet in the first operative state, with the first and third beams on a first side of the leaflet, and the second beam on a second opposing side of the leaflet; and (iii) while the first and third beams remain on the first side of the leaflet, and the second beam remains on the second opposing side of the leaflet, transforming the device from the first operative state toward the second operative state.

Example 331. The method of example 330, wherein (i) the device comprises an implant, (ii) the transluminally delivering comprises transluminally delivering the implant, (iii) the positioning comprises positioning the implant, and (iv) the transforming comprises transforming the implant.

Example 332. The method according to example 331, wherein the positioning of the implant comprises positioning the implant such that the first, second, and third beams are substantially perpendicular to a lip of the leaflet.

Example 333. The method according to any one of examples 331 to 332, wherein: (i) the implant comprises a base, from which extend a plurality of beam including the first, second, and third beams, each of the plurality of beams having a terminus distal to the base, the first, second, and third beams being substantially parallel to one another; (ii) the positioning of the implant in the first operative state comprises placing the implant such that the terminus of one of the first, second, and third beams is disposed outside of a plane defined by the base and termini of the other two of the first, second, and third beams, the one of the first, second, and third beams being disposed between the other two of the first, second, and third beams; (iii) in the second operative state, the base, as well as the termini of the first, second, and third beams, are in a single plane; and (iv) the transforming of the implant from the first operative state toward the second operative state comprises moving the termini of the first, second, and third beams, relative to each other, so that the termini of the first, second, and third beams are in the single plane.

Example 334. The method according to example 333, wherein: (i) the plurality of beams includes at least five beams; (ii) a first subset of the plurality of beams includes the one of the first, second, and third beams, and a second subset of the plurality of beams includes the other two of the first, second, and third beams, the first and second subsets of the plurality of beams being mutually exclusive; (iii) the positioning of the implant in the first operative state comprises positioning the implant such that the plane is defined by the termini of all beams in the second subset and by the base, and the termini of all beams in the first subset are disposed outside of the plane; and (iv) the transforming of the implant comprises moving the termini of all the beams in the first subset such that, in the second operative state, the base, as well as the termini of the beams in the first and second subsets, are in the single plane.

Example 335. The method according to example 334, wherein, the positioning of the implant in the first operative state comprises positioning the implant such that the termini of all beams in the first subset are disposed in a second plane, the second plane being angled relative to the plane.

Example 336. The method according to any one of examples 333 to 335, wherein the positioning of the implant comprises positioning of the implant while the first, second, and third beams are substantially perpendicular to the base.

Example 337. The method according to any one of examples 333 to 336, wherein the positioning of the implant onto the leaflet comprises sliding the implant onto the lip of the leaflet, when the implant is in the first operative state, such that the base extends along the lip of the leaflet.

Example 338. The method according to any one of examples 331 to 332, wherein the implant comprises: (A) first and second U-shaped portions, each including a base and first and second beams extending substantially perpendicularly from the base, and substantially parallel to each other; and (B) an engaging element holding together the second beam of each of the first and second U-shaped portions to form a unified beam, wherein (i) the unified beam is a central beam, disposed between the first beam of the first U-shaped portion and the first beam of the second U-shaped portion, (ii) the second beams of the first and second U-shaped portion are pivotable relative to the engaging element, about respective longitudinal axes of the second beams, such that the first and second U-shaped portions are pivotable relative to each other, and (iii) the positioning of the implant in the first operative state comprises positioning the implant such that the first beams of the first and second U-shaped portions are at a first distance from each other.

Example 339. The method according to any example 338, wherein (i) the positioning of the implant in the first operative state comprises positioning the implant onto the leaflet while the first and second U-shaped portions are substantially in a single plane, and (ii) the transforming comprises pivoting the first and second U-shaped portions relative to each other to move the unified beam to be disposed outside of a plane formed by the first beams of the first and second U-shaped portions.

Example 340. The method according to any one of examples 338 to 339, wherein, the transforming comprises pivoting the first and second U-shaped portions relative to each other to form a second distance between the first beams of the first and second U-shaped portions, the second distance being smaller than the first distance.

Example 341. The method according to any one of examples 338 to 340, wherein the positioning of the implant comprises sliding the implant onto the lip of the leaflet, when the implant is in the first operative state, such that (i) the bases of the first and second U-shaped portions extend along the lip of the leaflet, (ii) the unified beam functions as the second beam and is on the second opposing side of the leaflet, and (iii) the first beams of the first and second U-shaped portions function as the first and third beams of the device and are disposed on the first side of the leaflet.

Example 342. The method according to any one of examples 331 to 332, wherein the implant comprises: (A) a central U-shaped portion, including a pair of central beams and a connecting segment; and (B) first and second side portions, each including at least one beam and being connected to one of the central beams by a corresponding one of first and second bends, wherein (i) the at least one beam of each of the first and second side portions is substantially parallel to at least one of the central beams, (ii) the first and second bends facilitate motion of the corresponding first and second side portions, relative to the pair of central beams, in at least two directions, (iii) the positioning of the implant in the first operative state, comprises positioning the implant onto the leaflet while (1) the central U-shaped portion is in a first plane, and (2) each of the first and second side portions is in a plane different from the first plane, and is disposed exteriorly to the central U-shaped portion, and (iv) the transforming comprises moving the first and second side portions to be in the first plane, within the central U-shaped portion.

Example 343. The method according to example 342, wherein (i) the positioning of the implant in the first operative state comprises positioning the implant onto the leaflet while a first distance exists between the first and second side portions, and (ii) the transforming of the implant comprises reducing the distance between the first and second side portions to a second distance, smaller than the first distance.

Example 344. The method according to any one of examples 342 to 343, wherein, (i) the implant has an intermediate operative state, in which the first and second side portions are in the first plane, and are disposed exteriorly to the central U-shaped portion, and (ii) the transforming of the implant from the first operative state toward the second operative state includes: (A) transforming the implant from the first operative state to the intermediate operative state, by pivoting the first and second side portions in a first direction relative to the central U-shaped portion, to place the first and second side portions in the first plane; and (B) transforming the implant from the intermediate operative state to the second operative state by revolving the first and second side portions in a second direction relative to the central U-shaped portion, to move the first and second side portions to the interior of the central U-shaped portion.

Example 345. The method according to example 344, wherein (i) the pivoting in the first direction comprises pivoting about an axis perpendicular to longitudinal axes of the central beams, and (ii) the revolving in the second direction comprises revolving about axes parallel to the longitudinal axes of the central beams.

Example 346. The method according to any one of examples 342 to 345, wherein the positioning of the implant comprises sliding the implant onto the lip of the leaflet, when the implant is in the first operative state, such that (i) the first and second bends engage the lip of the leaflet, and (ii) the connecting segment of the U-shaped portion is distal to the lip of the leaflet.

Example 347. The method according to any one of examples 342 to 346, wherein the transluminally delivering comprises transluminally delivering the implant to the heart chamber when the implant is in the first operative state.

Example 348. The method according to any one of examples 342 to 346, wherein (i) the transluminally delivering comprises transluminally delivering the implant to the heart chamber when the implant is in a preparatory operative state, in which the first and second side portions are in the first plane and are exterior to the central U-shaped portion, and (ii) the method further comprising, prior to the placing of the implant, transitioning the implant from the preparatory operative state toward the first operative state.

Example 349. The method according to example 348, wherein the transitioning of the implant from the preparatory operative state comprises pivoting the first and second side portions relative to the U-shaped portion about an axis perpendicular to longitudinal axes of the central beams.

Example 350. The method according to any one of examples 331 to 332, wherein the implant comprises: (A) a tubular body arranged along a central longitudinal axis; (B) first and second substantially U-shaped clips, each having a first elongate portion disposed on, or near, an exterior surface of the tubular body, and a second elongate portion disposed on, or near, an interior surface of the tubular body, the first and second elongate portions connected by an end portion; and (C) an elastic element holding together the second elongate portions of the first and second clips, wherein, (i) in the first operative state, the clips are disposed on a first side of the tubular body, and in the second operative state the clips are disposed on a second side of the tubular body, the second side being opposite the first side, and (ii) the positioning of the implant comprises sliding the implant onto the lip of the leaflet, when the implant is in the first operative state, such that the end portions engage the lip of the leaflet, with the tubular body and the second elongate portions of the first and second clips functioning as the second beam, and the first elongate portions of the first and second clips functioning as the first and third beams.

Example 351. The method according to example 350, wherein the transluminally delivering comprises transluminally delivering the implant to the heart chamber when the implant is in the first operative state.

Example 352. The method according to example 350, wherein: (i) the transluminally delivering comprises transluminally delivering the implant to the heart chamber when the U-shaped clips are separate from the tubular body, and (ii) the method further comprises, prior to or during the positioning of the implant, sliding the first and second clips onto the tubular body to form the first operative state.

Example 353. The method according to any one of examples 350 to 352, wherein the transforming of the implant comprises drawing the first and second clips away from each other, about a perimeter of the tubular body and against a force of the elastic element.

Example 354. The method according to example 330, wherein: (i) the transforming of the device from the first operative state toward the second operative state comprises forming a bulge in the leaflet while the first and third beams remain on the first side of the leaflet and the second beam remains on the second opposing side of the leaflet, and (ii) the method further comprises, using an attaching device, at least temporarily securing two segments of the leaflet at the bulge.

Example 355. The method of example 354, further comprising holding the two segments of the leaflet together to hold the bulge by moving the first beam and the third beam toward each other.

Example 356. The method of example 354 or example 355, wherein the at least temporarily securing is carried out while the two segments of the leaflet are held together.

Example 357. The method according to any one of examples 354 to 356, further comprising transluminally delivering the attaching device to the heart chamber adjacent the cardiac valve.

Example 358. The method of any one of examples 354 to 357, wherein: (i) the attaching device comprises a helical needle defining a helical lumen, and (ii) the at least temporarily securing comprises rotationally advancing the helical needle to extend through, and secure together, the two segments at the bulge.

Example 359. The method according to example 358, wherein (i) a suture extends through the helical lumen of the helical needle, (ii) the suture is attached to a detachable needle tip of the helical needle, and (iii) the method further comprises, following the rotationally advancing of the helical needle: (A) anchoring the detachable needle tip of the helical needle to tissue of the leaflet; (B) detaching the detachable needle tip of the helical needle from a main body of the helical needle; and (C) helically retracting the main body of the helical needle away from the detachable needle tip while sliding the helical needle over the suture, thereby removing the helical needle from the anatomical site while leaving the suture securing the two segments at the bulge.

Example 360. The method according to example 359, wherein the detaching of the detachable needle tip comprises, using a pushing-wire extending through the lumen of the helical needle, pushing the detachable needle tip of the helical needle distally relative to the main body of the helical needle.

Example 361. The method according to any one of examples 354 to 360, further comprising: (A) transluminally delivering a cutting device to the heart chamber; and (B) using the cutting device, cutting a portion of the leaflet disposed between the two segments at the bulge, thereby to form cut edges.

Example 362. The method according to example 361, wherein the at least temporarily securing comprises at least temporarily securing the cut edges.

Example 363. The method according to example 361 or example 362, wherein (i) the transluminally delivering of the cutting device comprises advancing the cutting device distally out of the second beam of the device, and (ii) the cutting comprises drawing the device proximally such that during the drawing of the device the cutting device cuts the tissue of the leaflet between the two segments at the bulge.

Example 364. The method according to any one of examples 361 to 363, further comprising: (A) prior to completion of the cutting of the tissue between the two segments, clamping the cut tissue in a clamping end of a clamping device; and (B) removing the clamping device and the cut tissue clamped thereto from the heart chamber.

Example 365. The method according to example 364, further comprising, prior to clamping, advancing the clamping device distally out of the second beam of the device.

Example 366. The method according to example 365, wherein (i) the cutting device functions as the clamping device, and (ii) the advancing of the cutting device comprises advancing of the clamping device.

Example 367. A system for use with a leaflet of a cardiac valve of a subject, the system comprising: (A) a catheter configured to be transluminally advanced toward a heart chamber of the subject adjacent the cardiac valve, the catheter having a proximal part and a steerable distal part, and a longitudinal axis therebetween; (B) a cutting device, advanceable distally out of the catheter, adapted to cut through tissue of the leaflet of the cardiac valve thereby to form cut tissue and cut edges at the cardiac valve; (C) a helical needle defining a helical lumen, the helical needle configured to be advanced distally out of the catheter and to rotationally extend through, and secure together, two tissue segments of the leaflet; and (D) a suture extending through the helical lumen of the helical needle, wherein the helical needle and the suture are configured to extend along the two tissue segments of the leaflet to suture the two tissue segments to each other.

Example 368. The system according to example 367, wherein: (i) the cutting device comprises a wire forming a wire loop having a closed distal end, and a tightening element slidably coupled to the wire such that distal sliding of the tightening element over the wire causes contraction of the wire loop and cutting of the tissue disposed within the wire loop, thereby forming the cut tissue and the cut edges, and (ii) the helical needle and the suture are configured to secure together the cut edges.

Example 369. The system of example 367 or example 368, wherein the helical needle includes a needle body terminating at a detachable distal tip, the detachable distal tip being configured to be anchored to tissue at the cardiac valve and to detach from the needle body.

Example 370. The system according to example 369, wherein a distal end of the suture is attached to the detachable distal tip of the helical needle.

Example 371. The system according to example 370, wherein, following detachment of the detachable distal tip of the helical needle from the needle body, the needle body is adapted to be retracted helically away from the detachable distal tip while sliding over and along the suture.

Example 372. The system according to any one of examples 369 to 371, further comprising a pushing-wire extending through the helical lumen of the helical needle and configured to push the detachable distal tip of the helical needle distally relative to the needle body, thereby to detach the detachable distal tip from the needle body.

Example 373. The system according to any one of examples 367 to 372, wherein the cutting device and the helical needle are adapted to be removed from the heart chamber following cutting of the tissue by the cutting device, while leaving the suture in the leaflet of the cardiac valve securing the two tissue segments.

Example 374. The system according to any one of examples 367 to 373, further including a clamp having a distal clamping end, the clamp being advanceable distally out of the catheter to engage the cut tissue, prior to complete detachment of the cut tissue from the leaflet.

Example 375. The system according to example 374, wherein the clamp is adapted to be removed from the heart chamber, with the cut tissue clamped thereto during or following removal of the cutting device and the helical needle from the heart chamber.

Example 376. The system according to example 374 or example 375, wherein the clamp is configured to be advanced through the catheter alongside the helical needle.

Example 377. The system according to example 374 or example 375, wherein the cutting device functions as the clamp.

Example 378. The system according to any one of examples 367 to 377, further comprising a holding device including first, second, and third beams, and having a first operative state and a second operative state, the holding device being advanceable distally out of the catheter, wherein the catheter is adapted to: (A) transluminally deliver the securing device to the heart chamber; (B) position the holding device at the leaflet in the first operative state, with the first and third beams on a first side of the leaflet, and the second beam on a second opposing side of the leaflet; and (C) while the first and third beams remain on the first side of the leaflet, and the second beam remains on the second opposing side of the leaflet, transform the holding device from the first operative state toward the second operative state.

Example 379. The system according to example 378, wherein (i) the cutting device is adapted to be advanced distally out of the second beam of the holding device, and (ii) the catheter is further adapted, following transforming of the holding device from the first operative state toward the second operative state and following the securing together of the two tissue segments, to draw the holding device proximally, such that during the drawing of the holding device the cutting device cuts the tissue of the leaflet between the two segments at a bulge.

Example 380. The system according to example 378 or example 379, wherein: (i) the catheter is adapted to form the bulge in the leaflet by transforming the holding device from the first operative state toward the second operative state, and (ii) the helical needle and the suture are adapted to secure the two tissue segments of the leaflet at the bulge.

Example 381. The system of example 380, wherein the catheter is further adapted to hold the two segments of the leaflet together to hold the bulge by moving the first beam and the third beam toward each other.

Example 382. The system of example 380 or of example 381, wherein the helical needle is configured to secure the two tissue segments together when the two tissue segments are held together.

Example 383. A method for repairing a leaflet of a cardiac valve of a subject, the method comprising: (A) securing together two segments of tissue of the leaflet of the cardiac valve; and (B) excising tissue from between the two segments of tissue of the leaflet, with a cutting device, to form cut edges.

Example 384. The method according to example 383, wherein (i) the excising occurs before the securing, and (ii) the securing comprises securing the cut edges as the cut edges are formed, and prior to complete detachment of the tissue from the leaflet.

Example 385. The method according to example 384, wherein the excising is carried out using a cutting wire forming a wire loop, advanceable distally out of a catheter, and includes sliding a tightening element distally over the cutting wire to cause contraction of the wire loop and cutting of tissue disposed within the wire loop, thereby to form the cut edges.

Example 386. The method according to example 383, wherein (i)_the securing occurs before the excising, and (ii) the excising comprises excising the tissue from between the secured two segments, to form exposed cut edges.

Example 387. The method according to example 386, further comprising: (A) transluminally delivering to a heart chamber, adjacent the cardiac valve, a device including first, second, and third beams and having a first operative state and a second operative state; (B) positioning the device at the leaflet in the first operative state, with the first and third beams on a first side of the leaflet, and the second beam on a second opposing side of the leaflet; and
(C) while the first and third beams remain on the first side of the leaflet, and the second beam remains on the second opposing side of the leaflet, transforming the device from the first operative state toward the second operative state, thereby forming a bulge in the leaflet, wherein the securing together of the two segments comprises securing together the two segments at the bulge.

Example 388. The method according to any one of examples 383 to 387, wherein the securing together of the two segments comprises advancing a helical needle through the two segments.

Example 389. The method according to any one of examples 383 to 388, further comprising, prior to the excising, advancing the cutting device to the cardiac valve via a transluminal catheter.

Example 390. The method according to any one of examples 383 to 389, wherein (i) the helical needle includes a helical lumen and has a suture extending through the helical lumen, (ii) the method further comprising removing the helical needle from the cardiac valve while leaving the suture attaching the two segments.

Example 391. A system for use with a cardiac valve of a subject, the system comprising: (A) a catheter configured to be transluminally advanced toward a heart chamber of the subject adjacent the cardiac valve, the catheter having a proximal part and a steerable distal part, and a longitudinal axis therebetween; and (B) a helical implant defining a pair of turns, wherein in a first operative state of the helical implant the pair of turns has a first pitch and in a second operative state of the helical implant the pair of turns has a second pitch, the second pitch being smaller than the first pitch, wherein the catheter is adapted to: (i) transluminally deliver the helical implant to the heart chamber; (ii) place the helical implant, in the first operative state, onto tissue of the cardiac valve; (iii) while the helical implant remains in the first operative state, draw the tissue between the turns of the pair; and (iv) while the tissue remains between the turns of the pair, transform the helical implant from the first operative state toward the second operative state, thereby plicating the tissue.

Example 392. The system according to example 391, wherein the catheter is adapted to place the helical implant, in the first operative state, onto an annulus of the cardiac valve.

Example 393. The system according to example 391 or example 392, wherein (i) the helical implant comprises a plurality of helical implants, and (ii) the catheter is adapted to transluminally deliver, place, draw, and transform each of the plurality of helical implants at tissue in a different location along the annulus of the cardiac valve.

Example 394. The system according to any one of examples 391 to 393, further comprising a vacuum generator functionally associated with the catheter, and wherein the vacuum generator is configured to generate a vacuum drawing the tissue of the cardiac valve between the turns of the pair.

Example 395. The system according to any one of examples 391 to 394, wherein the helical implant is at least one of flexible and resilient.

Example 396. The system according to any one of examples 391 to 395, wherein the helical implant comprises a resilient metal.

Example 397. The system according to any one of examples 391 to 396, wherein the helical implant comprises a shape memory material.

Example 398. The system according to any one of example 394 to 397, further comprising a diaphragm disposed between a distal end of the catheter and the vacuum generator, the diaphragm adapted to prevent flow of fluids into a proximal end of the catheter during operation of the vacuum generator.

Example 399. The system according to any one of examples 391 to 398, wherein a distal end of the catheter is partially cut away to define an implant-deployment portal, such that during placement of the helical implant, a distal portion of the implant is disposed within the implant-deployment portal and is in contact with tissue of the cardiac valve, and a proximal portion of the helical implant is separated from the tissue of the cardiac valve by the catheter.

Example 400. A method for repairing a leaflet of a cardiac valve of a subject, the method comprising: (A) transluminally delivering to a heart chamber, adjacent the cardiac valve, a helical implant defining a pair of turns, wherein in a first operative state of the helical implant the pair of turns has a first pitch and in a second operative state of the helical implant the pair of turns has a second pitch, the second pitch being smaller than the first pitch; (B) placing the helical implant, in the first operative state, onto tissue of the cardiac valve; (C) while the helical implant remains in the first operative state, drawing the tissue between the turns of the pair; and (D) while the tissue remains between the turns of the pair, transforming the helical implant from the first operative state toward the second operative state, thereby plicating the tissue.

Example 401. The method according to example 400, wherein the placing comprises placing the helical implant, in the first operative state, onto an annulus of the cardiac valve.

Example 402. The method according to example 400 or example 401, wherein (i) the steps of delivering, placing, drawing, and transforming are carried out for each of a plurality of the helical implants, and (ii) the placing comprises placing each of the plurality of helical implants in a different location along the annulus of the cardiac valve.

Example 403. The method according to any one of examples 400 to 402, wherein the drawing comprises generating a vacuum drawing the tissue of the cardiac valve between the turns of the pair.

Example 404. The method according to example 403, further comprising a placing a diaphragm between the helical implant and a vacuum generator generating the vacuum, thereby to prevent flow of fluids from the cardiac valve into the vacuum generator during operation of the vacuum generator.

1. A system for use with a leaflet of a cardiac valve of a subject, the system comprising:
a catheter configured to be transluminally advanced toward a heart chamber of the subject adjacent the cardiac valve, the catheter having a proximal part and a steerable distal part, and a longitudinal axis therebetween;
a cutting device, advanceable distally out of the catheter, adapted to cut through tissue of the leaflet of the cardiac valve thereby to form cut tissue and cut edges at the cardiac valve;
a helical needle defining a helical lumen, the helical needle configured to be advanced distally out of the catheter and to rotationally extend through, and secure together, two tissue segments of the leaflet; and
a suture extending through the helical lumen of the helical needle,
wherein the helical needle and the suture are configured to extend along the two tissue segments of the leaflet to suture the two tissue segments to each other.

2. The system according to claim 1, wherein:
the cutting device comprises a wire forming a wire loop having a closed distal end, and a tightening element slidably coupled to the wire such that distal sliding of the tightening element over the wire causes contraction of the wire loop and cutting of the tissue disposed within the wire loop, thereby forming the cut tissue and the cut edges, and
the helical needle and the suture are configured to secure together the cut edges.

3. The system according to claim 1, wherein the helical needle includes a needle body terminating at a detachable distal tip, the detachable distal tip being configured to be anchored to tissue at the cardiac valve and to detach from the needle body.

4. The system according to claim 3, wherein a distal end of the suture is attached to the detachable distal tip of the helical needle.

5. The system according to claim 4, wherein, following detachment of the detachable distal tip of the helical needle from the needle body, the needle body is adapted to be retracted helically away from the detachable distal tip while sliding over and along the suture.

6. The system according to claim 3, further comprising a pushing-wire extending through the helical lumen of the helical needle and configured to push the detachable distal tip of the helical needle distally relative to the needle body, thereby to detach the detachable distal tip from the needle body.

7. The system according to claim 1, wherein the cutting device and the helical needle are adapted to be removed from the heart chamber following cutting of the tissue by the cutting device, while leaving the suture in the leaflet of the cardiac valve securing the two tissue segments.

8. The system according to claim 1, further including a clamp having a distal clamping end, the clamp being advanceable distally out of the catheter to engage the cut tissue, prior to complete detachment of the cut tissue from the leaflet.

9. The system according to claim 8, wherein the clamp is adapted to be removed from the heart chamber, with the cut tissue clamped thereto during or following removal of the cutting device and the helical needle from the heart chamber.

10. The system according to claim 8, wherein the clamp is configured to be advanced through the catheter alongside the helical needle.

11. The system according to claim 8, wherein the cutting device functions as the clamp.

12. The system according to claim 1, further comprising a holding device including first, second, and third beams, and having a first operative state and a second operative state, the securing device being advanceable distally out of the catheter, wherein the catheter is adapted to:
transluminally deliver the securing device to the heart chamber;
position the holding device at the leaflet in the first operative state, with the first and third beams on a first side of the leaflet, and the second beam on a second opposing side of the leaflet; and
while the first and third beams remain on the first side of the leaflet, and the second beam remains on the second opposing side of the leaflet, transform the holding device from the first operative state toward the second operative state.

13. The system according to claim 12, wherein the cutting device is adapted to be advanced distally out of the second beam of the holding device, and wherein the catheter is further adapted, following transforming of the holding device from the first operative state toward the second operative state and following the securing together of the two tissue segments, to draw the holding device proximally, such that during the drawing of the holding device the cutting device cuts the tissue of the leaflet between the two segments at a bulge.

14. The system according to claim 12, wherein:
the catheter is adapted to form the bulge in the leaflet by transforming the holding device from the first operative state toward the second operative state, and
the helical needle and the suture are adapted to secure the two tissue segments of the leaflet at the bulge.

15. The system according to claim 14, wherein the catheter is further adapted to hold the two segments of the leaflet together to hold the bulge by moving the first beam and the third beam toward each other.

16. The system according to claim 14, wherein the helical needle is configured to secure the two tissue segments together when the two tissue segments are held together.

17. A system for use with a subject, the system comprising:
a cutting device, advanceable and steerable toward an anatomical site of the subject, wherein motion of the cutting device at the anatomical site causes cutting of tissue engaged by the cutting device, thereby forming cut edges at the anatomical site;
an edge-attaching device configured to at least temporarily secure the cut edges at the anatomical site;
a user interface enabling a user to control the system, the user interface comprising:
a first engagement element that, when actuated, control motion of the cutting device,
a second engagement element that, when actuated, controls motion of the edge-attaching device, and
a unified engagement element that, when actuated, controls motion of the cutting device and of the edge-attaching device in unison,
wherein, in at least a first state of the system, the edge-attaching device is operably coupled to the cutting device such that the cutting device can be advanced distally at the anatomical site only when followed by advancement of the edge-attaching device, to secure the cut edges to one another as the cut edges are formed.

18. The system according to claim 17, wherein the unified engagement element, when actuated, controls motion of the cutting device and of the edge-attaching device at a fixed distance from one another.

19. The system according to claim 17, wherein the user interface comprises:
a first engagement element that, when actuated, control motion of the cutting device; and
a second engagement element that, when actuated, controls motion of the edge-attaching device.

20. The system according to claim 19, wherein the unified engagement element comprises a third engagement element, distinct from the first and second engagement elements.

21. The system according to claim 19, wherein:
the user interface further comprises a coupling element, functionally associated with the first and second engagement elements;
in a first operative state of the coupling element, the first and second engagement elements are coupled to form the unified engagement element, such that actuation of one of the first and second engagement elements controls motion of the cutting device and of the edge-attaching device in unison; and
in a second operative state of the coupling element, the first and second engagement elements are decoupled, such that actuation of the first engagement element controls motion only of the cutting device, and actuation of the second engagement element controls motion only of the edge-attaching device.

22. A system for use with a subject, the system comprising:
a cutting device, advanceable and steerable toward an anatomical site of the subject, wherein motion of the cutting device at the anatomical site causes cutting of tissue engaged by the cutting device, thereby forming cut edges at the anatomical site;
an edge-attaching device configured to at least temporarily secure the cut edges at the anatomical site as the cut edges are formed;
a longitudinal catheter configured to be transluminally advanced toward the anatomical site of the subject, the longitudinal catheter having a proximal part and a steerable distal part, wherein the cutting device and the edge-attaching device are adapted to be advanceable and steerable to the anatomical site distally out of the longitudinal catheter; and
a user interface enabling a user to control the system, the user interface including a unified engagement element that, when actuated, controls motion of the cutting device and of the edge-attaching device in unison.

23-25. (canceled)
